(19) United States
(12) Patent Application Publication
(10) Pub. No.: US 2024/0358797 A1
(43) Pub. Date: Oct. 31, 2024

(54) ONCOSELECTIVE CANCER THERAPY

(71) Applicant: Kernal Biologics, Inc., Cambridge, MA (US)

(72) Inventors: Monia DRAGHI, Cambridge, MA (US); Rudy CHRISTMAS, Cambridge, MA (US); Cafer OZDEMIR, Cambridge, MA (US); Burak YILMAZ, Cambridge, MA (US); Yusuf ERKUL, Cambridge, MA (US); Manfred KRAUS, Cambridge, MA (US)

(21) Appl. No.: 18/656,385

(22) Filed: May 6, 2024

Related U.S. Application Data
(63) Continuation of application No. PCT/US2022/079399, filed on Nov. 7, 2022.
(60) Provisional application No. 63/276,879, filed on Nov. 8, 2021.

(51) Int. Cl. A61K 38/21; A61K 9/51; A61K 38/17; A61K 38/20; A61K 48/00; A61P 35/00

(52) U.S. Cl. CPC A61K 38/212; A61K 9/5123; A61K 38/1709; A61K 38/2013; A61K 38/208; A61K 38/2086; A61K 48/005; A61P 35/00

(57) ABSTRACT

The present disclosure provides technologies for achieving treatment of cancer. The present disclosure provides methods and compositions for the treatment of cancer using oncoselective construct designs that can be used to encode payloads of high therapeutic interest. The present disclosure recognizes that treatment of individuals suffering from cancer with engineered nucleic acids of the disclosure in combination with immune checkpoint therapy provides unique advantages.

### CROSS-REFERENCE

This application is a continuation of International Application No. PCT/US2022/079399, filed Nov. 7, 2022, which claims the benefit of U.S. Provisional Application Ser. No. 63/276,879, filed on Nov. 8, 2021, the entirety of which is hereby incorporated by reference herein.

### BACKGROUND

There is a need to develop improved therapies for the treatment of cancer. Therapies tailored to specifically target cancer cells can provide an opportunity for unique treatment options.

### SEQUENCE LISTING

The instant application contains a Sequence Listing which has been submitted electronically and is hereby incorporated by reference in its entirety. Said copy, created on Apr. 8, 2024, is named 61600-702-301_SL.xml and is 216,742 bytes in size.

### SUMMARY

The present disclosure provides compositions and methods for the treatment of cancer. The present disclosure encompasses the discovery that treatment of subjects suffering from cancer can be improved by providing a combination of an engineered nucleic acid and an immune checkpoint inhibitor.

Delivery of nucleic acids for therapeutic purposes is a burgeoning and powerful field. Significant progress has recently been made in the field, including specifically with respect to technologies for stabilizing and/or effecting delivery of nucleic acids, particularly including translatable RNA molecules (e.g., mRNA). The present disclosure recognizes that therapy combining an engineered nucleic acid and an immune checkpoint inhibitor can provide significant benefits and synergy. In some embodiments, the present disclosure recognizes that therapy combining an engineered nucleic acid and an immune checkpoint inhibitor can sensitize an immune checkpoint inhibitor resistant cancer to immune checkpoint inhibitors.

Described herein, in some aspects, is a composition comprising: at least one engineered nucleic acid encoding at least one therapeutic, wherein the at least one engineered nucleic acid comprises at least one oncoselective modification. In some embodiments, the at least one therapeutic comprises at least one cytokine. In some embodiments, the at least one cytokine comprises at least one interleukin or at least one interferon. In some embodiments, the at least one cytokine comprises at least one interleukin. In some embodiments, the at least one cytokine comprises at least one interferon. In some embodiments, the at least one cytokine comprises at least one interleukin and at least one interferon. In some embodiments, the at least one cytokine comprises IL-2 and IFNα. In some embodiments, the at least one cytokine comprises IL-2, IL-12, IL-15, or IFNα. In some embodiments, the at least one cytokine comprises IL-2, IL-12, IL-15, and IFNα. In some embodiments, the at least one cytokine comprises a modified cytokine. In some embodiments, the modified cytokine comprises secreted cytokine, membrane tethered cytokine, masked cytokine, cytokine fusion, or a combination thereof. In some embodiments, the cytokine fusion comprises a cytokine coupled to an antibody or fragment thereof. In some embodiments, the cytokine fusion comprises a cytokine coupled to an Fc region of the antibody or fragment thereof. In some embodiments, the composition further comprises at least one additional active ingredient. In some embodiments, the at least one additional active ingredient comprises an immune checkpoint inhibitor. In some embodiments, the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof. In some embodiments, the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, ortoripalimab. In some embodiments, the at least one additional active ingredient comprises an oncolytic mRNA. In some embodiments, the oncolytic mRNA encodes constitutively active Gasdermin D. In some embodiments, the oncolytic mRNA encodes constitutively active RIPK3. In some embodiments, the composition comprises contacting the at least on engineered nucleic acid with a lipid. In some embodiments, the lipid comprises a lipid nanoparticle (LNP). In some embodiments, the at least one oncoselective modification comprises an oncoselective sequence motif. In some embodiments, the oncoselective sequence motif comprises a nucleic acid sequence of any one of SEQ ID NO: 41-110. In some embodiments, the oncoselective sequence motif comprises a combination of any one SEQ ID NO: 41-110. In some embodiments, the engineered nucleic acid comprises an open reading frame that encodes a suicide protein, cell surface antigen, an antibody agent, a toxin, a cytokine, a genetic modification protein, or a viral replication protein. In some embodiments, the open reading frame encodes a suicide protein. In some embodiments, the suicide protein induces necroptosis. In some embodiments, the at least one engineered nucleic acid encodes at least one messenger RNA (mRNA). In some embodiments, the at least one engineered nucleic acid comprises an mRNA. In some embodiments, the at least one engineered nucleic acid comprises reduced immunogenicity. In some embodiments, the least one oncoselective modification increases expression of the at least one therapeutic in a cancer cell compared to expression of the at least one therapeutic in a normal cell. In some embodiments, the at least one oncoselective modification does not increase expression of the at least one therapeutic in a cancer cell compared to expression of the at least one therapeutic in a cell.

Described herein, in some aspects, is a vector encoding the at least one engineered nucleic acid described herein.

Described herein, in some aspects, is a cell comprising the vector described herein or the at least one engineered nucleic acid described herein. In some embodiments, the cell comprises an autologous cell or an allogenic cell.

Described herein, in some aspects, is a pharmaceutical composition comprising the engineered nucleic acid described herein and at least one carrier, excipient, or diluent. In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof. In some embodiments, the pharmaceutical composition is for treating a disease or condition. In some embodiments, the disease or condition comprises solid tumor. In some embodiments, the disease or condition comprises cancer. In some embodiments, the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof.

Described herein, in some aspects, comprises a method of administering the least one engineered nucleic acid described herein, the vector described herein, the cell described herein, or the pharmaceutical composition described herein to the subject.

Described herein, in some aspects, is a method for treating a disease or condition in a subject comprising administering the least one engineered nucleic acid described herein, the vector described herein, the cell described herein, or the pharmaceutical composition described herein to the subject for treating the disease of condition.

Described herein, in some aspects, is a method for treating a disease or condition in a subject, comprising administering to the subject at least one engineered nucleic acid, said at least one engineered nucleic acid: comprises at least one oncoselective modification; and encode s at least one therapeutic. In some embodiments, the at least one therapeutic comprises least one cytokine. In some embodiments, the at least one cytokine comprises at least one interleukin or at least one interferon. In some embodiments, the at least one cytokine comprises at least one interleukin and at least one interferon. In some embodiments, the at least one cytokine comprises IL-2 and IFNα. In some embodiments, the at least one cytokine comprises IL-2, IL-12, IL-15, or IFNα. In some embodiments, the at least one cytokine comprises IL-2, IL-12, IL-15, and IFNα. In some embodiments, the disease or condition comprises solid tumor. In some embodiments, the disease or condition comprises cancer. In some embodiments, the cancer comprises melanoma, breast cancer, basal cell carcinoma (BCC), squamous cell carcinoma (SCC), cutaneous SCC (cSCC or CSCC), Head & Neck Cancer, Thyroid Cancer, Colorectal Cancer, Prostate Cancer, Liver cancer, Pancreatic Cancer, Renal Cell Carcinoma, Brain Cancer, Soft Tissue Sarcoma, Lung Cancer, or a combination thereof. In some embodiments, the cancer does not respond to immune checkpoint inhibitor treatment. In some embodiments, the at least one oncoselective modification comprises an oncoselective sequence motif. In some embodiments, the at least one oncoselective modification increases expression of the at least one therapeutic in a cancer cell compared to expression of the at least one therapeutic in a cell. In some embodiments, the at least one oncoselective modification does not increase expression of the at least one therapeutic in a cancer cell compared to expression of the at least one therapeutic in a cell.

In some embodiments the present disclosure provides a method of treating a subject suffering from cancer comprising administering to the subject one or more engineered nucleic acid(s) and an immune checkpoint inhibitor wherein the engineered nucleic acid comprises a nucleotide sequence that includes a sequence element that is or is a complement of an oncoselective translation sequence element. In some embodiments, the engineered nucleic acid and the immune checkpoint inhibitor are administered subsequently, concomitantly, or adjunctively.

In some embodiments, the present disclosure provides a method of treating a subject suffering from cancer wherein the subject has received or is receiving a first immune checkpoint inhibitor, the method comprising administering to the subject an one or more engineered nucleic acid(s) and a second immune checkpoint inhibitor; wherein the engineered nucleic acid(s) comprises a nucleotide sequence that includes a sequence element that is or is a complement of an oncoselective translation sequence element.

In some embodiments, methods and compositions of the present disclosure are useful for the treatment of subjects suffering from cancer. In some embodiments, methods and compositions of the present disclosure are useful for the treatment of subjects who have received or are receiving immune checkpoint inhibitor therapy. In some embodiments, subjects of the present disclosure are not responding or are refractory to immune checkpoint inhibitor treatment. In some embodiments, a subject has relapsed after immune checkpoint inhibitor therapy.

In some embodiments, an immune checkpoint inhibitor (e.g., either first or second) of the present disclosure comprises, one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, or CD137. In some embodiments, and the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

The present disclosure appreciates that many technologies described as “oncoselective” or “cancer cell specific” often achieve only modest discrimination between cancer and non-cancer contexts. For example, Wroblewska et al. used an mRNA circuit that is activated when intracellular miR-21 levels are high and miR-141, miR-142(3p) and miR-146a levels are low and obtained approximately only 6-fold higher in vitro cell killing in HeLa cells compared to HEK293 cells (Nat Biotechnol. 2015 August; 33(8):839-41). Likewise, Jain et al., 2018 (Nucleic Acid Ther. 2018 Oct. 1; 28(5): 285 96.) used miR-122 and miR-142 target site insertion to reduce in vivo mRNA activity by 89% and 85% (corresponding to a ˜6-10 fold decrease) in the liver and spleen, respectively. However, when intratumorally injected a significant portion of miRNA target site inserted mRNAs were also shown to be taken up by healthy cells, including tumor infiltrating immune cells (Hewitt et al., Sci Transl Med. 2019 Jan. 30; 11(477)). This activity in immune cells can be counter-productive for immuno-oncology applications of mRNAs encoding cell killing proteins.

In some embodiments, the present disclosure considers translation to be “oncoselective” when translation preferably occurs in cancer cell(s) as compared with appropriate comparable non-cancer cell(s). For example, in some embodiments, translation may be considered to be oncoselective when it is observed to be at least two (2)-fold higher in cancer cell(s) as compared with appropriate comparable non-cancer cells; in some embodiments, oncoselective translation may be at least 3, 4, 5, 6, 7, 8, 9, 10, 15, 20 or more—fold higher in cancer cell(s) as compared with appropriately comparable non-cancer cells.

In some embodiments, oncoselective translation may be considered to be oncospecific (e.g., when translation is not detectable in appropriate comparable non-cancer cell(s), but is detectable in the relevant cancer cell(s).

In some embodiments, the present disclosure provides engineered nucleic acids whose nucleotide sequence include a sequence element that is (or is a complement of) an oncoselective translation sequence element. Thus, in some embodiments, the present disclosure provides technologies that are or deliver a translatable nucleic acid (e.g., an RNA, and specifically an mRNA) that includes an oncoselective translation sequence element as described herein and/or otherwise shows oncoselective translation of a payload sequence.

In some embodiments, the present disclosure provides engineered nucleic acids whose nucleotide sequence includes a sequence element that is or is a complement of an oncoselective translation sequence element. In some embodiments, the engineered nucleic acid's nucleotide sequence includes an open reading frame or complement thereof. In some embodiments, the oncoselective translation sequence element is or comprises an oncoselective read-through motif within or upstream of the open reading frame. In some embodiments, the oncoselective readthrough motif comprises an upstream flanking sequence, a stop codon, and a downstream flanking sequence.

In some embodiments, an oncoselective readthrough motif comprises a sequence selected from the group comprising: VNNNNNNMNNMWK, NNNVWNNKGHHNH, DVHVNNNCWNNNB, MWBNNNNNNNNNN, WGNNSNHNHDNNN, VNNNNNNMNNMWK or VMNNWNKNNNNNN, wherein V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide, within the region that spans the readthrough stop codon and the first 14 nucleotides of the downstream flanking sequence. In some embodiments, the oncoselective read through motif comprises a stem loop; a bulge loop, a pseudoknot, or a combination thereof within the first 50 nucleotides of the downstream flanking sequence and part of this stem loop located preferably within stop codon and the first 16 nucleotides of the downstream flanking sequence, or a combination thereof. In some embodiments, the stem loop comprises more than 20 base paired nucleotides within first 50 nucleotides of the downstream flanking sequence.

In some embodiments, the oncoselective read through motif comprises a downstream flanking sequence with a GC content of more than 42%, more than 48%, preferably more than 54%. In some embodiments, an oncoselective read through motif comprises a codon that encodes proline residue. In some embodiments, the open reading frame of an engineered nucleic acid encodes a suicide protein. In some embodiments, the engineered nucleic acid has reduced immunogenicity.

In some embodiments, the present disclosure provides a nucleic acid whose sequence includes an open reading frame, or a complement thereof, into or before which an oncoselective read-through motif has been engineered, wherein the open reading frame encodes a payload protein selected from the group consisting of a suicide protein, cell surface antigen, an antibody agent, a toxin, a genetic modification protein, or a viral replication protein. In some embodiments, the present disclosure provides, a pharmaceutical composition comprising an engineered nucleic acid whose nucleotide sequence includes a sequence element that is or is a complement of an oncoselective translation sequence element. In some embodiments, the pharmaceutical composition comprises nanoparticles. In some embodiments, the engineered nucleic acid is expressed in a cell so that administration of the pharmaceutical composition delivers RNA to the cell.

In some embodiments, the present disclosure provides a method of treating cancer in a subject, wherein the method comprises administering a therapeutically effective amount of an engineered nucleic acid whose nucleotide sequence includes a sequence element that is or is a complement of an oncoselective translation sequence element or a pharmaceutical composition comprising the engineered nucleic acid. In some embodiments, the cancer in the treated subject comprises oncogenic ribosomes. In some embodiments, the oncogenic ribosomes comprise at least one of loss of p53 activity, loss of RB activity, FBL overexpression, or hemizygous loss of ribosomal protein genes.

In some embodiments, the present disclosure provides an oncoselective translation sequence element comprising a read-through consensus sequence, sequence with high G-C content; a codon encoding proline; a stem loop; a bulge loop, a pseudoknot or a combination thereof. In some embodiments, the present disclosure provides a method of identifying an onco-selective nucleic acid sequences the method comprising transcriptome-wide translatome analysis. In some embodiments, the present disclosure provides a method of engineering oncoselective nucleic acids by inserting a readthrough motif within or before the open reading frame.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A-1C demonstrate oncoselective killing of tumor cells

FIG. 2A-2C demonstrate oncoselective killing of tumor cells in a mouse model.

FIG. 3A-3C demonstrate oncoselective killing of tumor cells in a mouse model and sensitization of immune checkpoint inhibitor resistant cells to immune checkpoint inhibitor treatment.

FIG. 4A illustrates treatment of subcutaneous B16-F10 melanoma tumors with mKR-335 LNPs (mRNA encoding IL-2, IL-12, IL-15, and IFNα) and other mRNA treatment combinations.

FIG. 4B illustrates survival after treatment with mKR-335, a control mRNA (p<0.0001; Cox regression) and other mRNA combination treatments lacking an individual mRNA from mKR-335, cut-off 2000 mm³. mKR-335: (mRNA encoding IL-2, IL-12, IL-15, and IFNα). Control: mRNA encoding non-translated mCherry. mKR-335 minus mRNA-A: (mRNA encoding IL-12, IL-15, and IFNα). mKR-335 minus mRNA-B: (mRNA encoding IL-2, IL-12, and IFNα).

FIG. 5A-5B illustrate tolerability of intratumoral mKR-335 mRNA therapy. FIG. 5A illustrates percentage body weight changes after once every 3 days for 6 cycles (Q3Dx6) intratumoral B16-F10 dosing with a total of 20 mg (˜1 mg/kg) control mRNA or mKR-335 LNPs (mean+/−SD).

FIG. 5B illustrates liver function test: normal aspartate transaminase (AST) and alanine transaminase (ALT) serum levels 24 hours after a single or six doses with mKR-335.

FIG. 6A-FIG. 6C illustrates tolerability of systemic LNP administration. Female C57BL/6NCrl mice were treated subcutaneously with 1 mg/kg control mRNA LNPs or mKR-335. FIG. 6A illustrates H&E sections of 24 hours post single dose. FIG. 6B illustrates percentage body weight changes during a once every 3 days for 4 cycles (Q3Dx4) dosing regimen (mean+/−SD). FIG. 6C illustrates liver function test: normal AST and ALT serum levels 24 hours after a single or four doses and 7 days after last dose with mKR-335.

FIG. 7 illustrates efficacy and dose relationship of mKR-335. Dose titration treatment of subcutaneous B16-F10 melanoma tumors with mKR-335 LNPs, control mRNA LNP or mKR-335 in combination with anti-PD1 mAb treatment (200 mg administered iv on d0, d3, d10). Intratumoral injections started on day 0 in established tumors at an average size of 130 mm³. mKR-335 treatments were administered on days 0, 2, 4, 7, 9 and 11 (n=10/group).

FIG. 8 illustrates efficacious treatment of subcutaneous syngeneic murine MC38-GFP colon adenocarcinoma tumors with cytokine mRNA LNP combinations (IL-2, IL-12, IL-15, and IFNα).

FIG. 9A-FIG. 9C illustrates efficacious treatment of subcutaneous syngeneic murine B16-F10 melanoma tumors with cytokine (IL-12, IL-15, IFN-α) and immunogenic cell death (Gasdermin D or RIPK3) mRNALNPs in combination with anti-PD1. FIG. 9A. Tumor volumes of individual animals after tumor implantation. FIG. 9B. Body weight changes of animals following initiation of treatment. Values depict mean+/−S.D. FIG. 9C. Kaplan Meier survival curves of animals after tumor implantation. Statistical significance values (***) between GasD/RIPK3 mRNA treated groups vs. the control group were obtained log-rank (Mantel-Cox) tests. n=8/group.

FIG. 10 illustrates pharmacodynamic analysis of mRNAs encoding cytokines (IL-12, IL-15, IFN-alpha) formulated in saline vs. LNPs in subcutaneous B16-F10 melanoma tumors. Control mRNALNP: K143-001. Cytokine mRNA LNPs: K155-001, K156-001, or K157-001. Cytokine mRNA in Saline: Same mRNAs as Cytokine mRNA LNPs, without the LNP formulation.

### DETAILED DESCRIPTION

### Overview

In view of the difficulties for combating cancer, there remains an ongoing need for a composition or method for treating cancer in an effective manner, where the treatment decreases the progression of cancer, decreases presence of cancer or cancer cells, or increases response of cancer compared to cancer treatment modalities that are currently available. In some aspects, the composition or method for treating cancer described herein comprises an engineered nucleic acid combination, where the engineered nucleic acid encodes at least one, at least two, at least three, or at least four polypeptides for treating the cancer. In some aspects, the engineered nucleic acid can include an mRNA. For example, the engineered nucleic acid can increase expression of a therapeutic such as an interleukin or interferon for treating cancer in an oncoselective manner (e.g., increased expression of the polypeptide is only present in a tumor cell as opposed to a normal, healthy cell). In some aspects, the composition or method for treating cancer described herein utilizes an mRNA combination, where the mRNA combination increases the effectiveness of treating cancer. In some embodiments, the engineered nucleic acid combination, while increasing the effectiveness of treating cancer, does not lead to increased deleterious effects. Additionally, the engineered nucleic acid combination presents a solution to an ongoing need for treating cancer, where the cancer is unresponsive to other treatment (e.g., cancer or tumor also known as immunologically “cold” that is resistant to conventional treatments).

In some aspects, the composition or method for treating cancer described herein comprises an engineered nucleic acid combination encoding at least one polypeptide comprising a cytokine or an interferon. In some aspects, the at least polypeptide comprises a cytokine that is modified. For example, the cytokine encoded by the engineered nucleic acid combination can include an IL-12, where the IL-12 can be secreted IL-12, membrane tethered IL-12, masked IL-112, Fc-fusion IL-12, sushi IL-12, or a combination thereof. In some embodiments, the mRNA combination encoded at least one cytokine or at least one interferon. For example, the mRNA combination can encode IL-2, IL-12, IL-15, or IFNα. In some aspects, the engineered nucleic acid combination comprises mRNA modified for the oncoselective expression described herein. For example, the mRNA can be modified via: an oncoselective read-through motif within or upstream of the open reading frame; the oncoselective readthrough motif comprises an upstream flanking sequence, a stop codon, and a downstream flanking sequence; an oncoselective readthrough motif comprises a sequence selected from the group comprising: VNNNNNNMNNMWK, NNNVWNNKGHHNH, DVHVNNNCWNNNB, MWBNNNNNNNNNN, WGNNSNHNHDNNN, VNNNNNNMNNMWK or VMNNWNKNNNNNN, wherein V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide, within the region that spans the readthrough stop codon and the first 14 nucleotides of the downstream flanking sequence; the oncoselective read through motif comprises a stem loop; a bulge loop, a pseudoknot, or a combination thereof within the first 50 nucleotides of the downstream flanking sequence and part of this stem loop located preferably within stop codon and the first 16 nucleotides of the downstream flanking sequence, or a combination thereof; a stem loop comprises more than 20 base paired nucleotides within first 50 nucleotides of the downstream flanking sequence; an oncoselective read through motif comprises a downstream flanking sequence with a GC content of more than 42%, more than 48%, preferably more than 54%; an oncoselective read through motif comprises a codon that encodes proline residue, or a combination thereof.

In some aspects, described herein is an engineered nucleic acid comprising an oncoselective modification. In some embodiments, the oncoselective modification comprises an oncoselective sequence motif comprising an oncoselective readthrough motif, a 5′ UTR, a 3′ UTR, or a combination thereof. In some embodiments, the oncoselective modification comprises a nucleic acid sequence of any one of the nucleic acid sequences of SEQ ID NO: 41-110 (Table 6). In some embodiments, the oncoselective modification comprises at least one nucleic acid sequence of any one of the nucleic acid sequences of SEQ ID NO: 41-110. In some embodiments, the oncoselective modification comprises a combination of at least one of the nucleic acid sequences of SEQ ID NO: 41-110. In some embodiments, the oncoselective modification comprises a combination of two or more of the nucleic acid sequences of SEQ ID NO: 41-110.

Described herein, in some aspects, is a composition comprising at least one engineered nucleic acid encoding at least one therapeutic. In some embodiments, the at least one therapeutic comprises a cytokine such as an interleukin or an interferon. In some embodiments, the at least one therapeutic comprises at least one interleukin and at least interferon. In some embodiments, the engineered nucleic acid comprises at least one modification for increasing translation for oncoselective purpose. For example, the engineered nucleic acid can include a modification for increasing expression of the engineered nucleic acid in a tumor or cancer cell. In some embodiments, the engineered nucleic acid can be a vector. In some embodiments, the engineered nucleic can be an mRNA. In some embodiments, the composition can be formulated into a pharmaceutical composition. For example, the pharmaceutical composition can include at least one engineered nucleic acid described herein and at least one pharmaceutically acceptable excipient. In some embodiments, the composition or pharmaceutical composition described herein can be utilized for a method described herein. In some embodiments, the method comprises delivering the engineered nucleic acid to a tumor or cancer cell. In some embodiments, the method comprises treating a disease or condition by delivering the engineered nucleic acid to a cell.

### Composition

Described herein, in some aspects, is a composition comprising at least one engineered nucleic acid. In some embodiments, the at least one engineered nucleic acid encodes at least one therapeutic. In some embodiments, the at least one engineered nucleic acid comprises at least one oncoselective modification described herein (e.g., oncoselective translation, oncoselective ribosome, or a combination thereof), where the at least one oncoselective modification increases expression of the at least one engineered nucleic in a specific cell. For example, the oncoselective modification can increase expression of the at least one therapeutic encoded by the engineered nucleic acid in a cancer cell as opposed to expression of the at least one therapeutic in a cell (e.g., non-cancerous or health cell). Such oncoselective modification can be particularly useful for treating a disease or condition, where the at least one therapeutic exerts toxic side effects, and such toxic side effects should be restricted to the cell afflicted by the disease or condition. In some embodiments, the disease or condition is cancer, and the specific cell where increased expression induced by the at least one oncoselective modification is restricted is a cancer cell.

In some embodiments, the engineered nucleic acid encodes at least one therapeutic. In some embodiments, the at least one therapeutic comprises at least one cytokine. Non-example of the cytokine can include 4-1BBL, acylation stimulating protein, adipokine, albinterferon, APRIL, Arh, BAFF, Bc-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-30, IL-31, IL-32, IL-33, IL-34, IL-35, IL-36, IL-37, IL-38, IL-39, or IL-40, IL-4, IL-5, IL-6, IL-6-like, IL-7, IL-8/CXCL8, IL-9, inflammasome, interferome, interferon alpha (IFNα), interferon beta-la, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2. In some embodiments, cytokine encoded by the engineered nucleic acid described herein can include IL-2, IL-12, IL-15, IFNα, or a combination thereof. In some embodiments, cytokine encoded by the engineered nucleic acid described herein is IL-2, IL-12, IL-15, and IFNα.

In some embodiments, the at least one cytokine comprises a modified cytokine. For example the modified cytokine comprises secreted cytokine, membrane tethered cytokine, masked cytokine, cytokine fusion, or a combination thereof. In some embodiments, the cytokine fusion comprises a cytokine coupled to an antibody or fragment thereof. In some embodiments, the cytokine fusion comprises a cytokine coupled to an Fc region of the antibody or fragment thereof. In some embodiments, the composition further comprises at least one additional active ingredient. For example, the at least one additional active ingredient comprises an immune checkpoint inhibitor, where the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof. In some embodiments, the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

### Lipid

In some embodiments, the composition or the pharmaceutical composition described herein comprises at least one lipid or lipid derivative thereof. In some embodiments, an engineered nucleic acid described herein can be encapsulated or complexed with the at least one lipid or lipid derivative. For example, the composition or the pharmaceutical composition can comprise a liposome, a lipioid, a lipid nanoparticle, or a combination thereof. In some embodiments, the synthetipic polynucleotide or the vector described herein can be functionally coupled (e.g., crosslinked) to the lipid or the lipid derivative. Non-limiting example of a liposome can include 1, 2-dioleyloxy-N, N-dimethylaminopropane (DODMA) liposome, DiLa2 liposome, 1, 2-dilinoleyloxy-3-dimethylaminopropane (DLin-DMA), 2, 2-dilinoleyl-4-(2-dimethylaminoethyl)-[1,3]-dioxolane (DLin-KC2-DMA), or MC3. In some embodiments, the liposome comprises f different sizes such as a multilamellar vesicle, a small unicellular vesicle, or a large unilamellar vesicle. In some embodiments, the liposome comprises a neutral lipid such as cholesterol or dioleoyl phosphatidylethanolamine (DOPE). In some embodiments, the liposome comprises a cationic lipid such as, DLin-MC3-DMA, DLin-DMA, C12-200, or DLin-KC2-DMA. In some embodiments, the composition or the pharmaceutical composition comprises a lipid nanoparticle (LNP). Non-limiting example of LNP can include a combination of PEG-DMG 2000, DSPC, or cholesterol.

### Pharmaceutical Composition

Described herein are pharmaceutical compositions comprising the engineered nucleic acid described herein. In some embodiments, the pharmaceutical composition further comprise as pharmaceutically acceptable: carrier, excipient, or diluent. In some embodiments, the pharmaceutical composition comprises two or more active agents as disclosed herein. In some embodiments, the pharmaceutical composition comprising the engineered nucleic acid described herein treats a disease or condition described herein. In some embodiments, the disease or condition comprises a cancer. In some embodiments, the cancer comprises Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac(Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrointestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sezary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

In some embodiments, the cancer type is a solid cancer type or a hematologic malignant cancer type. In some embodiments, the cancer type is a metastatic cancer type or a relapsed or refractory cancer type. In some embodiments, the cancer type comprises acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma [DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor.

In some embodiments, the cancer type comprises acute lymphoblastic leukemia, acute myeloid leukemia, bladder cancer, breast cancer, brain cancer, cervical cancer, cholangiocarcinoma, colon cancer, colorectal cancer, endometrial cancer, esophageal cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, pheochromocytoma and paraganglioma, prostate cancer, rectal cancer, squamous cell carcinoma, testicular cancer, stomach cancer, or thyroid cancer.

For in vivo delivery, the engineered nucleic acid can be formulated into pharmaceutical compositions and can generally be administered intravitreally or parenterally (e.g., administered via an intramuscular, subcutaneous, intratumoral, transdermal, intrathecal, etc., route of administration). In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or supraocular injection.

In practicing the methods of treatment or use provided herein, therapeutically effective amounts of the pharmaceutical composition described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, levigating, emulsifying, encapsulating, entrapping or compression processes.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

### Cancer

The present disclosure provides, among other things, methods and compositions useful in the treatment of cancer, e.g., for the treatment of a tumor in a subject.

Cancer is among the leading causes of death worldwide; the number of new cancer cases diagnosed per year is expected to exceed 23 million by 2030. According to statistics released by the United States National Cancer Institute, in 2018, more than 1.7 million new cases of cancer were diagnosed in the United States, and more than 600 thousand people died from the disease.

The most common cancers, in descending order, are breast cancer, lung and bronchus cancer, prostate cancer, colon and rectum cancer, melanoma of the skin, bladder cancer, non-Hodgkin lymphoma, kidney and renal pelvis cancer, endometrial cancer, leukemia, pancreatic cancer, thyroid cancer, and liver cancer. More than 35% of men and women are expected to be diagnosed with cancer at some point during their lifetimes.

In some embodiments, a tumor or cancer suitable for treatment in accordance with the present disclosure includes, for example, Acute Lymphoblastic Leukemia (ALL), Acute Myeloid Leukemia (AML), Adrenal Cortex Cancer, Adrenocortical Carcinoma, AIDS-Related Cancer (e.g., Kaposi Sarcoma, AIDS-Related Lymphoma, Primary CNS Lymphoma), Anal Cancer, Appendix Cancer, Astrocytoma, Atypical Rhabdoid Tumor, Basal Cell Carcinoma, Bile Duct Cancer, Bladder Cancer, Bone Cancer, Brain Tumor, Breast Cancer, Bronchial Tumor, Burkitt Lymphoma, Carcinoid Tumor, Carcinoma, Cardiac (Heart) Tumor, Central Nervous System Tumor, Cervical Cancer, Cholangiocarcinoma, Chordoma, Chronic Lymphocytic Leukemia (CLL), Chronic Myelogenous Leukemia (CML), Chronic Myeloproliferative Neoplasm, Colorectal Cancer, Craniopharyngioma, Cutaneous T-Cell Lymphoma, Ductal Carcinoma In Situ (DCIS), Embryonal Tumor, Endometrial Cancer, Endometrial Sarcoma, Ependymoma, Esophageal, Esthesioneuroblastoma, Ewing Sarcoma, Extracranial Germ Cell Tumor, Extragonadal Germ Cell Tumor, Eye Cancer, Fallopian Tube Cancer, Gallbladder Cancer, Gastric (Stomach) Cancer, Gastrointestinal Carcinoid Tumor, Gastrointestinal Stromal Tumor (GIST), Germ Cell Tumor, Gestational Trophoblastic Disease, Glioma, Hairy Cell Leukemia, Head and Neck Cancer, Hepatocellular (Liver) Cancer, Hodgkin Lymphoma, Hypopharyngeal Cancer, Intraocular Melanoma, Islet Cell Tumor, Kaposi Sarcoma, Kidney Tumor, Langerhans Cell Histiocytosis, Laryngeal Cancer, Leukemia, Lip and Oral Cavity Cancer, Liver Cancer, Lung Cancer, Lymphoma, Male Breast Cancer, Malignant Fibrous Histiocytoma, Melanoma, Merkel Cell Carcinoma, Mesothelioma, Mouth Cancer, Multiple Endocrine Neoplasia Syndrome, Multiple Myeloma, Plasma Cell Neoplasm, Mycosis Fungoides, Myelodysplastic Syndrome, Myelodysplastic/Myeloproliferative Neoplasm, Nasal Cavity Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumor (Islet Cell Tumor), Paraganglioma, Paranasal Sinus Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Rectal Cancer, Renal Cell (Kidney) Cancer, Retinoblastoma, Retinoblastoma, Rhabdomyosarcoma, Rhabdomyosarcoma, Salivary Gland Cancer, Sarcoma, Szary Syndrome, Skin Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Testicular Cancer, Throat Cancer, Thymic Carcinoma, Thymoma, Thyroid Cancer, Urethral Cancer, Uterine Sarcoma, Uterine Sarcoma, Vaginal Cancer, Vascular Tumor, Vulvar Cancer, Waldenstrom Macroglobulinemia, Wilms' Tumor. In some preferred embodiments, a tumor or cancer suitable for treatment in accordance with the present disclosure comprises cancers with high frequency of p53 mutation or inactivation, including lung cancer (both non-small cell lung cancer and small cell lung cancer), colon cancer, pancreatic cancer, head and neck cancer, esophageal cancer, ovarian cancer (e.g. high-grade serous ovarian cancer), bladder cancer, liver cancer, gastric cancer, melanoma, AML (e.g. therapy related AML, complex Karyotype AML, AML with 17p deletion), chronic myeloid leukemia, and Burkitt's lymphoma.

### Immune Checkpoint Modulation.

Immune checkpoints refer to inhibitory pathways of the immune system that are responsible for maintaining self-tolerance and modulating the duration and amplitude of physiological immune responses.

Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, immune checkpoint modulators may be administered to overcome the inhibitory signals and permit and/or augment an immune attack against cancer cells. Immune checkpoint modulators may facilitate immune cell responses against cancer cells by decreasing, inhibiting, or abrogating signaling by negative immune response regulators (e.g. CTLA4), or may stimulate or enhance signaling of positive regulators of immune response (e.g. CD28).

Immunotherapy agents targeted to immune checkpoint inhibitors may be administered to encourage immune attack targeting cancer cells. Immunotherapy agents may be or include antibody agents that target (e.g., are specific for) immune checkpoint modulators. Examples of immunotherapy agents (e.g., immune checkpoint inhibitors) include antibody agents targeting one or more of CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, or CD137.

Specific examples of antibody agents may include monoclonal antibodies. Certain monoclonal antibodies targeting immune checkpoint modulators are available. For instance, pembrolizumab, nivolumab, atezolizumab, avelumab, durvalumab, ipilimumab, cemiplimab, spartalizumab, camrelizumab, cabiralizumab, dostarlimab, sintilnab, tisleliumab, or toripalimab.

Significant advances have been made in the treatment of cancer utilizing immune checkpoint inhibitors. However, systemic checkpoint blockade is ineffective in a significant number of patients. Novel therapies are needed that can be combined to increase the depth and breadth of immune checkpoint inhibitor responses in patients that are currently refractory to these treatments.

### Oncoselective Translation

The present disclosure provides methods and compositions comprising one or more engineered nucleic acid(s). In some embodiments an engineered nucleic acid comprises a nucleotide sequence that includes a sequence element that is or is a complement of an oncoselective translation sequence element. Methods and compositions useful for oncoselective translation are described in WO2020/257655 the entirety of which is incorporated herein by reference.

### Oncogenic Ribosomes

The present disclosure appreciates that studies have increasingly revealed alterations in ribosome structure and function that are associated with tumor development and/or progression. See, for example, Bastide and David Oncogenesis 2018 Apr. 7(4):34. Oncogenic ribosomes have a drastically altered translational landscape (“translatome”). In addition to more effectively translating various oncogenes, cancer ribosomes have been shown to be characterized by low translation fidelity and/or altered or increased stop codon read-through.

A variety of mechanisms have been described that may contribute to the altered function of oncogenic ribosomes. These include alterations in ribosome biogenesis, mutations in ribosomal protein genes, alterations in expression of ribosomal proteins, alterations in expression of rRNA, and/or alterations in the modification of rRNA. See, for example, Bastide and David Oncogenesis. 2018 April; 7(4): 34. Alterations in rRNA 2′-O-methylation patterns are also involved in cancer evolution. In some cancers, p53 inactivation triggers FBL overexpression and subsequent changes in rRNA methylation landscape (Marcel et al. Cancer Cell. 2013; 24:318-330). Such p53 inactivation (and/or FBL overexpression and/or changes in rRNA methylation) result(s) in impaired translational fidelity and increased translation of IRES-containing mRNAs. The gene encoding p53 protein, TP53 is the most commonly mutated tumor suppressor gene, Along with rRNA modifications, it is also closely connected with ribosome regulation through changes in ribosomal proteins. Ribosomal protein gene haploinsufficiency is found in about 43% of all cancers (Ajore et al., EMBO Mol Med. 2017; 9(4):498-507). In healthy cells, loss of both copies of any essential ribosomal protein gene is lethal. However, when a single copy of a ribosomal protein gene is lost, the stoichiometry of ribosomal proteins is altered and ribosomal proteins RPL5 and RPL11 have higher free (unbound) forms, which together with 5S rRNA, bind to MDM2 and stabilize p53 to stimulate growth arrest or apoptosis. This p53 mediated control mechanism in healthy cells is termed “impaired ribosome biogenesis checkpoint (Gentilella et al. Mol Cell. 2017; 67(1):55-70.e4).” In addition to TP53, retinoblastoma (RBI) gene, another commonly mutated tumor suppressor gene, is also involved in ribosome regulation, suppressing translational read-through in MYC oncogene-transformed senescent human cells (del Toro et al. BioRxiv. 2019; 10.1101/788380).

The present disclosure appreciates that oncoselective read-through can be harnessed as a powerful strategy for treatment of cancer. The present disclosure builds upon extensive work in the field of nucleic acid therapeutics (and particularly including RNA, such as mRNA therapeutics), among other things by providing technologies that ensure expression of a payload included in and/or encoded by such a nucleic acid is selectively or specifically expressed in tumor cells (relative to non-tumor cells).

By providing true oncoselective or oncospecific expression, the present disclosure reduces or obviates a need to develop and/or utilize targeted (e.g., oncoselective) delivery strategies that may be required in contexts where oncoselective or oncospecific payload expression cannot be achieved. Of course, those skilled in the art, reading the present disclosure, will appreciate that any available such oncoselective delivery technology may, in some embodiments, be desirably combined with provided technologies; it is simply not required.

Alternatively or additionally, by providing true oncoselective or oncospecific expression, the present disclosure creates an option to utilize payloads that might be inappropriate or undesirable without such a high degree of selectivity. For example, as discussed herein, cytotoxic payloads (e.g., such as toxins, and pro-necroptotic, pro-pyroptotic, and pro-apoptotic proteins) might have unacceptable side effect and/or toxicology profiles when utilized with technologies that cannot ensure oncoselectivity to the extent described herein.

### Oncoselective Translation Sequence Elements

### Read-Through Motifs

Among other things, the present disclosure encompasses the recognition that different ribosomes (e.g., ribosomes in tumor cells—e.g., oncogenic ribosomes—vs ribosomes in non-tumor cells—e.g., non-oncogenic ribosomes) have different processivity and/or read-through properties (e.g., different responses to pause structures and/or stop codons that impact processivity therethrough). In some embodiments, oncogenic ribosomes have frame shifts relative to non-oncogenic ribosomes. In some embodiments, frame shifts by oncogenic ribosomes can result in expression of payload sequences described herein.

In some embodiments, oncogenic ribosomes read-through, or process through, a canonical stop codon. In some embodiments, read-through of a stop codon by an oncogenic ribosome results in translation of a stop codon into an amino acid incorporated into a nascent polypeptide. In some embodiments, read-through of a stop codon by an oncogenic ribosome results in translation of some portion or all of the downstream (3′UTR) sequences following that stop codon.

Without wishing to be bound by any particular theory, the present disclosure observes that ribosome read-through of stop codons can be caused by interactions between the 18s rRNA and an RNA (e.g., an mRNA) bound by the ribosome. For example, helices of the rRNA may interact with mRNA sequences. See Namy et al. EMBO Rep. 2001 Sep. 15; 2(9): 787-793 describing interactions of helix 17 of rRNA in S. cerevisae with mRNA bound by the ribosome that leads to stop codon read-through. The present disclosure recognizes, among other things, that human rRNA helix 37 can interact with sequences of mRNA that contribute to stop codon read-through.

Alternatively or additionally, oncoselective ribosome stop codon read-through can be induced and/or enhanced by including one or more particular structural features in a translatable nucleic acid (e.g., an RNA such as an mRNA). In some embodiments, one or more primary structure features of a translatable nucleic acid (e.g., an RNA such as an mRNA) can be used to induce and/or enhance oncoselective stop codon read-through. Alternatively or additionally, in some embodiments, one or more secondary and/or tertiary structure features (e.g. stem loop, bulge loop, kissing loop, pseudoknots, or branch loop) of a translatable nucleic acid (e.g., an RNA such as an mRNA) can be used to induce and/or enhance oncoselective stop codon read-through. In some embodiments, a structural feature capable of inducing and/or enhancing stop codon read-through is within the first 50, 60, 70, 80, 90, 100, 110, or 120 nucleotides of the downstream flanking sequence. In some further embodiments, portions of a structural feature capable of inducing and/or enhancing stop codon read-through is comprised by the first 16 nucleotides of the downstream flanking sequence.

In some embodiments, a structural feature capable of inducing and/or enhancing stop codon read-through comprises 10, 20, 30, 40, 50 or more base paired nucleotides within the first 10, 20, 30, 40, 50, 60 or more nucleotides of the downstream flanking sequence.

In accordance with some embodiments of the present disclosure, stop codon read-through can be induced and/or enhanced through use of oncoselective read-through motifs as described herein.

Alternatively or additionally, in some embodiments, inclusion of one or more regions of high G-C content can be used to induce oncospecific stop codon read-through. For example, in some embodiments, high G-C content in the 3′UTR of a translatable nucleic acid (e.g., of an RNA such as an mRNA) can be used to induce and/or enhance oncospecific stop codon read through. In some embodiments, high G-C content in the nucleotides preceding a stop codon can be used to induce and/or enhance oncospecific stop codon read-through of that stop codon. In some embodiments, high G-C content in the 60 nucleotides preceding a stop codon can be used to induce and/or enhance oncospecific stop codon read-through of that stop codon. In some embodiments, high G-C content in 50 nucleotides following a stop codon can be used to induce and/or enhance oncospecific stop codon readthrough of that stop codon. In some embodiments, high G-C content in the first 120 nucleotides after a stop codon (i.e., in the 3′UTR) can be used to induce and/or enhance oncospecific stop codon readthrough of that stop codon. In some embodiments, high G-C content means a log-odds of binomial probability of 4 or greater relative to a non-readthrough transcript. In some embodiments, a readthrough motif comprises GC content of more than 42%, more than 48%, preferably more than 54% in the downstream flanking sequence.

In some embodiments, the readthrough motif comprises the amino acid sequence VNNNNNNMNNMWK (SEQ ID NO. 24), NNNVWNNKGHHNH (SEQ ID NO. 25), DVHVNNNCWNNNB (SEQ ID NO. 26), MWBNNNNNNNNNN (SEQ ID NO. 27), WGNNSNHNHDNNN(SEQ ID NO. 28), VNNNNNNMNNMWK (SEQ ID NO. 29) or VMNNWNKNNNNNN (SEQ ID NO. 30), wherein V stands for A, C or G, M stands for A or C, W stands for A or T/U, K stands for G or T/U, H stands for A, C or T/U, D stands for A,G or T/U, B stands for C, G or T/U, S stands for G or C, N stands for any nucleotide, within the region that spans the readthrough stop codon and the first 14 nucleotides of the downstream flanking sequence.

In some aspects, the oncoselective modification comprises an oncoselective sequence motif comprising an oncoselective readthrough motif, a 5′ UTR, a 3′ UTR, or a combination thereof. In some embodiments, the oncoselective modification comprises a nucleic acid sequence of any one of the nucleic acid sequences of SEQ ID NO: 41-110 (Table 6). In some embodiments, the oncoselective modification comprises at least one nucleic acid sequence of any one of the nucleic acid sequences of SEQ ID NO: 41-110. In some embodiments, the oncoselective modification comprises a combination of at least one of the nucleic acid sequences of SEQ ID NO: 41-110. In some embodiments, the oncoselective modification comprises a combination of two or more of the nucleic acid sequences of SEQ ID NO: 41-110.

TABLE 6
Exemplary nucleic acid sequences of the oncoselective sequence motif

| | | |
|---|---|---|
| SEQ ID | caRIP3K | AGGUCUUCUGGUCCCCACAGACUCAGAGAGAACCCGCCACCAUGAGCAGCGUG |
| NC: 41 | (mouse) | AAGCUGUGGCCUACCGGCGCCAGCGCCGUGCCCCUGGUGAGCCGGGAGGAGUU |
| | | AAAGAAGCUGGAGUUCGUGGGCAAGGGCGGCUUCGGCGUGGUGUUCCGGGCC |
| | | CACCACCGGACUUGGAACCACGAUGUGGCCGUGAAGAUCGUGAAUAGCAAGA |
| | | AGAUCAGCUGGGAGGUGAAGGCCAUGGUGAACCUGCGGAACGAGAACGUGCU |
| | | GCUGCUGCUGGGCGUGACCGAGGACCUGCAGUGGGACUUCGUGAGCGGCCAG |
| | | GCCCUGGUGACCCGGUUCAUGGAGAAUGGCAGCCUGGCCGGCCUGCUGCAGCC |
| | | CGAGUGCCCUCGGCCCUGGCCCCUCCUGUGCCGGCUGCUGCAGGAGGUGGUGC |
| | | UGGGCAUGUGCUACCUGCACAGCCUGAACCCACCCCUGCUGCACAGGGACCUG |
| | | AAGCCCAGCAACAUCCUGCUGGACCCUGAGCUGCACGCCAAGCUGGCCGAUUU |
| | | CGGCCUGAGCACCUUCCAGGGCGGCAGCCAGAGCGGCAGCGGCAGCGGAAGCG |
| | | GCUCACGGGACAGCGGCGGCACCCUGGCCUACCUGGACCCCGAGCUGCUGUUC |
| | | GACGUGAACCUGAAGGCCAGCAAAGCCAGCGACGUGUACAGCUUCGGCAUCCU |
| | | GGUGUGGGCCGUGCUGGCAGGCCGGGAGGCUGAGCUGGUGGACAAGACCAGC |
| | | CUGAUCCGGGAGACCGUGUGCGACCGGCAGAGCCGGCCCCCCCUGACCGAGCU |
| | | GCCCCCCGGCAGCCCCGAGACCCCCGGCCUGGAGAAGCUCAAGGAGCUGAUGA |
| | | UCCACUGCUGGGGCUCACAGUCCGAGAAUCGUCCAAGCUUCCAGGACUGCGAG |
| | | CCCAAGACCAACGAGGUGUACAACCUGGUGAAGGACAAGGUGGACGCCGCCGU |
| | | GAGCGAGGUGAAGCACUACCUGAGCCAGCACCGGAGCAGCGGCCGGAACCUGA |
| | | GCGCCCGGGAGCCCAGCCAGCGGGGCACCGAGAUGGACUGCCCCCGGGAGACC |
| | | AUGGUGAGCAAGAUGCUGGACCGGCUGCACCUGGAGGAGCCCAGCGGACCUG |
| | | UGCCCGGCAAGUGCCCCGAGCGGCAGGCCCAGGACACCAGCGUGGGCCCCGCC |
| | | ACCCCCGCCCGGACCAGCAGCGACCCCGUGGCCGGCACCCCUCAGAUCCCCCAC |
| | | ACCCUGCCCUUCCGGGGCACCACCCCCGGCCCCGUGUUCACCGAGACCCCAGGC |
| | | CCCCACCCCCAGAGGAAUCAGGGCGACGGCCGGCACGGCACCCCAUGGUACCC |
| | | UUGGACCCCCCCCAACCCCAUGACCGGCCCCCCCGCACUGGUGUUUAAUAACU |
| | | GUAGCGAGGUGCAGAUCGGCAACUACAACAGCCUGGUCGCCCCCCCCCGGACC |
| | | ACCGCCAGCAGCAGCGCCAAGUACGACCAGGCCCAGUUCGGCCGGGGCCGGGG |
| | | CUGGCAGCCCUUCCACAAGGGCACCAAGUACGAGAUCGAACGGAUGCUGAAGG |
| | | AGCUGAAGGACCUGCUGGAAGAGCUGAAGCGGAUGCUGGAGGAGCUUAAACG |
| | | GAGCCUGAAGGAACUGAAGAAGAACCCCAGCGAGGACGCCCUGGUGGAGAAU |
| | | AACCGGCUGAUCGUGAAGGUGCUGGAGGUGAUCGUGGAGGUGCUGCGGGCCA |
| | | UCAUCGAGCUGGCCGAGCUGAUCAUCCGGAGCGACUGAUUAAACCGGUCUGCA |
| | | GCUUUUGCCUUGGUUCUUCCUAGUGCCUACAAUGGGAAAACUUCACUCCAAA |
| | | GAGAAACCUAUUAAGUCAUCAUCUCCAAACUAAACCCUCACAAGUAACAGUU |
| | | GAAGAAAAAAUGGCAAGAGAUCAUAUCCUCAGACCAGGUGGAAUUACUUAAA |
| | | UUUUAAAGCCUGAAAAUUCCAAUUUGGGGGUGGGAGGUGGAAAAGGAACCCA |
| | | AUUUUCUUAUGAACAGAUAUUUUUAACUUAAUGGCACAAAGUCUUAGAAUAU |
| | | UAUUAUGUGCCCCGUGUUCCCUGUUCUUCGUUGCUGAAUUCGAUAUCCAGCG |
| | | GCCGCGCUAGCGCUGAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAA |
| SEQ ID | RIPK3 | AGGUCUUCUGGUCCCCACAGACUCAGAGAGAACCCGCCACCAUGAGCUGCGUG |
| NO: 42 | (human) | AAGCUGUGGCCCAGCGGCGCCCCCGCCCCACUGGUGAGCAUCGAGGAGCUGGA |
| | WU + | GAACCAGGAGCUGGUGGGCAAGGGCGGCUUCGGCACCGUGUUCCGGGCCCAGC |
| | coDomain | ACCGGAAGUGGGGCUACGACGUGGCCGUGAAGAUCGUGAACAGCAAGGCAAU |
| | | CAGCCGGGAGGUGAAGGCCAUGGCCAGCCUGGACAACGAGUUCGUGCUGCGGC |
| | | UGGAGGGCGUGAUCGAGAAGGUGAACUGGGACCAGGACCCAAAGCCUGCCCU |
| | | GGUGACCAAGUUCAUGGAGAACGGCAGCCUGAGCGGACUGCUGCAGAGCCAG |
| | | UGCCCCCGGCCCUGGCCACUGCUGUGCCGGCUGCUGAAGGAGGUGGUGCUGGG |
| | | CAUGUUCUACCUGCACGAUCAGAACCCCGUGCUGCUGCACCGUGACCUGAAGC |
| | | CCAGCAACGUGCUGCUGGACCCCGAGCUGCACGUGAAGCUGGCUGACUUCGGC |
| | | CUGAGCACCUUCCAGGGCGGCAGCCAGAGCGGCACCGGCAGCGGCGAGCCCGG |
| | | CGGCACCCUGGGCUACCUGGCCCCCGAGCUGUUCGUGAAUGUGAACCGGAAGG |
| | | CCAGCACCGCCAGCGACGUGUACAGCUUCGGCAUCCUGAUGUGGGCCGUGCUG |
| | | GCCGGCCGGGAGGUCGAGCUGCCCACCGAGCCCAGCCUGGUGUACGAGGCUGU |
| | | GUGCAACCGGCAGAACCGGCCCAGCCUGGCAGAGCUGCCCCAGGCCGGCCCCG |
| | | AGACCCCCGGCCUGGAGGGCCUUAAGGAGCUGAUGCAGCUGUGCUGGAGCAGC |
| | | GAGCCUAAGGACCGGCCCAGCUUCCAGGAGUGCCUGCCCAAGACCGACGAGGU |
| | | GUUCCAGAUGGUCGAGAAUAAUAUGAACGCAGCUGUGAGCACCGUGAAGGAC |
| | | UUCCUGAGCCAGCUGCGGAGCAGCAACCGGCGGUUCAGCAUCCCCGAGAGCGG |
| | | CCAGGGAGGCACCGAGAUGGAUGGCUUCCGGCGGACCAUCGAGAACCAGCACA |
| | | GCAGGAAUGACGUGAUGGUGAGCGAGUGGCUGAACAAGCUGAACCUGGAGGA |
| | | GCCCCCCAGCAGCGUGCCAAAGAAGUGCCCCAGCCUGACCAAGCGGAGCCGGG |
| | | CCCAGGAGGAGCAGGUGCCCCAGGCCUGGACCGCCGGCACCAGCAGCGACAGC |
| | | AUGGCCCAGCCCCCCCAGACCCCAGAGACCUCAACCUUCCGGAACCAGAUGCC |
| | | CAGCCCCACCAGCACCGGCACCCCAAGCCCCGGCCCCCGGGGCAACCAGGCCGC |
| | | CGAGAGGCAGGGCAUGAACUGGAGCUGCCGGACCCCCGAGCCCAACCCCGUGA |
| | | CCGGCCGGCCCCUGGUGAACAUCUACAACUGCAGCGGCGUGCAGGUGGGAGAC |
| | | AACAACUACCUGACCAUGCAGCAGACCACCGCUCUGCCCACCUGGGGCCUGGC |
| | | CCCAAGCGGCAAGGGCCGGGGCCUGCAGCACCCCCCCCCCGUGGGCAGCCAGG |
| | | AGGGCCCCAAGGACCCAGAGGCCUGGAGCCGGCCCCAGGGCUGGUACAACCAC |
| | | AGCGGCAAGGGGACCAAGUACGAGAUCGAGCGGAUGCUGAAGGAACUGAAGG |
| | | ACCUGCUGGAGGAGCUGAAACGGAUGCUCGAGGAGCUGAAGCGGAGCCUGAA |
| | | GGAGCUGAAGAAGAACCCAAGCGAGGACGCCCUGGUGGAGAACAACCGGCUG |
| | | AUCGUGAAGGUGCUGGAGGUGAUCGUGGAGGUGCUGCGGGCCAUCAUCGAGC |
| | | UGGCCGAGCUGAUAAUCCGGAGCGACUGAUUAAACCGGUCUGCAGCUUUUGC |
| | | CUUGGUUCUUCCUAGUGCCUACAAUGGGAAAACUUCACUCCAAAGAGAAACC |
| | | UAUUAAGUCAUCAUCUCCAAACUAAACCCUCACAAGUAACAGUUGAAGAAAA |
| | | AAUGGCAAGAGAUCAUAUCCUCAGACCAGGUGGAAUUACUUAAAUUUUAAAG |
| | | CCUGAAAAUUCCAAUUUGGGGGUGGGAGGUGGAAAAGGAACCCAAUUUUCUU |
| | | AUGAACAGAUAUUUUUAACUUAAUGGCACAAAGUCUUAGAAUAUUAUUAUGU |
| | | GCCCCGUGUUCCCUGUUCUUCGUUGCUGAAUUCGAUAUCCAGCGGCCGCGCUA |
| | | GCGCUGAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAA |
| SEQ ID | Human | AGGUCUUCUGGUCCCCACAGACUCAGAGAGAACCCGCCACCAUGUACCGGAUG |
| NO: 43 | IL-2 | CAGCUGCUGAGCUGCAUUGCCCUGAGCCUGGCCCUGGUGACCAACAGCGCCCC |
| | mutein | CACCAGCAGCAGCACCAAGAAGACCCAGCUGCAGCUAGAGCACCUGCUGCUGG |
| | F42A | ACCUGCAGAUGAUCCUGAAUGGCAUCAACAACUACAAGAACCCUAAGCUGACC |
| | Y45A | CGGAUGCUGACCGCAAAGUUCGCCAUGCCCAAGAAGGCCACUGAGCUGAAGCA |
| | L72G | CCUGCAGUGCCUGGAGGAGGAGCUGAAGCCCCUGGAGGAGGUGCUGAACGGC |
| | | GCCCAGAGCAAGAACUUCCACCUGCGGCCCCGGGACCUGAUCAGCAACAUUAA |
| | | CGUGAUCGUGCUGGAGCUGAAGGGCAGCGAGACCACCUUCAUGUGCGAAUAC |
| | | GCCGACGAAACCGCUACAAUCGUGGAAUUCCUGAAUCGGUGGAUCACCUUCUG |
| | | CCAGAGCAUCAUCAGCACCCUGACCUGAUUAAACCGGUCUGCAGCUUUUGCCU |
| | | UGGUUCUUCCUAGUGCCUACAAUGGGAAAACUUCACUCCAAAGAGAAACCUA |
| | | UUAAGUCAUCAUCUCCAAACUAAACCCUCACAAGUAACAGUUGAAGAAAAAA |
| | | UGGCAAGAGAUCAUAUCCUCAGACCAGGUGGAAUUACUUAAAUUUUAAAGCC |
| | | UGAAAAUUCCAAUUUGGGGGUGGGAGGUGGAAAAGGAACCCAAUUUUCUUAU |
| | | GAACAGAUAUUUUUAACUUAAUGGCACAAAGUCUUAGAAUAUUAUUAUGUGC |
| | | CCCGUGUUCCCUGUUCUUCGUUGCUGAAUUCGAUAUCCAGCGGCCGCGCUAGC |
| | | GCUGAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAA |
| SEQ ID | U27- | AGGUCUUCUGGUCCCCACAGACUCAGAGAGAACCCGCCACCaUgAAGUGAGGC |
| NO: 44 | RIP3K | UCUCCUCCCGCCCCGCCCCUCCCACGCCUCACCAGCCCCCCGCGCGCCCACCCU |
| | (mouse) | CCGGCGGGUGACAGCUCCGGGAUCAGCAACCCUUCCUGCUGCUGCUACUGCUG |
| | | CUGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAggaageggagcUacUaacUUcagccUgcUga |
| | | agcaggcUggagacgUggaggagaacccUggaccUUacUcgagcagACGCGUagaUcUgGGCUCCGGC |
| | | GAGGGCAGGGGAAGUCUUCUAACAUGCGGGGACGUGGAGGAAAAUCCCGGCC |
| | | CAAGCAGCGUGAAGCUGUGGCCUACCGGCGCCAGCGCCGUGCCCCUGGUGAGC |
| | | CGGGAGGAGUUAAAGAAGCUGGAGUUCGUGGGCAAGGGCGGCUUCGGCGUGG |
| | | UGUUCCGGGCCCACCACCGGACUUGGAACCACGAUGUGGCCGUGAAGAUCGUG |
| | | AAUAGCAAGAAGAUCAGCUGGGAGGUGAAGGCCAUGGUGAACCUGCGGAACG |
| | | AGAACGUGCUGCUGCUGCUGGGCGUGACCGAGGACCUGCAGUGGGACUUCGU |
| | | GAGCGGCCAGGCCCUGGUGACCCGGUUCAUGGAGAAUGGCAGCCUGGCCGGCC |
| | | UGCUGCAGCCCGAGUGCCCUCGGCCCUGGCCCCUCCUGUGCCGGCUGCUGCAG |
| | | GAGGUGGUGCUGGGCAUGUGCUACCUGCACAGCCUGAACCCACCCCUGCUGCA |
| | | CAGGGACCUGAAGCCCAGCAACAUCCUGCUGGACCCUGAGCUGCACGCCAAGC |
| | | UGGCCGAUUUCGGCCUGAGCACCUUCCAGGGCGGCAGCCAGAGCGGCAGCGGC |
| | | AGCGGAAGCGGCUCACGGGACAGCGGCGGCACCCUGGCCUACCUGGACCCCGA |
| | | GCUGCUGUUCGACGUGAACCUGAAGGCCAGCAAAGCCAGCGACGUGUACAGCU |
| | | UCGGCAUCCUGGUGUGGGCCGUGCUGGCAGGCCGGGAGGCUGAGCUGGUGGA |
| | | CAAGACCAGCCUGAUCCGGGAGACCGUGUGCGACCGGCAGAGCCGGCCCCCCC |
| | | UGACCGAGCUGCCCCCCGGCAGCCCCGAGACCCCCGGCCUGGAGAAGCUCAAG |
| | | GAGCUGAUGAUCCACUGCUGGGGCUCACAGUCCGAGAAUCGUCCAAGCUUCCA |
| | | GGACUGCGAGCCCAAGACCAACGAGGUGUACAACCUGGUGAAGGACAAGGUG |
| | | GACGCCGCCGUGAGCGAGGUGAAGCACUACCUGAGCCAGCACCGGAGCAGCGG |
| | | CCGGAACCUGAGCGCCCGGGAGCCCAGCCAGCGGGGCACCGAGAUGGACUGCC |
| | | CCCGGGAGACCAUGGUGAGCAAGAUGCUGGACCGGCUGCACCUGGAGGAGCCC |
| | | AGCGGACCUGUGCCCGGCAAGUGCCCCGAGCGGCAGGCCCAGGACACCAGCGU |
| | | GGGCCCCGCCACCCCCGCCCGGACCAGCAGCGACCCCGUGGCCGGCACCCCUCA |
| | | GAUCCCCCACACCCUGCCCUUCCGGGGCACCACCCCCGGCCCCGUGUUCACCGA |
| | | GACCCCAGGCCCCCACCCCCAGAGGAAUCAGGGCGACGGCCGGCACGGCACCC |
| | | CAUGGUACCCUUGGACCCCCCCCAACCCCAUGACCGGCCCCCCCGCACUGGUG |
| | | UUUAAUAACUGUAGCGAGGUGCAGAUCGGCAACUACAACAGCCUGGUCGCCCC |
| | | CCCCCGGACCACCGCCAGCAGCAGCGCCAAGUACGACCAGGCCCAGUUCGGCC |
| | | GGGGCCGGGGCUGGCAGCCCUUCCACAAGGGCACCAAGUACGAGAUCGAACGG |
| | | AUGCUGAAGGAGCUGAAGGACCUGCUGGAAGAGCUGAAGCGGAUGCUGGAGG |
| | | AGCUUAAACGGAGCCUGAAGGAACUGAAGAAGAACCCCAGCGAGGACGCCCU |
| | | GGUGGAGAAUAACCGGCUGAUCGUGAAGGUGCUGGAGGUGAUCGUGGAGGUG |
| | | CUGCGGGCCAUCAUCGAGCUGGCCGAGCUGAUCAUCCGGAGCGACUGAUUAAA |
| | | CCGGUCUGCAGCUUUUGCCUUGGUUCUUCCUAGUGCCUACAAUGGGAAAACU |
| | | UCACUCCAAAGAGAAACCUAUUAAGUCAUCAUCUCCAAACUAAACCCUCACAA |
| | | GUAACAGUUGAAGAAAAAAUGGCAAGAGAUCAUAUCCUCAGACCAGGUGGAA |
| | | UUACUUAAAUUUUAAAGCCUGAAAAUUCCAAUUUGGGGGUGGGAGGUGGAAA |
| | | AGGAACCCAAUUUUCUUAUGAACAGAUAUUUUUAACUUAAUGGCACAAAGUC |
| | | UUAGAAUAUUAUUAUGUGCCCCGUGUUCCCUGUUCUUCGUUGCUGAAUUCGA |
| | | UAUCCAGCGGCCGCGCUAGCGCUGAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAA |
| SEQ ID | U27- | AGGUCUUCUGGUCCCCACAGACUCAGAGAGAACCCGCCACCaUgAAGUGAGGC |
| NO: 45 | RIP3K | UCUCCUCCCGCCCCGCCCCUCCCACGCCUCACCAGCCCCCCGCGCGCCCACCCU |
| | (human) | CCGGCGGGUGACAGCUCCGGGAUCAGCAACCCUUCCUGCUGCUGCUACUGCUG |
| | WU + | CUGCUGCUGCCGCCGCCGCCGCCGCCGCUGCCCUUGGGUCCCCCCGAGUCUCCG |
| | coDomain | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAggaageggagcUacUaacUUcagccUgcUga |
| | | agcaggcUggagacgUggaggagaacccUggaccUUacUcgagcagACGCGUagaUcUgGGCUCCGGC |
| | | GAGGGCAGGGGAAGUCUUCUAACAUGCGGGGACGUGGAGGAAAAUCCCGGCC |
| | | CAAGCUGCGUGAAGCUGUGGCCCAGCGGCGCCCCCGCCCCACUGGUGAGCAUC |
| | | GAGGAGCUGGAGAACCAGGAGCUGGUGGGCAAGGGGGGCUUCGGCACCGUGU |
| | | UCCGGGCCCAGCACCGGAAGUGGGGCUACGACGUGGCCGUGAAGAUCGUGAAC |
| | | AGCAAGGCAAUCAGCCGGGAGGUGAAGGCCAUGGCCAGCCUGGACAACGAGU |
| | | UCGUGCUGCGGCUGGAGGGCGUGAUCGAGAAGGUGAACUGGGACCAGGACCC |
| | | AAAGCCUGCCCUGGUGACCAAGUUCAUGGAGAACGGCAGCCUGAGCGGACUGC |
| | | UGCAGAGCCAGUGCCCCCGGCCCUGGCCACUGCUGUGCCGGCUGCUGAAGGAG |
| | | GUGGUGCUGGGCAUGUUCUACCUGCACGAUCAGAACCCCGUGCUGCUGCACCG |
| | | UGACCUGAAGCCCAGCAACGUGCUGCUGGACCCCGAGCUGCACGUGAAGCUGG |
| | | CUGACUUCGGCCUGAGCACCUUCCAGGGCGGCAGCCAGAGCGGCACCGGCAGC |
| | | GGCGAGCCCGGCGGCACCCUGGGCUACCUGGCCCCCGAGCUGUUCGUGAAUGU |
| | | GAACCGGAAGGCCAGCACCGCCAGCGACGUGUACAGCUUCGGCAUCCUGAUGU |
| | | GGGCCGUGCUGGCCGGCCGGGAGGUCGAGCUGCCCACCGAGCCCAGCCUGGUG |
| | | UACGAGGCUGUGUGCAACCGGCAGAACCGGCCCAGCCUGGCAGAGCUGCCCCA |
| | | GGCCGGCCCCGAGACCCCCGGCCUGGAGGGCCUUAAGGAGCUGAUGCAGCUGU |
| | | GCUGGAGCAGCGAGCCUAAGGACCGGCCCAGCUUCCAGGAGUGCCUGCCCAAG |
| | | ACCGACGAGGUGUUCCAGAUGGUCGAGAAUAAUAUGAACGCAGCUGUGAGCA |
| | | CCGUGAAGGACUUCCUGAGCCAGCUGCGGAGCAGCAACCGGCGGUUCAGCAUC |
| | | CCCGAGAGCGGCCAGGGAGGCACCGAGAUGGAUGGCUUCCGGCGGACCAUCGA |
| | | GAACCAGCACAGCAGGAAUGACGUGAUGGUGAGCGAGUGGCUGAACAAGCUG |
| | | AACCUGGAGGAGCCCCCCAGCAGCGUGCCAAAGAAGUGCCCCAGCCUGACCAA |
| | | GCGGAGCCGGGCCCAGGAGGAGCAGGUGCCCCAGGCCUGGACCGCCGGCACCA |
| | | GCAGCGACAGCAUGGCCCAGCCCCCCCAGACCCCAGAGACCUCAACCUUCCGG |
| | | AACCAGAUGCCCAGCCCCACCAGCACCGGCACCCCAAGCCCCGGCCCCCGGGGC |
| | | AACCAGGGCGCCGAGAGGCAGGGCAUGAACUGGAGCUGCCGGACCCCCGAGCC |
| | | CAACCCCGUGACCGGCCGGCCCCUGGUGAACAUCUACAACUGCAGCGGCGUGC |
| | | AGGUGGGAGACAACAACUACCUGACCAUGCAGCAGACCACCGCUCUGCCCACC |
| | | UGGGGCCUGGCCCCAAGCGGCAAGGGCCGGGGCCUGCAGCACCCCCCCCCCGU |
| | | GGGCAGCCAGGAGGGCCCCAAGGACCCAGAGGCCUGGAGCCGGCCCCAGGGCU |
| | | GGUACAACCACAGCGGCAAGGGGACCAAGUACGAGAUCGAGCGGAUGCUGAA |
| | | GGAACUGAAGGACCUGCUGGAGGAGCUGAAACGGAUGCUCGAGGAGCUGAAG |
| | | CGGAGCCUGAAGGAGCUGAAGAAGAACCCAAGCGAGGACGCCCUGGUGGAGA |
| | | ACAACCGGCUGAUCGUGAAGGUGCUGGAGGUGAUCGUGGAGGUGCUGCGGGC |
| | | CAUCAUCGAGCUGGCCGAGCUGAUAAUCCGGAGCGACUGAUUAAACCGGUCU |
| | | GCAGCUUUUGCCUUGGUUCUUCCUAGUGCCUACAAUGGGAAAACUUCACUCCA |
| | | AAGAGAAACCUAUUAAGUCAUCAUCUCCAAACUAAACCCUCACAAGUAACAG |
| | | UUGAAGAAAAAAUGGCAAGAGAUCAUAUCCUCAGACCAGGUGGAAUUACUUA |
| | | AAUUUUAAAGCCUGAAAAUUCCAAUUUGGGGGUGGGAGGUGGAAAAGGAACC |
| | | CAAUUUUCUUAUGAACAGAUAUUUUUAACUUAAUGGCACAAAGUCUUAGAAU |
| | | AUUAUUAUGUGCCCCGUGUUCCCUGUUCUUCGUUGCUGAAUUCGAUAUCCAG |
| | | CGGCCGCGCUAGCGCUGAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAA |
| SEQ ID | U27- | AGGUCUUCUGGUCCCCACAGACUCAGAGAGAACCCGCCACCaUgAAGUGAGGC |
| NO: 46 | human- | UCUCCUCCCGCCCCGCCCCUCCCACGCCUCACCAGCCCCCCGCGCGCCCACCCU |
| | IL-2 | CCGGCGGGUGACAGCUCCGGGAUCAGCAACCCUUCCUGCUGCUGCUACUGCUG |
| | mutein | CUGCUGCUGCCGCCGCCGCCGCCGCCGCUGCCCUUGGGUCCCCCCGAGUCUCCG |
| | F42A | GGACUGCCCUCUCGACUGUCAGUGGGGCAGCCUCUCCGACUCUGCACCCGCCU |
| | Y45A | CGACCUCCCCACCCGCUCCCACACCCCUGUGCCCUCAUGUGGAGCCUAAGAGA |
| | L72G | ACAGAACAGGCCGUGAAGCCAGCAGAGAAAggaageggagcUacUaacUUcagccUgcUga |
| | | agcaggcUggagacgUggaggagaacccUggaccUUacUcgagcagACGCGUagaUcUgGGCUCCGGC |
| | | GAGGGCAGGGGAAGUCUUCUAACAUGCGGGGACGUGGAGGAAAAUCCCGGCC |
| | | CAUACCGGAUGCAGCUGCUGAGCUGCAUUGCCCUGAGCCUGGCCCUGGUGACC |
| | | AACAGCGCCCCCACCAGCAGCAGCACCAAGAAGACCCAGCUGCAGCUAGAGCA |
| | | CCUGCUGCUGGACCUGCAGAUGAUCCUGAAUGGCAUCAACAACUACAAGAACC |
| | | CUAAGCUGACCCGGAUGCUGACCGCAAAGUUCGCCAUGCCCAAGAAGGCCACU |
| | | GAGCUGAAGCACCUGCAGUGCCUGGAGGAGGAGCUGAAGCCCCUGGAGGAGG |
| | | UGCUGAACGGCGCCCAGAGCAAGAACUUCCACCUGCGGCCCCGGGACCUGAUC |
| | | AGCAACAUUAACGUGAUCGUGCUGGAGCUGAAGGGCAGCGAGACCACCUUCA |
| | | UGUGCGAAUACGCCGACGAAACCGCUACAAUCGUGGAAUUCCUGAAUCGGUG |
| | | GAUCACCUUCUGCCAGAGCAUCAUCAGCACCCUGACCUGAUUAAACCGGUCUG |
| | | CAGCUUUUGCCUUGGUUCUUCCUAGUGCCUACAAUGGGAAAACUUCACUCCAA |
| | | AGAGAAACCUAUUAAGUCAUCAUCUCCAAACUAAACCCUCACAAGUAACAGU |
| | | UGAAGAAAAAAUGGCAAGAGAUCAUAUCCUCAGACCAGGUGGAAUUACUUAA |
| | | AUUUUAAAGCCUGAAAAUUCCAAUUUGGGGGUGGGAGGUGGAAAAGGAACCC |
| | | AAUUUUCUUAUGAACAGAUAUUUUUAACUUAAUGGCACAAAGUCUUAGAAUA |
| | | UUAUUAUGUGCCCCGUGUUCCCUGUUCUUCGUUGCUGAAUUCGAUAUCCAGC |
| | | GGCCGCGCUAGCGCUGAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAA |
| SEQ ID | hIL- | AGGUCUUCUGGUCCCCACAGACUCAGAGAGAACCCGCCACCAUGGCCCCCCGG |
| NO: 47 | 15_verA_ | CGGGCCCGGGGCUGCCGGACCCUGGGCCUGCCUGCCCUGCUGCUGCUGCUUCU |
| | adm | GCUGCGGCCCCCCGCCACCCGGGGCAUCACCUGUCCCCCACCUAUGAGCGUGG |
| | | AGCACGCCGACAUCUGGGUGAAGAGCUACAGCCUGUACAGCCGGGAGCGGUAC |
| | | AUCUGCAACAGCGGCUUCAAGCGGAAGGCAGGCACCAGCAGCCUGACCGAGUG |
| | | CGUGCUGAACAAGGCCACCAACGUGGCCCACUGGACCACCCCCAGCCUUAAGU |
| | | GCAUCCGGGACCCUGCGCUGGUCCACCAGCGGCCUGCCCCCCCCAGCACCGUG |
| | | ACCACCGCAGGAGUGACCCCCCAGCCCGAGAGCCUGAGCCCCAGCGGCAAGGA |
| | | GCCCGCCGCCAGCUCACCCAGCAGCAACAACACCGCCGCCACCACCGCCGCCAU |
| | | CGUGCCCGGCAGCCAGCUGAUGCCUAGCAAGAGCCCCAGCACCGGCACCACCG |
| | | AGAUCAGCAGCCACGAGAGCAGCCACGGCACCCCCAGCCAGACCACCGCCAAG |
| | | AAUUGGGAGCUGACCGCCAGCGCCAGCCACCAGCCCCCAGGCGUGUACCCCCA |
| | | GGGGCACAGCGACACCACCGUGGCCAUCAGCACCAGCACCGUGCUGCUGUGCG |
| | | GCCUGAGCGCAGUGAGCCUGCUGGCCUGCUACCUGAAGAGCCGGCAGACCCCA |
| | | CCCCUGGCUAGCGUGGAGAUGGAGGCCAUGGAGGCCCUGCCCGUGACCUGGGG |
| | | AACCAGCAGCCGGGACGAGGACCUGGAGAACUGCAGCCACCACCUGUGAUUAA |
| | | ACCGGUCUGCAGCUUUUGCCUUGGUUCUUCCUAGUGCCUACAAUGGGAAAAC |
| | | UUCACUCCAAAGAGAAACCUAUUAAGUCAUCAUCUCCAAACUAAACCCUCACA |
| | | AGUAACAGUUGAAGAAAAAAUGGCAAGAGAUCAUAUCCUCAGACCAGGUGGA |
| | | AUUACUUAAAUUUUAAAGCCUGAAAAUUCCAAUUUGGGGGUGGGAGGUGGAA |
| | | AAGGAACCCAAUUUUCUUAUGAACAGAUAUUUUUAACUUAAUGGCACAAAGU |
| | | CUUAGAAUAUUAUUAUGUGCCCCGUGUUCCCUGUUCUUCGUUGCUGAAUUCG |
| | | AUAUCCAGCGGCCGCGCUAGCGCUGAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAA |
| SEQ ID | hIL- | AGGUCUUCUGGUCCCCACAGACUCAGAGAGAACCCGCCACCAUGGAGUUCGGC |
| NO: 48 | 15_verB_ | CUGAGCUGGGUGUUCCUGGUGGCCCUGUUCCGGGGAGUGCAGUGUAUCACCU |
| | adm | GCCCCCCCCCCAUGAGCGUGGAGCACGCUGACAUCUGGGUGAAGAGCUACAGC |
| | | CUGUACAGCCGGGAGCGGUACAUCUGCAACAGCGGCUUCAAGCGGAAGGCCGG |
| | | CACCAGCUCCCUGACCGAGUGCGUGCUGAACAAGGCCACCAACGUGGCCCACU |
| | | GGACCACCCCCAGCCUGAAGUGCAUCCGGGACCCCGCCCUGGUGGGCGGCUCA |
| | | GGCGGCAGCGGCGGCAGCGGGGGCAGCGGAGGCAGCGGCGGAAGCGGCGGCA |
| | | ACUGGGUGAACGUGAUCAGCGACCUGAAGAAGAUCGAGGACCUGAUCCAAAG |
| | | CAUGCACAUUGACGCCACCCUGUACACCGAGAGCGACGUGCACCCCAGCUGCA |
| | | AGGUGACCGCAAUGAAGUGCUUCCUGCUGGAGCUGCAGGUGAUCAGCCUGGA |
| | | GAGCGGCGACGCCAGCAUCCACGACACCGUGGAGAACCUGAUCAUCCUGGCGA |
| | | ACAACAGCCUGAGCAGCAACGGCAACGUGACCGAGAGCGGCUGUAAGGAGUG |
| | | CGAGGAGCUGGAGGAGAAGAAUAUCAAGGAGUUCCUGCAGAGCUUCGUGCAC |
| | | AUCGUGCAGAUGUUCAUCAACACCAGCUGAUUAAACCGGUCUGCAGCUUUUG |
| | | CCUUGGUUCUUCCUAGUGCCUACAAUGGGAAAACUUCACUCCAAAGAGAAACC |
| | | UAUUAAGUCAUCAUCUCCAAACUAAACCCUCACAAGUAACAGUUGAAGAAAA |
| | | AAUGGCAAGAGAUCAUAUCCUCAGACCAGGUGGAAUUACUUAAAUUUUAAAG |
| | | CCUGAAAAUUCCAAUUUGGGGGUGGGAGGUGGAAAAGGAACCCAAUUUUCUU |
| | | AUGAACAGAUAUUUUUAACUUAAUGGCACAAAGUCUUAGAAUAUUAUUAUGU |
| | | GCCCCGUGUUCCCUGUUCUUCGUUGCUGAAUUCGAUAUCCAGCGGCCGCGCUA |
| | | GCGCUGAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAA |
| SEQ ID | hIL- | AGGUCUUCUGGUCCCCACAGACUCAGAGAGAACCCGCCACCAUGGAGUUCGGC |
| NO: 49 | 15_verC_ | CUGAGCUGGGUGUUUCUGGUGGCCCUGUUCCGGGGCGUGCAGUGCAACUGGG |
| | adm | UGAACGUGAUCAGCGACCUGAAGAAGAUCGAGGACCUGAUCCAGUCAAUGCA |
| | | CAUCGAUGCAACCCUGUACACCGAGAGCGACGUGCACCCUAGCUGUAAGGUGA |
| | | CCGCAAUGAAGUGCUUCCUGCUGGAGCUGCAGGUGAUAAGCCUGGAGAGCGG |
| | | CGACGCCAGCAUCCACGACACAGUCGAGAAUCUGAUCAUCCUGGCUAACAACA |
| | | GCCUGAGCAGCAAUGGUAAUGUGACCGAGAGCGGCUGCAAGGAGUGCGAGGA |
| | | GCUGGAGGAGAAGAACAUCAAGGAGUUCCUGCAGAGCUUCGUGCACAUCGUG |
| | | CAGAUGUUCAUCAACACCAGCGGCGGCAGCGGGGGCAGCGGCGGCUCAGGUGG |
| | | CAGCGGAGGCAGCGGCGGAAGCGGCGGCAUCACCUGCCCUCCCCCCAUGAGCG |
| | | UGGAGCACGCAGACAUCUGGGUGAAGAGCUACAGCCUGUACAGCCGGGAGCG |
| | | GUACAUCUGCAACAGCGGCUUCAAGCGGAAGGCCGGCACCAGCAGCCUGACCG |
| | | AGUGUGUGCUGAACAAGGCCACCAACGUGGCCCACUGGACAACCCCCAGCCUG |
| | | AAGUGCAUCAGGGACCCCGCCCUGGUGUGAUUAAACCGGUCUGCAGCUUUUGC |
| | | CUUGGUUCUUCCUAGUGCCUACAAUGGGAAAACUUCACUCCAAAGAGAAACC |
| | | UAUUAAGUCAUCAUCUCCAAACUAAACCCUCACAAGUAACAGUUGAAGAAAA |
| | | AAUGGCAAGAGAUCAUAUCCUCAGACCAGGUGGAAUUACUUAAAUUUUAAAG |
| | | CCUGAAAAUUCCAAUUUGGGGGUGGGAGGUGGAAAAGGAACCCAAUUUUCUU |
| | | AUGAACAGAUAUUUUUAACUUAAUGGCACAAAGUCUUAGAAUAUUAUUAUGU |
| | | GCCCCGUGUUCCCUGUUCUUCGUUGCUGAAUUCGAUAUCCAGCGGCCGCGCUA |
| | | GCGCUGAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAA |
| SEQ ID | mIL- | AGGUCUUCUGGUCCCCACAGACUCAGAGAGAACCCGCCACCAUGGCUAGCCCC |
| NO: 50 | 15_verA_ | CAGCUGCGGGGCUACGGCGUGCAGGCCAUCCCUGUGCUGCUGUUACUGCUGCU |
| | adm | GCUCCUGCUGCUGCCCCUGCGGGUGACCCCCGGCACCACCUGCCCCCCCCCCGU |
| | | GAGCAUCGAGCACGCCGACAUCCGGGUGAAGAACUACAGCGUGAACAGCCGGG |
| | | AGAGGUACGUGUGCAAUAGCGGCUUCAAGCGGAAGGCCGGCACCAGCACCCUG |
| | | AUCGAGUGCGUGAUCAACAAGAACACCAACGUGGCCCACUGGACCACCCCCAG |
| | | CCUGAAGUGCAUCCGGGACCCCAGCUUAGCCCACUACAGCCCCGUGCCCACCG |
| | | UGGUGACCCCAAAGGUGACCAGCCAGCCCGAGAGCCCCAGCCCCAGCGCCAAG |
| | | GAGCCUGAGGCCUUCUCCCCAAAGAGCGACACCGCCAUGACCACCGAGACCGC |
| | | CAUCAUGCCCGGCAGCCGGCUGACCCCCAGCCAGACCACCAGCGCCGGCACCA |
| | | CAGGCACCGGCAGCCACAAGAGCAGCCGGGCACCCAGCCUGGCCGCCACCAUG |
| | | ACCCUGGAGCCUACCGCUAGCACCAGCCUGCGGAUCACCGAGAUCAGCCCCCA |
| | | CAGCAGCAAGAUGACCAAGGUGGCCAUCAGCACCAGCGUGCUGCUGGUGGGCG |
| | | CCGGCGUGGUGAUGGCUUUCCUGGCCUGGUACAUCAAGAGCCGGCAGCCCAGC |
| | | CAGCCUUGCCGGGUGGAGGUGGAGACCAUGGAGACUGUGCCCAUGACCGUGC |
| | | GGGCCAGCAGCAAGGAGGACGAGGACACCGGCGCCGGGGGCAGCGGCGGUAGC |
| | | GGAGGCAGCGGCGGCAGCGGCGGAAGCGGCGGCUCCGGCGGCAUGAAGAUCCU |
| | | GAAGCCCUACAUGCGGAACACCAGCAUCAGCUGCUACCUGUGCUUCCUGCUGA |
| | | ACAGCCACUUCCUGACCGAGGCCGGCAUCCACGUGUUCAUCCUGGGCUGCGUG |
| | | AGCGUGGGCCUGCCUAAGACCGAGGCCAACUGGAUCGACGUGCGGUACGACCU |
| | | GGAGAAGAUCGAGAGCCUGAUCCAGAGCAUCCACAUCGACACCACCCUGUACA |
| | | CCGACAGCGACUUCCACCCCAGCUGCAAGGUGACCGCCAUGAACUGCUUUCUG |
| | | CUGGAGCUGCAGGUGAUCCUGCACGAGUACAGCAACAUGACCCUGAACGAGAC |
| | | CGUGCGGAACGUGCUGUACCUGGCCAACAGCACCCUGAGCAGCAACAAGAACG |
| | | UGGCCGAGAGCGGCUGCAAGGAGUGCGAGGAGCUGGAGGAGAAGACCUUCAC |
| | | CGAGUUCCUGCAGAGCUUCAUCCGGAUCGUGCAGAUGUUCAUCAACACCAGCU |
| | | GAUUAAACCGGUCUGCAGCUUUUGCCUUGGUUCUUCCUAGUGCCUACAAUGG |
| | | GAAAACUUCACUCCAAAGAGAAACCUAUUAAGUCAUCAUCUCCAAACUAAACC |
| | | CUCACAAGUAACAGUUGAAGAAAAAAUGGCAAGAGAUCAUAUCCUCAGACCA |
| | | GGUGGAAUUACUUAAAUUUUAAAGCCUGAAAAUUCCAAUUUGGGGGUGGGAG |
| | | GUGGAAAAGGAACCCAAUUUUCUUAUGAACAGAUAUUUUUAACUUAAUGGCA |
| | | CAAAGUCUUAGAAUAUUAUUAUGUGCCCCGUGUUCCCUGUUCUUCGUUGCUG |
| | | AAUUCGAUAUCCAGCGGCCGCGCUAGCGCUGAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAA |
| SEQ ID | mIL- | AGGUCUUCUGGUCCCCACAGACUCAGAGAGAACCCGCCACCAUGAGCGUGCUG |
| NO: 51 | 15_verB | ACCCAGGUGCUGGCCCUGCUGCUGCUGUGGCUGACCGGCGCCCGUUGCACCAC |
| | adm | CUGCCCCCCCCCAGUGAGCAUCGAGCACGCCGAUAUCCGGGUGAAGAACUACA |
| | | GCGUGAACAGCCGGGAGCGGUACGUGUGCAACAGCGGCUUCAAGCGGAAGGC |
| | | CGGCACAAGCACCCUGAUCGAGUGCGUGAUCAACAAGAACACCAAUGUGGCCC |
| | | ACUGGACCACCCCCAGCCUGAAGUGUAUCCGGGACCCUAGCCUGGCCGGCGGC |
| | | AGCGGCGGGAGCGGUGGCAGCGGCGGAAGCGGCGGUAGCGGAGGCAGCGGCG |
| | | GCAACUGGAUCGACGUGCGGUACGACCUAGAGAAGAUCGAGAGCCUGAUCCA |
| | | GAGCAUCCACAUCGACACCACCCUGUACACCGACAGCGACUUCCACCCCAGCU |
| | | GCAAGGUGACCGCCAUGAACUGCUUCCUGCUGGAGCUGCAGGUGAUCCUGCAC |
| | | GAGUACAGCAACAUGACACUGAACGAGACCGUGCGGAACGUGCUGUACCUGG |
| | | CAAACAGCACCCUGAGCAGCAACAAGAAUGUGGCCGAGAGCGGCUGCAAGGA |
| | | AUGCGAGGAGCUGGAGGAGAAGACCUUCACCGAGUUCCUCCAGAGCUUCAUCC |
| | | GGAUCGUGCAGAUGUUCAUCAACACCAGCUGAUUAAACCGGUCUGCAGCUUU |
| | | UGCCUUGGUUCUUCCUAGUGCCUACAAUGGGAAAACUUCACUCCAAAGAGAA |
| | | ACCUAUUAAGUCAUCAUCUCCAAACUAAACCCUCACAAGUAACAGUUGAAGA |
| | | AAAAAUGGCAAGAGAUCAUAUCCUCAGACCAGGUGGAAUUACUUAAAUUUUA |
| | | AAGCCUGAAAAUUCCAAUUUGGGGGUGGGAGGUGGAAAAGGAACCCAAUUUU |
| | | CUUAUGAACAGAUAUUUUUAACUUAAUGGCACAAAGUCUUAGAAUAUUAUUA |
| | | UGUGCCCCGUGUUCCCUGUUCUUCGUUGCUGAAUUCGAUAUCCAGCGGCCGCG |
| | | CUAGCGCUGAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAA |
| SEQ ID | mIL- | AGGUCUUCUGGUCCCCACAGACUCAGAGAGAACCCGCCACCAUGAGCGUGCUG |
| NO: 52 | 15_verC_ | ACCCAGGUGCUGGCUCUACUGCUGCUGUGGUUAACAGGCGCCCGGUGCAACUG |
| | adm | GAUCGACGUGCGGUACGACCUGGAGAAGAUCGAGAGCCUGAUCCAGAGCAUC |
| | | CACAUCGACACCACCCUGUACACCGACAGCGACUUCCACCCUAGCUGCAAGGU |
| | | GACCGCCAUGAACUGCUUCCUGCUGGAGCUGCAGGUGAUCCUGCACGAGUACA |
| | | GCAAUAUGACCCUGAACGAGACCGUGCGGAACGUGCUGUACCUGGCCAACAGC |
| | | ACCCUGAGCAGCAAUAAGAAUGUGGCCGAGAGCGGCUGCAAGGAGUGUGAGG |
| | | AGCUGGAGGAGAAGACCUUCACUGAGUUCCUGCAGAGCUUUAUCCGGAUCGU |
| | | GCAGAUGUUCAUCAACACUAGCGGCGGAAGCGGCGGCUCAGGCGGCAGCGGA |
| | | GGCAGCGGCGGCAGCGGGGGCAGCGGCGGAACCACCUGUCCCCCCCCCGUGAG |
| | | CAUCGAGCACGCCGACAUCCGGGUGAAGAACUACAGCGUGAACUCACGGGAGC |
| | | GGUACGUGUGCAACUCAGGCUUCAAGCGGAAGGCCGGCACCAGCACCCUGAUC |
| | | GAGUGCGUGAUCAACAAGAACACAAACGUGGCCCACUGGACCACCCCCAGCCU |
| | | GAAGUGCAUCCGGGACCCCAGCCUGGCCUGAUUAAACCGGUCUGCAGCUUUUG |
| | | CCUUGGUUCUUCCUAGUGCCUACAAUGGGAAAACUUCACUCCAAAGAGAAACC |
| | | UAUUAAGUCAUCAUCUCCAAACUAAACCCUCACAAGUAACAGUUGAAGAAAA |
| | | AAUGGCAAGAGAUCAUAUCCUCAGACCAGGUGGAAUUACUUAAAUUUUAAAG |
| | | CCUGAAAAUUCCAAUUUGGGGGUGGGAGGUGGAAAAGGAACCCAAUUUUCUU |
| | | AUGAACAGAUAUUUUUAACUUAAUGGCACAAAGUCUUAGAAUAUUAUUAUGU |
| | | GCCCCGUGUUCCCUGUUCUUCGUUGCUGAAUUCGAUAUCCAGCGGCCGCGCUA |
| | | GCGCUGAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAA |
| SEQ ID | mIL- | AGGUCUUCUGGUCCCCACAGACUCAGAGAGAACCCGCCACCAUGAGCGUGCUG |
| NO: 53 | 15_verD_ | ACCCAGGUGCUGGCCCUGCUGCUGCUGUGGCUGACCGGCGCCCGGUGCAACUG |
| | adm | GAUCGACGUGCGGUACGAUCUGGAGAAGAUCGAGAGCCUGAUCCAGAGCAUC |
| | | CACAUCGACACCACCCUGUACACCGAUAGCGACUUCCACCCCAGCUGCAAGGU |
| | | GACCGCCAUGAACUGCUUCCUGCUGGAACUGCAGGUGAUCCUGCACGAGUACA |
| | | GCAACAUGACCCUGAACGAGACCGUGCGGAACGUCCUGUACCUGGCCAACAGC |
| | | ACACUGAGCAGCAACAAGAACGUGGCCGAGAGCGGCUGCAAGGAGUGCGAGG |
| | | AGCUGGAGGAGAAGACCUUCACCGAGUUCCUGCAGAGCUUCAUCCGUAUCGU |
| | | GCAGAUGUUCAUCAACACCAGCGGGGGCAGCGGCGGAAGCGGGGGCAGCGGC |
| | | GGAAGCGGCGGCAGCGGAGGCAGCGGAGGCACCACAUGCCCCCCACCCGUGAG |
| | | CAUCGAGCACGCCGACAUACGGGUGAAGAAUUACAGCGUGAACAGCCGGGAG |
| | | CGGUACGUGUGCAACAGCGGCUUCAAGCGGAAGGCCGGCACCAGCACCCUGAU |
| | | CGAGUGCGUGAUCAACAAGAACACUAACGUGGCACACUGGACCACCCCCAGCC |
| | | UCAAGUGCAUCCGGGACCCAAGCCUGGCCGGGGGCGGCGGCUCCGGCGGAGGC |
| | | GGCAGCGGCGGUGGAGGCAGCGGCCCUACAAUCAAGCCCUGCCCCCCCUGCAA |
| | | GUGCCCCGCCCCCAAUGCCGCCGGCGGCCCCAGCGUGUUCAUCUUCCCCCCCAA |
| | | GAUCAAGGACGUGCUGAUGAUCUCACUAAGCCCCAUAGUGACCUGCGUGGUG |
| | | GUGGACGUGAGCGAGGACGACCCUGACGUGCAGAUCAGCUGGUUCGUGAACA |
| | | ACGUGGAGGUGCACACCGCCCAGACCCAGACCCACCGGGAGGACUACAACAGC |
| | | ACCCUGCGGGUGGUGAGCGCCCUGCCCAUCCAGCACCAGGACUGGAUGAGCGG |
| | | CAAGGAGUUCAAGUGUAAGGUGAAUAAUAAGGACCUGGGCGCCCCAAUAGAG |
| | | CGGACCAUUAGCAAGCCCAAGGGGAGCGUGCGGGCCCCCCAGGUGUACGUGCU |
| | | GCCACCCCCUGAGGAGGAGAUGACCAAAAAGCAGGUGACCCUGACCUGCAUGG |
| | | UGACCGAUUUCAUGCCAGAGGACAUCUACGUGGAGUGGACCAACAACGGCAA |
| | | GACCGAGCUGAACUACAAGUGAUUAAACCGGUCUGCAGCUUUUGCCUUGGUU |
| | | CUUCCUAGUGCCUACAAUGGGAAAACUUCACUCCAAAGAGAAACCUAUUAAG |
| | | UCAUCAUCUCCAAACUAAACCCUCACAAGUAACAGUUGAAGAAAAAAUGGCA |
| | | AGAGAUCAUAUCCUCAGACCAGGUGGAAUUACUUAAAUUUUAAAGCCUGAAA |
| | | AUUCCAAUUUGGGGGUGGGAGGUGGAAAAGGAACCCAAUUUUCUUAUGAACA |
| | | GAUAUUUUUAACUUAAUGGCACAAAGUCUUAGAAUAUUAUUAUGUGCCCCGU |
| | | GUUCCCUGUUCUUCGUUGCUGAAUUCGAUAUCCAGCGGCCGCGCUAGCGCUGA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAA |
| SEQ ID | hIL- | AGGUCUUCUGGUCCCCACAGACUCAGAGAGAACCCGCCACCAUGUGCCACCAG |
| NO: 54 | 12_adm | CAGCUGGUGAUCAGCUGGUUCAGCCUGGUGUUCCUCGCCAGCCCCCUGGUGGC |
| | | UAUCUGGGAGCUGAAGAAGGACGUGUACGUGGUGGAGCUGGACUGGUACCCC |
| | | GACGCCCCCGGCGAGAUGGUGGUGCUCACCUGCGAUACCCCCGAGGAGGACGG |
| | | AAUCACCUGGACCCUGGACCAGAGCAGCGAGGUGUUAGGCAGCGGCAAGACCC |
| | | UGACCAUCCAGGUGAAGGAGUUCGGCGACGCCGGCCAGUACACCUGCCACAAG |
| | | GGCGGGGAGGUGCUGAGCCACAGCCUGCUGCUGCUGCACAAGAAGGAGGACG |
| | | GCAUCUGGAGCACCGACAUCCUGAAGGACCAGAAGGAGCCCAAGAACAAGACC |
| | | UUCCUGCGGUGCGAGGCCAAGAAUUACAGCGGCCGGUUCACCUGCUGGUGGCU |
| | | GACCACCAUCAGCACCGACCUGACCUUCAGCGUGAAGAGCAGCCGGGGCAGCA |
| | | GCGACCCCCAGGGCGUGACCUGCGGCGCCGCCACCCUGAGCGCCGAGCGGGUG |
| | | AGGGGCGACAAUAAGGAGUACGAGUACAGCGUGGAGUGUCAGGAGGAUAGCG |
| | | CAUGCCCCGCCGCCGAGGAGAGCCUGCCCAUCGAGGUGAUGGUGGACGCCGUG |
| | | CACAAGCUGAAGUACGAGAACUACACCAGCAGCUUCUUCAUCCGGGACAUCAU |
| | | CAAGCCCGACCCCCCCAAGAACCUGCAGCUGAAGCCCCUGAAGAACAGCCGGC |
| | | AGGUGGAGGUGUCAUGGGAGUACCCCGACACCUGGAGCACCCCCCACAGCUAC |
| | | UUCAGCCUGACAUUCUGUGUGCAGGUGCAGGGCAAGAGCAAGCGGGAGAAGA |
| | | AGGACCGGGUGUUCACCGACAAGACCAGCGCCACCGUGAUCUGCCGGAAGAAU |
| | | GCCAGCAUCAGCGUGCGGGCCCAGGACCGGUACUACAGCAGCAGCUGGAGCGA |
| | | GUGGGCCAGCGUGCCUUGCUCCGGCGGCGGAGGAAGCGGAGGCGGGGGCAGC |
| | | GGGGGCGGCGGAAGCCGGAACCUGCCCGUGGCCACCCCAGACCCCGGCAUGUU |
| | | CCCCUGCCUGCACCACAGCCAGAACCUGCUGCGGGCCGUGAGCAACAUGCUGC |
| | | AGAAGGCUCGGCAGACCCUGGAGUUCUACCCCUGCACCAGCGAGGAGAUCGAC |
| | | CACGAGGACAUCACCAAGGACAAGACUAGCACCGUGGAGGCCUGCCUGCCCCU |
| | | GGAGCUGACCAAGAAUGAGAGCUGCCUGAACAGCCGGGAGACCAGCUUUAUC |
| | | ACCAACGGCAGCUGCCUGGCCAGCCGGAAGACCUCAUUCAUGAUGGCCCUGUG |
| | | CCUGAGCAGCAUCUACGAGGACCUGAAGAUGUACCAGGUGGAGUUCAAGACC |
| | | AUGAACGCCAAGCUGCUGAUGGACCCUAAGCGGCAAAUCUUCCUGGACCAGAA |
| | | UAUGCUGGCCGUGAUCGACGAGCUCAUGCAGGCCCUGAACUUUAACAGCGAG |
| | | ACCGUGCCCCAGAAGAGCAGCCUGGAGGAGCCAGACUUCUACAAGACCAAGAU |
| | | CAAGCUGUGCAUCCUGCUGCACGCUUUCCGGAUCCGGGCCGUGACCAUCGACC |
| | | GGGUGAUGAGCUACCUGAACGCGAGCAGCGGAGGCGGCAGCGGUGGCGGCGG |
| | | CUCAGGCGGCGGAGGCAGCGGCGGAGGCGGAAGCGGCGGCGGCAGCCUGCAGG |
| | | UGGUGAUCAGCGCCAUCCUGGCUCUGGUGGUGCUGACCAUCAUCUCCCUGAUC |
| | | AUCCUGAUCGGCGGAGGUGGCAGCUGAUUAAACCGGUCUGCAGCUUUUGCCU |
| | | UGGUUCUUCCUAGUGCCUACAAUGGGAAAACUUCACUCCAAAGAGAAACCUA |
| | | UUAAGUCAUCAUCUCCAAACUAAACCCUCACAAGUAACAGUUGAAGAAAAAA |
| | | UGGCAAGAGAUCAUAUCCUCAGACCAGGUGGAAUUACUUAAAUUUUAAAGCC |
| | | UGAAAAUUCCAAUUUGGGGGUGGGAGGUGGAAAAGGAACCCAAUUUUCUUAU |
| | | GAACAGAUAUUUUUAACUUAAUGGCACAAAGUCUUAGAAUAUUAUUAUGUGC |
| | | CCCGUGUUCCCUGUUCUUCGUUGCUGAAUUCGAUAUCCAGCGGCCGCGCUAGC |
| | | GCUGAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAA |
| SEQ ID | mIL- | AGGUCUUCUGGUCCCCACAGACUCAGAGAGAACCCGCCACCAUGUGCCCCCAG |
| NO: 55 | 12_adm | AAGCUGACCAUCAGCUGGUUCGCCAUCGUGCUGCUGGUGAGCCCUCUGAUGGC |
| | | CAUGUGGGAGCUGGAGAAGGACGUGUACGUGGUGGAGGUGGAUUGGACCCCC |
| | | GACGCACCCGGCGAGACCGUGAACCUGACCUGCGAUACCCCUGAGGAGGACGA |
| | | CAUCACCUGGACUAGCGAUCAGCGGCACGGCGUGAUCGGCAGCGGCAAGACCC |
| | | UGACCAUCACCGUGAAGGAGUUCCUGGAUGCCGGCCAGUACACCUGCCACAAG |
| | | GGCGGGGAGACCCUGAGCCACAGCCACCUGCUGCUGCACAAAAAGGAGAACGG |
| | | CAUCUGGAGCACCGAGAUCCUGAAGAACUUCAAGAACAAGACCUUUCUGAAG |
| | | UGCGAGGCUCCCAACUACAGCGGUCGGUUCACCUGCAGCUGGCUGGUGCAGCG |
| | | GAAUAUGGACCUGAAGUUUAACAUCAAGUCAAGCAGCAGCAGCCCCGACAGCC |
| | | GGGCCGUGACCUGUGGCAUGGCCAGCCUGAGCGCCGAGAAGGUGACCCUGGAC |
| | | CAGAGGGAUUACGAGAAGUACAGCGUGAGCUGCCAGGAGGACGUGACCUGCC |
| | | CAACCGCCGAGGAGACCCUGCCCAUCGAGCUGGCCCUGGAGGCCCGGCAGCAG |
| | | AACAAGUACGAGAACUACAGCACCAGCUUCUUCAUCCGGGACAUCAUCAAGCC |
| | | UGACCCCCCCAAGAACCUGCAGAUGAAGCCCCUGAAGAACAGCCAGGUGGAGG |
| | | UGAGCUGGGAGUACCCCGACAGCUGGAGCACCCCCCACAGCUACUUCAGCCUG |
| | | AAGUUCUUCGUGCGGAUCCAGCGGAAGAAGGAGAAGAUGAAGGAGACCGAGG |
| | | AGGGCUGCAACCAGAAGGGCGCCUUCCUGGUGGAGAAGACCAGCACCGAGGU |
| | | GCAGUGUAAGGGCGGCAACGUGUGCGUGCAGGCCCAGGACCGGUACUACAAC |
| | | AGCUCAUGCAGCAAGUGGGCCUGCGUGCCCUGCCGGGUGAGGAGCGGGGGGGG |
| | | CGGCUCCGGCGGAGGAGGCAGCGGAGGAGGCGGCAGCCGGGUGAUCCCCGUGA |
| | | GCGGCCCUGCCCGGUGCCUGAGCCAGAGCCGGAACCUGCUGAAGACCACCGAC |
| | | GACAUGGUGAAGACCGCUCGGGAGAAGCUGAAGCACUACAGCUGCACCGCCGA |
| | | GGACAUCGACCACGAGGACAUCACCCGGGACCAGACCAGCACCUUAAAGACCU |
| | | GCCUGCCCCUGGAGCUGCACAAGAAUGAGAGCUGCCUGGCCACCCGGGAGACC |
| | | AGCAGCACCACCCGGGGCAGCUGCCUGCCCCCCCAGAAGACCAGCCUGAUGAU |
| | | GACCCUGUGCCUGGGCAGCAUCUACGAGGACCUGAAGAUGUACCAGACCGAGU |
| | | UCCAGGCAAUCAACGCUGCUCUGCAGAAUCACAACCACCAGCAGAUCAUCCUG |
| | | GACAAGGGAAUGCUGGUGGCCAUCGACGAGCUGAUGCAGAGCCUGAACCACA |
| | | ACGGCGAGACCCUGCGGCAGAAGCCCCCCGUGGGCGAGGCCGACCCCUACCGU |
| | | GUGAAGAUGAAGCUGUGCAUCCUGCUGCACGCUUUCAGCACCCGGGUGGUGA |
| | | CCAUCAACCGGGUGAUGGGCUACCUGAGCAGCGCAAGCGGUGGCGGCAGCGGC |
| | | GGCGGGGGCUCCGGCGGCGGUGGGAGCGGCGGAGGCGGUUCAGGCGGCGGCA |
| | | GCCUGCAGGUGGUGAUCAGCGCCAUCCUGGCCCUGGUGGUGCUCACCGUGAUC |
| | | AGCCUGAUCAUCCUGAUCGGGGGCGGCGGCUCAGGCAAGCCCAUCCCAAAUCC |
| | | ACUGCUGGGCCUGGACAGCACCUGAUUAAACCGGUCUGCAGCUUUUGCCUUGG |
| | | UUCUUCCUAGUGCCUACAAUGGGAAAACUUCACUCCAAAGAGAAACCUAUUA |
| | | AGUCAUCAUCUCCAAACUAAACCCUCACAAGUAACAGUUGAAGAAAAAAUGG |
| | | CAAGAGAUCAUAUCCUCAGACCAGGUGGAAUUACUUAAAUUUUAAAGCCUGA |
| | | AAAUUCCAAUUUGGGGGUGGGAGGUGGAAAAGGAACCCAAUUUUCUUAUGAA |
| | | CAGAUAUUUUUAACUUAAUGGCACAAAGUCUUAGAAUAUUAUUAUGUGCCCC |
| | | GUGUUCCCUGUUCUUCGUUGCUGAAUUCGAUAUCCAGCGGCCGCGCUAGCGCU |
| | | GAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAA |
| SEQ ID | TET-IL- | AGGUCUUCUGGUCCCCACAGACUCAGAGAGAACCCGCCACCAUGUGCCCCCAG |
| NO: 56 | 12_adm | AAGCUGACCAUCAGCUGGUUCGCCAUCGUGCUGCUGGUGAGCCCUCUGAUGGC |
| | | CAUGUGGGAGCUCGAGAAGGACGUGUACGUGGUGGAGGUGGACUGGACCCCC |
| | | GACGCCCCCGGCGAGACCGUGAACCUGACCUGUGACACCCCCGAGGAGGACGA |
| | | CAUCACCUGGACCAGCGACCAGCGGCACGGCGUGAUCGGAAGCGGCAAGACCC |
| | | UGACCAUCACCGUCAAGGAGUUCCUGGACGCCGGCCAGUACACCUGCCACAAG |
| | | GGCGGCGAGACACUGAGCCACAGCCACCUGCUGUUACACAAGAAGGAGAACGG |
| | | CAUCUGGAGCACCGAGAUUCUGAAGAACUUCAAGAACAAGACCUUCCUGAAG |
| | | UGCGAAGCCCCCAAUUACAGCGGCCGGUUCACCUGCAGCUGGCUGGUGCAGCG |
| | | GAACAUGGACCUGAAGUUCAAUAUCAAGUCAAGCAGCAGCAGCCCCGAUAGCC |
| | | GGGCCGUGACCUGCGGCAUGGCCAGCCUGAGCGCCGAGAAGGUGACCCUGGAC |
| | | CAGAGGGACUACGAGAAGUACAGCGUGAGCUGUCAGGAGGACGUGACAUGCC |
| | | CAACCGCCGAGGAGACCCUGCCCAUCGAGCUGGCCCUGGAGGCCCGGCAGCAG |
| | | AAUAAGUACGAGAAUUACAGCACCAGCUUCUUCAUCCGGGACAUCAUCAAGCC |
| | | CGACCCCCCUAAGAACCUGCAGAUGAAGCCCCUGAAGAAUAGCCAGGUCGAGG |
| | | UGAGCUGGGAGUACCCCGAUAGCUGGAGCACCCCCCACAGCUACUUCAGCCUG |
| | | AAGUUCUUCGUGCGGAUCCAGCGGAAGAAGGAGAAGAUGAAGGAGACCGAGG |
| | | AGGGCUGCAAUCAGAAGGGCGCAUUCCUGGUGGAGAAGACCAGCACCGAGGU |
| | | GCAGUGCAAGGGCGGCAACGUGUGCGUGCAGGCCCAGGAUCGGUACUACAAC |
| | | AGCAGCUGCAGCAAGUGGGCCUGCGUGCCAUGCCGGGUGCGGAGCGGCGGCGG |
| | | AGGCUCAGGUGGCGGCGGCUCAGGCGGGGGGGGCAGCCGGGUGAUCCCCGUGA |
| | | GCGGCCCCGCCCGGUGCCUUAGCCAGAGCCGGAAUCUGCUGAAGACCACCGAC |
| | | GACAUGGUGAAGACCGCCCGGGAGAAGCUGAAGCACUACAGCUGCACCGCCGA |
| | | GGACAUCGACCACGAGGACAUCACCCGGGACCAGACCAGCACCCUGAAGACCU |
| | | GCCUGCCCCUGGAGCUGCACAAGAACGAGAGCUGCCUGGCAACCCGGGAGACC |
| | | AGCAGCACCACCCGGGGCAGCUGCCUCCCCCCCCAGAAGACCAGCCUGAUGAU |
| | | GACCCUGUGCCUGGGGAGCAUCUACGAGGACCUGAAGAUGUACCAGACCGAG |
| | | UUCCAGGCUAUAAACGCCGCCCUGCAGAACCACAACCACCAGCAGAUCAUCCU |
| | | GGACAAGGGCAUGCUGGUGGCCAUCGACGAGCUGAUGCAGAGCCUGAACCAC |
| | | AACGGCGAGACUCUGCGGCAGAAGCCCCCUGUGGGCGAGGCCGAUCCCUACCG |
| | | GGUGAAGAUGAAGCUGUGCAUCCUGCUGCACGCCUUCAGCACCCGGGUGGUG |
| | | ACCAUCAACAGGGUGAUGGGCUACCUGAGCAGCGCUAGCGGCGGAGGUAGCG |
| | | GAGGCGGCGGCUCCGGCGGCGGCGGCAGCGGUGGAGGCGGCAGCGGCGGCGGU |
| | | AGCCUGCAAGUGGUGAUCAGCGCCAUCCUGGCCCUGGUGGUGCUGACCGUGAU |
| | | CAGCCUGAUCAUCCUGAUCGGCGGUGGCGGGAGCGGCAAGCCCAUCCCCAACC |
| | | CACUGCUGGGCCUGGACAGCACCUGAUUAAACCGGUCUGCAGCUUUUGCCUUG |
| | | GUUCUUCCUAGUGCCUACAAUGGGAAAACUUCACUCCAAAGAGAAACCUAUU |
| | | AAGUCAUCAUCUCCAAACUAAACCCUCACAAGUAACAGUUGAAGAAAAAAUG |
| | | GCAAGAGAUCAUAUCCUCAGACCAGGUGGAAUUACUUAAAUUUUAAAGCCUG |
| | | AAAAUUCCAAUUUGGGGGUGGGAGGUGGAAAAGGAACCCAAUUUUCUUAUGA |
| | | ACAGAUAUUUUUAACUUAAUGGCACAAAGUCUUAGAAUAUUAUUAUGUGCCC |
| | | CGUGUUCCCUGUUCUUCGUUGCUGAAUUCGAUAUCCAGCGGCCGCGCUAGCGC |
| | | UGAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAA |
| SEQ ID | hOX40L_ | AGGUCUUCUGGUCCCCACAGACUCAGAGAGAACCCGCCACCUGGAGCGGGUGC |
| NO: 57 | adm | AGCCCCUGGAGGAGAACGUGGGCAACGCCGCCCGGCCCCGGUUCGAGCGGAAC |
| | | AAGCUGCUGCUGGUGGCCAGCGUGAUCCAGGGCCUGGGCCUGCUGCUGUGCUU |
| | | CACCUACAUCUGCCUGCACUUCAGCGCCCUGCAGGUGAGCCACAGGUACCCCC |
| | | GGAUCCAGAGCAUCAAGGUGCAGUUCACCGAGUACAAGAAGGAGAAGGGCUU |
| | | CAUCCUGACCAGCCAGAAGGAGGACGAGAUCAUGAAGGUGCAGAACAACAGC |
| | | GUGAUCAUCAACUGCGACGGCUUCUACCUGAUCUCACUGAAGGGCUACUUCAG |
| | | CCAGGAGGUGAACAUCAGCCUGCACUACCAAAAGGACGAGGAGCCCCUGUUCC |
| | | AGCUGAAGAAGGUGCGGAGCGUGAACAGCCUGAUGGUGGCCUCACUGACCUA |
| | | CAAGGACAAGGUGUACCUUAACGUGACCACCGACAACACCAGCCUGGACGACU |
| | | UCCACGUGAACGGCGGCGAGCUGAUCCUGAUUCACCAGAACCCUGGCGAGUUC |
| | | UGCGUGCUGUGAUUAAACCGGUCUGCAGCUUUUGCCUUGGUUCUUCCUAGUG |
| | | CCUACAAUGGGAAAACUUCACUCCAAAGAGAAACCUAUUAAGUCAUCAUCUCC |
| | | AAACUAAACCCUCACAAGUAACAGUUGAAGAAAAAAUGGCAAGAGAUCAUAU |
| | | CCUCAGACCAGGUGGAAUUACUUAAAUUUUAAAGCCUGAAAAUUCCAAUUUG |
| | | GGGGUGGGAGGUGGAAAAGGAACCCAAUUUUCUUAUGAACAGAUAUUUUUAA |
| | | CUUAAUGGCACAAAGUCUUAGAAUAUUAUUAUGUGCCCCGUGUUCCCUGUUC |
| | | UUCGUUGCUGAAUUCGAUAUCCAGCGGCCGCGCUAGCGCUGAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAA |
| SEQ ID | mOX40L_ | AGGUCUUCUGGUCCCCACAGACUCAGAGAGAACCCGCCACCAUGGAGGGCGAG |
| NO: 58 | adm | GGCGUGCAGCCACUGGAUGAGAACCUGGAGAACGGCAGCCGGCCCCGGUUCAA |
| | | GUGGAAGAAGACCUUACGUCUGGUGGUGAGCGGCAUCAAGGGCGCCGGCAUG |
| | | CUGCUGUGCUUCAUCUACGUGUGUCUGCAGCUGAGCAGCAGCCCCGCCAAGGA |
| | | CCCCCCUAUCCAGCGGCUGCGGGGCGCCGUGACCCGGUGCGAGGACGGCCAGC |
| | | UGUUCAUCAGCAGCUACAAGAACGAGUACCAGACCAUGGAGGUGCAGAACAA |
| | | CAGCGUGGUGAUCAAGUGCGACGGCCUGUACAUCAUCUACCUGAAGGGCAGC |
| | | UUCUUCCAGGAAGUGAAGAUCGACCUACACUUCAGGGAGGAUCACAACCCCAU |
| | | CAGCAUCCCCAUGCUGAACGACGGCCGGCGGAUCGUGUUCACCGUGGUGGCCA |
| | | GCCUGGCCUUCAAGGACAAGGUGUACCUGACCGUGAACGCCCCCGACACCCUG |
| | | UGUGAGCACCUGCAGAUCAACGACGGCGAGCUGAUCGUGGUGCAGCUGACUCC |
| | | CGGCUACUGCGCCCCCGAGGGCAGCUACCACAGCACCGUGAACCAGGUGCCCC |
| | | UUUGAUUAAACCGGUCUGCAGCUUUUGCCUUGGUUCUUCCUAGUGCCUACAA |
| | | UGGGAAAACUUCACUCCAAAGAGAAACCUAUUAAGUCAUCAUCUCCAAACUA |
| | | AACCCUCACAAGUAACAGUUGAAGAAAAAAUGGCAAGAGAUCAUAUCCUCAG |
| | | ACCAGGUGGAAUUACUUAAAUUUUAAAGCCUGAAAAUUCCAAUUUGGGGGUG |
| | | GGAGGUGGAAAAGGAACCCAAUUUUCUUAUGAACAGAUAUUUUUAACUUAAU |
| | | GGCACAAAGUCUUAGAAUAUUAUUAUGUGCCCCGUGUUCCCUGUUCUUCGUU |
| | | GCUGAAUUCGAUAUCCAGCGGCCGCGCUAGCGCUGAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAA |
| SEQ ID | hIFN- | AGGUCUUCUGGUCCCCACAGACUCAGAGAGAACCCGCCACCAUGGCCCUGACC |
| NO: 59 | A_adm | UUCGCCCUGUUAGUGGCGCUGCUGGUGCUGAGCUGCAAGAGCAGCUGCAGCG |
| | | UGGGCUGCGACCUGCCUCAGACCCACAGCCUGGGCAGCCGGCGGACCCUGAUG |
| | | CUGCUGGCCCAGAUGCGGAAGAUCAGCCUGUUCAGCUGUCUGAAGGACCGGCA |
| | | CGACUUCGGCUUCCCCCAGGAGGAAUUCGGCAACCAGUUCCAGAAGGCCGAGA |
| | | CCAUCCCCGUGCUGCACGAGAUGAUCCAGCAGAUCUUCAACCUGUUCAGCACC |
| | | AAGGAUAGCAGCGCCGCUUGGGACGAGACCCUUCUGGACAAGUUCUACACCGA |
| | | GCUGUACCAGCAGCUGAACGACCUGGAGGCCUGCGUGAUCCAGGGCGUGGGCG |
| | | UGACCGAGACCCCUCUGAUGAAGGAGGACAGCAUCCUGGCCGUGCGGAAGUAC |
| | | UUUCAGCGGAUCACCCUGUACCUGAAGGAGAAGAAGUACAGCCCAUGCGCCUG |
| | | GGAGGUGGUGCGGGCCGAGAUCAUGCGGAGCUUCAGCCUGAGCACCAACCUGC |
| | | AGGAGAGCCUGCGGAGCAAGGAGUGAUUAAACCGGUCUGCAGCUUUUGCCUU |
| | | GGUUCUUCCUAGUGCCUACAAUGGGAAAACUUCACUCCAAAGAGAAACCUAU |
| | | UAAGUCAUCAUCUCCAAACUAAACCCUCACAAGUAACAGUUGAAGAAAAAAU |
| | | GGCAAGAGAUCAUAUCCUCAGACCAGGUGGAAUUACUUAAAUUUUAAAGCCU |
| | | GAAAAUUCCAAUUUGGGGGUGGGAGGUGGAAAAGGAACCCAAUUUUCUUAUG |
| | | AACAGAUAUUUUUAACUUAAUGGCACAAAGUCUUAGAAUAUUAUUAUGUGCC |
| | | CCGUGUUCCCUGUUCUUCGUUGCUGAAUUCGAUAUCCAGCGGCCGCGCUAGCG |
| | | CUGAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAA |
| SEQ ID | IFN- | AGGUCUUCUGGUCCCCACAGACUCAGAGAGAACCCGCCACCAUGGCUCGGCUG |
| NO: 60 | a_adm | UGCGCCUUCCUGAUGAUCCUGGUGAUGAUGAGCUACUACUGGAGCGCAUGCA |
| | | GCCUGGGCUGCGACCUGCCACACACCUACAACCUGGGCAAUAAGCGGGCUCUG |
| | | ACCGUGCUGGAGGAGAUGCGGCGGCUGCCCCCCCUGAGCUGCCUGAAGGACCG |
| | | GAAGGACUUCGGCUUCCCCCUGGAGAAGGUGGACAACCAGCAGAUCCAAAAG |
| | | GCACAGGCCAUCCUGGUGCUGCGGGACCUGACCCAGCAGAUCCUGAACCUGUU |
| | | CACCAGCAAGGACCUGAGCGCCACCUGGAACGCCACCCUGCUGGACAGCUUCU |
| | | GCAACGACCUGCACCAGCAGCUGAACGACCUGAAGGCCUGCGUCAUGCAGGAG |
| | | CCACCACUGACCCAGGAGGACAGCCUGUUAGCUGUGCGGACUUACUUCCACCG |
| | | GAUCACCGUGUACCUGCGGAAGAAGAAGCACAGCCUGUGCGCUUGGGAGGUG |
| | | AUCCGGGCCGAGGUGUGGCGGGCCCUGAGCAGCAGCACCAACCUGCUGGCCCG |
| | | GCUGAGCGAGGAGAAGGAGUGAUUAAACCGGUCUGCAGCUUUUGCCUUGGUU |
| | | CUUCCUAGUGCCUACAAUGGGAAAACUUCACUCCAAAGAGAAACCUAUUAAG |
| | | UCAUCAUCUCCAAACUAAACCCUCACAAGUAACAGUUGAAGAAAAAAUGGCA |
| | | AGAGAUCAUAUCCUCAGACCAGGUGGAAUUACUUAAAUUUUAAAGCCUGAAA |
| | | AUUCCAAUUUGGGGGUGGGAGGUGGAAAAGGAACCCAAUUUUCUUAUGAACA |
| | | GAUAUUUUUAACUUAAUGGCACAAAGUCUUAGAAUAUUAUUAUGUGCCCCGU |
| | | GUUCCCUGUUCUUCGUUGCUGAAUUCGAUAUCCAGCGGCCGCGCUAGCGCUGA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAA |
| SEQ ID | IL- | AGGUCUUCUGGUCCCCACAGACUCAGAGAGAACCCGCCACCAUGUACCGGAUG |
| NO: 61 | 2_adm | CAGCUGCUGAGCUGCAUCGCCCUGAGCCUGGCCCUGGUGACCAACAGCGCCCC |
| | | CACCAGCAGCAGCACCAAGAAGACCCAGCUGCAACUGGAGCACCUGCUGCUGG |
| | | AUCUGCAGAUGAUCCUGAACGGCAUCAACAAUUACAAGAAUCCCAAGCUGACC |
| | | CGGAUGCUGACCUUCAAGUUCUACAUGCCCAAGAAGGCUACCGAGCUGAAGCA |
| | | CCUGCAGUGCCUGGAGGAGGAGCUGAAGCCCCUGGAGGAGGUGCUGAACCUG |
| | | GCCCAGAGCAAGAACUUCCACUUCGACCCCCGGGACGUGGUGAGCAACAUCAA |
| | | CGUGUUCGUGCUGGAGCUGAAGGGCAGCGAGACCACCUUCAUGUGCGAGUAC |
| | | GCCGACGAGACCGCCACCAUCGUGGAGUUCCUGAACCGGUGGAUCACCUUCUG |
| | | CCAGAGCAUCAUCAGCUGAUUAAACCGGUCUGCAGCUUUUGCCUUGGUUCUUC |
| | | CUAGUGCCUACAAUGGGAAAACUUCACUCCAAAGAGAAACCUAUUAAGUCAU |
| | | CAUCUCCAAACUAAACCCUCACAAGUAACAGUUGAAGAAAAAAUGGCAAGAG |
| | | AUCAUAUCCUCAGACCAGGUGGAAUUACUUAAAUUUUAAAGCCUGAAAAUUC |
| | | CAAUUUGGGGGUGGGAGGUGGAAAAGGAACCCAAUUUUCUUAUGAACAGAUA |
| | | UUUUUAACUUAAUGGCACAAAGUCUUAGAAUAUUAUUAUGUGCCCCGUGUUC |
| | | CCUGUUCUUCGUUGCUGAAUUCGAUAUCCAGCGGCCGCGCUAGCGCUGAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAA |
| SEQ ID | mIL- | AGGUCUUCUGGUCCCCACAGACUCAGAGAGAACCCGCCACCAUGUACAGCAUG |
| NO: 62 | 2_adm | CAGCUGGCAAGCUGCGUGACCCUGACCCUGGUGCUGCUAGUGAACAGCGCCCC |
| | | CACCAGCAGCAGCACCAGCAGCUCCACCGCCGAGGCCCAGCAGCAACAACAGC |
| | | AACAGCAGCAGCAGCAACAGCACCUGGAGCAGCUCCUGAUGGACCUGCAGGAG |
| | | CUGCUGAGCCGGAUGGAGAACUACCGGAACCUGAAGCUGCCCCGGAUGCUGAC |
| | | CUUCAAGUUCUACCUGCCCAAGCAGGCCACAGAGCUGAAGGACCUGCAGUGCC |
| | | UGGAGGACGAGCUGGGCCCCCUGCGGCACGUGCUGGAUCUGACCCAGAGCAAG |
| | | AGCUUCCAGCUGGAGGACGCCGAGAACUUCAUCAGCAACAUCCGGGUGACCGU |
| | | GGUGAAGCUGAAGGGCAGCGACAACACCUUCGAGUGCCAGUUCGACGACGAG |
| | | AGCGCAACCGUGGUGGACUUCCUGCGGCGGUGGAUCGCCUUCUGCCAGAGCAU |
| | | CAUCAGCACCAGCCCCCAGUGAUUAAACCGGUCUGCAGCUUUUGCCUUGGUUC |
| | | UUCCUAGUGCCUACAAUGGGAAAACUUCACUCCAAAGAGAAACCUAUUAAGU |
| | | CAUCAUCUCCAAACUAAACCCUCACAAGUAACAGUUGAAGAAAAAAUGGCAA |
| | | GAGAUCAUAUCCUCAGACCAGGUGGAAUUACUUAAAUUUUAAAGCCUGAAAA |
| | | UUCCAAUUUGGGGGUGGGAGGUGGAAAAGGAACCCAAUUUUCUUAUGAACAG |
| | | AUAUUUUUAACUUAAUGGCACAAAGUCUUAGAAUAUUAUUAUGUGCCCCGUG |
| | | UUCCCUGUUCUUCGUUGCUGAAUUCGAUAUCCAGCGGCCGCGCUAGCGCUGAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAA |
| SEQ ID | hIL- | AGGUCUUCUGGUCCCCACAGACUCAGAGAGAACCCGCCACCAUGGCCCCCCGG |
| NO: 63 | 15_verA_ | CGGGCCCGGGGCUGCCGGACCCUGGGCCUGCCUGCCCUGCUGCUGCUGCUUCU |
| | down1 | GCUGCGGCCCCCCGCCACCCGGGGCAUCACCUGUCCCCCACCUAUGAGCGUGG |
| | | AGCACGCCGACAUCUGGGUGAAGAGCUACAGCCUGUACAGCCGGGAGCGGUAC |
| | | AUCUGCAACAGCGGCUUCAAGCGGAAGGCAGGCACCAGCAGCCUGACCGAGUG |
| | | CGUGCUGAACAAGGCCACCAACGUGGCCCACUGGACCACCCCCAGCCUUAAGU |
| | | GCAUCCGGGACCCUGCGCUGGUCCACCAGCGGCCUGCCCCCCCCAGCACCGUG |
| | | ACCACCGCAGGAGUGACCCCCCAGCCCGAGAGCCUGAGCCCCAGCGGCAAGGA |
| | | GCCCGCCGCCAGCUCACCCAGCAGCAACAACACCGCCGCCACCACCGCCGCCAU |
| | | CGUGCCCGGCAGCCAGCUGAUGCCUAGCAAGAGCCCCAGCACCGGCACCACCG |
| | | AGAUCAGCAGCCACGAGAGCAGCCACGGCACCCCCAGCCAGACCACCGCCAAG |
| | | AAUUGGGAGCUGACCGCCAGCGCCAGCCACCAGCCCCCAGGCGUGUACCCCCA |
| | | GGGGCACAGCGACACCACCGUGGCCAUCAGCACCAGCACCGUGCUGCUGUGCG |
| | | GCCUGAGCGCAGUGAGCCUGCUGGCCUGCUACCUGAAGAGCCGGCAGACCCCA |
| | | CCCCUGGCUAGCGUGGAGAUGGAGGCCAUGGAGGCCCUGCCCGUGACCUGGGG |
| | | AACCAGCAGCCGGGACGAGGACCUGGAGAACUGCAGCCACCACCUGUGAUUAA |
| | | ACCGGUACCGGUUGAUAAUAGCAGACACCAUUGUCACACUCCAGCUGGAGCCU |
| | | CGGUGGCCAUGCUUCUUGCCCCUUGGGCCCAGACACCAUUGUCACACUCCAUC |
| | | CCCCCAGCCCCUCCUCCCCUUCCUGCACCCGUACCCCCCAGACACCAUUGUCAC |
| | | ACUCCAGUGGUCUUUGAAUAAAGUCUGAGUGGGGGCGAAUUCGAUAUCCAG |
| | | CGGCCGCGCUAGCGCUGGCUAGCGAAUUCGAUAUCCAGCGGCCGCAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAA |
| SEQ ID | hIL- | AGGUCUUCUGGUCCCCACAGACUCAGAGAGAACCCGCCACCAUGGAGUUCGGC |
| NO: 64 | 15_verB_ | CUGAGCUGGGUGUUCCUGGUGGCCCUGUUCCGGGGAGUGCAGUGUAUCACCU |
| | down1 | GCCCCCCCCCCAUGAGCGUGGAGCACGCUGACAUCUGGGUGAAGAGCUACAGC |
| | | CUGUACAGCCGGGAGCGGUACAUCUGCAACAGCGGCUUCAAGCGGAAGGCCGG |
| | | CACCAGCUCCCUGACCGAGUGCGUGCUGAACAAGGCCACCAACGUGGCCCACU |
| | | GGACCACCCCCAGCCUGAAGUGCAUCCGGGACCCCGCCCUGGUGGGGGCUCA |
| | | GGCGGCAGCGGCGGCAGCGGGGGCAGCGGAGGCAGCGGCGGAAGCGGCGGCA |
| | | ACUGGGUGAACGUGAUCAGCGACCUGAAGAAGAUCGAGGACCUGAUCCAAAG |
| | | CAUGCACAUUGACGCCACCCUGUACACCGAGAGCGACGUGCACCCCAGCUGCA |
| | | AGGUGACCGCAAUGAAGUGCUUCCUGCUGGAGCUGCAGGUGAUCAGCCUGGA |
| | | GAGCGGCGACGCCAGCAUCCACGACACCGUGGAGAACCUGAUCAUCCUGGCGA |
| | | ACAACAGCCUGAGCAGCAACGGCAACGUGACCGAGAGCGGCUGUAAGGAGUG |
| | | CGAGGAGCUGGAGGAGAAGAAUAUCAAGGAGUUCCUGCAGAGCUUCGUGCAC |
| | | AUCGUGCAGAUGUUCAUCAACACCAGCUGAUUAAACCGGUACCGGUUGAUAA |
| | | UAGCAGACACCAUUGUCACACUCCAGCUGGAGCCUCGGUGGCCAUGCUUCUUG |
| | | CCCCUUGGGCCCAGACACCAUUGUCACACUCCAUCCCCCCAGCCCCUCCUCCCC |
| | | UUCCUGCACCCGUACCCCCCAGACACCAUUGUCACACUCCAGUGGUCUUUGAA |
| | | UAAAGUCUGAGUGGGCGGCGAAUUCGAUAUCCAGCGGCCGCGCUAGCGCUGG |
| | | CUAGCGAAUUCGAUAUCCAGCGGCCGCAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAA |
| SEQ ID | hIL- | AGGUCUUCUGGUCCCCACAGACUCAGAGAGAACCCGCCACCAUGGAGUUCGGC |
| NO: 65 | 15_verC_ | CUGAGCUGGGUGUUUCUGGUGGCCCUGUUCCGGGGCGUGCAGUGCAACUGGG |
| | down1 | UGAACGUGAUCAGCGACCUGAAGAAGAUCGAGGACCUGAUCCAGUCAAUGCA |
| | | CAUCGAUGCAACCCUGUACACCGAGAGCGACGUGCACCCUAGCUGUAAGGUGA |
| | | CCGCAAUGAAGUGCUUCCUGCUGGAGCUGCAGGUGAUAAGCCUGGAGAGCGG |
| | | CGACGCCAGCAUCCACGACACAGUCGAGAAUCUGAUCAUCCUGGCUAACAACA |
| | | GCCUGAGCAGCAAUGGUAAUGUGACCGAGAGCGGCUGCAAGGAGUGCGAGGA |
| | | GCUGGAGGAGAAGAACAUCAAGGAGUUCCUGCAGAGCUUCGUGCACAUCGUG |
| | | CAGAUGUUCAUCAACACCAGCGGCGGCAGCGGGGGCAGCGGCGGCUCAGGUGG |
| | | CAGCGGAGGCAGCGGCGGAAGCGGCGGCAUCACCUGCCCUCCCCCCAUGAGCG |
| | | UGGAGCACGCAGACAUCUGGGUGAAGAGCUACAGCCUGUACAGCCGGGAGCG |
| | | GUACAUCUGCAACAGCGGCUUCAAGCGGAAGGCCGGCACCAGCAGCCUGACCG |
| | | AGUGUGUGCUGAACAAGGCCACCAACGUGGCCCACUGGACAACCCCCAGCCUG |
| | | AAGUGCAUCAGGGACCCCGCCCUGGUGUGAUUAAACCGGUACCGGUUGAUAA |
| | | UAGCAGACACCAUUGUCACACUCCAGCUGGAGCCUCGGUGGCCAUGCUUCUUG |
| | | CCCCUUGGGCCCAGACACCAUUGUCACACUCCAUCCCCCCAGCCCCUCCUCCCC |
| | | UUCCUGCACCCGUACCCCCCAGACACCAUUGUCACACUCCAGUGGUCUUUGAA |
| | | UAAAGUCUGAGUGGGCGGCGAAUUCGAUAUCCAGCGGCCGCGCUAGCGCUGG |
| | | CUAGCGAAUUCGAUAUCCAGCGGCCGCAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAA |
| SEQ ID | mIL- | AGGUCUUCUGGUCCCCACAGACUCAGAGAGAACCCGCCACCAUGGCUAGCCCC |
| NO: 66 | 15_verA_ | CAGCUGCGGGGCUACGGCGUGCAGGCCAUCCCUGUGCUGCUGUUACUGCUGCU |
| | down1 | GCUCCUGCUGCUGCCCCUGCGGGUGACCCCCGGCACCACCUGCCCCCCCCCCGU |
| | | GAGCAUCGAGCACGCCGACAUCCGGGUGAAGAACUACAGCGUGAACAGCCGGG |
| | | AGAGGUACGUGUGCAAUAGCGGCUUCAAGCGGAAGGCCGGCACCAGCACCCUG |
| | | AUCGAGUGCGUGAUCAACAAGAACACCAACGUGGCCCACUGGACCACCCCCAG |
| | | CCUGAAGUGCAUCCGGGACCCCAGCUUAGCCCACUACAGCCCCGUGCCCACCG |
| | | UGGUGACCCCAAAGGUGACCAGCCAGCCCGAGAGCCCCAGCCCCAGCGCCAAG |
| | | GAGCCUGAGGCCUUCUCCCCAAAGAGCGACACCGCCAUGACCACCGAGACCGC |
| | | CAUCAUGCCCGGCAGCCGGCUGACCCCCAGCCAGACCACCAGCGCCGGCACCA |
| | | CAGGCACCGGCAGCCACAAGAGCAGCCGGGCACCCAGCCUGGCCGCCACCAUG |
| | | ACCCUGGAGCCUACCGCUAGCACCAGCCUGCGGAUCACCGAGAUCAGCCCCCA |
| | | CAGCAGCAAGAUGACCAAGGUGGCCAUCAGCACCAGCGUGCUGCUGGUGGGCG |
| | | CCGGCGUGGUGAUGGCUUUCCUGGCCUGGUACAUCAAGAGCCGGCAGCCCAGC |
| | | CAGCCUUGCCGGGUGGAGGUGGAGACCAUGGAGACUGUGCCCAUGACCGUGC |
| | | GGGCCAGCAGCAAGGAGGACGAGGACACCGGCGCCGGGGGCAGCGGCGGUAGC |
| | | GGAGGCAGCGGCGGCAGCGGCGGAAGCGGCGGCUCCGGCGGCAUGAAGAUCCU |
| | | GAAGCCCUACAUGCGGAACACCAGCAUCAGCUGCUACCUGUGCUUCCUGCUGA |
| | | ACAGCCACUUCCUGACCGAGGCCGGCAUCCACGUGUUCAUCCUGGGCUGCGUG |
| | | AGCGUGGGCCUGCCUAAGACCGAGGCCAACUGGAUCGACGUGCGGUACGACCU |
| | | GGAGAAGAUCGAGAGCCUGAUCCAGAGCAUCCACAUCGACACCACCCUGUACA |
| | | CCGACAGCGACUUCCACCCCAGCUGCAAGGUGACCGCCAUGAACUGCUUUCUG |
| | | CUGGAGCUGCAGGUGAUCCUGCACGAGUACAGCAACAUGACCCUGAACGAGAC |
| | | CGUGCGGAACGUGCUGUACCUGGCCAACAGCACCCUGAGCAGCAACAAGAACG |
| | | UGGCCGAGAGCGGCUGCAAGGAGUGCGAGGAGCUGGAGGAGAAGACCUUCAC |
| | | CGAGUUCCUGCAGAGCUUCAUCCGGAUCGUGCAGAUGUUCAUCAACACCAGCU |
| | | GAUUAAACCGGUACCGGUUGAUAAUAGCAGACACCAUUGUCACACUCCAGCU |
| | | GGAGCCUCGGUGGCCAUGCUUCUUGCCCCUUGGGCCCAGACACCAUUGUCACA |
| | | CUCCAUCCCCCCAGCCCCUCCUCCCCUUCCUGCACCCGUACCCCCCAGACACCA |
| | | UUGUCACACUCCAGUGGUCUUUGAAUAAAGUCUGAGUGGGCGGCGAAUUCGA |
| | | UAUCCAGCGGCCGCGCUAGCGCUGGCUAGCGAAUUCGAUAUCCAGCGGCCGCA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAA |
| SEQ ID | mIL- | AGGUCUUCUGGUCCCCACAGACUCAGAGAGAACCCGCCACCAUGAGCGUGCUG |
| NO: 67 | 15_verB_ | ACCCAGGUGCUGGCCCUGCUGCUGCUGUGGCUGACCGGCGCCCGUUGCACCAC |
| | down1 | CUGCCCCCCCCCAGUGAGCAUCGAGCACGCCGAUAUCCGGGUGAAGAACUACA |
| | | GCGUGAACAGCCGGGAGCGGUACGUGUGCAACAGCGGCUUCAAGCGGAAGGC |
| | | CGGCACAAGCACCCUGAUCGAGUGCGUGAUCAACAAGAACACCAAUGUGGCCC |
| | | ACUGGACCACCCCCAGCCUGAAGUGUAUCCGGGACCCUAGCCUGGCCGGCGGC |
| | | AGCGGCGGGAGCGGUGGCAGCGGCGGAAGCGGCGGUAGCGGAGGCAGCGGCG |
| | | GCAACUGGAUCGACGUGCGGUACGACCUAGAGAAGAUCGAGAGCCUGAUCCA |
| | | GAGCAUCCACAUCGACACCACCCUGUACACCGACAGCGACUUCCACCCCAGCU |
| | | GCAAGGUGACCGCCAUGAACUGCUUCCUGCUGGAGCUGCAGGUGAUCCUGCAC |
| | | GAGUACAGCAACAUGACACUGAACGAGACCGUGCGGAACGUGCUGUACCUGG |
| | | CAAACAGCACCCUGAGCAGCAACAAGAAUGUGGCCGAGAGCGGCUGCAAGGA |
| | | AUGCGAGGAGCUGGAGGAGAAGACCUUCACCGAGUUCCUCCAGAGCUUCAUCC |
| | | GGAUCGUGCAGAUGUUCAUCAACACCAGCUGAUUAAACCGGUACCGGUUGAU |
| | | AAUAGCAGACACCAUUGUCACACUCCAGCUGGAGCCUCGGUGGCCAUGCUUCU |
| | | UGCCCCUUGGGCCCAGACACCAUUGUCACACUCCAUCCCCCCAGCCCCUCCUCC |
| | | CCUUCCUGCACCCGUACCCCCCAGACACCAUUGUCACACUCCAGUGGUCUUUG |
| | | AAUAAAGUCUGAGUGGGCGGCGAAUUCGAUAUCCAGCGGCCGCGCUAGCGCU |
| | | GGCUAGCGAAUUCGAUAUCCAGCGGCCGCAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAA |
| SEQ ID | mIL- | AGGUCUUCUGGUCCCCACAGACUCAGAGAGAACCCGCCACCAUGAGCGUGCUG |
| NO: 68 | 15_verC_ | ACCCAGGUGCUGGCUCUACUGCUGCUGUGGUUAACAGGCGCCCGGUGCAACUG |
| | down1 | GAUCGACGUGCGGUACGACCUGGAGAAGAUCGAGAGCCUGAUCCAGAGCAUC |
| | | CACAUCGACACCACCCUGUACACCGACAGCGACUUCCACCCUAGCUGCAAGGU |
| | | GACCGCCAUGAACUGCUUCCUGCUGGAGCUGCAGGUGAUCCUGCACGAGUACA |
| | | GCAAUAUGACCCUGAACGAGACCGUGCGGAACGUGCUGUACCUGGCCAACAGC |
| | | ACCCUGAGCAGCAAUAAGAAUGUGGCCGAGAGCGGCUGCAAGGAGUGUGAGG |
| | | AGCUGGAGGAGAAGACCUUCACUGAGUUCCUGCAGAGCUUUAUCCGGAUCGU |
| | | GCAGAUGUUCAUCAACACUAGCGGCGGAAGCGGCGGCUCAGGCGGCAGCGGA |
| | | GGCAGCGGCGGCAGCGGGGGCAGCGGCGGAACCACCUGUCCCCCCCCCGUGAG |
| | | CAUCGAGCACGCCGACAUCCGGGUGAAGAACUACAGCGUGAACUCACGGGAGC |
| | | GGUACGUGUGCAACUCAGGCUUCAAGCGGAAGGCCGGCACCAGCACCCUGAUC |
| | | GAGUGCGUGAUCAACAAGAACACAAACGUGGCCCACUGGACCACCCCCAGCCU |
| | | GAAGUGCAUCCGGGACCCCAGCCUGGCCUGAUUAAACCGGUACCGGUUGAUAA |
| | | UAGCAGACACCAUUGUCACACUCCAGCUGGAGCCUCGGUGGCCAUGCUUCUUG |
| | | CCCCUUGGGCCCAGACACCAUUGUCACACUCCAUCCCCCCAGCCCCUCCUCCCC |
| | | UUCCUGCACCCGUACCCCCCAGACACCAUUGUCACACUCCAGUGGUCUUUGAA |
| | | UAAAGUCUGAGUGGGCGGCGAAUUCGAUAUCCAGCGGCCGCGCUAGCGCUGG |
| | | CUAGCGAAUUCGAUAUCCAGCGGCCGCAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAA |
| SEQ ID | mIL- | AGGUCUUCUGGUCCCCACAGACUCAGAGAGAACCCGCCACCAUGAGCGUGCUG |
| NO: 69 | 15_verD_ | ACCCAGGUGCUGGCCCUGCUGCUGCUGUGGCUGACCGGCGCCCGGUGCAACUG |
| | down1 | GAUCGACGUGCGGUACGAUCUGGAGAAGAUCGAGAGCCUGAUCCAGAGCAUC |
| | | CACAUCGACACCACCCUGUACACCGAUAGCGACUUCCACCCCAGCUGCAAGGU |
| | | GACCGCCAUGAACUGCUUCCUGCUGGAACUGCAGGUGAUCCUGCACGAGUACA |
| | | GCAACAUGACCCUGAACGAGACCGUGCGGAACGUCCUGUACCUGGCCAACAGC |
| | | ACACUGAGCAGCAACAAGAACGUGGCCGAGAGCGGCUGCAAGGAGUGCGAGG |
| | | AGCUGGAGGAGAAGACCUUCACCGAGUUCCUGCAGAGCUUCAUCCGUAUCGU |
| | | GCAGAUGUUCAUCAACACCAGCGGGGGCAGCGGCGGAAGCGGGGGCAGCGGC |
| | | GGAAGCGGCGGCAGCGGAGGCAGCGGAGGCACCACAUGCCCCCCACCCGUGAG |
| | | CAUCGAGCACGCCGACAUACGGGUGAAGAAUUACAGCGUGAACAGCCGGGAG |
| | | CGGUACGUGUGCAACAGCGGCUUCAAGCGGAAGGCCGGCACCAGCACCCUGAU |
| | | CGAGUGCGUGAUCAACAAGAACACUAACGUGGCACACUGGACCACCCCCAGCC |
| | | UCAAGUGCAUCCGGGACCCAAGCCUGGCCGGGGGCGGCGGCUCCGGCGGAGGC |
| | | GGCAGCGGCGGUGGAGGCAGCGGCCCUACAAUCAAGCCCUGCCCCCCCUGCAA |
| | | GUGCCCCGCCCCCAAUGCCGCCGGCGGCCCCAGCGUGUUCAUCUUCCCCCCCAA |
| | | GAUCAAGGACGUGCUGAUGAUCUCACUAAGCCCCAUAGUGACCUGCGUGGUG |
| | | GUGGACGUGAGCGAGGACGACCCUGACGUGCAGAUCAGCUGGUUCGUGAACA |
| | | ACGUGGAGGUGCACACCGCCCAGACCCAGACCCACCGGGAGGACUACAACAGC |
| | | ACCCUGCGGGUGGUGAGCGCCCUGCCCAUCCAGCACCAGGACUGGAUGAGCGG |
| | | CAAGGAGUUCAAGUGUAAGGUGAAUAAUAAGGACCUGGGCGCCCCAAUAGAG |
| | | CGGACCAUUAGCAAGCCCAAGGGGAGCGUGCGGGCCCCCCAGGUGUACGUGCU |
| | | GCCACCCCCUGAGGAGGAGAUGACCAAAAAGCAGGUGACCCUGACCUGCAUGG |
| | | UGACCGAUUUCAUGCCAGAGGACAUCUACGUGGAGUGGACCAACAACGGCAA |
| | | GACCGAGCUGAACUACAAGUGAUUAAACCGGUACCGGUUGAUAAUAGCAGAC |
| | | ACCAUUGUCACACUCCAGCUGGAGCCUCGGUGGCCAUGCUUCUUGCCCCUUGG |
| | | GCCCAGACACCAUUGUCACACUCCAUCCCCCCAGCCCCUCCUCCCCUUCCUGCA |
| | | CCCGUACCCCCCAGACACCAUUGUCACACUCCAGUGGUCUUUGAAUAAAGUCU |
| | | GAGUGGGCGGCGAAUUCGAUAUCCAGCGGCCGCGCUAGCGCUGGCUAGCGAA |
| | | UUCGAUAUCCAGCGGCCGCAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAA |
| SEQ ID | hIL- | AGGUCUUCUGGUCCCCACAGACUCAGAGAGAACCCGCCACCAUGUGCCACCAG |
| NO: 70 | 12_down1 | CAGCUGGUGAUCAGCUGGUUCAGCCUGGUGUUCCUCGCCAGCCCCCUGGUGGC |
| | | UAUCUGGGAGCUGAAGAAGGACGUGUACGUGGUGGAGCUGGACUGGUACCCC |
| | | GACGCCCCCGGCGAGAUGGUGGUGCUCACCUGCGAUACCCCCGAGGAGGACGG |
| | | AAUCACCUGGACCCUGGACCAGAGCAGCGAGGUGUUAGGCAGCGGCAAGACCC |
| | | UGACCAUCCAGGUGAAGGAGUUCGGCGACGCCGGCCAGUACACCUGCCACAAG |
| | | GGCGGGGAGGUGCUGAGCCACAGCCUGCUGCUGCUGCACAAGAAGGAGGACG |
| | | GCAUCUGGAGCACCGACAUCCUGAAGGACCAGAAGGAGCCCAAGAACAAGACC |
| | | UUCCUGCGGUGCGAGGCCAAGAAUUACAGCGGCCGGUUCACCUGCUGGUGGCU |
| | | GACCACCAUCAGCACCGACCUGACCUUCAGCGUGAAGAGCAGCCGGGGCAGCA |
| | | GCGACCCCCAGGGCGUGACCUGCGGCGCCGCCACCCUGAGCGCCGAGCGGGUG |
| | | AGGGGCGACAAUAAGGAGUACGAGUACAGCGUGGAGUGUCAGGAGGAUAGCG |
| | | CAUGCCCCGCCGCCGAGGAGAGCCUGCCCAUCGAGGUGAUGGUGGACGCCGUG |
| | | CACAAGCUGAAGUACGAGAACUACACCAGCAGCUUCUUCAUCCGGGACAUCAU |
| | | CAAGCCCGACCCCCCCAAGAACCUGCAGCUGAAGCCCCUGAAGAACAGCCGGC |
| | | AGGUGGAGGUGUCAUGGGAGUACCCCGACACCUGGAGCACCCCCCACAGCUAC |
| | | UUCAGCCUGACAUUCUGUGUGCAGGUGCAGGGCAAGAGCAAGCGGGAGAAGA |
| | | AGGACCGGGUGUUCACCGACAAGACCAGCGCCACCGUGAUCUGCCGGAAGAAU |
| | | GCCAGCAUCAGCGUGCGGGCCCAGGACCGGUACUACAGCAGCAGCUGGAGCGA |
| | | GUGGGCCAGCGUGCCUUGCUCCGGCGGCGGAGGAAGCGGAGGCGGGGGCAGC |
| | | GGGGGCGGCGGAAGCCGGAACCUGCCCGUGGCCACCCCAGACCCCGGCAUGUU |
| | | CCCCUGCCUGCACCACAGCCAGAACCUGCUGCGGGCCGUGAGCAACAUGCUGC |
| | | AGAAGGCUCGGCAGACCCUGGAGUUCUACCCCUGCACCAGCGAGGAGAUCGAC |
| | | CACGAGGACAUCACCAAGGACAAGACUAGCACCGUGGAGGCCUGCCUGCCCCU |
| | | GGAGCUGACCAAGAAUGAGAGCUGCCUGAACAGCCGGGAGACCAGCUUUAUC |
| | | ACCAACGGCAGCUGCCUGGCCAGCCGGAAGACCUCAUUCAUGAUGGCCCUGUG |
| | | CCUGAGCAGCAUCUACGAGGACCUGAAGAUGUACCAGGUGGAGUUCAAGACC |
| | | AUGAACGCCAAGCUGCUGAUGGACCCUAAGCGGCAAAUCUUCCUGGACCAGAA |
| | | UAUGCUGGCCGUGAUCGACGAGCUCAUGCAGGCCCUGAACUUUAACAGCGAG |
| | | ACCGUGCCCCAGAAGAGCAGCCUGGAGGAGCCAGACUUCUACAAGACCAAGAU |
| | | CAAGCUGUGCAUCCUGCUGCACGCUUUCCGGAUCCGGGCCGUGACCAUCGACC |
| | | GGGUGAUGAGCUACCUGAACGCGAGCAGCGGAGGCGGCAGCGGUGGCGGCGG |
| | | CUCAGGCGGCGGAGGCAGCGGCGGAGGCGGAAGCGGCGGCGGCAGCCUGCAGG |
| | | UGGUGAUCAGCGCCAUCCUGGCUCUGGUGGUGCUGACCAUCAUCUCCCUGAUC |
| | | AUCCUGAUCGGCGGAGGUGGCAGCUGAUUAAACCGGUACCGGUUGAUAAUAG |
| | | CAGACACCAUUGUCACACUCCAGCUGGAGCCUCGGUGGCCAUGCUUCUUGCCC |
| | | CUUGGGCCCAGACACCAUUGUCACACUCCAUCCCCCCAGCCCCUCCUCCCCUUC |
| | | CUGCACCCGUACCCCCCAGACACCAUUGUCACACUCCAGUGGUCUUUGAAUAA |
| | | AGUCUGAGUGGGCGGCGAAUUCGAUAUCCAGCGGCCGCGCUAGCGCUGGCUA |
| | | GCGAAUUCGAUAUCCAGCGGCCGCAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAA |
| SEQ ID | mIL- | AGGUCUUCUGGUCCCCACAGACUCAGAGAGAACCCGCCACCAUGUGCCCCCAG |
| NO: 71 | 12_down | AAGCUGACCAUCAGCUGGUUCGCCAUCGUGCUGCUGGUGAGCCCUCUGAUGGC |
| | 1 | CAUGUGGGAGCUGGAGAAGGACGUGUACGUGGUGGAGGUGGAUUGGACCCCC |
| | | GACGCACCCGGCGAGACCGUGAACCUGACCUGCGAUACCCCUGAGGAGGACGA |
| | | CAUCACCUGGACUAGCGAUCAGCGGCACGGCGUGAUCGGCAGCGGCAAGACCC |
| | | UGACCAUCACCGUGAAGGAGUUCCUGGAUGCCGGCCAGUACACCUGCCACAAG |
| | | GGCGGGGAGACCCUGAGCCACAGCCACCUGCUGCUGCACAAAAAGGAGAACGG |
| | | CAUCUGGAGCACCGAGAUCCUGAAGAACUUCAAGAACAAGACCUUUCUGAAG |
| | | UGCGAGGCUCCCAACUACAGCGGUCGGUUCACCUGCAGCUGGCUGGUGCAGCG |
| | | GAAUAUGGACCUGAAGUUUAACAUCAAGUCAAGCAGCAGCAGCCCCGACAGCC |
| | | GGGCCGUGACCUGUGGCAUGGCCAGCCUGAGCGCCGAGAAGGUGACCCUGGAC |
| | | CAGAGGGAUUACGAGAAGUACAGCGUGAGCUGCCAGGAGGACGUGACCUGCC |
| | | CAACCGCCGAGGAGACCCUGCCCAUCGAGCUGGCCCUGGAGGCCCGGCAGCAG |
| | | AACAAGUACGAGAACUACAGCACCAGCUUCUUCAUCCGGGACAUCAUCAAGCC |
| | | UGACCCCCCCAAGAACCUGCAGAUGAAGCCCCUGAAGAACAGCCAGGUGGAGG |
| | | UGAGCUGGGAGUACCCCGACAGCUGGAGCACCCCCCACAGCUACUUCAGCCUG |
| | | AAGUUCUUCGUGCGGAUCCAGCGGAAGAAGGAGAAGAUGAAGGAGACCGAGG |
| | | AGGGCUGCAACCAGAAGGGCGCCUUCCUGGUGGAGAAGACCAGCACCGAGGU |
| | | GCAGUGUAAGGGCGGCAACGUGUGCGUGCAGGCCCAGGACCGGUACUACAAC |
| | | AGCUCAUGCAGCAAGUGGGCCUGCGUGCCCUGCCGGGUGAGGAGCGGCGGCGG |
| | | CGGCUCCGGCGGAGGAGGCAGCGGAGGAGGCGGCAGCCGGGUGAUCCCCGUGA |
| | | GCGGCCCUGCCCGGUGCCUGAGCCAGAGCCGGAACCUGCUGAAGACCACCGAC |
| | | GACAUGGUGAAGACCGCUCGGGAGAAGCUGAAGCACUACAGCUGCACCGCCGA |
| | | GGACAUCGACCACGAGGACAUCACCCGGGACCAGACCAGCACCUUAAAGACCU |
| | | GCCUGCCCCUGGAGCUGCACAAGAAUGAGAGCUGCCUGGCCACCCGGGAGACC |
| | | AGCAGCACCACCCGGGGCAGCUGCCUGCCCCCCCAGAAGACCAGCCUGAUGAU |
| | | GACCCUGUGCCUGGGCAGCAUCUACGAGGACCUGAAGAUGUACCAGACCGAGU |
| | | UCCAGGCAAUCAACGCUGCUCUGCAGAAUCACAACCACCAGCAGAUCAUCCUG |
| | | GACAAGGGAAUGCUGGUGGCCAUCGACGAGCUGAUGCAGAGCCUGAACCACA |
| | | ACGGCGAGACCCUGCGGCAGAAGCCCCCCGUGGGCGAGGCCGACCCCUACCGU |
| | | GUGAAGAUGAAGCUGUGCAUCCUGCUGCACGCUUUCAGCACCCGGGUGGUGA |
| | | CCAUCAACCGGGUGAUGGGCUACCUGAGCAGCGCAAGCGGUGGCGGCAGCGGC |
| | | GGCGGGGGCUCCGGCGGCGGUGGGAGCGGCGGAGGCGGUUCAGGCGGCGGCA |
| | | GCCUGCAGGUGGUGAUCAGCGCCAUCCUGGCCCUGGUGGUGCUCACCGUGAUC |
| | | AGCCUGAUCAUCCUGAUCGGGGGGGGCGGCUCAGGCAAGCCCAUCCCAAAUCC |
| | | ACUGCUGGGCCUGGACAGCACCUGAUUAAACCGGUACCGGUUGAUAAUAGCA |
| | | GACACCAUUGUCACACUCCAGCUGGAGCCUCGGUGGCCAUGCUUCUUGCCCCU |
| | | UGGGCCCAGACACCAUUGUCACACUCCAUCCCCCCAGCCCCUCCUCCCCUUCCU |
| | | GCACCCGUACCCCCCAGACACCAUUGUCACACUCCAGUGGUCUUUGAAUAAAG |
| | | UCUGAGUGGGGGCGAAUUCGAUAUCCAGCGGCCGCGCUAGCGCUGGCUAGCG |
| | | AAUUCGAUAUCCAGCGGCCGCAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAA |
| SEQ ID | TET-IL- | AGGUCUUCUGGUCCCCACAGACUCAGAGAGAACCCGCCACCAUGUGCCCCCAG |
| NO: 72 | 12_down1 | AAGCUGACCAUCAGCUGGUUCGCCAUCGUGCUGCUGGUGAGCCCUCUGAUGGC |
| | | CAUGUGGGAGCUCGAGAAGGACGUGUACGUGGUGGAGGUGGACUGGACCCCC |
| | | GACGCCCCCGGCGAGACCGUGAACCUGACCUGUGACACCCCCGAGGAGGACGA |
| | | CAUCACCUGGACCAGCGACCAGCGGCACGGCGUGAUCGGAAGCGGCAAGACCC |
| | | UGACCAUCACCGUCAAGGAGUUCCUGGACGCCGGCCAGUACACCUGCCACAAG |
| | | GGCGGCGAGACACUGAGCCACAGCCACCUGCUGUUACACAAGAAGGAGAACGG |
| | | CAUCUGGAGCACCGAGAUUCUGAAGAACUUCAAGAACAAGACCUUCCUGAAG |
| | | UGCGAAGCCCCCAAUUACAGCGGCCGGUUCACCUGCAGCUGGCUGGUGCAGCG |
| | | GAACAUGGACCUGAAGUUCAAUAUCAAGUCAAGCAGCAGCAGCCCCGAUAGCC |
| | | GGGCCGUGACCUGCGGCAUGGCCAGCCUGAGCGCCGAGAAGGUGACCCUGGAC |
| | | CAGAGGGACUACGAGAAGUACAGCGUGAGCUGUCAGGAGGACGUGACAUGCC |
| | | CAACCGCCGAGGAGACCCUGCCCAUCGAGCUGGCCCUGGAGGCCCGGCAGCAG |
| | | AAUAAGUACGAGAAUUACAGCACCAGCUUCUUCAUCCGGGACAUCAUCAAGCC |
| | | CGACCCCCCUAAGAACCUGCAGAUGAAGCCCCUGAAGAAUAGCCAGGUCGAGG |
| | | UGAGCUGGGAGUACCCCGAUAGCUGGAGCACCCCCCACAGCUACUUCAGCCUG |
| | | AAGUUCUUCGUGCGGAUCCAGCGGAAGAAGGAGAAGAUGAAGGAGACCGAGG |
| | | AGGGCUGCAAUCAGAAGGGCGCAUUCCUGGUGGAGAAGACCAGCACCGAGGU |
| | | GCAGUGCAAGGGCGGCAACGUGUGCGUGCAGGCCCAGGAUCGGUACUACAAC |
| | | AGCAGCUGCAGCAAGUGGGCCUGCGUGCCAUGCCGGGUGCGGAGCGGCGCCGG |
| | | AGGCUCAGGUGGCGGCGGCUCAGGCGGGGGCGGCAGCCGGGUGAUCCCCGUGA |
| | | GCGGCCCCGCCCGGUGCCUUAGCCAGAGCCGGAAUCUGCUGAAGACCACCGAC |
| | | GACAUGGUGAAGACCGCCCGGGAGAAGCUGAAGCACUACAGCUGCACCGCCGA |
| | | GGACAUCGACCACGAGGACAUCACCCGGGACCAGACCAGCACCCUGAAGACCU |
| | | GCCUGCCCCUGGAGCUGCACAAGAACGAGAGCUGCCUGGCAACCCGGGAGACC |
| | | AGCAGCACCACCCGGGGCAGCUGCCUCCCCCCCCAGAAGACCAGCCUGAUGAU |
| | | GACCCUGUGCCUGGGGAGCAUCUACGAGGACCUGAAGAUGUACCAGACCGAG |
| | | UUCCAGGCUAUAAACGCCGCCCUGCAGAACCACAACCACCAGCAGAUCAUCCU |
| | | GGACAAGGGCAUGCUGGUGGCCAUCGACGAGCUGAUGCAGAGCCUGAACCAC |
| | | AACGGCGAGACUCUGCGGCAGAAGCCCCCUGUGGGCGAGGCCGAUCCCUACCG |
| | | GGUGAAGAUGAAGCUGUGCAUCCUGCUGCACGCCUUCAGCACCCGGGUGGUG |
| | | ACCAUCAACAGGGUGAUGGGCUACCUGAGCAGCGCUAGCGGCGGAGGUAGCG |
| | | GAGGCGGCGGCUCCGGCGGCGGCGGCAGCGGUGGAGGCGGCAGCGGCGGCGGU |
| | | AGCCUGCAAGUGGUGAUCAGCGCCAUCCUGGCCCUGGUGGUGCUGACCGUGAU |
| | | CAGCCUGAUCAUCCUGAUCGGCGGUGGCGGGAGCGGCAAGCCCAUCCCCAACC |
| | | CACUGCUGGGCCUGGACAGCACCUGAUUAAACCGGUACCGGUUGAUAAUAGC |
| | | AGACACCAUUGUCACACUCCAGCUGGAGCCUCGGUGGCCAUGCUUCUUGCCCC |
| | | UUGGGCCCAGACACCAUUGUCACACUCCAUCCCCCCAGCCCCUCCUCCCCUUCC |
| | | UGCACCCGUACCCCCCAGACACCAUUGUCACACUCCAGUGGUCUUUGAAUAAA |
| | | GUCUGAGUGGGCGGCGAAUUCGAUAUCCAGCGGCCGCGCUAGCGCUGGCUAGC |
| | | GAAUUCGAUAUCCAGCGGCCGCAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAA |
| SEQ ID | hCX40L_ | AGGUCUUCUGGUCCCCACAGACUCAGAGAGAACCCGCCACCUGGAGCGGGUGC |
| NO: 73 | down1 | AGCCCCUGGAGGAGAACGUGGGCAACGCCGCCCGGCCCCGGUUCGAGCGGAAC |
| | | AAGCUGCUGCUGGUGGCCAGCGUGAUCCAGGGCCUGGGCCUGCUGCUGUGCUU |
| | | CACCUACAUCUGCCUGCACUUCAGCGCCCUGCAGGUGAGCCACAGGUACCCCC |
| | | GGAUCCAGAGCAUCAAGGUGCAGUUCACCGAGUACAAGAAGGAGAAGGGCUU |
| | | CAUCCUGACCAGCCAGAAGGAGGACGAGAUCAUGAAGGUGCAGAACAACAGC |
| | | GUGAUCAUCAACUGCGACGGCUUCUACCUGAUCUCACUGAAGGGCUACUUCAG |
| | | CCAGGAGGUGAACAUCAGCCUGCACUACCAAAAGGACGAGGAGCCCCUGUUCC |
| | | AGCUGAAGAAGGUGCGGAGCGUGAACAGCCUGAUGGUGGCCUCACUGACCUA |
| | | CAAGGACAAGGUGUACCUUAACGUGACCACCGACAACACCAGCCUGGACGACU |
| | | UCCACGUGAACGGCGGCGAGCUGAUCCUGAUUCACCAGAACCCUGGCGAGUUC |
| | | UGCGUGCUGUGAUUAAACCGGUACCGGUUGAUAAUAGCAGACACCAUUGUCA |
| | | CACUCCAGCUGGAGCCUCGGUGGCCAUGCUUCUUGCCCCUUGGGCCCAGACAC |
| | | CAUUGUCACACUCCAUCCCCCCAGCCCCUCCUCCCCUUCCUGCACCCGUACCCC |
| | | CCAGACACCAUUGUCACACUCCAGUGGUCUUUGAAUAAAGUCUGAGUGGGCG |
| | | GCGAAUUCGAUAUCCAGCGGCCGCGCUAGCGCUGGCUAGCGAAUUCGAUAUCC |
| | | AGCGGCCGCAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAA |
| SEQ ID | mCX40L_ | AGGUCUUCUGGUCCCCACAGACUCAGAGAGAACCCGCCACCAUGGAGGGCGAG |
| NO: 74 | down1 | GGCGUGCAGCCACUGGAUGAGAACCUGGAGAACGGCAGCCGGCCCCGGUUCAA |
| | | GUGGAAGAAGACCUUACGUCUGGUGGUGAGCGGCAUCAAGGGCGCCGGCAUG |
| | | CUGCUGUGCUUCAUCUACGUGUGUCUGCAGCUGAGCAGCAGCCCCGCCAAGGA |
| | | CCCCCCUAUCCAGCGGCUGCGGGGCGCCGUGACCCGGUGCGAGGACGGCCAGC |
| | | UGUUCAUCAGCAGCUACAAGAACGAGUACCAGACCAUGGAGGUGCAGAACAA |
| | | CAGCGUGGUGAUCAAGUGCGACGGCCUGUACAUCAUCUACCUGAAGGGCAGC |
| | | UUCUUCCAGGAAGUGAAGAUCGACCUACACUUCAGGGAGGAUCACAACCCCAU |
| | | CAGCAUCCCCAUGCUGAACGACGGCCGGCGGAUCGUGUUCACCGUGGUGGCCA |
| | | GCCUGGCCUUCAAGGACAAGGUGUACCUGACCGUGAACGCCCCCGACACCCUG |
| | | UGUGAGCACCUGCAGAUCAACGACGGCGAGCUGAUCGUGGUGCAGCUGACUCC |
| | | CGGCUACUGCGCCCCCGAGGGCAGCUACCACAGCACCGUGAACCAGGUGCCCC |
| | | UUUGAUUAAACCGGUACCGGUUGAUAAUAGCAGACACCAUUGUCACACUCCA |
| | | GCUGGAGCCUCGGUGGCCAUGCUUCUUGCCCCUUGGGCCCAGACACCAUUGUC |
| | | ACACUCCAUCCCCCCAGCCCCUCCUCCCCUUCCUGCACCCGUACCCCCCAGACA |
| | | CCAUUGUCACACUCCAGUGGUCUUUGAAUAAAGUCUGAGUGGGCGGCGAAUU |
| | | CGAUAUCCAGCGGCCGCGCUAGCGCUGGCUAGCGAAUUCGAUAUCCAGCGGCC |
| | | GCAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAA |
| SEQ ID | hIFN- | AGGUCUUCUGGUCCCCACAGACUCAGAGAGAACCCGCCACCAUGGCCCUGACC |
| NO: 75 | A_down1 | UUCGCCCUGUUAGUGGCGCUGCUGGUGCUGAGCUGCAAGAGCAGCUGCAGCG |
| | | UGGGCUGCGACCUGCCUCAGACCCACAGCCUGGGCAGCCGGCGGACCCUGAUG |
| | | CUGCUGGCCCAGAUGCGGAAGAUCAGCCUGUUCAGCUGUCUGAAGGACCGGCA |
| | | CGACUUCGGCUUCCCCCAGGAGGAAUUCGGCAACCAGUUCCAGAAGGCCGAGA |
| | | CCAUCCCCGUGCUGCACGAGAUGAUCCAGCAGAUCUUCAACCUGUUCAGCACC |
| | | AAGGAUAGCAGCGCCGCUUGGGACGAGACCCUUCUGGACAAGUUCUACACCGA |
| | | GCUGUACCAGCAGCUGAACGACCUGGAGGCCUGCGUGAUCCAGGGCGUGGGCG |
| | | UGACCGAGACCCCUCUGAUGAAGGAGGACAGCAUCCUGGCCGUGCGGAAGUAC |
| | | UUUCAGCGGAUCACCCUGUACCUGAAGGAGAAGAAGUACAGCCCAUGCGCCUG |
| | | GGAGGUGGUGCGGGCCGAGAUCAUGCGGAGCUUCAGCCUGAGCACCAACCUGC |
| | | AGGAGAGCCUGCGGAGCAAGGAGUGAUUAAACCGGUACCGGUUGAUAAUAGC |
| | | AGACACCAUUGUCACACUCCAGCUGGAGCCUCGGUGGCCAUGCUUCUUGCCCC |
| | | UUGGGCCCAGACACCAUUGUCACACUCCAUCCCCCCAGCCCCUCCUCCCCUUCC |
| | | UGCACCCGUACCCCCCAGACACCAUUGUCACACUCCAGUGGUCUUUGAAUAAA |
| | | GUCUGAGUGGGCGGCGAAUUCGAUAUCCAGCGGCCGCGCUAGCGCUGGCUAGC |
| | | GAAUUCGAUAUCCAGCGGCCGCAAAAAAAAAAAAAAAAAAAAAAAAAAAAAA |
| | | AAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAA |
| SEQ ID | IFN- | AGGUCUUCUGGUCCCCACAGACUCAGAGAGAACCCGCCACCAUGGCUCGGCUG |
| NO: 76 | down1 | UGCGCCUUCCUGAUGAUCCUGGUGAUGAUGAGCUACUACUGGAGCGCAUGCA |
| | | GCCUGGGCUGCGACCUGCCACACACCUACAACCUGGGCAAUAAGCGGGCUCUG |
| | | ACCGUGCUGGAGGAGAUGCGGCGGCUGCCCCCCCUGAGCUGCCUGAAGGACCG |
| | | GAAGGACUUCGGCUUCCCCCUGGAGAAGGUGGACAACCAGCAGAUCCAAAAG |
| | | GCACAGGCCAUCCUGGUGCUGCGGGACCUGACCCAGCAGAUCCUGAACCUGUU |
| | | CACCAGCAAGGACCUGAGCGCCACCUGGAACGCCACCCUGCUGGACAGCUUCU |
| | | GCAACGACCUGCACCAGCAGCUGAACGACCUGAAGGCCUGCGUCAUGCAGGAG |
| | | CCACCACUGACCCAGGAGGACAGCCUGUUAGCUGUGCGGACUUACUUCCACCG |
| | | GAUCACCGUGUACCUGCGGAAGAAGAAGCACAGCCUGUGCGCUUGGGAGGUG |
| | | AUCCGGGCCGAGGUGUGGCGGGCCCUGAGCAGCAGCACCAACCUGCUGGCCCG |
| | | GCUGAGCGAGGAGAAGGAGUGAUUAAACCGGUACCGGUUGAUAAUAGCAGAC |
| | | ACCAUUGUCACACUCCAGCUGGAGCCUCGGUGGCCAUGCUUCUUGCCCCUUGG |
| | | GCCCAGACACCAUUGUCACACUCCAUCCCCCCAGCCCCUCCUCCCCUUCCUGCA |
| | | CCCGUACCCCCCAGACACCAUUGUCACACUCCAGUGGUCUUUGAAUAAAGUCU |
| | | GAGUGGGCGGCGAAUUCGAUAUCCAGCGGCCGCGCUAGCGCUGGCUAGCGAA |
| | | UUCGAUAUCCAGCGGCCGCAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAA |
| SEQ ID | IL- | AGGUCUUCUGGUCCCCACAGACUCAGAGAGAACCCGCCACCAUGUACCGGAUG |
| NO: 77 | 2_down1 | CAGCUGCUGAGCUGCAUCGCCCUGAGCCUGGCCCUGGUGACCAACAGCGCCCC |
| | | CACCAGCAGCAGCACCAAGAAGACCCAGCUGCAACUGGAGCACCUGCUGCUGG |
| | | AUCUGCAGAUGAUCCUGAACGGCAUCAACAAUUACAAGAAUCCCAAGCUGACC |
| | | CGGAUGCUGACCUUCAAGUUCUACAUGCCCAAGAAGGCUACCGAGCUGAAGCA |
| | | CCUGCAGUGCCUGGAGGAGGAGCUGAAGCCCCUGGAGGAGGUGCUGAACCUG |
| | | GCCCAGAGCAAGAACUUCCACUUCGACCCCCGGGACGUGGUGAGCAACAUCAA |
| | | CGUGUUCGUGCUGGAGCUGAAGGGCAGCGAGACCACCUUCAUGUGCGAGUAC |
| | | GCCGACGAGACCGCCACCAUCGUGGAGUUCCUGAACCGGUGGAUCACCUUCUG |
| | | CCAGAGCAUCAUCAGCUGAUUAAACCGGUACCGGUUGAUAAUAGCAGACACC |
| | | AUUGUCACACUCCAGCUGGAGCCUCGGUGGCCAUGCUUCUUGCCCCUUGGGCC |
| | | CAGACACCAUUGUCACACUCCAUCCCCCCAGCCCCUCCUCCCCUUCCUGCACCC |
| | | GUACCCCCCAGACACCAUUGUCACACUCCAGUGGUCUUUGAAUAAAGUCUGAG |
| | | UGGGCGGCGAAUUCGAUAUCCAGCGGCCGCGCUAGCGCUGGCUAGCGAAUUCG |
| | | AUAUCCAGCGGCCGCAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAA |
| SEQ ID | mIL- | AGGUCUUCUGGUCCCCACAGACUCAGAGAGAACCCGCCACCAUGUACAGCAUG |
| NO: 78 | 2_down1 | CAGCUGGCAAGCUGCGUGACCCUGACCCUGGUGCUGCUAGUGAACAGCGCCCC |
| | | CACCAGCAGCAGCACCAGCAGCUCCACCGCCGAGGCCCAGCAGCAACAACAGC |
| | | AACAGCAGCAGCAGCAACAGCACCUGGAGCAGCUCCUGAUGGACCUGCAGGAG |
| | | CUGCUGAGCCGGAUGGAGAACUACCGGAACCUGAAGCUGCCCCGGAUGCUGAC |
| | | CUUCAAGUUCUACCUGCCCAAGCAGGCCACAGAGCUGAAGGACCUGCAGUGCC |
| | | UGGAGGACGAGCUGGGCCCCCUGCGGCACGUGCUGGAUCUGACCCAGAGCAAG |
| | | AGCUUCCAGCUGGAGGACGCCGAGAACUUCAUCAGCAACAUCCGGGUGACCGU |
| | | GGUGAAGCUGAAGGGCAGCGACAACACCUUCGAGUGCCAGUUCGACGACGAG |
| | | AGCGCAACCGUGGUGGACUUCCUGCGGCGGUGGAUCGCCUUCUGCCAGAGCAU |
| | | CAUCAGCACCAGCCCCCAGUGAUUAAACCGGUACCGGUUGAUAAUAGCAGACA |
| | | CCAUUGUCACACUCCAGCUGGAGCCUCGGUGGCCAUGCUUCUUGCCCCUUGGG |
| | | CCCAGACACCAUUGUCACACUCCAUCCCCCCAGCCCCUCCUCCCCUUCCUGCAC |
| | | CCGUACCCCCCAGACACCAUUGUCACACUCCAGUGGUCUUUGAAUAAAGUCUG |
| | | AGUGGGCGGCGAAUUCGAUAUCCAGCGGCCGCGCUAGCGCUGGCUAGCGAAU |
| | | UCGAUAUCCAGCGGCCGCAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAA |
| SEQ ID | U27-hIL- | AGGUCUUCUGGUCCCCACAGACUCAGAGAGAACCCGCCACCaugAAGUGAGGCU |
| NO: 79 | 15_verA_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCCCCCCGGCGGGCCCGGGG |
| | | CUGCCGGACCCUGGGCCUGCCUGCCCUGCUGCUGCUGCUUCUGCUGCGGCCCC |
| | | CCGCCACCCGGGGCAUCACCUGUCCCCCACCUAUGAGCGUGGAGCACGCCGAC |
| | | AUCUGGGUGAAGAGCUACAGCCUGUACAGCCGGGAGCGGUACAUCUGCAACA |
| | | GCGGCUUCAAGCGGAAGGCAGGCACCAGCAGCCUGACCGAGUGCGUGCUGAAC |
| | | AAGGCCACCAACGUGGCCCACUGGACCACCCCCAGCCUUAAGUGCAUCCGGGA |
| | | CCCUGCGCUGGUCCACCAGCGGCCUGCCCCCCCCAGCACCGUGACCACCGCAGG |
| | | AGUGACCCCCCAGCCCGAGAGCCUGAGCCCCAGCGGCAAGGAGCCCGCCGCCA |
| | | GCUCACCCAGCAGCAACAACACCGCCGCCACCACCGCCGCCAUCGUGCCCGGCA |
| | | GCCAGCUGAUGCCUAGCAAGAGCCCCAGCACCGGCACCACCGAGAUCAGCAGC |
| | | CACGAGAGCAGCCACGGCACCCCCAGCCAGACCACCGCCAAGAAUUGGGAGCU |
| | | GACCGCCAGCGCCAGCCACCAGCCCCCAGGCGUGUACCCCCAGGGGCACAGCG |
| | | ACACCACCGUGGCCAUCAGCACCAGCACCGUGCUGCUGUGCGGCCUGAGCGCA |
| | | GUGAGCCUGCUGGCCUGCUACCUGAAGAGCCGGCAGACCCCACCCCUGGCUAG |
| | | CGUGGAGAUGGAGGCCAUGGAGGCCCUGCCCGUGACCUGGGGAACCAGCAGCC |
| | | GGGACGAGGACCUGGAGAACUGCAGCCACCACCUGUGAUUAAACCGGUCUGCA |
| | | GCUUUUGCCUUGGUUCUUCCUAGUGCCUACAAUGGGAAAACUUCACUCCAAA |
| | | GAGAAACCUAUUAAGUCAUCAUCUCCAAACUAAACCCUCACAAGUAACAGUU |
| | | GAAGAAAAAAUGGCAAGAGAUCAUAUCCUCAGACCAGGUGGAAUUACUUAAA |
| | | UUUUAAAGCCUGAAAAUUCCAAUUUGGGGGUGGGAGGUGGAAAAGGAACCCA |
| | | AUUUUCUUAUGAACAGAUAUUUUUAACUUAAUGGCACAAAGUCUUAGAAUAU |
| | | UAUUAUGUGCCCCGUGUUCCCUGUUCUUCGUUGCUGAAUUCGAUAUCCAGCG |
| | | GCCGCGCUAGCGCUGAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAA |
| SEQ ID | U27-hIL- | AGGUCUUCUGGUCCCCACAGACUCAGAGAGAACCCGCCACCaugAAGUGAGGCU |
| NO: 80 | 15_verB_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGAGUUCGGCCUGAGCUGGG |
| | | UGUUCCUGGUGGCCCUGUUCCGGGGAGUGCAGUGUAUCACCUGCCCCCCCCCC |
| | | AUGAGCGUGGAGCACGCUGACAUCUGGGUGAAGAGCUACAGCCUGUACAGCC |
| | | GGGAGCGGUACAUCUGCAACAGCGGCUUCAAGCGGAAGGCCGGCACCAGCUCC |
| | | CUGACCGAGUGCGUGCUGAACAAGGCCACCAACGUGGCCCACUGGACCACCCC |
| | | CAGCCUGAAGUGCAUCCGGGACCCCGCCCUGGUGGGCGGCUCAGGCGGCAGCG |
| | | GCGGCAGCGGGGGCAGCGGAGGCAGCGGCGGAAGCGGCGGCAACUGGGUGAA |
| | | CGUGAUCAGCGACCUGAAGAAGAUCGAGGACCUGAUCCAAAGCAUGCACAUU |
| | | GACGCCACCCUGUACACCGAGAGCGACGUGCACCCCAGCUGCAAGGUGACCGC |
| | | AAUGAAGUGCUUCCUGCUGGAGCUGCAGGUGAUCAGCCUGGAGAGCGGCGAC |
| | | GCCAGCAUCCACGACACCGUGGAGAACCUGAUCAUCCUGGCGAACAACAGCCU |
| | | GAGCAGCAACGGCAACGUGACCGAGAGCGGCUGUAAGGAGUGCGAGGAGCUG |
| | | GAGGAGAAGAAUAUCAAGGAGUUCCUGCAGAGCUUCGUGCACAUCGUGCAGA |
| | | UGUUCAUCAACACCAGCUGAUUAAACCGGUCUGCAGCUUUUGCCUUGGUUCU |
| | | UCCUAGUGCCUACAAUGGGAAAACUUCACUCCAAAGAGAAACCUAUUAAGUC |
| | | AUCAUCUCCAAACUAAACCCUCACAAGUAACAGUUGAAGAAAAAAUGGCAAG |
| | | AGAUCAUAUCCUCAGACCAGGUGGAAUUACUUAAAUUUUAAAGCCUGAAAAU |
| | | UCCAAUUUGGGGGUGGGAGGUGGAAAAGGAACCCAAUUUUCUUAUGAACAGA |
| | | UAUUUUUAACUUAAUGGCACAAAGUCUUAGAAUAUUAUUAUGUGCCCCGUGU |
| | | UCCCUGUUCUUCGUUGCUGAAUUCGAUAUCCAGCGGCCGCGCUAGCGCUGAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAA |
| SEQ ID | U27-hIL- | AGGUCUUCUGGUCCCCACAGACUCAGAGAGAACCCGCCACCaugAAGUGAGGCU |
| NO: 81 | 15_verC_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGAGUUCGGCCUGAGCUGGG |
| | | UGUUUCUGGUGGCCCUGUUCCGGGGCGUGCAGUGCAACUGGGUGAACGUGAU |
| | | CAGCGACCUGAAGAAGAUCGAGGACCUGAUCCAGUCAAUGCACAUCGAUGCA |
| | | ACCCUGUACACCGAGAGCGACGUGCACCCUAGCUGUAAGGUGACCGCAAUGAA |
| | | GUGCUUCCUGCUGGAGCUGCAGGUGAUAAGCCUGGAGAGCGGCGACGCCAGC |
| | | AUCCACGACACAGUCGAGAAUCUGAUCAUCCUGGCUAACAACAGCCUGAGCAG |
| | | CAAUGGUAAUGUGACCGAGAGCGGCUGCAAGGAGUGCGAGGAGCUGGAGGAG |
| | | AAGAACAUCAAGGAGUUCCUGCAGAGCUUCGUGCACAUCGUGCAGAUGUUCA |
| | | UCAACACCAGCGGCGGCAGCGGGGGCAGCGGCGGCUCAGGUGGCAGCGGAGGC |
| | | AGCGGCGGAAGCGGCGGCAUCACCUGCCCUCCCCCCAUGAGCGUGGAGCACGC |
| | | AGACAUCUGGGUGAAGAGCUACAGCCUGUACAGCCGGGAGCGGUACAUCUGC |
| | | AACAGCGGCUUCAAGCGGAAGGCCGGCACCAGCAGCCUGACCGAGUGUGUGCU |
| | | GAACAAGGCCACCAACGUGGCCCACUGGACAACCCCCAGCCUGAAGUGCAUCA |
| | | GGGACCCCGCCCUGGUGUGAUUAAACCGGUCUGCAGCUUUUGCCUUGGUUCUU |
| | | CCUAGUGCCUACAAUGGGAAAACUUCACUCCAAAGAGAAACCUAUUAAGUCA |
| | | UCAUCUCCAAACUAAACCCUCACAAGUAACAGUUGAAGAAAAAAUGGCAAGA |
| | | GAUCAUAUCCUCAGACCAGGUGGAAUUACUUAAAUUUUAAAGCCUGAAAAUU |
| | | CCAAUUUGGGGGUGGGAGGUGGAAAAGGAACCCAAUUUUCUUAUGAACAGAU |
| | | AUUUUUAACUUAAUGGCACAAAGUCUUAGAAUAUUAUUAUGUGCCCCGUGUU |
| | | CCCUGUUCUUCGUUGCUGAAUUCGAUAUCCAGCGGCCGCGCUAGCGCUGAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAA |
| SEQ ID | U27-mIL- | AGGUCUUCUGGUCCCCACAGACUCAGAGAGAACCCGCCACCaugAAGUGAGGCU |
| NO: 82 | 15_verA_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCUAGCCCCCAGCUGCGGGG |
| | | CUACGGCGUGCAGGCCAUCCCUGUGCUGCUGUUACUGCUGCUGCUCCUGCUGC |
| | | UGCCCCUGCGGGUGACCCCCGGCACCACCUGCCCCCCCCCCGUGAGCAUCGAGC |
| | | ACGCCGACAUCCGGGUGAAGAACUACAGCGUGAACAGCCGGGAGAGGUACGU |
| | | GUGCAAUAGCGGCUUCAAGCGGAAGGCCGGCACCAGCACCCUGAUCGAGUGCG |
| | | UGAUCAACAAGAACACCAACGUGGCCCACUGGACCACCCCCAGCCUGAAGUGC |
| | | AUCCGGGACCCCAGCUUAGCCCACUACAGCCCCGUGCCCACCGUGGUGACCCC |
| | | AAAGGUGACCAGCCAGCCCGAGAGCCCCAGCCCCAGCGCCAAGGAGCCUGAGG |
| | | CCUUCUCCCCAAAGAGCGACACCGCCAUGACCACCGAGACCGCCAUCAUGCCC |
| | | GGCAGCCGGCUGACCCCCAGCCAGACCACCAGCGCCGGCACCACAGGCACCGG |
| | | CAGCCACAAGAGCAGCCGGGCACCCAGCCUGGCCGCCACCAUGACCCUGGAGC |
| | | CUACCGCUAGCACCAGCCUGCGGAUCACCGAGAUCAGCCCCCACAGCAGCAAG |
| | | AUGACCAAGGUGGCCAUCAGCACCAGCGUGCUGCUGGUGGGCGCCGGCGUGGU |
| | | GAUGGCUUUCCUGGCCUGGUACAUCAAGAGCCGGCAGCCCAGCCAGCCUUGCC |
| | | GGGUGGAGGUGGAGACCAUGGAGACUGUGCCCAUGACCGUGCGGGCCAGCAG |
| | | CAAGGAGGACGAGGACACCGGCGCCGGGGGCAGCGGCGGUAGCGGAGGCAGC |
| | | GGCGGCAGCGGCGGAAGCGGCGGCUCCGGCGGCAUGAAGAUCCUGAAGCCCUA |
| | | CAUGCGGAACACCAGCAUCAGCUGCUACCUGUGCUUCCUGCUGAACAGCCACU |
| | | UCCUGACCGAGGCCGGCAUCCACGUGUUCAUCCUGGGCUGCGUGAGCGUGGGC |
| | | CUGCCUAAGACCGAGGCCAACUGGAUCGACGUGCGGUACGACCUGGAGAAGA |
| | | UCGAGAGCCUGAUCCAGAGCAUCCACAUCGACACCACCCUGUACACCGACAGC |
| | | GACUUCCACCCCAGCUGCAAGGUGACCGCCAUGAACUGCUUUCUGCUGGAGCU |
| | | GCAGGUGAUCCUGCACGAGUACAGCAACAUGACCCUGAACGAGACCGUGCGGA |
| | | ACGUGCUGUACCUGGCCAACAGCACCCUGAGCAGCAACAAGAACGUGGCCGAG |
| | | AGCGGCUGCAAGGAGUGCGAGGAGCUGGAGGAGAAGACCUUCACCGAGUUCC |
| | | UGCAGAGCUUCAUCCGGAUCGUGCAGAUGUUCAUCAACACCAGCUGAUUAAA |
| | | CCGGUCUGCAGCUUUUGCCUUGGUUCUUCCUAGUGCCUACAAUGGGAAAACU |
| | | UCACUCCAAAGAGAAACCUAUUAAGUCAUCAUCUCCAAACUAAACCCUCACAA |
| | | GUAACAGUUGAAGAAAAAAUGGCAAGAGAUCAUAUCCUCAGACCAGGUGGAA |
| | | UUACUUAAAUUUUAAAGCCUGAAAAUUCCAAUUUGGGGGUGGGAGGUGGAAA |
| | | AGGAACCCAAUUUUCUUAUGAACAGAUAUUUUUAACUUAAUGGCACAAAGUC |
| | | UUAGAAUAUUAUUAUGUGCCCCGUGUUCCCUGUUCUUCGUUGCUGAAUUCGA |
| | | UAUCCAGCGGCCGCGCUAGCGCUGAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAA |
| SEQ ID | U27-mIL- | AGGUCUUCUGGUCCCCACAGACUCAGAGAGAACCCGCCACCaugAAGUGAGGCU |
| NO: 83 | 15_verB_ | CUCCUCCCGCCCCGCCCCUCCCACGCCUCACCAGCCCCCCGCGCGCCCACCCUC |
| | adn | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGAGCGUGCUGACCCAGGUGC |
| | | UGGCCCUGCUGCUGCUGUGGCUGACCGGCGCCCGUUGCACCACCUGCCCCCCC |
| | | CCAGUGAGCAUCGAGCACGCCGAUAUCCGGGUGAAGAACUACAGCGUGAACA |
| | | GCCGGGAGCGGUACGUGUGCAACAGCGGCUUCAAGCGGAAGGCCGGCACAAGC |
| | | ACCCUGAUCGAGUGCGUGAUCAACAAGAACACCAAUGUGGCCCACUGGACCAC |
| | | CCCCAGCCUGAAGUGUAUCCGGGACCCUAGCCUGGCCGGCGGCAGCGGCGGGA |
| | | GCGGUGGCAGCGGCGGAAGCGGCGGUAGCGGAGGCAGCGGCGGCAACUGGAU |
| | | CGACGUGCGGUACGACCUAGAGAAGAUCGAGAGCCUGAUCCAGAGCAUCCACA |
| | | UCGACACCACCCUGUACACCGACAGCGACUUCCACCCCAGCUGCAAGGUGACC |
| | | GCCAUGAACUGCUUCCUGCUGGAGCUGCAGGUGAUCCUGCACGAGUACAGCAA |
| | | CAUGACACUGAACGAGACCGUGCGGAACGUGCUGUACCUGGCAAACAGCACCC |
| | | UGAGCAGCAACAAGAAUGUGGCCGAGAGCGGCUGCAAGGAAUGCGAGGAGCU |
| | | GGAGGAGAAGACCUUCACCGAGUUCCUCCAGAGCUUCAUCCGGAUCGUGCAGA |
| | | UGUUCAUCAACACCAGCUGAUUAAACCGGUCUGCAGCUUUUGCCUUGGUUCU |
| | | UCCUAGUGCCUACAAUGGGAAAACUUCACUCCAAAGAGAAACCUAUUAAGUC |
| | | AUCAUCUCCAAACUAAACCCUCACAAGUAACAGUUGAAGAAAAAAUGGCAAG |
| | | AGAUCAUAUCCUCAGACCAGGUGGAAUUACUUAAAUUUUAAAGCCUGAAAAU |
| | | UCCAAUUUGGGGGUGGGAGGUGGAAAAGGAACCCAAUUUUCUUAUGAACAGA |
| | | UAUUUUUAACUUAAUGGCACAAAGUCUUAGAAUAUUAUUAUGUGCCCCGUGU |
| | | UCCCUGUUCUUCGUUGCUGAAUUCGAUAUCCAGCGGCCGCGCUAGCGCUGAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAA |
| SEQ ID | U27-mIL- | AGGUCUUCUGGUCCCCACAGACUCAGAGAGAACCCGCCACCaugAAGUGAGGCU |
| NO: 84 | 15_verC_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGAGCGUGCUGACCCAGGUGC |
| | | UGGCUCUACUGCUGCUGUGGUUAACAGGCGCCCGGUGCAACUGGAUCGACGU |
| | | GCGGUACGACCUGGAGAAGAUCGAGAGCCUGAUCCAGAGCAUCCACAUCGACA |
| | | CCACCCUGUACACCGACAGCGACUUCCACCCUAGCUGCAAGGUGACCGCCAUG |
| | | AACUGCUUCCUGCUGGAGCUGCAGGUGAUCCUGCACGAGUACAGCAAUAUGA |
| | | CCCUGAACGAGACCGUGCGGAACGUGCUGUACCUGGCCAACAGCACCCUGAGC |
| | | AGCAAUAAGAAUGUGGCCGAGAGCGGCUGCAAGGAGUGUGAGGAGCUGGAGG |
| | | AGAAGACCUUCACUGAGUUCCUGCAGAGCUUUAUCCGGAUCGUGCAGAUGUU |
| | | CAUCAACACUAGCGGCGGAAGCGGCGGCUCAGGCGGCAGCGGAGGCAGCGGCG |
| | | GCAGCGGGGGCAGCGGCGGAACCACCUGUCCCCCCCCCGUGAGCAUCGAGCAC |
| | | GCCGACAUCCGGGUGAAGAACUACAGCGUGAACUCACGGGAGCGGUACGUGU |
| | | GCAACUCAGGCUUCAAGCGGAAGGCCGGCACCAGCACCCUGAUCGAGUGCGUG |
| | | AUCAACAAGAACACAAACGUGGCCCACUGGACCACCCCCAGCCUGAAGUGCAU |
| | | CCGGGACCCCAGCCUGGCCUGAUUAAACCGGUCUGCAGCUUUUGCCUUGGUUC |
| | | UUCCUAGUGCCUACAAUGGGAAAACUUCACUCCAAAGAGAAACCUAUUAAGU |
| | | CAUCAUCUCCAAACUAAACCCUCACAAGUAACAGUUGAAGAAAAAAUGGCAA |
| | | GAGAUCAUAUCCUCAGACCAGGUGGAAUUACUUAAAUUUUAAAGCCUGAAAA |
| | | UUCCAAUUUGGGGGUGGGAGGUGGAAAAGGAACCCAAUUUUCUUAUGAACAG |
| | | AUAUUUUUAACUUAAUGGCACAAAGUCUUAGAAUAUUAUUAUGUGCCCCGUG |
| | | UUCCCUGUUCUUCGUUGCUGAAUUCGAUAUCCAGCGGCCGCGCUAGCGCUGAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAA |
| SEQ ID | U27-mIL- | AGGUCUUCUGGUCCCCACAGACUCAGAGAGAACCCGCCACCaugAAGUGAGGCU |
| NO: 85 | 15_verD_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGAGCGUGCUGACCCAGGUGC |
| | | UGGCCCUGCUGCUGCUGUGGCUGACCGGCGCCCGGUGCAACUGGAUCGACGUG |
| | | CGGUACGAUCUGGAGAAGAUCGAGAGCCUGAUCCAGAGCAUCCACAUCGACAC |
| | | CACCCUGUACACCGAUAGCGACUUCCACCCCAGCUGCAAGGUGACCGCCAUGA |
| | | ACUGCUUCCUGCUGGAACUGCAGGUGAUCCUGCACGAGUACAGCAACAUGACC |
| | | CUGAACGAGACCGUGCGGAACGUCCUGUACCUGGCCAACAGCACACUGAGCAG |
| | | CAACAAGAACGUGGCCGAGAGCGGCUGCAAGGAGUGCGAGGAGCUGGAGGAG |
| | | AAGACCUUCACCGAGUUCCUGCAGAGCUUCAUCCGUAUCGUGCAGAUGUUCAU |
| | | CAACACCAGCGGGGGCAGCGGCGGAAGCGGGGGCAGCGGCGGAAGCGGCGGCA |
| | | GCGGAGGCAGCGGAGGCACCACAUGCCCCCCACCCGUGAGCAUCGAGCACGCC |
| | | GACAUACGGGUGAAGAAUUACAGCGUGAACAGCCGGGAGCGGUACGUGUGCA |
| | | ACAGCGGCUUCAAGCGGAAGGCCGGCACCAGCACCCUGAUCGAGUGCGUGAUC |
| | | AACAAGAACACUAACGUGGCACACUGGACCACCCCCAGCCUCAAGUGCAUCCG |
| | | GGACCCAAGCCUGGCCGGGGGGGGCGGCUCCGGCGGAGGCGGCAGCGGCGGUG |
| | | GAGGCAGCGGCCCUACAAUCAAGCCCUGCCCCCCCUGCAAGUGCCCCGCCCCC |
| | | AAUGCCGCCGGCGGCCCCAGCGUGUUCAUCUUCCCCCCCAAGAUCAAGGACGU |
| | | GCUGAUGAUCUCACUAAGCCCCAUAGUGACCUGCGUGGUGGUGGACGUGAGC |
| | | GAGGACGACCCUGACGUGCAGAUCAGCUGGUUCGUGAACAACGUGGAGGUGC |
| | | ACACCGCCCAGACCCAGACCCACCGGGAGGACUACAACAGCACCCUGCGGGUG |
| | | GUGAGCGCCCUGCCCAUCCAGCACCAGGACUGGAUGAGCGGCAAGGAGUUCAA |
| | | GUGUAAGGUGAAUAAUAAGGACCUGGGCGCCCCAAUAGAGCGGACCAUUAGC |
| | | AAGCCCAAGGGGAGCGUGCGGGCCCCCCAGGUGUACGUGCUGCCACCCCCUGA |
| | | GGAGGAGAUGACCAAAAAGCAGGUGACCCUGACCUGCAUGGUGACCGAUUUC |
| | | AUGCCAGAGGACAUCUACGUGGAGUGGACCAACAACGGCAAGACCGAGCUGA |
| | | ACUACAAGUGAUUAAACCGGUCUGCAGCUUUUGCCUUGGUUCUUCCUAGUGC |
| | | CUACAAUGGGAAAACUUCACUCCAAAGAGAAACCUAUUAAGUCAUCAUCUCC |
| | | AAACUAAACCCUCACAAGUAACAGUUGAAGAAAAAAUGGCAAGAGAUCAUAU |
| | | CCUCAGACCAGGUGGAAUUACUUAAAUUUUAAAGCCUGAAAAUUCCAAUUUG |
| | | GGGGUGGGAGGUGGAAAAGGAACCCAAUUUUCUUAUGAACAGAUAUUUUUAA |
| | | CUUAAUGGCACAAAGUCUUAGAAUAUUAUUAUGUGCCCCGUGUUCCCUGUUC |
| | | UUCGUUGCUGAAUUCGAUAUCCAGCGGCCGCGCUAGCGCUGAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAA |
| SEQ ID | U27-hIL- | AGGUCUUCUGGUCCCCACAGACUCAGAGAGAACCCGCCACCaugAAGUGAGGCU |
| NO: 86 | 12_adm | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUGCCACCAGCAGCUGGUGA |
| | | UCAGCUGGUUCAGCCUGGUGUUCCUCGCCAGCCCCCUGGUGGCUAUCUGGGAG |
| | | CUGAAGAAGGACGUGUACGUGGUGGAGCUGGACUGGUACCCCGACGCCCCCGG |
| | | CGAGAUGGUGGUGCUCACCUGCGAUACCCCCGAGGAGGACGGAAUCACCUGGA |
| | | CCCUGGACCAGAGCAGCGAGGUGUUAGGCAGCGGCAAGACCCUGACCAUCCAG |
| | | GUGAAGGAGUUCGGCGACGCCGGCCAGUACACCUGCCACAAGGGGGGGGAGG |
| | | UGCUGAGCCACAGCCUGCUGCUGCUGCACAAGAAGGAGGACGGCAUCUGGAGC |
| | | ACCGACAUCCUGAAGGACCAGAAGGAGCCCAAGAACAAGACCUUCCUGCGGUG |
| | | CGAGGCCAAGAAUUACAGCGGCCGGUUCACCUGCUGGUGGCUGACCACCAUCA |
| | | GCACCGACCUGACCUUCAGCGUGAAGAGCAGCCGGGGCAGCAGCGACCCCCAG |
| | | GGCGUGACCUGCGGCGCCGCCACCCUGAGCGCCGAGCGGGUGAGGGGCGACAA |
| | | UAAGGAGUACGAGUACAGCGUGGAGUGUCAGGAGGAUAGCGCAUGCCCCGCC |
| | | GCCGAGGAGAGCCUGCCCAUCGAGGUGAUGGUGGACGCCGUGCACAAGCUGA |
| | | AGUACGAGAACUACACCAGCAGCUUCUUCAUCCGGGACAUCAUCAAGCCCGAC |
| | | CCCCCCAAGAACCUGCAGCUGAAGCCCCUGAAGAACAGCCGGCAGGUGGAGGU |
| | | GUCAUGGGAGUACCCCGACACCUGGAGCACCCCCCACAGCUACUUCAGCCUGA |
| | | CAUUCUGUGUGCAGGUGCAGGGCAAGAGCAAGCGGGAGAAGAAGGACCGGGU |
| | | GUUCACCGACAAGACCAGCGCCACCGUGAUCUGCCGGAAGAAUGCCAGCAUCA |
| | | GCGUGCGGGCCCAGGACCGGUACUACAGCAGCAGCUGGAGCGAGUGGGCCAGC |
| | | GUGCCUUGCUCCGGCGGCGGAGGAAGCGGAGGCGGGGGCAGCGGGGGGGGCG |
| | | GAAGCCGGAACCUGCCCGUGGCCACCCCAGACCCCGGCAUGUUCCCCUGCCUG |
| | | CACCACAGCCAGAACCUGCUGCGGGCCGUGAGCAACAUGCUGCAGAAGGCUCG |
| | | GCAGACCCUGGAGUUCUACCCCUGCACCAGCGAGGAGAUCGACCACGAGGACA |
| | | UCACCAAGGACAAGACUAGCACCGUGGAGGCCUGCCUGCCCCUGGAGCUGACC |
| | | AAGAAUGAGAGCUGCCUGAACAGCCGGGAGACCAGCUUUAUCACCAACGGCA |
| | | GCUGCCUGGCCAGCCGGAAGACCUCAUUCAUGAUGGCCCUGUGCCUGAGCAGC |
| | | AUCUACGAGGACCUGAAGAUGUACCAGGUGGAGUUCAAGACCAUGAACGCCA |
| | | AGCUGCUGAUGGACCCUAAGCGGCAAAUCUUCCUGGACCAGAAUAUGCUGGCC |
| | | GUGAUCGACGAGCUCAUGCAGGCCCUGAACUUUAACAGCGAGACCGUGCCCCA |
| | | GAAGAGCAGCCUGGAGGAGCCAGACUUCUACAAGACCAAGAUCAAGCUGUGC |
| | | AUCCUGCUGCACGCUUUCCGGAUCCGGGCCGUGACCAUCGACCGGGUGAUGAG |
| | | CUACCUGAACGCGAGCAGCGGAGGCGGCAGCGGUGGCGGCGGCUCAGGCGGCG |
| | | GAGGCAGCGGCGGAGGCGGAAGCGGCGGCGGCAGCCUGCAGGUGGUGAUCAG |
| | | CGCCAUCCUGGCUCUGGUGGUGCUGACCAUCAUCUCCCUGAUCAUCCUGAUCG |
| | | GCGGAGGUGGCAGCUGAUUAAACCGGUCUGCAGCUUUUGCCUUGGUUCUUCC |
| | | UAGUGCCUACAAUGGGAAAACUUCACUCCAAAGAGAAACCUAUUAAGUCAUC |
| | | AUCUCCAAACUAAACCCUCACAAGUAACAGUUGAAGAAAAAAUGGCAAGAGA |
| | | UCAUAUCCUCAGACCAGGUGGAAUUACUUAAAUUUUAAAGCCUGAAAAUUCC |
| | | AAUUUGGGGGUGGGAGGUGGAAAAGGAACCCAAUUUUCUUAUGAACAGAUAU |
| | | UUUUAACUUAAUGGCACAAAGUCUUAGAAUAUUAUUAUGUGCCCCGUGUUCC |
| | | CUGUUCUUCGUUGCUGAAUUCGAUAUCCAGCGGGCCGCGCUAGCGCUGAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAA |
| SEQ ID | U27-mIL- | AGGUCUUCUGGUCCCCACAGACUCAGAGAGAACCCGCCACCaugAAGUGAGGCU |
| NO: 87 | 12_adm | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUGCCCCCAGAAGCUGACCAU |
| | | CAGCUGGUUCGCCAUCGUGCUGCUGGUGAGCCCUCUGAUGGCCAUGUGGGAGC |
| | | UGGAGAAGGACGUGUACGUGGUGGAGGUGGAUUGGACCCCCGACGCACCCGG |
| | | CGAGACCGUGAACCUGACCUGCGAUACCCCUGAGGAGGACGACAUCACCUGGA |
| | | CUAGCGAUCAGCGGCACGGCGUGAUCGGCAGCGGCAAGACCCUGACCAUCACC |
| | | GUGAAGGAGUUCCUGGAUGCCGGCCAGUACACCUGCCACAAGGGGGGGGAGA |
| | | CCCUGAGCCACAGCCACCUGCUGCUGCACAAAAAGGAGAACGGCAUCUGGAGC |
| | | ACCGAGAUCCUGAAGAACUUCAAGAACAAGACCUUUCUGAAGUGCGAGGCUC |
| | | CCAACUACAGCGGUCGGUUCACCUGCAGCUGGCUGGUGCAGCGGAAUAUGGAC |
| | | CUGAAGUUUAACAUCAAGUCAAGCAGCAGCAGCCCCGACAGCCGGGCCGUGAC |
| | | CUGUGGCAUGGCCAGCCUGAGCGCCGAGAAGGUGACCCUGGACCAGAGGGAU |
| | | UACGAGAAGUACAGCGUGAGCUGCCAGGAGGACGUGACCUGCCCAACCGCCGA |
| | | GGAGACCCUGCCCAUCGAGCUGGCCCUGGAGGCCCGGCAGCAGAACAAGUACG |
| | | AGAACUACAGCACCAGCUUCUUCAUCCGGGACAUCAUCAAGCCUGACCCCCCC |
| | | AAGAACCUGCAGAUGAAGCCCCUGAAGAACAGCCAGGUGGAGGUGAGCUGGG |
| | | AGUACCCCGACAGCUGGAGCACCCCCCACAGCUACUUCAGCCUGAAGUUCUUC |
| | | GUGCGGAUCCAGCGGAAGAAGGAGAAGAUGAAGGAGACCGAGGAGGGCUGCA |
| | | ACCAGAAGGGCGCCUUCCUGGUGGAGAAGACCAGCACCGAGGUGCAGUGUAA |
| | | GGGCGGCAACGUGUGCGUGCAGGCCCAGGACCGGUACUACAACAGCUCAUGCA |
| | | GCAAGUGGGCCUGCGUGCCCUGCCGGGUGAGGAGCGGCGGCGGCGGCUCCGGC |
| | | GGAGGAGGCAGCGGAGGAGGCGGCAGCCGGGUGAUCCCCGUGAGCGGCCCUGC |
| | | CCGGUGCCUGAGCCAGAGCCGGAACCUGCUGAAGACCACCGACGACAUGGUGA |
| | | AGACCGCUCGGGAGAAGCUGAAGCACUACAGCUGCACCGCCGAGGACAUCGAC |
| | | CACGAGGACAUCACCCGGGACCAGACCAGCACCUUAAAGACCUGCCUGCCCCU |
| | | GGAGCUGCACAAGAAUGAGAGCUGCCUGGCCACCCGGGAGACCAGCAGCACCA |
| | | CCCGGGGCAGCUGCCUGCCCCCCCAGAAGACCAGCCUGAUGAUGACCCUGUGC |
| | | CUGGGCAGCAUCUACGAGGACCUGAAGAUGUACCAGACCGAGUUCCAGGCAA |
| | | UCAACGCUGCUCUGCAGAAUCACAACCACCAGCAGAUCAUCCUGGACAAGGGA |
| | | AUGCUGGUGGCCAUCGACGAGCUGAUGCAGAGCCUGAACCACAACGGCGAGAC |
| | | CCUGCGGCAGAAGCCCCCCGUGGGCGAGGCCGACCCCUACCGUGUGAAGAUGA |
| | | AGCUGUGCAUCCUGCUGCACGCUUUCAGCACCCGGGUGGUGACCAUCAACCGG |
| | | GUGAUGGGCUACCUGAGCAGCGCAAGCGGUGGCGGCAGCGGCGGCGGGGGCU |
| | | CCGGCGGCGGUGGGAGCGGCGGAGGCGGUUCAGGCGGCGGCAGCCUGCAGGU |
| | | GGUGAUCAGCGCCAUCCUGGCCCUGGUGGUGCUCACCGUGAUCAGCCUGAUCA |
| | | UCCUGAUCGGGGGGGGCGGCUCAGGCAAGCCCAUCCCAAAUCCACUGCUGGGC |
| | | CUGGACAGCACCUGAUUAAACCGGUCUGCAGCUUUUGCCUUGGUUCUUCCUAG |
| | | UGCCUACAAUGGGAAAACUUCACUCCAAAGAGAAACCUAUUAAGUCAUCAUC |
| | | UCCAAACUAAACCCUCACAAGUAACAGUUGAAGAAAAAAUGGCAAGAGAUCA |
| | | UAUCCUCAGACCAGGUGGAAUUACUUAAAUUUUAAAGCCUGAAAAUUCCAAU |
| | | UUGGGGGUGGGAGGUGGAAAAGGAACCCAAUUUUCUUAUGAACAGAUAUUUU |
| | | UAACUUAAUGGCACAAAGUCUUAGAAUAUUAUUAUGUGCCCCGUGUUCCCUG |
| | | UUCUUCGUUGCUGAAUUCGAUAUCCAGCGGCCGCGCUAGCGCUGAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAA |
| SEQ ID | U27-TET- | AGGUCUUCUGGUCCCCACAGACUCAGAGAGAACCCGCCACCaugAAGUGAGGCU |
| NO: 88 | IL- | CUCCUCCCGCCCCGCCCCUCCCACGCCUCACCAGCCCCCCGCGCGCCCACCCUC |
| | 12_adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUGCCCCCAGAAGCUGACCAU |
| | | CAGCUGGUUCGCCAUCGUGCUGCUGGUGAGCCCUCUGAUGGCCAUGUGGGAGC |
| | | UCGAGAAGGACGUGUACGUGGUGGAGGUGGACUGGACCCCCGACGCCCCCGGC |
| | | GAGACCGUGAACCUGACCUGUGACACCCCCGAGGAGGACGACAUCACCUGGAC |
| | | CAGCGACCAGCGGCACGGCGUGAUCGGAAGCGGCAAGACCCUGACCAUCACCG |
| | | UCAAGGAGUUCCUGGACGCCGGCCAGUACACCUGCCACAAGGGCGGCGAGACA |
| | | CUGAGCCACAGCCACCUGCUGUUACACAAGAAGGAGAACGGCAUCUGGAGCAC |
| | | CGAGAUUCUGAAGAACUUCAAGAACAAGACCUUCCUGAAGUGCGAAGCCCCCA |
| | | AUUACAGCGGCCGGUUCACCUGCAGCUGGCUGGUGCAGCGGAACAUGGACCUG |
| | | AAGUUCAAUAUCAAGUCAAGCAGCAGCAGCCCCGAUAGCCGGGCCGUGACCUG |
| | | CGGCAUGGCCAGCCUGAGCGCCGAGAAGGUGACCCUGGACCAGAGGGACUACG |
| | | AGAAGUACAGCGUGAGCUGUCAGGAGGACGUGACAUGCCCAACCGCCGAGGA |
| | | GACCCUGCCCAUCGAGCUGGCCCUGGAGGCCCGGCAGCAGAAUAAGUACGAGA |
| | | AUUACAGCACCAGCUUCUUCAUCCGGGACAUCAUCAAGCCCGACCCCCCUAAG |
| | | AACCUGCAGAUGAAGCCCCUGAAGAAUAGCCAGGUCGAGGUGAGCUGGGAGU |
| | | ACCCCGAUAGCUGGAGCACCCCCCACAGCUACUUCAGCCUGAAGUUCUUCGUG |
| | | CGGAUCCAGCGGAAGAAGGAGAAGAUGAAGGAGACCGAGGAGGGCUGCAAUC |
| | | AGAAGGGCGCAUUCCUGGUGGAGAAGACCAGCACCGAGGUGCAGUGCAAGGG |
| | | CGGCAACGUGUGCGUGCAGGCCCAGGAUCGGUACUACAACAGCAGCUGCAGCA |
| | | AGUGGGCCUGCGUGCCAUGCCGGGUGCGGAGCGGCGGCGGAGGCUCAGGUGG |
| | | CGGCGGCUCAGGCGGGGGGGGCAGCCGGGUGAUCCCCGUGAGCGGCCCCGCCC |
| | | GGUGCCUUAGCCAGAGCCGGAAUCUGCUGAAGACCACCGACGACAUGGUGAA |
| | | GACCGCCCGGGAGAAGCUGAAGCACUACAGCUGCACCGCCGAGGACAUCGACC |
| | | ACGAGGACAUCACCCGGGACCAGACCAGCACCCUGAAGACCUGCCUGCCCCUG |
| | | GAGCUGCACAAGAACGAGAGCUGCCUGGCAACCCGGGAGACCAGCAGCACCAC |
| | | CCGGGGCAGCUGCCUCCCCCCCCAGAAGACCAGCCUGAUGAUGACCCUGUGCC |
| | | UGGGGAGCAUCUACGAGGACCUGAAGAUGUACCAGACCGAGUUCCAGGCUAU |
| | | AAACGCCGCCCUGCAGAACCACAACCACCAGCAGAUCAUCCUGGACAAGGGCA |
| | | UGCUGGUGGCCAUCGACGAGCUGAUGCAGAGCCUGAACCACAACGGCGAGACU |
| | | CUGCGGCAGAAGCCCCCUGUGGGCGAGGCCGAUCCCUACCGGGUGAAGAUGAA |
| | | GCUGUGCAUCCUGCUGCACGCCUUCAGCACCCGGGUGGUGACCAUCAACAGGG |
| | | UGAUGGGCUACCUGAGCAGCGCUAGCGGCGGAGGUAGCGGAGGCGGCGGCUC |
| | | CGGCGGCGGCGGCAGCGGUGGAGGCGGCAGCGGCGGCGGUAGCCUGCAAGUG |
| | | GUGAUCAGCGCCAUCCUGGCCCUGGUGGUGCUGACCGUGAUCAGCCUGAUCAU |
| | | CCUGAUCGGCGGUGGCGGGAGCGGCAAGCCCAUCCCCAACCCACUGCUGGGCC |
| | | UGGACAGCACCUGAUUAAACCGGUCUGCAGCUUUUGCCUUGGUUCUUCCUAG |
| | | UGCCUACAAUGGGAAAACUUCACUCCAAAGAGAAACCUAUUAAGUCAUCAUC |
| | | UCCAAACUAAACCCUCACAAGUAACAGUUGAAGAAAAAAUGGCAAGAGAUCA |
| | | UAUCCUCAGACCAGGUGGAAUUACUUAAAUUUUAAAGCCUGAAAAUUCCAAU |
| | | UUGGGGGUGGGAGGUGGAAAAGGAACCCAAUUUUCUUAUGAACAGAUAUUUU |
| | | UAACUUAAUGGCACAAAGUCUUAGAAUAUUAUUAUGUGCCCCGUGUUCCCUG |
| | | UUCUUCGUUGCUGAAUUCGAUAUCCAGCGGCCGCGCUAGCGCUGAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAA |
| SEQ ID | U27- | AGGUCUUCUGGUCCCCACAGACUCAGAGAGAACCCGCCACCaugAAGUGAGGCU |
| NO: 89 | hCX40L_ | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAUGGAGCGGGUGCAGCCCCUGGA |
| | | GGAGAACGUGGGCAACGCCGCCCGGCCCCGGUUCGAGCGGAACAAGCUGCUGC |
| | | UGGUGGCCAGCGUGAUCCAGGGCCUGGGCCUGCUGCUGUGCUUCACCUACAUC |
| | | UGCCUGCACUUCAGCGCCCUGCAGGUGAGCCACAGGUACCCCCGGAUCCAGAG |
| | | CAUCAAGGUGCAGUUCACCGAGUACAAGAAGGAGAAGGGCUUCAUCCUGACC |
| | | AGCCAGAAGGAGGACGAGAUCAUGAAGGUGCAGAACAACAGCGUGAUCAUCA |
| | | ACUGCGACGGCUUCUACCUGAUCUCACUGAAGGGCUACUUCAGCCAGGAGGUG |
| | | AACAUCAGCCUGCACUACCAAAAGGACGAGGAGCCCCUGUUCCAGCUGAAGAA |
| | | GGUGCGGAGCGUGAACAGCCUGAUGGUGGCCUCACUGACCUACAAGGACAAG |
| | | GUGUACCUUAACGUGACCACCGACAACACCAGCCUGGACGACUUCCACGUGAA |
| | | CGGCGGCGAGCUGAUCCUGAUUCACCAGAACCCUGGCGAGUUCUGCGUGCUGU |
| | | GAUUAAACCGGUCUGCAGCUUUUGCCUUGGUUCUUCCUAGUGCCUACAAUGG |
| | | GAAAACUUCACUCCAAAGAGAAACCUAUUAAGUCAUCAUCUCCAAACUAAACC |
| | | CUCACAAGUAACAGUUGAAGAAAAAAUGGCAAGAGAUCAUAUCCUCAGACCA |
| | | GGUGGAAUUACUUAAAUUUUAAAGCCUGAAAAUUCCAAUUUGGGGGUGGGAG |
| | | GUGGAAAAGGAACCCAAUUUUCUUAUGAACAGAUAUUUUUAACUUAAUGGCA |
| | | CAAAGUCUUAGAAUAUUAUUAUGUGCCCCGUGUUCCCUGUUCUUCGUUGCUG |
| | | AAUUCGAUAUCCAGCGGCCGCGCUAGCGCUGAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAA |
| SEQ ID | U27- | AGGUCUUCUGGUCCCCACAGACUCAGAGAGAACCCGCCACCaugAAGUGAGGCU |
| NO: 90 | mCX40L | CUCCUCCCGCCCCGCCCCUCCCACGCCUCACCAGCCCCCCGCGCGCCCACCCUC |
| | adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGAGGGCGAGGGCGUGCAGC |
| | | CACUGGAUGAGAACCUGGAGAACGGCAGCCGGCCCCGGUUCAAGUGGAAGAA |
| | | GACCUUACGUCUGGUGGUGAGCGGCAUCAAGGGCGCCGGCAUGCUGCUGUGC |
| | | UUCAUCUACGUGUGUCUGCAGCUGAGCAGCAGCCCCGCCAAGGACCCCCCUAU |
| | | CCAGCGGCUGCGGGGCGCCGUGACCCGGUGCGAGGACGGCCAGCUGUUCAUCA |
| | | GCAGCUACAAGAACGAGUACCAGACCAUGGAGGUGCAGAACAACAGCGUGGU |
| | | GAUCAAGUGCGACGGCCUGUACAUCAUCUACCUGAAGGGCAGCUUCUUCCAGG |
| | | AAGUGAAGAUCGACCUACACUUCAGGGAGGAUCACAACCCCAUCAGCAUCCCC |
| | | AUGCUGAACGACGGCCGGCGGAUCGUGUUCACCGUGGUGGCCAGCCUGGCCUU |
| | | CAAGGACAAGGUGUACCUGACCGUGAACGCCCCCGACACCCUGUGUGAGCACC |
| | | UGCAGAUCAACGACGGCGAGCUGAUCGUGGUGCAGCUGACUCCCGGCUACUGC |
| | | GCCCCCGAGGGCAGCUACCACAGCACCGUGAACCAGGUGCCCCUUUGAUUAAA |
| | | CCGGUCUGCAGCUUUUGCCUUGGUUCUUCCUAGUGCCUACAAUGGGAAAACU |
| | | UCACUCCAAAGAGAAACCUAUUAAGUCAUCAUCUCCAAACUAAACCCUCACAA |
| | | GUAACAGUUGAAGAAAAAAUGGCAAGAGAUCAUAUCCUCAGACCAGGUGGAA |
| | | UUACUUAAAUUUUAAAGCCUGAAAAUUCCAAUUUGGGGGUGGGAGGUGGAAA |
| | | AGGAACCCAAUUUUCUUAUGAACAGAUAUUUUUAACUUAAUGGCACAAAGUC |
| | | UUAGAAUAUUAUUAUGUGCCCCGUGUUCCCUGUUCUUCGUUGCUGAAUUCGA |
| | | UAUCCAGCGGCCGCGCUAGCGCUGAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAA |
| SEQ ID | U27- | AGGUCUUCUGGUCCCCACAGACUCAGAGAGAACCCGCCACCaugAAGUGAGGCU |
| NO: 91 | hIFN- | CUCCUCCCGCCCCGCCCCUCCCACGCCUCACCAGCCCCCCGCGCGCCCACCCUC |
| | A_adm | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCCCUGACCUUCGCCCUGUU |
| | | AGUGGCGCUGCUGGUGCUGAGCUGCAAGAGCAGCUGCAGCGUGGGCUGCGAC |
| | | CUGCCUCAGACCCACAGCCUGGGCAGCCGGCGGACCCUGAUGCUGCUGGCCCA |
| | | GAUGCGGAAGAUCAGCCUGUUCAGCUGUCUGAAGGACCGGCACGACUUCGGC |
| | | UUCCCCCAGGAGGAAUUCGGCAACCAGUUCCAGAAGGCCGAGACCAUCCCCGU |
| | | GCUGCACGAGAUGAUCCAGCAGAUCUUCAACCUGUUCAGCACCAAGGAUAGCA |
| | | GCGCCGCUUGGGACGAGACCCUUCUGGACAAGUUCUACACCGAGCUGUACCAG |
| | | CAGCUGAACGACCUGGAGGCCUGCGUGAUCCAGGGCGUGGGCGUGACCGAGAC |
| | | CCCUCUGAUGAAGGAGGACAGCAUCCUGGCCGUGCGGAAGUACUUUCAGCGG |
| | | AUCACCCUGUACCUGAAGGAGAAGAAGUACAGCCCAUGCGCCUGGGAGGUGG |
| | | UGCGGGCCGAGAUCAUGCGGAGCUUCAGCCUGAGCACCAACCUGCAGGAGAGC |
| | | CUGCGGAGCAAGGAGUGAUUAAACCGGUCUGCAGCUUUUGCCUUGGUUCUUC |
| | | CUAGUGCCUACAAUGGGAAAACUUCACUCCAAAGAGAAACCUAUUAAGUCAU |
| | | CAUCUCCAAACUAAACCCUCACAAGUAACAGUUGAAGAAAAAAUGGCAAGAG |
| | | AUCAUAUCCUCAGACCAGGUGGAAUUACUUAAAUUUUAAAGCCUGAAAAUUC |
| | | CAAUUUGGGGGUGGGAGGUGGAAAAGGAACCCAAUUUUCUUAUGAACAGAUA |
| | | UUUUUAACUUAAUGGCACAAAGUCUUAGAAUAUUAUUAUGUGCCCCGUGUUC |
| | | CCUGUUCUUCGUUGCUGAAUUCGAUAUCCAGCGGCCGCGCUAGCGCUGAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAA |
| SEQ ID | U27-IFN- | AGGUCUUCUGGUCCCCACAGACUCAGAGAGAACCCGCCACCaugAAGUGAGGCU |
| NO: 92 | A_adm | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCUCGGCUGUGCGCCUUCCU |
| | | GAUGAUCCUGGUGAUGAUGAGCUACUACUGGAGCGCAUGCAGCCUGGGCUGC |
| | | GACCUGCCACACACCUACAACCUGGGCAAUAAGCGGGCUCUGACCGUGCUGGA |
| | | GGAGAUGCGGCGGCUGCCCCCCCUGAGCUGCCUGAAGGACCGGAAGGACUUCG |
| | | GCUUCCCCCUGGAGAAGGUGGACAACCAGCAGAUCCAAAAGGCACAGGCCAUC |
| | | CUGGUGCUGCGGGACCUGACCCAGCAGAUCCUGAACCUGUUCACCAGCAAGGA |
| | | CCUGAGCGCCACCUGGAACGCCACCCUGCUGGACAGCUUCUGCAACGACCUGC |
| | | ACCAGCAGCUGAACGACCUGAAGGCCUGCGUCAUGCAGGAGCCACCACUGACC |
| | | CAGGAGGACAGCCUGUUAGCUGUGCGGACUUACUUCCACCGGAUCACCGUGUA |
| | | CCUGCGGAAGAAGAAGCACAGCCUGUGCGCUUGGGAGGUGAUCCGGGCCGAG |
| | | GUGUGGCGGGCCCUGAGCAGCAGCACCAACCUGCUGGCCCGGCUGAGCGAGGA |
| | | GAAGGAGUGAUUAAACCGGUCUGCAGCUUUUGCCUUGGUUCUUCCUAGUGCC |
| | | UACAAUGGGAAAACUUCACUCCAAAGAGAAACCUAUUAAGUCAUCAUCUCCA |
| | | AACUAAACCCUCACAAGUAACAGUUGAAGAAAAAAUGGCAAGAGAUCAUAUC |
| | | CUCAGACCAGGUGGAAUUACUUAAAUUUUAAAGCCUGAAAAUUCCAAUUUGG |
| | | GGGUGGGAGGUGGAAAAGGAACCCAAUUUUCUUAUGAACAGAUAUUUUUAAC |
| | | UUAAUGGCACAAAGUCUUAGAAUAUUAUUAUGUGCCCCGUGUUCCCUGUUCU |
| | | UCGUUGCUGAAUUCGAUAUCCAGCGGCCGCGCUAGCGCUGAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAA |
| SEQ ID | U27-IL- | AGGUCUUCUGGUCCCCACAGACUCAGAGAGAACCCGCCACCaugAAGUGAGGCU |
| NO: 93 | 2_adm | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUACCGGAUGCAGCUGCUGA |
| | | GCUGCAUCGCCCUGAGCCUGGCCCUGGUGACCAACAGCGCCCCCACCAGCAGC |
| | | AGCACCAAGAAGACCCAGCUGCAACUGGAGCACCUGCUGCUGGAUCUGCAGAU |
| | | GAUCCUGAACGGCAUCAACAAUUACAAGAAUCCCAAGCUGACCCGGAUGCUGA |
| | | CCUUCAAGUUCUACAUGCCCAAGAAGGCUACCGAGCUGAAGCACCUGCAGUGC |
| | | CUGGAGGAGGAGCUGAAGCCCCUGGAGGAGGUGCUGAACCUGGCCCAGAGCA |
| | | AGAACUUCCACUUCGACCCCCGGGACGUGGUGAGCAACAUCAACGUGUUCGUG |
| | | CUGGAGCUGAAGGGCAGCGAGACCACCUUCAUGUGCGAGUACGCCGACGAGAC |
| | | CGCCACCAUCGUGGAGUUCCUGAACCGGUGGAUCACCUUCUGCCAGAGCAUCA |
| | | UCAGCUGAUUAAACCGGUCUGCAGCUUUUGCCUUGGUUCUUCCUAGUGCCUAC |
| | | AAUGGGAAAACUUCACUCCAAAGAGAAACCUAUUAAGUCAUCAUCUCCAAAC |
| | | UAAACCCUCACAAGUAACAGUUGAAGAAAAAAUGGCAAGAGAUCAUAUCCUC |
| | | AGACCAGGUGGAAUUACUUAAAUUUUAAAGCCUGAAAAUUCCAAUUUGGGGG |
| | | UGGGAGGUGGAAAAGGAACCCAAUUUUCUUAUGAACAGAUAUUUUUAACUUA |
| | | AUGGCACAAAGUCUUAGAAUAUUAUUAUGUGCCCCGUGUUCCCUGUUCUUCG |
| | | UUGCUGAAUUCGAUAUCCAGCGGCCGCGCUAGCGCUGAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAA |
| SEQ ID | U27-mIL- | AGGUCUUCUGGUCCCCACAGACUCAGAGAGAACCCGCCACCaugAAGUGAGGCU |
| NO: 94 | 2_adm | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUACAGCAUGCAGCUGGCAA |
| | | GCUGCGUGACCCUGACCCUGGUGCUGCUAGUGAACAGCGCCCCCACCAGCAGC |
| | | AGCACCAGCAGCUCCACCGCCGAGGCCCAGCAGCAACAACAGCAACAGCAGCA |
| | | GCAGCAACAGCACCUGGAGCAGCUCCUGAUGGACCUGCAGGAGCUGCUGAGCC |
| | | GGAUGGAGAACUACCGGAACCUGAAGCUGCCCCGGAUGCUGACCUUCAAGUUC |
| | | UACCUGCCCAAGCAGGCCACAGAGCUGAAGGACCUGCAGUGCCUGGAGGACGA |
| | | GCUGGGCCCCCUGCGGCACGUGCUGGAUCUGACCCAGAGCAAGAGCUUCCAGC |
| | | UGGAGGACGCCGAGAACUUCAUCAGCAACAUCCGGGUGACCGUGGUGAAGCU |
| | | GAAGGGCAGCGACAACACCUUCGAGUGCCAGUUCGACGACGAGAGCGCAACCG |
| | | UGGUGGACUUCCUGCGGCGGUGGAUCGCCUUCUGCCAGAGCAUCAUCAGCACC |
| | | AGCCCCCAGUGAUUAAACCGGUCUGCAGCUUUUGCCUUGGUUCUUCCUAGUGC |
| | | CUACAAUGGGAAAACUUCACUCCAAAGAGAAACCUAUUAAGUCAUCAUCUCC |
| | | AAACUAAACCCUCACAAGUAACAGUUGAAGAAAAAAUGGCAAGAGAUCAUAU |
| | | CCUCAGACCAGGUGGAAUUACUUAAAUUUUAAAGCCUGAAAAUUCCAAUUUG |
| | | GGGGUGGGAGGUGGAAAAGGAACCCAAUUUUCUUAUGAACAGAUAUUUUUAA |
| | | CUUAAUGGCACAAAGUCUUAGAAUAUUAUUAUGUGCCCCGUGUUCCCUGUUC |
| | | UUCGUUGCUGAAUUCGAUAUCCAGCGGCCGCGCUAGCGCUGAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAA |
| SEQ ID | U27-hIL- | AGGUCUUCUGGUCCCCACAGACUCAGAGAGAACCCGCCACCaugAAGUGAGGCU |
| NO: 95 | 15_verA | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCCCCCCGGGGGGCCCGGGG |
| | | CUGCCGGACCCUGGGCCUGCCUGCCCUGCUGCUGCUGCUUCUGCUGCGGCCCC |
| | | CCGCCACCCGGGGCAUCACCUGUCCCCCACCUAUGAGCGUGGAGCACGCCGAC |
| | | AUCUGGGUGAAGAGCUACAGCCUGUACAGCCGGGAGCGGUACAUCUGCAACA |
| | | GCGGCUUCAAGCGGAAGGCAGGCACCAGCAGCCUGACCGAGUGCGUGCUGAAC |
| | | AAGGCCACCAACGUGGCCCACUGGACCACCCCCAGCCUUAAGUGCAUCCGGGA |
| | | CCCUGCGCUGGUCCACCAGCGGCCUGCCCCCCCCAGCACCGUGACCACCGCAGG |
| | | AGUGACCCCCCAGCCCGAGAGCCUGAGCCCCAGCGGCAAGGAGCCCGCCGCCA |
| | | GCUCACCCAGCAGCAACAACACCGCCGCCACCACCGCCGCCAUCGUGCCCGGCA |
| | | GCCAGCUGAUGCCUAGCAAGAGCCCCAGCACCGGCACCACCGAGAUCAGCAGC |
| | | CACGAGAGCAGCCACGGCACCCCCAGCCAGACCACCGCCAAGAAUUGGGAGCU |
| | | GACCGCCAGCGCCAGCCACCAGCCCCCAGGCGUGUACCCCCAGGGGCACAGCG |
| | | ACACCACCGUGGCCAUCAGCACCAGCACCGUGCUGCUGUGCGGCCUGAGCGCA |
| | | GUGAGCCUGCUGGCCUGCUACCUGAAGAGCCGGCAGACCCCACCCCUGGCUAG |
| | | CGUGGAGAUGGAGGCCAUGGAGGCCCUGCCCGUGACCUGGGGAACCAGCAGCC |
| | | GGGACGAGGACCUGGAGAACUGCAGCCACCACCUGUGAUUAAACCGGUACCGG |
| | | UUGAUAAUAGCAGACACCAUUGUCACACUCCAGCUGGAGCCUCGGUGGCCAUG |
| | | CUUCUUGCCCCUUGGGCCCAGACACCAUUGUCACACUCCAUCCCCCCAGCCCC |
| | | UCCUCCCCUUCCUGCACCCGUACCCCCCAGACACCAUUGUCACACUCCAGUGG |
| | | UCUUUGAAUAAAGUCUGAGUGGGCGGCGAAUUCGAUAUCCAGCGGCCGCGCU |
| | | AGCGCUGGCUAGCGAAUUCGAUAUCCAGCGGCCGCAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAA |
| SEQ ID | U27-hIL- | AGGUCUUCUGGUCCCCACAGACUCAGAGAGAACCCGCCACCaugAAGUGAGGCU |
| NO: 96 | 15_verB | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGAGUUCGGCCUGAGCUGGG |
| | | UGUUCCUGGUGGCCCUGUUCCGGGGAGUGCAGUGUAUCACCUGCCCCCCCCCC |
| | | AUGAGCGUGGAGCACGCUGACAUCUGGGUGAAGAGCUACAGCCUGUACAGCC |
| | | GGGAGCGGUACAUCUGCAACAGCGGCUUCAAGCGGAAGGCCGGCACCAGCUCC |
| | | CUGACCGAGUGCGUGCUGAACAAGGCCACCAACGUGGCCCACUGGACCACCCC |
| | | CAGCCUGAAGUGCAUCCGGGACCCCGCCCUGGUGGGCGGCUCAGGCGGCAGCG |
| | | GCGGCAGCGGGGGCAGCGGAGGCAGCGGCGGAAGCGGCGGCAACUGGGUGAA |
| | | CGUGAUCAGCGACCUGAAGAAGAUCGAGGACCUGAUCCAAAGCAUGCACAUU |
| | | GACGCCACCCUGUACACCGAGAGCGACGUGCACCCCAGCUGCAAGGUGACCGC |
| | | AAUGAAGUGCUUCCUGCUGGAGCUGCAGGUGAUCAGCCUGGAGAGCGGCGAC |
| | | GCCAGCAUCCACGACACCGUGGAGAACCUGAUCAUCCUGGCGAACAACAGCCU |
| | | GAGCAGCAACGGCAACGUGACCGAGAGCGGCUGUAAGGAGUGCGAGGAGCUG |
| | | GAGGAGAAGAAUAUCAAGGAGUUCCUGCAGAGCUUCGUGCACAUCGUGCAGA |
| | | UGUUCAUCAACACCAGCUGAUUAAACCGGUACCGGUUGAUAAUAGCAGACAC |
| | | CAUUGUCACACUCCAGCUGGAGCCUCGGUGGCCAUGCUUCUUGCCCCUUGGGC |
| | | CCAGACACCAUUGUCACACUCCAUCCCCCCAGCCCCUCCUCCCCUUCCUGCACC |
| | | CGUACCCCCCAGACACCAUUGUCACACUCCAGUGGUCUUUGAAUAAAGUCUGA |
| | | GUGGGCGGCGAAUUCGAUAUCCAGCGGCCGCGCUAGCGCUGGCUAGCGAAUUC |
| | | GAUAUCCAGCGGCCGCAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAA |
| SEQ ID | U27-hIL- | AGGUCUUCUGGUCCCCACAGACUCAGAGAGAACCCGCCACCaugAAGUGAGGCU |
| NO: 97 | 15_verC | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGAGUUCGGCCUGAGCUGGG |
| | | UGUUUCUGGUGGCCCUGUUCCGGGGCGUGCAGUGCAACUGGGUGAACGUGAU |
| | | CAGCGACCUGAAGAAGAUCGAGGACCUGAUCCAGUCAAUGCACAUCGAUGCA |
| | | ACCCUGUACACCGAGAGCGACGUGCACCCUAGCUGUAAGGUGACCGCAAUGAA |
| | | GUGCUUCCUGCUGGAGCUGCAGGUGAUAAGCCUGGAGAGCGGCGACGCCAGC |
| | | AUCCACGACACAGUCGAGAAUCUGAUCAUCCUGGCUAACAACAGCCUGAGCAG |
| | | CAAUGGUAAUGUGACCGAGAGCGGCUGCAAGGAGUGCGAGGAGCUGGAGGAG |
| | | AAGAACAUCAAGGAGUUCCUGCAGAGCUUCGUGCACAUCGUGCAGAUGUUCA |
| | | UCAACACCAGCGGCGGCAGCGGGGGCAGCGGCGGCUCAGGUGGCAGCGGAGGC |
| | | AGCGGCGGAAGCGGCGGCAUCACCUGCCCUCCCCCCAUGAGCGUGGAGCACGC |
| | | AGACAUCUGGGUGAAGAGCUACAGCCUGUACAGCCGGGAGCGGUACAUCUGC |
| | | AACAGCGGCUUCAAGCGGAAGGCCGGCACCAGCAGCCUGACCGAGUGUGUGCU |
| | | GAACAAGGCCACCAACGUGGCCCACUGGACAACCCCCAGCCUGAAGUGCAUCA |
| | | GGGACCCCGCCCUGGUGUGAUUAAACCGGUACCGGUUGAUAAUAGCAGACACC |
| | | AUUGUCACACUCCAGCUGGAGCCUCGGUGGCCAUGCUUCUUGCCCCUUGGGCC |
| | | CAGACACCAUUGUCACACUCCAUCCCCCCAGCCCCUCCUCCCCUUCCUGCACCC |
| | | GUACCCCCCAGACACCAUUGUCACACUCCAGUGGUCUUUGAAUAAAGUCUGAG |
| | | UGGGCGGCGAAUUCGAUAUCCAGCGGCCGCGCUAGCGCUGGCUAGCGAAUUCG |
| | | AUAUCCAGCGGCCGCAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAA |
| SEQ ID | U27-mIL- | AGGUCUUCUGGUCCCCACAGACUCAGAGAGAACCCGCCACCaugAAGUGAGGCU |
| NO: 98 | 15_verA_ | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCUAGCCCCCAGCUGCGGGG |
| | | CUACGGCGUGCAGGCCAUCCCUGUGCUGCUGUUACUGCUGCUGCUCCUGCUGC |
| | | UGCCCCUGCGGGUGACCCCCGGCACCACCUGCCCCCCCCCCGUGAGCAUCGAGC |
| | | ACGCCGACAUCCGGGUGAAGAACUACAGCGUGAACAGCCGGGAGAGGUACGU |
| | | GUGCAAUAGCGGCUUCAAGCGGAAGGCCGGCACCAGCACCCUGAUCGAGUGCG |
| | | UGAUCAACAAGAACACCAACGUGGCCCACUGGACCACCCCCAGCCUGAAGUGC |
| | | AUCCGGGACCCCAGCUUAGCCCACUACAGCCCCGUGCCCACCGUGGUGACCCC |
| | | AAAGGUGACCAGCCAGCCCGAGAGCCCCAGCCCCAGCGCCAAGGAGCCUGAGG |
| | | CCUUCUCCCCAAAGAGCGACACCGCCAUGACCACCGAGACCGCCAUCAUGCCC |
| | | GGCAGCCGGCUGACCCCCAGCCAGACCACCAGCGCCGGCACCACAGGCACCGG |
| | | CAGCCACAAGAGCAGCCGGGCACCCAGCCUGGCCGCCACCAUGACCCUGGAGC |
| | | CUACCGCUAGCACCAGCCUGCGGAUCACCGAGAUCAGCCCCCACAGCAGCAAG |
| | | AUGACCAAGGUGGCCAUCAGCACCAGCGUGCUGCUGGUGGGCGCCGGCGUGGU |
| | | GAUGGCUUUCCUGGCCUGGUACAUCAAGAGCCGGCAGCCCAGCCAGCCUUGCC |
| | | GGGUGGAGGUGGAGACCAUGGAGACUGUGCCCAUGACCGUGCGGGCCAGCAG |
| | | CAAGGAGGACGAGGACACCGGCGCCGGGGGCAGCGGCGGUAGCGGAGGCAGC |
| | | GGCGGCAGCGGCGGAAGCGGCGGCUCCGGCGGCAUGAAGAUCCUGAAGCCCUA |
| | | CAUGCGGAACACCAGCAUCAGCUGCUACCUGUGCUUCCUGCUGAACAGCCACU |
| | | UCCUGACCGAGGCCGGCAUCCACGUGUUCAUCCUGGGCUGCGUGAGCGUGGGC |
| | | CUGCCUAAGACCGAGGCCAACUGGAUCGACGUGCGGUACGACCUGGAGAAGA |
| | | UCGAGAGCCUGAUCCAGAGCAUCCACAUCGACACCACCCUGUACACCGACAGC |
| | | GACUUCCACCCCAGCUGCAAGGUGACCGCCAUGAACUGCUUUCUGCUGGAGCU |
| | | GCAGGUGAUCCUGCACGAGUACAGCAACAUGACCCUGAACGAGACCGUGCGGA |
| | | ACGUGCUGUACCUGGCCAACAGCACCCUGAGCAGCAACAAGAACGUGGCCGAG |
| | | AGCGGCUGCAAGGAGUGCGAGGAGCUGGAGGAGAAGACCUUCACCGAGUUCC |
| | | UGCAGAGCUUCAUCCGGAUCGUGCAGAUGUUCAUCAACACCAGCUGAUUAAA |
| | | CCGGUACCGGUUGAUAAUAGCAGACACCAUUGUCACACUCCAGCUGGAGCCUC |
| | | GGUGGCCAUGCUUCUUGCCCCUUGGGCCCAGACACCAUUGUCACACUCCAUCC |
| | | CCCCAGCCCCUCCUCCCCUUCCUGCACCCGUACCCCCCAGACACCAUUGUCACA |
| | | CUCCAGUGGUCUUUGAAUAAAGUCUGAGUGGGCGGCGAAUUCGAUAUCCAGC |
| | | GGCCGCGCUAGCGCUGGCUAGCGAAUUCGAUAUCCAGCGGCCGCAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAA |
| SEQ ID | U27-mIL- | AGGUCUUCUGGUCCCCACAGACUCAGAGAGAACCCGCCACCaugAAGUGAGGCU |
| NO: 99 | 15_verB_ | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGAGCGUGCUGACCCAGGUGC |
| | | UGGCCCUGCUGCUGCUGUGGCUGACCGGCGCCCGUUGCACCACCUGCCCCCCC |
| | | CCAGUGAGCAUCGAGCACGCCGAUAUCCGGGUGAAGAACUACAGCGUGAACA |
| | | GCCGGGAGCGGUACGUGUGCAACAGCGGCUUCAAGCGGAAGGCCGGCACAAGC |
| | | ACCCUGAUCGAGUGCGUGAUCAACAAGAACACCAAUGUGGCCCACUGGACCAC |
| | | CCCCAGCCUGAAGUGUAUCCGGGACCCUAGCCUGGCCGGCGGCAGCGGCGGGA |
| | | GCGGUGGCAGCGGCGGAAGCGGCGGUAGCGGAGGCAGCGGCGGCAACUGGAU |
| | | CGACGUGCGGUACGACCUAGAGAAGAUCGAGAGCCUGAUCCAGAGCAUCCACA |
| | | UCGACACCACCCUGUACACCGACAGCGACUUCCACCCCAGCUGCAAGGUGACC |
| | | GCCAUGAACUGCUUCCUGCUGGAGCUGCAGGUGAUCCUGCACGAGUACACCAA |
| | | CAUGACACUGAACGAGACCGUGCGGAACGUGCUGUACCUGGCAAACAGCACCC |
| | | UGAGCAGCAACAAGAAUGUGGCCGAGAGCGGCUGCAAGGAAUGCGAGGAGCU |
| | | GGAGGAGAAGACCUUCACCGAGUUCCUCCAGAGCUUCAUCCGGAUCGUGCAGA |
| | | UGUUCAUCAACACCAGCUGAUUAAACCGGUACCGGUUGAUAAUAGCAGACAC |
| | | CAUUGUCACACUCCAGCUGGAGCCUCGGUGGCCAUGCUUCUUGCCCCUUGGGC |
| | | CCAGACACCAUUGUCACACUCCAUCCCCCCAGCCCCUCCUCCCCUUCCUGCACC |
| | | CGUACCCCCCAGACACCAUUGUCACACUCCAGUGGUCUUUGAAUAAAGUCUGA |
| | | GUGGGCGGCGAAUUCGAUAUCCAGCGGCCGCGCUAGCGCUGGCUAGCGAAUUC |
| | | GAUAUCCAGCGGCCGCAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAA |
| SEQ ID | U27-mIL- | AGGUCUUCUGGUCCCCACAGACUCAGAGAGAACCCGCCACCaugAAGUGAGGCU |
| NO: 100 | 15_verC | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGAGCGUGCUGACCCAGGUGC |
| | | UGGCUCUACUGCUGCUGUGGUUAACAGGCGCCCGGUGCAACUGGAUCGACGU |
| | | GCGGUACGACCUGGAGAAGAUCGAGAGCCUGAUCCAGAGCAUCCACAUCGACA |
| | | CCACCCUGUACACCGACAGCGACUUCCACCCUAGCUGCAAGGUGACCGCCAUG |
| | | AACUGCUUCCUGCUGGAGCUGCAGGUGAUCCUGCACGAGUACAGCAAUAUGA |
| | | CCCUGAACGAGACCGUGCGGAACGUGCUGUACCUGGCCAACAGCACCCUGAGC |
| | | AGCAAUAAGAAUGUGGCCGAGAGCGGCUGCAAGGAGUGUGAGGAGCUGGAGG |
| | | AGAAGACCUUCACUGAGUUCCUGCAGAGCUUUAUCCGGAUCGUGCAGAUGUU |
| | | CAUCAACACUAGCGGCGGAAGCGGCGGCUCAGGCGGCAGCGGAGGCAGCGGCG |
| | | GCAGCGGGGGCAGCGGCGGAACCACCUGUCCCCCCCCCGUGAGCAUCGAGCAC |
| | | GCCGACAUCCGGGUGAAGAACUACAGCGUGAACUCACGGGAGCGGUACGUGU |
| | | GCAACUCAGGCUUCAAGCGGAAGGCCGGCACCAGCACCCUGAUCGAGUGCGUG |
| | | AUCAACAAGAACACAAACGUGGCCCACUGGACCACCCCCAGCCUGAAGUGCAU |
| | | CCGGGACCCCAGCCUGGCCUGAUUAAACCGGUACCGGUUGAUAAUAGCAGACA |
| | | CCAUUGUCACACUCCAGCUGGAGCCUCGGUGGCCAUGCUUCUUGCCCCUUGGG |
| | | CCCAGACACCAUUGUCACACUCCAUCCCCCCAGCCCCUCCUCCCCUUCCUGCAC |
| | | CCGUACCCCCCAGACACCAUUGUCACACUCCAGUGGUCUUUGAAUAAAGUCUG |
| | | AGUGGGCGGCGAAUUCGAUAUCCAGCGGCCGCGCUAGCGCUGGCUAGCGAAU |
| | | UCGAUAUCCAGCGGCCGCAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAA |
| SEQ ID | U27-mIL- | AGGUCUUCUGGUCCCCACAGACUCAGAGAGAACCCGCCACCaugAAGUGAGGCU |
| NO: 101 | 15_verD_ | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGAGCGUGCUGACCCAGGUGC |
| | | UGGCCCUGCUGCUGCUGUGGCUGACCGGCGCCCGGUGCAACUGGAUCGACGUG |
| | | CGGUACGAUCUGGAGAAGAUCGAGAGCCUGAUCCAGAGCAUCCACAUCGACAC |
| | | CACCCUGUACACCGAUAGCGACUUCCACCCCAGCUGCAAGGUGACCGCCAUGA |
| | | ACUGCUUCCUGCUGGAACUGCAGGUGAUCCUGCACGAGUACAGCAACAUGACC |
| | | CUGAACGAGACCGUGCGGAACGUCCUGUACCUGGCCAACAGCACACUGAGCAG |
| | | CAACAAGAACGUGGCCGAGAGCGGCUGCAAGGAGUGCGAGGAGCUGGAGGAG |
| | | AAGACCUUCACCGAGUUCCUGCAGAGCUUCAUCCGUAUCGUGCAGAUGUUCAU |
| | | CAACACCAGCGGGGGCAGCGGCGGAAGCGGGGGCAGCGGCGGAAGCGGCGGCA |
| | | GCGGAGGCAGCGGAGGCACCACAUGCCCCCCACCCGUGAGCAUCGAGCACGCC |
| | | GACAUACGGGUGAAGAAUUACAGCGUGAACAGCCGGGAGCGGUACGUGUGCA |
| | | ACAGCGGCUUCAAGCGGAAGGCCGGCACCAGCACCCUGAUCGAGUGCGUGAUC |
| | | AACAAGAACACUAACGUGGCACACUGGACCACCCCCAGCCUCAAGUGCAUCCG |
| | | GGACCCAAGCCUGGCCGGGGGGGGCGGCUCCGGCGGAGGCGGCAGCGGCGGUG |
| | | GAGGCAGCGGCCCUACAAUCAAGCCCUGCCCCCCCUGCAAGUGCCCCGCCCCC |
| | | AAUGCCGCCGGCGGCCCCAGCGUGUUCAUCUUCCCCCCCAAGAUCAAGGACGU |
| | | GCUGAUGAUCUCACUAAGCCCCAUAGUGACCUGCGUGGUGGUGGACGUGAGC |
| | | GAGGACGACCCUGACGUGCAGAUCAGCUGGUUCGUGAACAACGUGGAGGUGC |
| | | ACACCGCCCAGACCCAGACCCACCGGGAGGACUACAACAGCACCCUGCGGGUG |
| | | GUGAGCGCCCUGCCCAUCCAGCACCAGGACUGGAUGAGCGGCAAGGAGUUCAA |
| | | GUGUAAGGUGAAUAAUAAGGACCUGGGCGCCCCAAUAGAGCGGACCAUUAGC |
| | | AAGCCCAAGGGGAGCGUGCGGGCCCCCCAGGUGUACGUGCUGCCACCCCCUGA |
| | | GGAGGAGAUGACCAAAAAGCAGGUGACCCUGACCUGCAUGGUGACCGAUUUC |
| | | AUGCCAGAGGACAUCUACGUGGAGUGGACCAACAACGGCAAGACCGAGCUGA |
| | | ACUACAAGUGAUUAAACCGGUACCGGUUGAUAAUAGCAGACACCAUUGUCAC |
| | | ACUCCAGCUGGAGCCUCGGUGGCCAUGCUUCUUGCCCCUUGGGCCCAGACACC |
| | | AUUGUCACACUCCAUCCCCCCAGCCCCUCCUCCCCUUCCUGCACCCGUACCCCC |
| | | CAGACACCAUUGUCACACUCCAGUGGUCUUUGAAUAAAGUCUGAGUGGGCGG |
| | | CGAAUUCGAUAUCCAGCGGCCGCGCUAGCGCUGGCUAGCGAAUUCGAUAUCCA |
| | | GCGGCCGCAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAA |
| SEQ ID | U27-hIL- | AGGUCUUCUGGUCCCCACAGACUCAGAGAGAACCCGCCACCaugAAGUGAGGCU |
| NO: 102 | 12_down1 | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUGCCACCAGCAGCUGGUGA |
| | | UCAGCUGGUUCAGCCUGGUGUUCCUCGCCAGCCCCCUGGUGGCUAUCUGGGAG |
| | | CUGAAGAAGGACGUGUACGUGGUGGAGCUGGACUGGUACCCCGACGCCCCCGG |
| | | CGAGAUGGUGGUGCUCACCUGCGAUACCCCCGAGGAGGACGGAAUCACCUGGA |
| | | CCCUGGACCAGAGCAGCGAGGUGUUAGGCAGCGGCAAGACCCUGACCAUCCAG |
| | | GUGAAGGAGUUCGGCGACGCCGGCCAGUACACCUGCCACAAGGGGGGGGAGG |
| | | UGCUGAGCCACAGCCUGCUGCUGCUGCACAAGAAGGAGGACGGCAUCUGGAGC |
| | | ACCGACAUCCUGAAGGACCAGAAGGAGCCCAAGAACAAGACCUUCCUGCGGUG |
| | | CGAGGCCAAGAAUUACAGCGGCCGGUUCACCUGCUGGUGGCUGACCACCAUCA |
| | | GCACCGACCUGACCUUCAGCGUGAAGAGCAGCCGGGGCAGCAGCGACCCCCAG |
| | | GGCGUGACCUGCGGCGCCGCCACCCUGAGCGCCGAGCGGGUGAGGGGCGACAA |
| | | UAAGGAGUACGAGUACAGCGUGGAGUGUCAGGAGGAUAGCGCAUGCCCCGCC |
| | | GCCGAGGAGAGCCUGCCCAUCGAGGUGAUGGUGGACGCCGUGCACAAGCUGA |
| | | AGUACGAGAACUACACCAGCAGCUUCUUCAUCCGGGACAUCAUCAAGCCCGAC |
| | | CCCCCCAAGAACCUGCAGCUGAAGCCCCUGAAGAACAGCCGGCAGGUGGAGGU |
| | | GUCAUGGGAGUACCCCGACACCUGGAGCACCCCCCACAGCUACUUCAGCCUGA |
| | | CAUUCUGUGUGCAGGUGCAGGGCAAGAGCAAGCGGGAGAAGAAGGACCGGGU |
| | | GUUCACCGACAAGACCAGCGCCACCGUGAUCUGCCGGAAGAAUGCCAGCAUCA |
| | | GCGUGCGGGCCCAGGACCGGUACUACAGCAGCAGCUGGAGCGAGUGGGCCAGC |
| | | GUGCCUUGCUCCGGCGGCGGAGGAAGCGGAGGCGGGGGCAGCGGGGGGGGCG |
| | | GAAGCCGGAACCUGCCCGUGGCCACCCCAGACCCCGGCAUGUUCCCCUGCCUG |
| | | CACCACAGCCAGAACCUGCUGCGGGCCGUGAGCAACAUGCUGCAGAAGGCUCG |
| | | GCAGACCCUGGAGUUCUACCCCUGCACCAGCGAGGAGAUCGACCACGAGGACA |
| | | UCACCAAGGACAAGACUAGCACCGUGGAGGCCUGCCUGCCCCUGGAGCUGACC |
| | | AAGAAUGAGAGCUGCCUGAACAGCCGGGAGACCAGCUUUAUCACCAACGGCA |
| | | GCUGCCUGGCCAGCCGGAAGACCUCAUUCAUGAUGGCCCUGUGCCUGAGCAGC |
| | | AUCUACGAGGACCUGAAGAUGUACCAGGUGGAGUUCAAGACCAUGAACGCCA |
| | | AGCUGCUGAUGGACCCUAAGCGGCAAAUCUUCCUGGACCAGAAUAUGCUGGCC |
| | | GUGAUCGACGAGCUCAUGCAGGCCCUGAACUUUAACAGCGAGACCGUGCCCCA |
| | | GAAGAGCAGCCUGGAGGAGCCAGACUUCUACAAGACCAAGAUCAAGCUGUGC |
| | | AUCCUGCUGCACGCUUUCCGGAUCCGGGCCGUGACCAUCGACCGGGUGAUGAG |
| | | CUACCUGAACGCGAGCAGCGGAGGCGGCAGCGGUGGCGGCGGCUCAGGCGGCG |
| | | GAGGCAGCGGCGGAGGCGGAAGCGGCGGCGGCAGCCUGCAGGUGGUGAUCAG |
| | | CGCCAUCCUGGCUCUGGUGGUGCUGACCAUCAUCUCCCUGAUCAUCCUGAUCG |
| | | GCGGAGGUGGCAGCUGAUUAAACCGGUACCGGUUGAUAAUAGCAGACACCAU |
| | | UGUCACACUCCAGCUGGAGCCUCGGUGGCCAUGCUUCUUGCCCCUUGGGCCCA |
| | | GACACCAUUGUCACACUCCAUCCCCCCAGCCCCUCCUCCCCUUCCUGCACCCGU |
| | | ACCCCCCAGACACCAUUGUCACACUCCAGUGGUCUUUGAAUAAAGUCUGAGUG |
| | | GGCGGCGAAUUCGAUAUCCAGCGGCCGCGCUAGCGCUGGCUAGCGAAUUCGAU |
| | | AUCCAGCGGCCGCAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAA |
| SEQ ID | U27-mIL- | AGGUCUUCUGGUCCCCACAGACUCAGAGAGAACCCGCCACCaugAAGUGAGGCU |
| NO: 103 | 12_down1 | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUGCCCCCAGAAGCUGACCAU |
| | | CAGCUGGUUCGCCAUCGUGCUGCUGGUGAGCCCUCUGAUGGCCAUGUGGGAGC |
| | | UGGAGAAGGACGUGUACGUGGUGGAGGUGGAUUGGACCCCCGACGCACCCGG |
| | | CGAGACCGUGAACCUGACCUGCGAUACCCCUGAGGAGGACGACAUCACCUGGA |
| | | CUAGCGAUCAGCGGCACGGCGUGAUCGGCAGCGGCAAGACCCUGACCAUCACC |
| | | GUGAAGGAGUUCCUGGAUGCCGGCCAGUACACCUGCCACAAGGGGGGGGAGA |
| | | CCCUGAGCCACAGCCACCUGCUGCUGCACAAAAAGGAGAACGGCAUCUGGAGC |
| | | ACCGAGAUCCUGAAGAACUUCAAGAACAAGACCUUUCUGAAGUGCGAGGCUC |
| | | CCAACUACAGCGGUCGGUUCACCUGCAGCUGGCUGGUGCAGCGGAAUAUGGAC |
| | | CUGAAGUUUAACAUCAAGUCAAGCAGCAGCAGCCCCGACAGCCGGGCCGUGAC |
| | | CUGUGGCAUGGCCAGCCUGAGCGCCGAGAAGGUGACCCUGGACCAGAGGGAU |
| | | UACGAGAAGUACAGCGUGAGCUGCCAGGAGGACGUGACCUGCCCAACCGCCGA |
| | | GGAGACCCUGCCCAUCGAGCUGGCCCUGGAGGCCCGGCAGCAGAACAAGUACG |
| | | AGAACUACAGCACCAGCUUCUUCAUCCGGGACAUCAUCAAGCCUGACCCCCCC |
| | | AAGAACCUGCAGAUGAAGCCCCUGAAGAACAGCCAGGUGGAGGUGAGCUGGG |
| | | AGUACCCCGACAGCUGGAGCACCCCCCACAGCUACUUCAGCCUGAAGUUCUUC |
| | | GUGCGGAUCCAGCGGAAGAAGGAGAAGAUGAAGGAGACCGAGGAGGGCUGCA |
| | | ACCAGAAGGGCGCCUUCCUGGUGGAGAAGACCAGCACCGAGGUGCAGUGUAA |
| | | GGGCGGCAACGUGUGCGUGCAGGCCCAGGACCGGUACUACAACAGCUCAUGCA |
| | | GCAAGUGGGCCUGCGUGCCCUGCCGGGUGAGGAGCGGCGGCGGCGGCUCCGGC |
| | | GGAGGAGGCAGCGGAGGAGGCGGCAGCCGGGUGAUCCCCGUGAGCGGCCCUGC |
| | | CCGGUGCCUGAGCCAGAGCCGGAACCUGCUGAAGACCACCGACGACAUGGUGA |
| | | AGACCGCUCGGGAGAAGCUGAAGCACUACAGCUGCACCGCCGAGGACAUCGAC |
| | | CACGAGGACAUCACCCGGGACCAGACCAGCACCUUAAAGACCUGCCUGCCCCU |
| | | GGAGCUGCACAAGAAUGAGAGCUGCCUGGCCACCCGGGAGACCAGCAGCACCA |
| | | CCCGGGGCAGCUGCCUGCCCCCCCAGAAGACCAGCCUGAUGAUGACCCUGUGC |
| | | CUGGGCAGCAUCUACGAGGACCUGAAGAUGUACCAGACCGAGUUCCAGGCAA |
| | | UCAACGCUGCUCUGCAGAAUCACAACCACCAGCAGAUCAUCCUGGACAAGGGA |
| | | AUGCUGGUGGCCAUCGACGAGCUGAUGCAGAGCCUGAACCACAACGGCGAGAC |
| | | CCUGCGGCAGAAGCCCCCCGUGGGCGAGGCCGACCCCUACCGUGUGAAGAUGA |
| | | AGCUGUGCAUCCUGCUGCACGCUUUCAGCACCCGGGUGGUGACCAUCAACCGG |
| | | GUGAUGGGCUACCUGAGCAGCGCAAGCGGUGGCGGCAGCGGCGGCGGGGGCU |
| | | CCGGCGGCGGUGGGAGCGGCGGAGGCGGUUCAGGCGGCGGCAGCCUGCAGGU |
| | | GGUGAUCAGCGCCAUCCUGGCCCUGGUGGUGCUCACCGUGAUCAGCCUGAUCA |
| | | UCCUGAUCGGGGGCGGCGGCUCAGGCAAGCCCAUCCCAAAUCCACUGCUGGGC |
| | | CUGGACAGCACCUGAUUAAACCGGUACCGGUUGAUAAUAGCAGACACCAUUG |
| | | UCACACUCCAGCUGGAGCCUCGGUGGCCAUGCUUCUUGCCCCUUGGGCCCAGA |
| | | CACCAUUGUCACACUCCAUCCCCCCAGCCCCUCCUCCCCUUCCUGCACCCGUAC |
| | | CCCCCAGACACCAUUGUCACACUCCAGUGGUCUUUGAAUAAAGUCUGAGUGGG |
| | | CGGCGAAUUCGAUAUCCAGCGGCCGCGCUAGCGCUGGCUAGCGAAUUCGAUAU |
| | | CCAGCGGCCGCAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAA |
| SEQ ID | U27-TET- | AGGUCUUCUGGUCCCCACAGACUCAGAGAGAACCCGCCACCaugAAGUGAGGCU |
| NO: 104 | IL- | CUCCUCCCGCCCCGCCCCUCCCACGCCUCACCAGCCCCCCGCGCGCCCACCCUC |
| | 12_down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUGCCCCCAGAAGCUGACCAU |
| | | CAGCUGGUUCGCCAUCGUGCUGCUGGUGAGCCCUCUGAUGGCCAUGUGGGAGC |
| | | UCGAGAAGGACGUGUACGUGGUGGAGGUGGACUGGACCCCCGACGCCCCCGGC |
| | | GAGACCGUGAACCUGACCUGUGACACCCCCGAGGAGGACGACAUCACCUGGAC |
| | | CAGCGACCAGCGGCACGGCGUGAUCGGAAGCGGCAAGACCCUGACCAUCACCG |
| | | UCAAGGAGUUCCUGGACGCCGGCCAGUACACCUGCCACAAGGGCGGCGAGACA |
| | | CUGAGCCACAGCCACCUGCUGUUACACAAGAAGGAGAACGGCAUCUGGAGCAC |
| | | CGAGAUUCUGAAGAACUUCAAGAACAAGACCUUCCUGAAGUGCGAAGCCCCCA |
| | | AUUACAGCGGCCGGUUCACCUGCAGCUGGCUGGUGCAGCGGAACAUGGACCUG |
| | | AAGUUCAAUAUCAAGUCAAGCAGCAGCAGCCCCGAUAGCCGGGCCGUGACCUG |
| | | CGGCAUGGCCAGCCUGAGCGCCGAGAAGGUGACCCUGGACCAGAGGGACUACG |
| | | AGAAGUACAGCGUGAGCUGUCAGGAGGACGUGACAUGCCCAACCGCCGAGGA |
| | | GACCCUGCCCAUCGAGCUGGCCCUGGAGGCCCGGCAGCAGAAUAAGUACGAGA |
| | | AUUACAGCACCAGCUUCUUCAUCCGGGACAUCAUCAAGCCCGACCCCCCUAAG |
| | | AACCUGCAGAUGAAGCCCCUGAAGAAUAGCCAGGUCGAGGUGAGCUGGGAGU |
| | | ACCCCGAUAGCUGGAGCACCCCCCACAGCUACUUCAGCCUGAAGUUCUUCGUG |
| | | CGGAUCCAGCGGAAGAAGGAGAAGAUGAAGGAGACCGAGGAGGGCUGCAAUC |
| | | AGAAGGGCGCAUUCCUGGUGGAGAAGACCAGCACCGAGGUGCAGUGCAAGGG |
| | | CGGCAACGUGUGCGUGCAGGCCCAGGAUCGGUACUACAACAGCAGCUGCAGCA |
| | | AGUGGGCCUGCGUGCCAUGCCGGGUGCGGAGCGGCGGCGGAGGCUCAGGUGG |
| | | CGGCGGCUCAGGCGGGGGCGGCAGCCGGGUGAUCCCCGUGAGCGGCCCCGCCC |
| | | GGUGCCUUAGCCAGAGCCGGAAUCUGCUGAAGACCACCGACGACAUGGUGAA |
| | | GACCGCCCGGGAGAAGCUGAAGCACUACAGCUGCACCGCCGAGGACAUCGACC |
| | | ACGAGGACAUCACCCGGGACCAGACCAGCACCCUGAAGACCUGCCUGCCCCUG |
| | | GAGCUGCACAAGAACGAGAGCUGCCUGGCAACCCGGGAGACCAGCAGCACCAC |
| | | CCGGGGCAGCUGCCUCCCCCCCCAGAAGACCAGCCUGAUGAUGACCCUGUGCC |
| | | UGGGGAGCAUCUACGAGGACCUGAAGAUGUACCAGACCGAGUUCCAGGCUAU |
| | | AAACGCCGCCCUGCAGAACCACAACCACCAGCAGAUCAUCCUGGACAAGGGCA |
| | | UGCUGGUGGCCAUCGACGAGCUGAUGCAGAGCCUGAACCACAACGGCGAGACU |
| | | CUGCGGCAGAAGCCCCCUGUGGGCGAGGCCGAUCCCUACCGGGUGAAGAUGAA |
| | | GCUGUGCAUCCUGCUGCACGCCUUCAGCACCCGGGUGGUGACCAUCAACAGGG |
| | | UGAUGGGCUACCUGAGCAGCGCUAGCGGCGGAGGUAGCGGAGGGGGGGGCUC |
| | | CGGCGGCGGCGGCAGCGGUGGAGGCGGCAGCGGCGGCGGUAGCCUGCAAGUG |
| | | GUGAUCAGCGCCAUCCUGGCCCUGGUGGUGCUGACCGUGAUCAGCCUGAUCAU |
| | | CCUGAUCGGCGGUGGCGGGAGCGGCAAGCCCAUCCCCAACCCACUGCUGGGCC |
| | | UGGACAGCACCUGAUUAAACCGGUACCGGUUGAUAAUAGCAGACACCAUUGU |
| | | CACACUCCAGCUGGAGCCUCGGUGGCCAUGCUUCUUGCCCCUUGGGCCCAGAC |
| | | ACCAUUGUCACACUCCAUCCCCCCAGCCCCUCCUCCCCUUCCUGCACCCGUACC |
| | | CCCCAGACACCAUUGUCACACUCCAGUGGUCUUUGAAUAAAGUCUGAGUGGGC |
| | | GGCGAAUUCGAUAUCCAGCGGCCGCGCUAGCGCUGGCUAGCGAAUUCGAUAUC |
| | | CAGCGGCCGCAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAA |
| SEQ ID | U27- | AGGUCUUCUGGUCCCCACAGACUCAGAGAGAACCCGCCACCaugAAGUGAGGCU |
| NO: 105 | hCX40L_ | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAUGGAGCGGGUGCAGCCCCUGGA |
| | | GGAGAACGUGGGCAACGCCGCCCGGCCCCGGUUCGAGCGGAACAAGCUGCUGC |
| | | UGGUGGCCAGCGUGAUCCAGGGCCUGGGCCUGCUGCUGUGCUUCACCUACAUC |
| | | UGCCUGCACUUCAGCGCCCUGCAGGUGAGCCACAGGUACCCCCGGAUCCAGAG |
| | | CAUCAAGGUGCAGUUCACCGAGUACAAGAAGGAGAAGGGCUUCAUCCUGACC |
| | | AGCCAGAAGGAGGACGAGAUCAUGAAGGUGCAGAACAACAGCGUGAUCAUCA |
| | | ACUGCGACGGCUUCUACCUGAUCUCACUGAAGGGCUACUUCAGCCAGGAGGUG |
| | | AACAUCAGCCUGCACUACCAAAAGGACGAGGAGCCCCUGUUCCAGCUGAAGAA |
| | | GGUGCGGAGCGUGAACAGCCUGAUGGUGGCCUCACUGACCUACAAGGACAAG |
| | | GUGUACCUUAACGUGACCACCGACAACACCAGCCUGGACGACUUCCACGUGAA |
| | | CGGCGGCGAGCUGAUCCUGAUUCACCAGAACCCUGGCGAGUUCUGCGUGCUGU |
| | | GAUUAAACCGGUACCGGUUGAUAAUAGCAGACACCAUUGUCACACUCCAGCU |
| | | GGAGCCUCGGUGGCCAUGCUUCUUGCCCCUUGGGCCCAGACACCAUUGUCACA |
| | | CUCCAUCCCCCCAGCCCCUCCUCCCCUUCCUGCACCCGUACCCCCCAGACACCA |
| | | UUGUCACACUCCAGUGGUCUUUGAAUAAAGUCUGAGUGGGCGGCGAAUUCGA |
| | | UAUCCAGCGGCCGCGCUAGCGCUGGCUAGCGAAUUCGAUAUCCAGCGGCCGCA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAA |
| SEQ ID | U27- | AGGUCUUCUGGUCCCCACAGACUCAGAGAGAACCCGCCACCaugAAGUGAGGCU |
| NO: 106 | mCX40L_ | CUCCUCCCGCCCCGCCCCUCCCACGCCUCACCAGCCCCCCGCGCGCCCACCCUC |
| | down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGAGGGCGAGGGCGUGCAGC |
| | | CACUGGAUGAGAACCUGGAGAACGGCAGCCGGCCCCGGUUCAAGUGGAAGAA |
| | | GACCUUACGUCUGGUGGUGAGCGGCAUCAAGGGCGCCGGCAUGCUGCUGUGC |
| | | UUCAUCUACGUGUGUCUGCAGCUGAGCAGCAGCCCCGCCAAGGACCCCCCUAU |
| | | CCAGCGGCUGCGGGGCGCCGUGACCCGGUGCGAGGACGGCCAGCUGUUCAUCA |
| | | GCAGCUACAAGAACGAGUACCAGACCAUGGAGGUGCAGAACAACAGCGUGGU |
| | | GAUCAAGUGCGACGGCCUGUACAUCAUCUACCUGAAGGGCAGCUUCUUCCAGG |
| | | AAGUGAAGAUCGACCUACACUUCAGGGAGGAUCACAACCCCAUCAGCAUCCCC |
| | | AUGCUGAACGACGGCCGGCGGAUCGUGUUCACCGUGGUGGCCAGCCUGGCCUU |
| | | CAAGGACAAGGUGUACCUGACCGUGAACGCCCCCGACACCCUGUGUGAGCACC |
| | | UGCAGAUCAACGACGGCGAGCUGAUCGUGGUGCAGCUGACUCCCGGCUACUGC |
| | | GCCCCCGAGGGCAGCUACCACAGCACCGUGAACCAGGUGCCCCUUUGAUUAAA |
| | | CCGGUACCGGUUGAUAAUAGCAGACACCAUUGUCACACUCCAGCUGGAGCCUC |
| | | GGUGGCCAUGCUUCUUGCCCCUUGGGCCCAGACACCAUUGUCACACUCCAUCC |
| | | CCCCAGCCCCUCCUCCCCUUCCUGCACCCGUACCCCCCAGACACCAUUGUCACA |
| | | CUCCAGUGGUCUUUGAAUAAAGUCUGAGUGGGCGGCGAAUUCGAUAUCCAGC |
| | | GGCCGCGCUAGCGCUGGCUAGCGAAUUCGAUAUCCAGCGGCCGCAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAA |
| SEQ ID | U27- | AGGUCUUCUGGUCCCCACAGACUCAGAGAGAACCCGCCACCaugAAGUGAGGCU |
| NO: 107 | hIFN- | CUCCUCCCGCCCCGCCCCUCCCACGCCUCACCAGCCCCCCGCGCGCCCACCCUC |
| | A_down1 | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCCCUGACCUUCGCCCUGUU |
| | | AGUGGCGCUGCUGGUGCUGAGCUGCAAGAGCAGCUGCAGCGUGGGCUGCGAC |
| | | CUGCCUCAGACCCACAGCCUGGGCAGCCGGCGGACCCUGAUGCUGCUGGCCCA |
| | | GAUGCGGAAGAUCAGCCUGUUCAGCUGUCUGAAGGACCGGCACGACUUCGGC |
| | | UUCCCCCAGGAGGAAUUCGGCAACCAGUUCCAGAAGGCCGAGACCAUCCCCGU |
| | | GCUGCACGAGAUGAUCCAGCAGAUCUUCAACCUGUUCAGCACCAAGGAUAGCA |
| | | GCGCCGCUUGGGACGAGACCCUUCUGGACAAGUUCUACACCGAGCUGUACCAG |
| | | CAGCUGAACGACCUGGAGGCCUGCGUGAUCCAGGGCGUGGGCGUGACCGAGAC |
| | | CCCUCUGAUGAAGGAGGACAGCAUCCUGGCCGUGCGGAAGUACUUUCAGCGG |
| | | AUCACCCUGUACCUGAAGGAGAAGAAGUACAGCCCAUGCGCCUGGGAGGUGG |
| | | UGCGGGCCGAGAUCAUGCGGAGCUUCAGCCUGAGCACCAACCUGCAGGAGAGC |
| | | CUGCGGAGCAAGGAGUGAUUAAACCGGUACCGGUUGAUAAUAGCAGACACCA |
| | | UUGUCACACUCCAGCUGGAGCCUCGGUGGCCAUGCUUCUUGCCCCUUGGGCCC |
| | | AGACACCAUUGUCACACUCCAUCCCCCCAGCCCCUCCUCCCCUUCCUGCACCCG |
| | | UACCCCCCAGACACCAUUGUCACACUCCAGUGGUCUUUGAAUAAAGUCUGAGU |
| | | GGGCGGCGAAUUCGAUAUCCAGCGGCCGCGCUAGCGCUGGCUAGCGAAUUCGA |
| | | UAUCCAGCGGCCGCAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAAAA |
| SEQ ID | U27-IFN- | AGGUCUUCUGGUCCCCACAGACUCAGAGAGAACCCGCCACCaugAAGUGAGGCU |
| NO: 108 | down1 | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGGCUCGGCUGUGCGCCUUCCU |
| | | GAUGAUCCUGGUGAUGAUGAGCUACUACUGGAGCGCAUGCAGCCUGGGCUGC |
| | | GACCUGCCACACACCUACAACCUGGGCAAUAAGCGGGCUCUGACCGUGCUGGA |
| | | GGAGAUGCGGCGGCUGCCCCCCCUGAGCUGCCUGAAGGACCGGAAGGACUUCG |
| | | GCUUCCCCCUGGAGAAGGUGGACAACCAGCAGAUCCAAAAGGCACAGGCCAUC |
| | | CUGGUGCUGCGGGACCUGACCCAGCAGAUCCUGAACCUGUUCACCAGCAAGGA |
| | | CCUGAGCGCCACCUGGAACGCCACCCUGCUGGACAGCUUCUGCAACGACCUGC |
| | | ACCAGCAGCUGAACGACCUGAAGGCCUGCGUCAUGCAGGAGCCACCACUGACC |
| | | CAGGAGGACAGCCUGUUAGCUGUGCGGACUUACUUCCACCGGAUCACCGUGUA |
| | | CCUGCGGAAGAAGAAGCACAGCCUGUGCGCUUGGGAGGUGAUCCGGGCCGAG |
| | | GUGUGGCGGGCCCUGAGCAGCAGCACCAACCUGCUGGCCCGGCUGAGCGAGGA |
| | | GAAGGAGUGAUUAAACCGGUACCGGUUGAUAAUAGCAGACACCAUUGUCACA |
| | | CUCCAGCUGGAGCCUCGGUGGCCAUGCUUCUUGCCCCUUGGGCCCAGACACCA |
| | | UUGUCACACUCCAUCCCCCCAGCCCCUCCUCCCCUUCCUGCACCCGUACCCCCC |
| | | AGACACCAUUGUCACACUCCAGUGGUCUUUGAAUAAAGUCUGAGUGGGCGGC |
| | | GAAUUCGAUAUCCAGCGGCCGCGCUAGCGCUGGCUAGCGAAUUCGAUAUCCAG |
| | | CGGCCGCAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAA |
| SEQ ID | U27-IL- | AGGUCUUCUGGUCCCCACAGACUCAGAGAGAACCCGCCACCaugAAGUGAGGCU |
| NO: 109 | 2_down1 | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUACCGGAUGCAGCUGCUGA |
| | | GCUGCAUCGCCCUGAGCCUGGCCCUGGUGACCAACAGCGCCCCCACCAGCAGC |
| | | AGCACCAAGAAGACCCAGCUGCAACUGGAGCACCUGCUGCUGGAUCUGCAGAU |
| | | GAUCCUGAACGGCAUCAACAAUUACAAGAAUCCCAAGCUGACCCGGAUGCUGA |
| | | CCUUCAAGUUCUACAUGCCCAAGAAGGCUACCGAGCUGAAGCACCUGCAGUGC |
| | | CUGGAGGAGGAGCUGAAGCCCCUGGAGGAGGUGCUGAACCUGGCCCAGAGCA |
| | | AGAACUUCCACUUCGACCCCCGGGACGUGGUGAGCAACAUCAACGUGUUCGUG |
| | | CUGGAGCUGAAGGGCAGCGAGACCACCUUCAUGUGCGAGUACGCCGACGAGAC |
| | | CGCCACCAUCGUGGAGUUCCUGAACCGGUGGAUCACCUUCUGCCAGAGCAUCA |
| | | UCAGCUGAUUAAACCGGUACCGGUUGAUAAUAGCAGACACCAUUGUCACACU |
| | | CCAGCUGGAGCCUCGGUGGCCAUGCUUCUUGCCCCUUGGGCCCAGACACCAUU |
| | | GUCACACUCCAUCCCCCCAGCCCCUCCUCCCCUUCCUGCACCCGUACCCCCCAG |
| | | ACACCAUUGUCACACUCCAGUGGUCUUUGAAUAAAGUCUGAGUGGGCGGCGA |
| | | AUUCGAUAUCCAGCGGCCGCGCUAGCGCUGGCUAGCGAAUUCGAUAUCCAGCG |
| | | GCCGCAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAA |
| SEQ ID | U27-mIL- | AGGUCUUCUGGUCCCCACAGACUCAGAGAGAACCCGCCACCaugAAGUGAGGCU |
| NO: 110 | 2_down1 | CUCCUCCCGCCCCGCCCCUCCCACGCCUCACCAGCCCCCCGCGCGCCCACCCUC |
| | | CGGCGGGUGACAGCUCCGGGAUCAGCAACCCUUCCUGCUGCUGCUACUGCUGC |
| | | UGCUGCUGCCGCCGCCGCCGCCGCCGCUGCCCUUGGGUCCCCCCGAGUCUCCG |
| | | GGACUGCCCUCUCGACUGUCAGUGGGGCAGCCUCUCCGACUCUGCACCCGCCU |
| | | CGACCUCCCCACCCGCUCCCACACCCCUGUGCCCUCAUGUGGAGCCUAAGAGA |
| | | ACAGAACAGGCCGUGAAGCCAGCAGAGAAAAUGUACAGCAUGCAGCUGGCAA |
| | | GCUGCGUGACCCUGACCCUGGUGCUGCUAGUGAACAGCGCCCCCACCAGCAGC |
| | | AGCACCAGCAGCUCCACCGCCGAGGCCCAGCAGCAACAACAGCAACAGCAGCA |
| | | GCAGCAACAGCACCUGGAGCAGCUCCUGAUGGACCUGCAGGAGCUGCUGAGCC |
| | | GGAUGGAGAACUACCGGAACCUGAAGCUGCCCCGGAUGCUGACCUUCAAGUUC |
| | | UACCUGCCCAAGCAGGCCACAGAGCUGAAGGACCUGCAGUGCCUGGAGGACGA |
| | | GCUGGGCCCCCUGCGGCACGUGCUGGAUCUGACCCAGAGCAAGAGCUUCCAGC |
| | | UGGAGGACGCCGAGAACUUCAUCAGCAACAUCCGGGUGACCGUGGUGAAGCU |
| | | GAAGGGCAGCGACAACACCUUCGAGUGCCAGUUCGACGACGAGAGCGCAACCG |
| | | UGGUGGACUUCCUGCGGCGGUGGAUCGCCUUCUGCCAGAGCAUCAUCAGCACC |
| | | AGCCCCCAGUGAUUAAACCGGUACCGGUUGAUAAUAGCAGACACCAUUGUCAC |
| | | ACUCCAGCUGGAGCCUCGGUGGCCAUGCUUCUUGCCCCUUGGGCCCAGACACC |
| | | AUUGUCACACUCCAUCCCCCCAGCCCCUCCUCCCCUUCCUGCACCCGUACCCCC |
| | | CAGACACCAUUGUCACACUCCAGUGGUCUUUGAAUAAAGUCUGAGUGGGCGG |
| | | CGAAUUCGAUAUCCAGCGGCCGCGCUAGCGCUGGCUAGCGAAUUCGAUAUCCA |
| | | GCGGCCGCAAAAAAAAAAAAAAAAAAAAAAAAAA AAAA AAA AAAAAAAAA |
| | | AAAAAAAA AA AAAAAAAA LAAAAA |
| | | AAAAAAAAAAAAAAAAAAAAAAAAA |

The present disclosure further provides an insight that inclusion of a codon resulting in introduction of proline to the nascent polypeptide can induce kinking of the nascent polypeptide, and that such kinking can be used to induce and/or enhance oncoselective stop codon read-through. Thus, in some embodiments, oncoselective stop-codon read through can be induced and/or enhanced by inclusion of one or more proline-encoding codons in a translatable nucleic acid, as an alternative to or in addition to one or more of the other strategies described herein for inducing and/or enhancing oncoselective stop codon read-through.

In some embodiments, a stem loop in the mRNA can induce and/or enhance stop codon read-through. In some embodiments, a stem loop inducing and/or enhancing stop codon readthrough is within approximately 20, 40, 60, 80 or 120 nucleotides of the stop codon. In some embodiments, a stem loop inducing and/or enhancing stop codon read-through is in the coding sequence just prior to the stop codon. In some embodiments, a stem loop inducing and/or enhancing stop codon read-through is in the 3′UTR. In some embodiments, a stem loop inducing and/or enhancing stop codon read-through is in the region spanning the coding region and 3′UTR boundary. In some embodiments, a bulge loop or a pseudoknot in the mRNA can induce and/or enhance stop codon read-through. In some embodiments, nucleic acid structures inducing and/or enhancing stop codon read-through have a low Gibbs free energy relative to nucleic acid structures that do not result in read-through. In some embodiments, the first 25, 50, or 75 nucleotides of the 3′UTR of a nucleic acid inducing stop codon read-through have a delta G of 5 kcal/mole; 10 kcal/mole; 15 kcal/mole; 20 kcal/mole; 25 kcal/mole; 30 kcal/mole lower than non-cancer stop codon read-through counterparts. In some embodiments, the first 25, 50, or 75 nucleotides of the 3′UTR of a nucleic acid inducing stop codon read-through have a delta G in the range of 5 kcal/mole to 20 kcal/mole; 5 kcal/mole to 10 kcal/mole; or 10 kcal/mole to 20 kcal/mole; 25 kcal/mole; 30 kcal/mole lower than non-cancer stop codon read-through counterparts.

In some embodiments, aminoglycosides (e.g., gentamicin) and macrolides (e.g. erythromycin) can induce stop codon read-through. Without wishing to be bound by any theory, aminoglycosides can induce stop codon read-through by binding 18s rRNA and macrolides can induce stop codon read-through by binding the peptide channel within large ribosomal subunit. In some embodiments, aminoglycosides and macrolides can induce stop codon read-through in healthy (normal) cells. In some embodiments, subjects treated with aminoglycosides or macrolides should not be treated with a nucleic acid comprising a stop codon read-through motif.

In some embodiments, the present disclosure encompasses the recognition that an oncoselective translation sequence element can be oncospecific and result in translation and payload expression only in cancer cells (i.e., no detectable expression in non-cancer cells). Alternatively or additionally, in some embodiments, an oncoselective translation sequence element is translated 2, 5, 10, 15, 20, 30 or more—fold higher in cancer cell(s) as compared with appropriately comparable non-cancer cells.

In some embodiments, an oncoselective translation sequence element can comprise an internal ribosome entry segment/site (IRES). In some embodiments, an oncogenic ribosome, or RNA binding protein, preferentially binds an IRES in an oncoselective translation sequence element. In some embodiments, an oncoselective translation sequence element can be bound by or direct the binding of translation initiating RNA binding proteins (RBPs). In some embodiments, an oncoselective translation sequence element can comprise and IRES and be bound by or direct the binding of RBPs.

In some embodiments, an oncoselective read-through motifs is one listed in Table 1.

TABLE 1
Exemplary nucleic acid sequences of oncoselective read-through motifs

| | ENS | Sequence |
|---|---|---|
| U1 | 380715 | AAACGCAAGCAAGAGGAACAAATGGAAACTGAGCATTTTG |
| SEQ ID | | CTCTGTAAAGCATTTCAGATGCAGCTTGTGTG |
| NO. 1 | | |
| U2 | 428608 | GCGCTGACCCAGCTGATCCAGCTCTATCATCGCTTCCACCG |
| SEQ ID | | GGTGCTGTCCCAGCCGCAGCTCCGAGCCCTCCCTGCCCGG |
| NO. 2 | | GCTGAGCTCATCAACATTCACCACCTTATGGTGGAGCTCAA |
| | | GAAGCATAAGCCCAACTTCTGATGTGCCAGAAACCGCCCT |
| | | GAGATCTGCCGGTCATCTCCATGGACTTCTGCACCCCATTC |
| | | CATACCCTTCTTCACCTGGGGTACCCCTTCCAGTTTTCCCCT |
| | | TGCTTCCCAGGCCCTTGACATGGCTTACCTGCCTTCACTCC |
| | | CAGCACCTTGCCCAACAGGA |
| U3 | 409841 | CTTCCAGCAGAGGAGAACATGTCTAACACGTGCCTCAAAA |
| SEQ ID | | GCACTGGGGAGTTAGTAGTGCAGTGGCATCTGAAACCTGT |
| NO. 3 | | GGAGCAGAAAGCACATGAGTCCTAATGCCCCAGCAGCTTC |
| | | CGATTG |
| U4 | 274358 | ATAGTATACCCTGGAATTGCTGTATTTTTCCAGAATGCCTTC |
| SEQ ID | | ATCTTTTTTGGAGGGCTGGTTTTTAAGTTTGGCCGCACTGA |
| NO. 4 | | AGACTTATGGCAGTGAACACATCTGATTTCCCACAGCACAA |
| | | CAGCCCTGCATGGGTTTGTTTGTTTTTTTACTGCTCACTCCC |
| | | AACCTTTTGTAATGCCATTTTCTAAACTTATTTCTGAGTGTA |
| | | GTCTCAGCTTAAAG |
| U5 | 425191 | GGTGACAAATCCATTCGTTTCCGTCCCACGCTGGTCTTCAG |
| SEQ ID | | GGATCACCACGCTCACCTGTTCCTCAATATTTTCAGTGACAT |
| NO. 5 | | CTTAGCAGACTTCAAGTAAAGAAGCCATTTCCACTACAGTG |
| | | AGAAAGCCCGGATCCCAACAGTTGTCAAATTGATTAGTTTG |
| | | CCTAATTCATGTTTTCACTTAAAAGTATCAGAGGTG |
| U6 | 332780 | TGTCCTTTGGGCTCTGTGGAGAGCTTTAGCCTTGCACGGCG |
| SEQ ID | | GCGCTGGGAGGCATTGCCTGCCATGCCCACTGCCCGCTGCT |
| NO. 6 | | CCTGCTCTAGTCTGCAGGCTGGGCCCCGGCTGTTTGTTATT |
| | | GGGGGTGTGGCCCAGGGCCCCAGTCAAGCCGTGGAGGCA |
| | | CTGTGTCTGCGTGATGGGGTCTGAAGGCTTGGTGGGAGCT |
| | | GTCCACTGGAGCAGCTCATTGCCAGAGGCAGCTATTTCTAT |
| | | GGCTCCTTTTGCTGCTGAGGA |
| U7 | 360534 | ATATATACCCACATGAGCCACTTCATAAAACAATGTTTCTCT |
| SEQ ID | | TTACCTTAGCACCTCAAAATACCATGCCATTTAAAGCTTATT |
| NO. 7 | | AAAACTCATTTTTGTTTTCATTATCTCAAAACTGCACTGTCA |
| | | AGATGATGATGATCTTTAAAATACACACTCAAATCAAGAAA |
| | | CTTAAGGTTACCTTTGTTCCCAAATTTCATACCT |
| U8 | 450195 | GAGGTTCTCCAGGACCTTAGGTTTGATGCGGAATCTGCCGA |
| SEQ ID | | GTGATGGCGGCTCCCCAGGGATGCGCCGAGGGAGATGGGA |
| NO. 8 | | AACGGGGCGGATGGCGCCCAGCCCAGCCCTAACTGCCAGC |
| | | TGGCTGGGGTTGCGCCCCACTGCGCTGCTGACCTTCCTGCA |
| | | GTTCCAGACACCTCCCACAATAAAGAGCTCCTCCTCTGT |
| U9 | 337231 | GGTGAGGTCAAGGCAGGAGAGAAGAGCCTGAGCCAGCAC |
| SEQ ID | | GTGGAGGCCGTGGACAAGCGGCTGGAACAGAGCCAGCCC |
| NO. 9 | | GAGTACAAGGCGCTCTTCAAAGAGATCTTCTCCAGGATCCA |
| | | GAAGACCAAGGCTGACATCAACGCCACCAAAGTCAAGAC |
| | | GCACAGCAGCAAGTGACCCTTCTCCGGCCTGCAGCCTCCC |
| U10 | 358607 | GAGGCGATCAGCGAGGTTCTCCAGGACCTTAGGTTTGATGC |
| SEQ ID | | GGAATCTGCCGAGTGATGGCGGCTCCCCAGGGATGCGCCG |
| NO. 10 | | AGGGAGATGGGAAACGGGGCGGATGGCGCCCAGCCCAGC |
| | | CCTAACTGCCAGCTGGCTGGGGTTGCGCCCCACTGCGCTG |
| | | CTGACCTTCCTGCAGTTCCAGACACCTCCCACAATAAAGAG |
| | | CTCCTCCTCTGT |

In some embodiments, a putative oncoselective read-through motifs is one listed in Table 2.

TABLE 2
Exemplary sequences of putative oncoselective read-through motifs

| | GC | | |
|---|---|---|---|
| Construct | Content(%)* | Linear Motif* | Motif Sequence |
| U11 | 72 | VNNNNNNMNNMWK | GAGGTTCTCCAGGACCTTAGGT |
| SEQ ID NO. 11 | | | TTGATGCGGAATCTGCCGAGTG |
| | | | ATGGCGGCTCCCCAGGGATGOG |
| | | | CCGAGGGAGATGGGAAACGGG |
| | | | GCGGATGGCGCCCAGCCCAGC |
| | | | CCTAACTGCCAGCTGGCTGGGG |
| | | | TTGCGCCCCACTGCGCTGCTGA |
| | | | CCTTCCTGCAGTTCCAGACACCT |
| | | | CCCACAATAAAGAGCTCCTCCTC |
| | | | TGT |
| U12 | 58 | WGNNSNHNHDNNN | ATCCTAGCTTOGGTGCTGGCAG |
| SEQ ID NO. 12 | | | TGTCCCAACAGGAATACCTAGA |
| | | | CAGTATGAAGAAAAACAAAGTG |
| | | | CACAGAGACCCGCCCCCAGACA |
| | | | AGAGTTGATGGAGACCCAGGG |
| | | | ATTGGACACCATCTOCCAACCCC |
| | | | AGTACTCCTGCTCTCCGGTGCCA |
| | | | CCTCACCTTCTTTGGCTTCTTCC |
| | | | CTCTTGCCTCCTTCTGTTCTTTC |
| U13 | 50 | MWBNNNNNNNNNN, | ACTGTTTCCTATGATCCTAGGAA |
| SEQ ID NO. 13 | | VMNNWNKNNNNNN | ACCCACTGTGAAGAAAATTGCC |
| | | | CCAATGATGGCCAAGACAATTA |
| | | | AAGCTTTCAAGAACAGATTCTC |
| | | | CCGACGATAAACTGAGGACTTG |
| | | | CCTTGGAAATGGAATCTGGGGA |
| | | | GGCAGGAATACAAGGACAGTG |
| | | | GGGGTTGGGGAATGGAATTCTA |
| | | | CAGGAGACTGGAGTCTTGCTTT |
| | | | GTG |
| U14 | 52 | VNNNNNNMNNMWK, | TTGTCATGTGTACAGGAAATCA |
| SEQ ID NO. 14 | | MWBNNNNNNNNNN, | GTGATGTGGTGCAGAGGTAGCC |
| | | VMNNWNKNNNNNN | ACTGTTAGCCTGGTGGGAAAAT |
| | | | GCACACATTTCTGAGGGGAGA |
| | | | GGGAAAAGGACTTGTTTTCCTG |
| | | | TGTTCTTGTTTTCAGAAAATGAA |
| | | | AGACTCATACTTGAGTGTGTTTA |
| | | | TGTG |
| U16 | 60 | VMNNWNKNNNNNN | GTGCAGCACAAAAAGCCCGCC |
| SEQ ID NO. 16 | | | GACATCCCTCAGGGCTCCTTGG |
| | | | CCTACCTGGAGCAGGCATCTGC |
| | | | CAACATCCCTGCACCTCTGAAG |
| | | | CCAACGTGAGCAAAGGGCAGA |
| | | | GGCAGTTGGCCTATGAGTGGGC |
| | | | TGATGCGTGAGGTTGGCCACAC |
| | | | ATTCCTTCCTGTGGACTTGACAT |
| | | | TTTGGAAGAACTCTTTGCCAGA |
| | | | TAATGAGTTCATTTTAGTTTTATG |
| | | | CTCCCAT |
| U17 | 72 | DVHVNNNCWNNNB, | TGGGGTCCCCAGTGGGGAATG |
| SEQ ID NO. 17 | | VMNNWNKNNNNNN | AACGGGTACTTCCTCATCGAGC |
| | | | GCGGAAAGAACATGTGTGGCC |
| | | | TGGCTGCCTGCGCCTCCTACCCC |
| | | | ATCCCTCTGGTGTGAGCCGTGG |
| | | | CAGCCGCAGCGCAGACTGGCG |
| | | | GAGAAGGAGAGGAACGGGCA |
| | | | GCCTGGGCCTGGGTGGAAATCC |
| | | | TGCCCTGGAGGAAGTTGTGGG |
| | | | GAGATCCACTGGGACCCCCAAC |
| | | | ATTCTGCCCTCACCTCTGTGCCC |
| | | | AGCCTGGAAACCTACAGACAAG |
| | | | GAGG |
| U22 | 28 | MWBNNNNNNNNNN | GAAGAAACAAAGAATAAACTCA |
| SEQ ID NO. 18 | | | AAGCCGGCAATGCCCTGGCATT |
| | | | TTTGGGTCTTGAGAGAAAACAA |
| | | | TTTGAATGACTGAATTTACTACA |
| | | | AAGGCAAACTTTCAAAAGGATA |
| | | | TCTCTTTTTTGTTTCCAAATATGT |
| | | | ATCAACAGGTATCAACAAAATCC |
| | | | TATTTTGAACTATTTTACTCAGA |
| | | | AAAGAATATCCCAAATATCCCAA |
| | | | ATTATTCATAATAAAAATGATTTG |
| | | | AAAGTGTTTTCATTCTTAAAA |
| U24 | 28 | MWBNNNNNNNNNN | TTTTACTGTAAGCTGTGTTCACT |
| SEQ ID NO. 19 | | | CTTTTATACAAATGAAGAAGTTG |
| | | | CAAAGAATACTCATTGCAGCAG |
| | | | CCTTCCTCATTATCAGAAATTAA |
| | | | AGAAATTTCTGAATAAATTGGC |
| | | | AGAAGAACGCAGACAGAAGAA |
| | | | GGAAACTTAAGATGTGCAAGG |
| | | | AGATTTAAGGATTTCAAAGAAA |
| | | | ATAAAGGTTCTTTGTTTT |
| U27 | 84 | MWBNNNNNNNNNN, | AAGTGAGGCTCTCCTCCCGCCC |
| SEQ ID NO. 20 | | VNNNNNNMNNMWK | CGCCCCTOCCACGCCTCACCAG |
| | | | CCCCCCGCGCGCCCACCCTCCG |
| | | | GCGGGTGACAGCTCCGGGATC |
| | | | AGCAACCCTTCCTGCTGCTGCTA |
| | | | CTGCTGCTGCTGCTGCCGCCGC |
| | | | CGCCGCCGCCGCTGCCCTTGGG |
| | | | TCCCCCCGAGTCTCCGGGACTG |
| | | | CCCTCTCGACTGTCAGTGGGGC |
| | | | AGCCTCTCCGACTCTGCACCCG |
| | | | CCTCGACCTCCCCACCCGCTCCC |
| | | | ACACCCCTGTGCCCTCATGTGG |
| | | | AGCCTAAGAGAACAGAACAGG |
| | | | CCGTGAAGCCAGCAGAGAAA |
| U28 | | | CCCCGCCCTCCCAGTTTCCGCG |
| SEQ ID NO. 21 | | | CGCCTCTTTGGCAGCTGGTCAC |
| | | | ATGGTGAGGGTGGGGGTGAGG |
| | | | GGGCCTCTCTAGCTTGCGGCCT |
| | | | GTGTCTATGGTCGGGCCCTCTG |
| | | | CGTCCAGCTGCTOCGGACCGAG |
| | | | CTCGGGTGTATGGGGCCGTAGG |
| | | | AACCGGCTCCGGGGCCCCGATA |
| | | | ACGGGCCGCCCCCACAGCACCC |
| | | | CGGGCTGGCGTGAGGGTCTCC |
| | | | CTTGATCTGAGA |

*determined for the first 50 nucleotides of the oncoselective construct.

Onco-333 fLuc sequence (UTR sequences and the polyA tail are capitalized):

(SEQ ID NO. 22)
GAAATAAGAGAGAAAAGAAGAGTAAGAAGAAATATAAGAGCCACCatggaagacgccaaaaa
cataaagaaaggcccggcgccattctatccgctggaagatggaaccgctggatagcaactgcataa
ggctatgaagagatacgccctggttcctggaacaattgcttttacagatgcacatatcgaggtgga
catcacttacgctgagtacttcgaaatgtccgttcggttggcagaagctatgaaacgatatgggct
gaatacaaatcacagaatcgtcgtatgcagtgaaaactctcttcaattctttatgccggtgttggg
cgcgttatttatcggagttgcagttgcgcccgcgaacgacatttataatgaacgtgaattgctcaa
cagtatgggcatttcgcagcctaccgtggtgttcgtttccaaaaaggggttgcaaaaaattttgaa
cgtgcaaaaaaagctcccaatcatccaaaaaattattatcatggattctaaaacggattaccaggg
atttcagtcgatgtacacgttcgtcacatctcatctacctcccggttttaatgaatacgattttgt
gccagagtccttcgatagggacaagacaattgcactgatcatgaactcctctggatctactggtct
gcctaaaggtgtcgctctgcctcatagaactgcctgcgtgagattctcgcatgccagagatcctat
ttttggcaatcaaatcattccggatactgcgattttaagtgttgttccattccatcacggttttgg
aatgtttactacactcggatatttgatatgtggatttcgagtcgtcttaatgtatagatttgaaga
agagctgtttctgaggagccttcaggattacaagattcaaagtgcgctgctggtgccaaccctatt
ctccttcttcgccaaaagcactctgattgacaaatacgatttatctaatttacacgaaattgcttc
tggtggcgctcccctctctaaggaagtcggggaagcggttgccaagaggttccatctgccaggtat
caggcaaggatatgggctcactgagactacatcagctattctgattacacccgagggggatgataa
accgggcgcggtcggtaaagttgttccattttttgaagcgaaggttgtggatctggataccgggaa
aacgctgggcgttaatcaaagaggcgaactgtgtgtgagaggtcctatgattatgtccggttatgt
aaacaatccggaagcgaccaacgccttgattgacaaggatggatggctacattctggagacatagc
ttactgggacgaagacgaacacttcttcatcgttgaccgcctgaagtctctgattaagtacaaagg
ctatcaggtggctcccgctgaattggaatccatcttgctccaacaccccaacatcttcgacgcagg
tgtcgcaggtcttcccgacgatgacgccggtgaacttcccgccgccgttgttgttttggagcacgg
aaagacgatgacggaaaaagagatcgtggattacgtcgccagtcaagtaacaaccgcgaaaaagtt
gcgcggaggagttgtgtttgtggacgaagtaccgaaaggtcttaccggaaaactcgacgcaagaaa
aatcagagagatcctcataaaggccaagaagggcggaaagatcgccgtgtaaGCTGCCTTCTGCGG
GGCTTGCCTTCTGGCCATGCCCTTCTTCTCTCCCTTGCACCTGTACCTCTTGGTCTTTGAATAAAG
CCTGAGTAGGAAAA
A
AA
AAAA
AAAAAA
AAA
AAAAAAAAAAAAA
A
AAAAAAAAAAAAA
AAAAAAAAAAA
AAAAA
Onco-333 RT motif:
(SEQ ID NO. 23)
ctatccgctggaagatggaaccgctggaTAGcaactgcataaggctatgaagaga

### Nucleic Acids

Among other things, the present disclosure provides nucleic acids (e.g., engineered nucleic acids) that participate in and/or are otherwise related to oncoselective translation as described herein. In some embodiments the present disclosure provides nucleic acids that are or include or deliver a translatable nucleic acid comprising an oncoselective read-through motif. In some embodiments, the present disclosure provides nucleic acids that are or include or deliver a translatable nucleic acid encoding a payload of interest and including an oncoselective translation sequence element as described herein.

In some embodiments, a provided engineered nucleic acid may be or comprise DNA (e.g., single or double-stranded DNA), e.g., that, when introduced into a cell, is transcribed, or generates a template strand that is transcribed) to produce a translatable nucleic acid (e.g., an RNA such as an mRNA) as described herein. In some embodiments, a provided engineered nucleic acid may be or comprise RNA (e.g., mRNA), which may be or comprise (or may be or comprise the complement of) a translatable nucleic acid described herein (e.g., may be or comprise a coding sequence and an oncoselective translation sequence element(s)).

In some embodiments, a provided nucleic acid is or comprises DNA or RNA or both. In some embodiments, a provided nucleic acid is chemically modified relative to naturally occurring DNA and/or RNA. In some embodiments, a provided nucleic acid is not modified with pseudouridine.

In some embodiments, a provided nucleic acid is a translatable nucleic acid as described herein. In some embodiments, a provided nucleic acid is expressible (e.g., can be transcribed to express) to produce a translatable nucleic acid as described herein. In some embodiments, a provided nucleic acid is a complement of a translatable nucleic acid as described herein, or of a nucleic acid that is expressible to produce such a translatable nucleic acid (or its complement).

Thus, in some embodiments, the present disclosure builds upon and enhances recent developments in the field of RNA (e.g., mRNA) therapeutics. Several groups have done important work developing technologies for, for example, improving RNA production and/or stability; providing encapsulating or other systems to facilitate RNA administration and/or delivery to mammalian (e.g., human) subjects; etc. Recent work by companies such as BioNTech AG, CureVac AG, Ethris AG, Moderna Therapeutics, Translate Bio, Inc., and others have led to development of several clinical candidates, and, recently, the first siRNA therapeutic and mRNA vaccine approved by the US Food and Drug Administration; those skilled in the art will appreciate that any or all of the available technologies for production, stability, administration, etc. of RNA therapeutics may be applicable to and/or utilized with those embodiments of the present disclosure that administer a translatable RNA to mammalian (e.g., human) subjects.

Analogously, the present disclosure builds upon and enhances various developments in the field of gene therapy, e.g., involving development of DNA and/or RNA vectors that can deliver translatable nucleic acids to cells in mammalian (e.g., human) subjects. Recent work on oncolytic viruses have demonstrated efficient gene delivery and cell killing in various malignancies (Raman et al., Immunotherapy. 2019 June; 11(8):705-723; Mahalingam et al., Cancers (Basel). 2018 May 25; 10(6)). In addition, groups working on self-amplifying mRNA replicons have demonstrated efficient local delivery and improved pharmacokinetic profile with prolonged protein expression (Avogadri et al, Cancer Immunol Res. 2014 May; 2(5):448-58; Huysmans et al., 2019 bioRxiv 10.1101/528612). In some embodiments of the present disclosure, a provided nucleic acid comprises an oncolytic virus particle or an oncolytic DNA or RNA or a self-amplifying mRNA formulated in polymer or lipid nanoparticle.

In some embodiments, a provided nucleic acid is engineered to show low or reduced (relative to an appropriate reference) immunogenicity when introduced, produced, and/or expressed in a subject. Those skilled in the art are aware of certain sequence elements and/or chemical modifications that can increase or decrease immunogenicity of a nucleic acid that contains them as compared with one that does not. In many embodiments, provided nucleic acids are engineered so that those that are or will be introduced, produced, and/or expressed in a subject are characterized by low expected or observed immunogenicity. For example, provided m RNAs can be engineered by increasing GC content (Thess et al., 2015, Mol Ther 23:1456-64) or decreasing U content (Kariko & Sahin, 2017, WIPO Patent App No: WO 2017/036889 A1; Vaidyanathan, et al., 2018. 12: 530-542). The provided mRNAs can be modified by incorporation of non-canonical nucleotides, such as pseudouridine, N1-methyl-pseudouridine, methoxy-uridine, and 2-thiouridine into mRNA (Kariko, 2005, Immunity. 23:165-75; Kariko, 2008, Mol Ther 16:1833-40; Kormann et al., 2011, Nat Biotechnol. 29:154-157; Andries et al., 2015, J Control Release. 217:337-344).

Alternatively or additionally, in some embodiments, a provided nucleic acid that includes or encodes a translatable payload is engineered so that the payload, when introduced and/or produced in a subject, shows relatively low immunogenicity. For example, in some embodiments, immunogenic epitope(s) may have been defined for a particular payload, and a less-immunogenic variant (e.g., having a sequence alteration within, or that otherwise impacts immunogenicity of such as by altering a pattern of post-translational modification, one or more such immunogenic epitope(s)) may be utilized in accordance with the present disclosure.

In some embodiments, a nucleic acid (e.g., an RNA) may be sequence engineered, for example to remove immunogenic sequence motifs. In some embodiments, a nucleic acid is sequence engineered to remove TLR7 or TLR8 stimulation motifs. In some embodiments, a nucleic acid is sequence engineered to remove motifs selected from the group consisting of KNUNDK motifs, UCW motifs, UNU motifs, UWN motifs, USU motifs, KWUNDK motifs, KNUWDK motifs, UNUNDK motifs, KNUNUK motifs, and combinations thereof. In some embodiments, a nucleic acid is sequence engineered as described in WO2020/033720 the entirety of which is incorporated herein by reference.

### Coding Sequence

As described herein, the present disclosure relates particularly to translatable nucleic acids that comprise a coding sequence (e.g., a payload coding sequence) and an oncoselective translation sequence element.

Those of ordinary skill in the art, reading the present disclosure, will appreciate that a wide variety of useful payload sequences are known and can be utilized in accordance with the teachings herein. In some embodiments, the payload is a gene product (e.g., a polypeptide) that, when expressed in cancer cells, reduces their ability to survive and/or to proliferate within a subject.

In some embodiments, a payload sequence may be toxic to cells and/or may generate (e.g., enzymatically) a toxic agent.

In some embodiments, a payload sequence may render cells more susceptible to immunological attack and/or clearance. For example, in some such embodiments, a payload sequence may be or comprise an antigen, antibody, antibody fragment, or their chimeric versions fused to a transmembrane protein and/or an intracellular signaling molecule (e.g. ITAM or costimulatory molecule endodomains) that is particularly attractive to a subject's immune system and/or to an immunological therapy (e.g., CAR-T or CAR-NK cells, proliferated T-cells, etc) that has been or will be administered to the subject. Alternatively or additionally, in some such embodiments, a payload sequence may be or comprise an agent that relieves or inhibits an immunological checkpoint.

As noted herein, one feature of the provided disclosure is that it achieves an extent of oncoselectivity such that payloads that would be unacceptable and/or inadvisable without such oncorestricted expression may be effectively utilized.

In some embodiments, a payload sequence for use in accordance with the present disclosure is selectively active in cancer cells and/or under particular circumstances (e.g., in the presence of a separate agent). However, in some embodiments, particularly in light of the degree of oncoselectivity provided by the present disclosure, in some embodiments, a payload comprises a protein that is constitutively active and/does not require post-translational modifications such as cleavage or phosphorylation.

In some embodiments, a payload is not secreted from a cell in which it is produced (e.g., by translation). In some other embodiments, a payload is a protein that is secreted into the tumor microenvironment.

In some embodiments, a polypeptide payload may be or comprise an antibody, a cell surface protein (e.g., that is or comprises an antigen or epitope targeted by endogenous or administered immune cells—such as T cells, NK cells, etc), an enzyme, a genetic modification protein, a suicide protein, a toxin, a viral replication protein, a viral surface antigen, etc. In some embodiments, a polypeptide payload may be or comprise a biologic agent approved for treatment of cancer.

In some embodiments a linker may be present between an oncoselective translation sequence element and a payload sequence. In some embodiments, a linker comprises 2A linker. In some embodiments, a linker comprises a PT2A linker. In some embodiments, a linker comprises a F2Am linker.

### Antibody Agents

Several antibody therapeutics useful in the treatment of cancer are known in the art. Recent developments in the mRNA therapeutic field indicate that delivery of a translatable nucleic acid encoding an antibody agent of interest can be a viable and effective strategy for administering antibody therapeutics (see, for example, Van Hocke & Roose, J. Translational Med. 17:54, Feb. 22, 2019). Those skilled in the art, reading the present disclosure, will appreciate that its teachings are applicable to therapeutic antibody agents; in some embodiments, a translatable nucleic acid as described herein encodes a polypeptide that is or is a component of a therapeutic antibody agent.

### Engineered Nucleic Acid

Described herein, in some aspects, is an engineered nucleic acid. In some embodiments, the engineered nucleic acid comprises a coding nucleic acid sequence, where the coding nucleic acid sequence encodes a protein or an antigen or a fragment thereof. In some embodiments, the engineered nucleic acid comprises ribonucleic acid. In some embodiments, the engineered nucleic acid comprises an mRNA. In some embodiments, the engineered nucleic acid comprises at least one untranslated region. In some embodiments, the at least one untranslated region is a 3′-UTR. In some embodiments, the at least one untranslated region is a 5′ untranslated region a 5′-UTR. In some embodiments, the engineered nucleic acid comprises both a 3′-UTR and a 5′-UTR. In some embodiments, the engineered nucleic acid comprises at least one nucleic acid modification. For example, the engineered nucleic acid can comprise at least one nucleotide analogue. In some embodiments, the engineered nucleic acid comprises a degenerative sequence. For example, the engineered nucleic acid can comprise at least degenerative codon. Table 3 illustrates an example nomenclature denoting the identity of the nucleotide or degenerative nucleotide.

TABLE 3
Nucleotide base code

| Nucleic acid code | Definition | Mnemonic |
|---|---|---|
| Y | C, T or U | Pyrimidines |
| X | Masked | |
| W | A, T, or U | Weak interaction |
| V | Neither T nor U | V comes after U |
| | (i.e. A, C, or G) | |
| U | U | Uracil |
| T | T | Thymine |
| S | C or G | Strong interaction |
| R | A or G | Purine |
| N | ACGTU | Nucleic acid |
| M | A or C | Bases with amino groups |
| K | G, T or U | Bases which are ketones |
| H | Not G | H comes after G |
| | (i.e., A, C, T, or U) | |
| G | G | Guanine |
| D | Not C | D comes after C |
| | (i.e. A, G, T, or U) | |
| C | C | Cytosine |
| B | Not A | B comes after A |
| | (i.e. C, G, T, or U) | |
| A | A | Adenine |
| — | Gap of | |
| | indeterminate length | |

In some embodiments, the engineered nucleic acid is a vector. In some embodiments, the engineered nucleic acid is a viral vector. In some embodiments, the vector is an expression vector. In the context of an expression vector, the vector can be readily introduced into a host cell, e.g., a mammalian, bacterial, yeast, or insect cell by any known method. For example, the expression vector can be transferred into a host cell by physical, chemical, or biological means. Physical methods for introducing a polynucleotide into a host cell include calcium phosphate precipitation, lipofection, particle bombardment, microinjection, gene gun, electroporation, and the like. Methods for producing cells comprising vectors and/or exogenous nucleic acids are suitable for methods herein. One method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Example viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, viral vector is a chimeric viral vector, comprising viral portions from two or more viruses. In additional instances, the viral vector is a recombinant viral vector.

In some embodiments, the engineered nucleic acid comprises or is operatively coupled to a heterologous sequence. In some embodiments, the heterologous sequence comprises an exon sequence, an intron sequence, an exon-intronjunction, a splice acceptor-splice donor site, a start codon sequence, a stop codon sequence, a promoter site, an alternative promoter site, 5′ regulatory element, enhancer, 5′ UTR region, 3′ UTR region, poly adenylation site, or binding site of a polymerase, nuclease, gyrase, topoisomerase, methy lase or methyl transferase, transcription factors, etc. In some embodiments, the heterologous sequence comprises an expression control sequence such as a promoter.

In some embodiments, the engineered nucleic acid comprises a coding nucleic acid sequence encoding a protein or an antigen or a fragment thereof. In some embodiments, the protein or the antigen or the fragment thereof is a pathogen protein or fragment thereof. In some embodiments, the pathogen protein or fragment thereof is a viral protein or fragment thereof. The virus can be a DNA virus or an RNA virus.

### Untranslated Region

In some aspects, the engineered nucleic acid described herein comprises at least one untranslated region (UTR). In some embodiments, the engineered nucleic acid comprises a 3′-UTR. In some embodiments, the engineered nucleic acid comprises a 5′-UTR. In some embodiments, the engineered nucleic acid comprises both a 3′-UTR and a 5′-UTR. In some embodiments, the at least one UTR is a naturally occurring UTR. In some embodiments, the at least one UTR is a synthetic UTR or a heterologous UTR. In some embodiments, the at least one UTR comprises at least 10, at least 50, at least 100, at least 500, at least 1000, at least 5000, or at least 10000 nucleotides. In some embodiments, the at least one UTR comprises at least one nucleic acid structure such as a secondary structure or an RNA motif. In some embodiments, the at least one UTR does not yield a nucleic acid structure (e.g., the at least one UTR does not form an RNA motif).

### Nucleic Acid Modification

In some embodiments, the engineered nucleic acid described herein comprises at least one nucleic acid modification. In some embodiments, the at least one nucleic acid modification comprises substituting one or more nucleotide with one or more nucleotide analogues. In some embodiments, the nucleic acid modification comprises modifying A, G, U, or C ribonucleotides. In some embodiments, the modification can be made to a coding nucleic acid sequence of the engineered nucleic acid. In some embodiments, the modification can be made to a non-coding nucleic acid sequence of the engineered nucleic acid. In some embodiments, the modification can be made to both coding and non-coding nucleic acid sequence of the engineered nucleic acid. In some embodiments, the at least one nucleic acid modification increases resistant to degradation (e.g., hydrolysis or nuclease digestion) after in vivo administration of the engineered nucleic acid. In some embodiments, the at least one nucleic acid modification decreases immunogenicity after in vivo administration of the engineered nucleic acid as compared to a comparable nucleic acid sequence comprising a coding nucleic acid sequence that encodes an identical protein as the protein encoded by the engineered nucleic acid. In some aspects, the engineered nucleic acid, upon in vivo administration, increases expression of the protein encoded by the engineered nucleic acid compared to an expression of the same protein encoded by a comparable nucleic acid sequence. In some aspects, the engineered nucleic acid, upon in vivo administration, increases expression of the protein encoded by the engineered nucleic acid in a specific cell type compared to an expression of the same protein encoded by a comparable nucleic acid sequence in the same specific cell type.

In some embodiments, the at least one modification can be modification to 3′OH, group, 5′OH group, sugar, nucleobase, internucleotide linkage, or a combination thereof. Nucleic acid modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some aspects, chemically modified backbone comprises a backbone other than a phosphodiester backbone. In some aspects, a modified sugar comprises a sugar other than deoxyribose (in modified DNA) or other than ribose (modified RNA). In some aspects, a modified base comprises a base other than adenine, guanine, cytosine, thymine or uracil. In some aspects, the engineered nucleic acid comprises at least one chemically modified base. In some instances, the comprises at least one, two, three, four, five, six, seven, eight, nine, 10, 15, 20, 50, 100, or more modified bases. In some cases, nucleic acid modifications to the base moiety include natural and synthetic modifications of adenine, guanine, cytosine, thymine, or uracil, and purine or pyrimidine bases. For example, the nucleic acid modification comprises modifying at least one uracil of the engineered nucleic acid to 5′-methoxyuridine.

In some aspects, the at least one nucleic acid modification of the engineered nucleic acid comprises a modification of any one of or any combination of: 2′ modified nucleotide comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non limiting examples of nucleic acid modification to the engineered nucleic acid can include: modification of one or both of non-linking or linking phosphate oxygens in the phosphodiester backbone linkage (e.g., sulfur (S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap-OH) or 3′ end of the nucleic acid sequence (3′ tail or modification of 3′ end-OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some aspects, the nucleic acid modification of the engineered nucleic acid comprises at least one substitution of one or both of non-linking phosphate oxygen atoms in a phosphodiester backbone linkage of the engineered nucleic acid. In some aspects, the at least one nucleic acid modification of the engineered nucleic acid comprises a substitution of one or more of linking phosphate oxygen atoms in a phosphodiester backbone linkage of the engineered nucleic acid. Anon-limiting example of a nucleic acid modification of a phosphate oxygen atom is a sulfur atom. In some aspects, the nucleic acid modification of the engineered nucleic acid comprises at least one nucleic acid modification to a sugar of a nucleotide of the engineered nucleic acid. In some aspects, the nucleic acid modification of the engineered nucleic acid comprises at least one nucleic acid modification to the sugar of the nucleotide, where the nucleic acid modification comprises at least one locked nucleic acid (LNA). In some aspects, the nucleic acid modification of the engineered nucleic acid comprises at least one nucleic acid modification to the sugar of the nucleotide of the engineered nucleic acid comprising at least one unlocked nucleic acid (UNA). In some aspects, the nucleic acid modification of the engineered nucleic acid comprises at least one nucleic acid modification to the sugar of the nucleotide of the engineered nucleic acid comprising at least one ethylene nucleic acid (ENA). In some aspects, the nucleic acid modification of the engineered nucleic acid comprises at least one nucleic acid modification to the sugar comprising a modification of a constituent of the sugar, where the sugar is a ribose sugar. In some aspects, the nucleic acid modification of the engineered nucleic acid comprises at least one nucleic acid modification to the constituent of the ribose sugar of the nucleotide of the engineered nucleic acid comprising a 2′-O-Methyl group. In some aspects, the nucleic acid modification of the engineered nucleic acid comprises at least one nucleic acid modification comprising replacement of a phosphate moiety of the engineered nucleic acid with a dephospho linker. In some aspects, the nucleic acid modification of the engineered nucleic acid comprises at least one nucleic acid modification of a phosphate backbone of the engineered nucleic acid. In some aspects, the engineered nucleic acid comprises a phosphothioate group. In some aspects, the nucleic acid modifications of the engineered nucleic acid comprises at least one nucleic acid modification comprising a modification to a base of a nucleotide of the engineered nucleic acid. In some aspects, the nucleic acid modifications of the engineered nucleic acid comprises at least one nucleic acid modification comprising an unnatural base of a nucleotide. In some aspects, the nucleic acid modifications of the engineered nucleic acid comprises at least one nucleic acid modification comprising a morpholino group (e.g., a phosphorodiamidate morpholino oligomer, PMO), a cyclobutyl group, pyrrolidine group, or peptide nucleic acid (PNA) nucleoside surrogate. In some aspects, the nucleic acid modifications of the engineered nucleic acid comprises at least one nucleic acid modification comprising at least one stereopure nucleic acid. In some aspects, the at least one nucleic acid modification can be positioned proximal to a 5′ end of the engineered nucleic acid. In some aspects, the at least one nucleic acid modification can be positioned proximal to a 3′ end of the engineered nucleic acid. In some aspects, the at least one nucleic acid modification can be positioned proximal to both 5′ and 3′ ends of the engineered nucleic acid.

In some aspects, an engineered nucleic acid comprises a backbone comprising a plurality of sugar and phosphate moieties covalently linked together. In some cases, a backbone of an engineered nucleic acid comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some aspects, a backbone of an engineered nucleic acid can lack a 5′ reducing hydroxyl, a 3′ reducing hydroxyl, or both, capable of being exposed to a solvent. In some aspects, a backbone of an engineered nucleic acid can lack a 5′ reducing hydroxyl, a 3′ reducing hydroxyl, or both, capable of being exposed to nucleases. In some aspects, a backbone of an engineered nucleic acid can lack a 5′ reducing hydroxyl, a 3′ reducing hydroxyl, or both, capable of being exposed to hydrolytic enzymes. In some instances, a backbone of an engineered nucleic acid can be represented as a polynucleotide sequence in a circular 2-dimensional format with one nucleotide after the other. In some instances, a backbone of an engineered nucleic acid can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some aspects, the engineered nucleic acid described herein comprises at least one nucleic acid modification. A nucleic acid modification can be a substitution, insertion, deletion, nucleic acid modification, physical modification, stabilization, purification, or any combination thereof. In some cases, a modification is a nucleic acid modification. Suitable nucleic acid modifications comprise any one of: 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxy ribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, LNA, cEt, pseudouridine-5′-triphosphate, 5-methylcytidine-5′-triphosphate, 2-O-methyl-phosphorothioate or any combinations thereof.

In some cases, a modification can be permanent. In other cases, a modification can be transient. In some cases, multiple modifications are made to the engineered nucleic acid, the engineered nucleic acid modification can alter physio-chemical properties of a nucleotide, such as their conformation, polarity, hydrophobicity, chemical reactivity, base-pairing interactions, or any combination thereof.

In some embodiments, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the engineered nucleic acid comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some aspects, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; proengineered nucleic acids; neutral internucleoside linkages such as methylphosphonates; amide linked DNA; methylene(methylimino) linkages; formacetal and thioformacetal linkages; backbones containing sulfonyl groups; morpholino oligos: peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some aspects, the nucleic acid modification described herein comprises modification of a phosphate backbone. In some aspects, the engineered nucleic acid described herein comprises at least one chemically modified phosphate backbone. Example chemically modification of the phosphate group or backbone can include replacing one or more of the oxygens with a different substituent. Furthermore, the modified nucleotide present in the engineered nucleic acid can include the replacement of an unmodified phosphate moiety with a modified phosphate as described herein. In some aspects, the modification of the phosphate backbone can include alterations resulting in either an uncharged linker or a charged linker with unsymmetrical charge distribution. Example modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified engineered nucleic acid can be stereopure (e.g. S or R confirmation). In some cases, the chemically modified engineered nucleic acid comprises stereopure phosphate modification. For example, the chemically modified engineered nucleic acid comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

In some aspects, at least one phosphate group of the engineered nucleic acid can be chemically modified. In some aspects, the phosphate group can be replaced by non-phosphorus containing connectors. In some aspects, the phosphate moiety can be replaced by dephospho linker. In some aspects, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazoand methyleneoxymethylimino. In some aspects, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

In some aspects, the nucleic acid modification described herein comprises modification by replacement of a phosphate group. In some aspects, the engineered nucleic acid described herein comprises at least one chemically modification comprising a phosphate group substitution or replacement. Example phosphate group replacement can include non-phosphorus containing connectors. In some aspects, the phosphate group substitution or replacement can include replacing charged phosphate group can by a neutral moiety. Example moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

In some aspects, the nucleic acid modification described herein comprises modifying ribophosphate backbone of the engineered nucleic acid. In some aspects, the engineered nucleic acid described herein comprises at least one chemically modified ribophosphate backbone. Example chemically modified ribophosphate backbone can include scaffolds that can mimic nucleic acids can also be constructed wherein the phosphate linker and ribose sugar are replaced by nuclease resistant nucleoside or nucleotide surrogates. In some aspects, the nucleobases can be tethered by a surrogate backbone. Examples can include morpholino such as a phosphorodiamidatemorpholino oligomer (PMO), cyclobutyl, pyrrolidine and peptide nucleic acid (PNA) nucleoside surrogates.

In some aspects, the nucleic acid modification described herein comprises modification of sugar. In some aspects, the engineered nucleic acid described herein comprises at least one chemically modified sugar. Example chemically modified sugar can include 2′ hydroxyl group (OH) modified or replaced with a number of different “oxy” or “deoxy” substituents. In some aspects, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH2CH₂OR, wherein R can be, e.g., H or optionally substituted alkyl, and n can be an integer from 0 to 20. In some aspects, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where example bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some aspects, the “oxy”-2′ hydroxyl group modification can include the methoxyethyl group (MOE), (OCH₂CH₂OCH₃, e.g., a PEG derivative). In some cases, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the F position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the engineered nucleic acid described herein includes the sugar group ribose, which is a 5-membered ring having an oxygen. Example modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl orcyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some aspects, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some aspects, the modifications to the sugar of the engineered nucleic acid comprises modifying the engineered nucleic acid to include locked nucleic acid (LNA), unlocked nucleic acid (UNA), ethylene nucleic acid (ENA), constrained ethyl (cEt) sugar, or bridged nucleic acid (BNA).

In some aspects, the engineered nucleic acid described herein comprises at least one nucleic acid modification of a constituent of the ribose sugar. In some aspects, the nucleic acid modification of the constituent of the ribose sugar can include 2′-O-methyl, 2′-O-methoxyethyl (2′-O-MOE), 2′-fluoro, 2′-aminoethyl, 2′-deoxy-2′-fuloarabinou-cleic acid, 2′-deoxy, 2′-deoxy-2′-fluoro, 2′-O-methyl, 3′-phosphorothioate, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) 3′-phosphonoacetate (PACE), or 3′-phosphonothioacetate (thioPACE). In some aspects, the nucleic acid modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into engineered nucleic acids wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some aspects, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R=H, C₁-C₁₂ alkyl or a protecting group); and combinations thereof.

In some instances, the engineered nucleic acid described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m are from 1 to about 10. Other nucleic acid modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an engineered nucleic acid, or a group for improving the pharmacodynamic properties of an engineered nucleic acid, and other substituents having similar properties. Similar modifications may also be made at other positions on the sugar, particularly the 3′ position of the sugar on the 3′ terminal nucleotide or in 2′-5′ linked engineered nucleic acids and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′(LNA); 4′-(CH₂)—S-2′; 4′—(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

In some aspects, the nucleic acid modification described herein comprises modification of the base of nucleotide (e.g. the nucleobase). Example nucleobases can include adenine (A), thymine (T), guanine (G), cytosine (C), and uracil (U). These nucleobases can be modified or replaced to in the engineered nucleic acid described herein. The nucleobase of the nucleotide can be independently selected from a purine, a pyrimidine, a purine or pyrimidine analog. In some aspects, the nucleobase can be naturally-occurring or synthetic derivatives of a base.

### Immune Checkpoint Inhibitors and Modulators

Immune checkpoint are the regulators of immune system. They play a significant role in the immune evasion and escape of human tumors. Their modulators have shown significant efficacy in the cancer therapeutics field (see Wei et al., Cancer Discov. 2018. 10.1158/2159-8290). When secreted from tumors the intratumoral concentrations of such immune modulators are higher and their systemic concentrations are lower. This improved pharmacokinetic profile can boost the efficacy and lower the toxicity associated with these agents. In some embodiments, a payload may be or comprises an immune checkpoint inhibitor, i.e. an antagonist antibody agent against immune checkpoint proteins, e.g. anti-PD1, anti-PDL1, anti-CTLA-4, anti-TIM3, anti-BTLA, anti-VISTA, anti-LAG-3, anti-TIGIT, anti-CD39, anti-SIRP-α. In some other embodiments, a payload may be or comprises an agonist antibody against CD-28, OX40, GITR, CD137, CD27, HVEM, or CD27. In some other embodiments, the payload may be a costimulatory molecule such as CD80, CD86, and OX40L.

### Cytokines

Cytokines have critical roles in regulation of immune cells. IL-2 and IFN-alpha were the first two immunotherapy cytokines that were FDA approved for the treatment of metastatic melanoma and renal cell carcinoma (high dose, bolus I1-2) and Stage III melanoma (IFN-alpha) (Lee and Margolin, Cancers (Basel). 2011 December; 3(4): 3856-3893). However, their clinical use is limited by systemic toxicity issues (Rosenberg, J Immunol, 2014, 192 (12) 5451-5458). Those skilled in the art will appreciate that onco-selective production and secretion of cytokines can greatly improve their therapeutic window. In some embodiments, a payload for use in accordance with the present disclosure may be IL-2, IL-2 superkine/mutein, IL-12, IL15, IL15, IL15R-alpha fusion, I1-23, IL-36, TNF-alpha, IFN-alpha, IFN-gamma, FLT3 ligand, CCL4, RANTES, GM-CSF, or engineered variants or fusions thereof.

In some aspects, described herein is a composition or method utilizing at least one mRNA, where the mRNA encodes at least one cytokine (e.g., at least one interleukin or at least one interferon). Non-limiting example of cytokine encoded by the mRNA described herein can include 4-1BBL, acylation stimulating protein, adipokine, albinterferon, APRIL, Arh, BAFF, Bc-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL1l, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-30, IL-31, IL-32, IL-33, IL-34, IL-35, IL-36, IL-37, IL-38, IL-39, or IL-40, IL-4, IL-5, IL-6, IL-6-like, IL-7, IL-8/CXCL8, IL-9, inflammasome, interferome, interferon alpha (IFNα), interferon beta-la, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-01, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2. In some embodiments, cytokine encoded by the mRNA described herein can include IL-2, IL-12, IL-15, IFNα, or a combination thereof. In some embodiments, cytokine encoded by the mRNA described herein is IL-2, IL-12, IL-15, and IFNα.

### Modulators of Tumor Microenvironment

In human cancer, the tumor microenvironment is frequently altered to prevent or suppress anti-tumor immune response (Binnewies et al., Nature Medicine, 24, 541-550, 2018; Valkenburg et al., Nature Reviews Clinical Oncology, 15, 366-381, 2018). There are various modulators of tumor microenvironment that alter the extracellular matrix to enhance immune cell infiltration or that inflame the milieu to turn cold tumors into hot tumors. Some of these modulators have shown signs of efficacy in the preclinical models. However, some others were not dropped during preclinical or clinical development due to systemic toxicity issues (see, for example, Ramanathan et al, Journal of Clinical Oncology, Jan. 18-20, 2018 36.4_suppl.208). Present disclosure teach ways, to those skilled in the art, that would allow for the local secretion of such immune modulators, which can enhance intratumoral activity while minimizing systemic effects. In some embodiments, a payload may be a protein such as a kynureninase, adenosine deaminase (ADA2) and 15-hydroxyprostaglandin dehydrogenase (15-PGDH). In some other embodiments, a payload may be an enzyme, such as hyaluronidase and collagenase, which degrades the extracellular matrix and alters the tumor stroma.

### Cell Surface Antigens

Those skilled in the art are aware of various therapeutic technologies that have been developed for treating cancer by immunologically targeting antigens or epitopes expressed on surfaces of tumor cells. In some embodiments, a payload encoded by a translatable nucleic acid for use in accordance with the present disclosure encodes such an antigen or epitope, that may be immunologically targeted by a subject's immune system and/or by immune therapy (e.g., cell therapy such as CAR-T or CAR-NK therapy, or adoptive immunotherapy) administered to the subject. In some embodiments, such a cell surface antigen or epitope may be or comprise an antigen or epitope already expressed by relevant cancer cells; without wishing to be bound by any particular theory, the present disclosure proposes that increased expression of such an antigen or epitope may facilitate its targeting. In some embodiments, such an antigen or epitope may be one not already expressed by relevant tumor cells; in some such embodiments, it may be selected to permit targeting by an existing immune response or therapy.

### Genetic Modification Proteins

Those skilled in the art, reading the present disclosure, will be aware of the relevance of its teachings to genetic modification enzymes and their use, for example, to modify or destroy one or more aspects of a cancer cells' genome, transcriptome, etc.

For example, in some embodiments, a payload encoded by a translatable nucleic acid as described herein may be or comprise a genetic modification protein (e.g., that is or comprises a nuclease). In some embodiments, a genetic modification enzyme may be or comprise a transcription activator-like effector nuclease (TALEN), a zinc finger nuclease (ZFN), one or more components of a CRISPR based gene modification system (e.g., a Cas enzyme).

In some embodiments, a genetic modification protein (e.g., a nuclease) that targets sequences found preferentially or only in relevant cancer cells. However, those skilled in the art, reading the present disclosure, will appreciate that the degree of oncoselectivity in achieves permits use of genetic modification proteins that target sequences that are not particularly specific to cancer cells, as the genetic modification protein itself will be preferentially expressed only in those cells.

### Suicide Proteins

Those skilled in the art will be aware of various proteins commonly referred to as “suicide proteins” (encoded by “suicide genes”) and will appreciate that, in some embodiments, a payload sequence included in a translatable nucleic acid as described herein is or comprises a suicide protein.

In some embodiments, a suicide protein is a protein that induces cell death. In some embodiments, a suicide protein is a protein that induces immunogenic cell death, such as necroptosis, pyroptosis or ferroptosis. The present disclosure provides an insight that certain suicide proteins that induce necroptosis may be particularly advantageous for use in accordance with the present disclosure. For example, the present disclosure observes that necroptosis can induce and/or promote an adaptive immune response. Without wishing to be bound by any particular theory, the present disclosure observes that necroptosis involves immune ligands including Fas, TNF, and LPS leading to activation of RIPK. Dhuriya and Sharma J Neuroinflammation. 2018 Jul. 6; 15(1):199: Linkermann and Green N Engl J Med. 2014 Jan. 30; 370(5). 455-465. The present disclosure teachesthat use of a necroptotic suicide protein, which may induce and/or promote an adaptive immune response, may facilitate inhibition, destruction and/or removal of tumor cells. In some embodiments, a suicide protein induces apoptosis; in some such embodiments, a suicide protein is p53, or is a protein involved in a p53-mediated apoptosis pathway (e.g. PUMA, BIM, BAX, BAK, tBID, CASPASE-3, CASPASE-7, CASPASE-8, CASPASE-9).

In some embodiments, a suicide protein is or comprises a protein that renders cells expressing it more susceptible to killing by a separate agent. To give but one example, those skilled in the art are aware of certain viral and/or bacterial enzymes that are not naturally found in mammals and that convert a substance that may be harmless to cells that do not express the enzyme(s) into a toxin. In some embodiments, such a suicide protein is or comprises an enzyme that converts an otherwise inactive agent (e.g., drug) into a toxic antimetabolite, e.g., that inhibits nucleic acid synthesis. In some such embodiments, a suicide protein is a thymidine kinase, wherein the payload sequence encoding thymidine kinase is co-administered with or administered before ganciclovir or valacyclovir treatment.

In some embodiments, a suicide protein payload for use in accordance with the present disclosure is Mixed Lineage Kinase Domain Like Pseudokinase (MLKL), Receptor-interacting serine/threonine-protein kinase 3 (RIPK3), Receptor-interacting serine/threonine-protein kinase 1 (RIPK1), Fas-associated protein with death domain (FADD), or gasdermin D (GSDMD), cysteine-aspartic proteases, cysteine aspartases or cysteine-dependent aspartate-directed proteases (CASPASE-1 or CASP-1), CASPASE-4, CASPASE-5, CASPASE-12, PYCARD/ASC (PYD and CARD domain containing/Fas-associated protein with death domain) or variants thereof.

### Toxins

In some embodiments, a payload for use in accordance with the present disclosure may be or include a toxin protein. Those skilled in the art will be aware of a variety of toxin proteins that may be useful to kill cancer cells. As noted herein, it is one feature of the present disclosure that the degree of oncoselectivity achieved is such that even very potent payloads may be utilized notwithstanding that such payloads might have significant deleterious effects if expressed in non-cancer cells. In some embodiments, a payload is a toxin that is not secreted from a cancer cell.

In some embodiments, a toxin may be or comprise a bacterial toxin. In some embodiments, a toxin may be or comprise a toxin produced by a venomous animal (see, for example, Kozlov et al Rec Pat DNA Gene Sequ1:200, 2007). In some embodiments, a toxin may be or comprise a plant toxin.

In some embodiments, a toxin that may be utilized as a payload in accordance with the present disclosure may be or comprise a phospholipase or a lecithinase. In some embodiments, a useful toxin may be or comprise a lethal toxin. In some embodiments, a useful toxin may be or comprise an exotoxin. In some embodiments, a useful toxin may be or comprise a pore-forming toxin. In some embodiments, a useful toxin may be or comprise a pyrogenic exotoxin.

In some embodiments, a toxin that may be utilized as a payload is one found in (or derived from) a bacterium that is a Bacillus (e.g., Bacillus anthracis), Bortadella (e.g., Bortadella pertussis), Clostridium (Clostridium botulinum), Corynebacterium (e.g., Corynebacterium diphtheriae), Eschericia (e.g., Eschericia coli), listeria (e.g., Listeria monocytogenes), Pseudomonas (Pseudomonas aeruginosa), Staphylococcus (e.g., Staphylococcus aureus), Streptococcus, Shigella (e.g. Shigella dyseniteriae),

In some embodiments, a toxin may be or comprise cholera toxin (e.g., A-5B), diphtheria toxin (e.g., A/B), pertussis toxin (e.g., A-5B), E. coli heat-labile toxin LT (e.g., A-5B), shiga toxin (e.g., A-5B), Pseudomonas exotoxin (e.g., A/B), botulinum toxin (e.g., A/B), tetanus toxin (e.g., A/B), anthrax toxin (e.g., lethal factor [LF]), Staphylococcus aureaus exfoliatin B.

In some embodiments, a toxin may be or comprise perfringiolysin (e.g., from Clostridium perfringens), hemolysin (e.g., from Eschericia coli), listeriolysin (e.g., from Listeria monocytogenes), anthrax EF (e.g., from Bacillys anthracis), alpha toxin (e.g., from Staphylococcus aureaus), pneumolysin (e.g., from Streptococcus pneumoniae), streptolysin PO (e.g., from Streptococcus pyogenes), leucocidin (e.g., from Staphylococcus aureus).

In some embodiments, a toxin may be a component of an exotoxin (e.g. Lethal Factor of anthrax toxin), that is, on its own, not capable of being internalized into mammalian cells.

In some embodiments, a toxin may be or comprise ricin or an amanitin. In some embodiments, a toxin may be or comprise alpha-amanitin.

### Inducible or Repressible Proteins

Recent advances in genetic engineering and synthetic biology allow for proteins that are inducible or repressible via small molecule modulators. In some embodiments, a repressible protein can be fused to a Ligand-Induced Degradation (LID) domain, which results in the proteolytic cleavage of the protein upon treatment with the small molecule Shield-1. In some other embodiments, an inducible protein may be inducible Caspase-9, which is activated by the small molecule rimiducid by dimerization. The activated Caspase-9 leads to rapid apoptosis of cells. In some other embodiments, the induction or repression may be achieved via other degradation domains (e.g. dihydrofolatereductase based destabilization domain) or dimerization domains (e.g. FKBP—FRB) and/or other small molecules (e.g. doxycycline, rapamycin, trimethoprim). In some embodiments, a payload for use in accordance with the present disclosure may be or include an inducible or repressible protein.

### Viral Proteins

Those skilled in the art are aware of a variety of viruses that produce proteins useful as payloads as described herein. In some embodiments, a payload may be or comprise a viral protein. In some embodiments, a payload may be LMP1 protein of Epstein-Barr virus. In some embodiments a payload may be or comprise an oncolytic virus protein.

In some embodiments, a payload may be or comprise a viral replication protein. In some embodiments, the viral replication protein is a protein needed for the viral replication cycle. In some embodiments, the viral replication protein is an enzyme. In some embodiments, the viral replication protein is a protease, a polymerase, or a transcriptase.

### Production

Those skilled in the art, reading the present disclosure, will appreciate that a variety of technologies are available that can usefully be employed to produce a translatable nucleic acid as described herein. In some embodiments, such production may be ex vivo (i.e., outside of a subject in need of cancer treatment as described herein); in some embodiments, such production may be in vivo.

In some embodiments, a translatable nucleic acid may be produced, wholly or partially, by chemical synthesis and/or chemical modification (e.g., capping)

In some embodiments, a translatable nucleic acid may be produced, wholly or partially, by copying (e.g., via replication or transcription) of a template nucleic acid. In some embodiments, such copying may be ex vivo; in some embodiments, it may be in vivo.

### Delivery

Those skilled in the art, reading the present disclosure, will appreciate that a variety of technologies are available to achieve delivery of a translatable nucleic acid to (at least) cancer cells in accordance with the present disclosure, and furthermore will appreciate that some modes of delivery involve administration of a composition comprising the translatable nucleic acid (e.g., mRNA), and some modes of delivery involve administration of a composition from which the translatable nucleic acid is generated after administration (e.g., via administration of a vector that encodes or templates the translatable nucleic acid.

### Nanoparticle Delivery

As noted herein, those skilled in the art will be aware that a variety of administration systems have been developed to achieve effective delivery of nucleic acids into cells, including within mammalian (e.g., human) subjects.

Among such available technologies are various nanoparticle technologies including, for example, hydrogel, lipid, and/or polymer nanoparticle technologies.

In some embodiments, a nucleic acid is delivered to a subject in accordance with the present disclosure using a lipid nanoparticle (LNP). As used herein, the phrase “lipid nanoparticle” refers to a transfer vehicle comprising one or more lipids (e.g., cationic lipids, non-cationic lipids, and PEG-modified lipids). In some embodiments, lipid nanoparticles are formulated to deliver one or more copies of the nucleic acid to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

In some embodiments, a nucleic acid is delivered to a subject in accordance with the present disclosure using a polymer nanoparticle. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

In some embodiments, lipids for use in the delivery of a nucleic acid of the present invention include those described in international patent publication WO 2010/053572, incorporated herein by reference. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g., (15Z, 18Z)—N,N-dimethyl-6-(9Z, 12Z)-octadeca-9, 12-dien-1-yl)tetracosa-15,18-dien-1-amine (HGT5000), (15Z, 18Z)—N,N-dimethyl-6-((9Z, 12Z)-octadeca-9, 12-dien-1-yl)tetracosa-4,15,18-trien-1-amine (HGT5001), and (15Z,18Z)—N,N-dimethyl-6-((9Z, 12Z)-octadeca-9, 12-dien-1-yl)tetracosa-5, 15, 18-trien-1-amine (HGT5002).

In some embodiments, the lipid N-[l-(2,3-dioleyloxy)propyl]-N,N,N-trimethylammonium chloride or “DOTMA” is used. (Feigner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Contemplated lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylarnrnonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethy 1-1-(cis,cis-9′, 1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-dimethylethanamine (DLin-KC2-DMA))(See, WO 2010/042877; Semple et al., Nature Biotech. 28: 172-176 (2010) (Heyes, J., et al., J Controlled Release 107: 276-287 (2005); Morrissey, D V., et al., Nat. Biotechnol. 23(8): 1003-1007 (2005); PCT Publication WO2005/121348A1), DLin-MC3-DMA (See WO2015199952A1 Tam and Cullis Pharmaceutics. 2013 September; 5(3): 498-507) or mixtures thereof. The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments, an LNP comprises one or more: (1) “cationic” and/or amino (ionizable) lipids, (2) phospholipids and/or polyunsaturated lipids (helper lipids), (3) structural lipids (e.g., sterols), and/or (4) lipids containing polyethylene glycol (PEG lipids). In some embodiments, an LNP is one as described in WO2021026358.

In some embodiments, an LNP of the present disclosure comprises at least one cationic lipid. In some embodiments, the present disclosure provides LNPs comprising at least one cationic ionizable lipid. In some embodiments the term “cationic ionizable lipid” refers to lipid and lipid-like molecules with nitrogen atoms that can acquire charge (pKa). In some embodiments, a cationic ionizable lipid for use in accordance with the present disclosure has a pKa of 5, 6, 7, 8, 9, 10, 11 at physiological pH. In some embodiments a cationic ionizable lipid comprises one or more groups which is protonated at physiological pH but deprotonates and has no charge at a pH above 5, 6, 7, 8, 9, 10, 11, or 12. In some embodiments a cationic ionizable lipid comprises one or more groups which are protonated and have a charge at a pH above 5, 6, 7, 8, 9, 10, 11, or 12. In some embodiments, the ionizable cationic group may contain one or more protonatable amines which are able to form a cationic group at physiological pH.

One insight provided by the present disclosure is that use of a cationic ionizable lipid with a pKa within a particular range as described herein may be particularly useful for delivery of nucleic acids, for example in LNP preparations as described herein. Specifically, in some embodiments, a cationic ionizable lipid has a high pKA. In some embodiments a high pKa is a pKa greater than 7. In some embodiments a high pKa is a pKa greater than 7.4. In some embodiments, a cationic ionizable lipid for use in accordance with the present disclosure has a pKa of between 7 and 8, 7.5 and 8.5, 8 and 9, 8.5 and 9.5, 9 and 10. In some embodiments, a cationic ionizable lipid for use in accordance with the present disclosure has a pKa of between 7.2 and 8.2, 7.4 and 8.4, 7.6 and 8.6, 7.8 and 8.8, 8.0 to 9.0, 8.2 to 9.2, 8.4 to 9.4, 8.6 to 9.6, 8.8 to 9.8, 9.0 to 10.0. In some embodiments, a useful cationic ionizable lipid has a pKa of 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. This insight is particularly surprising in light of the understanding in the field that pKas >7 are not optimum for LNP payload delivery. See Jayaraman et al., Angew. Chem. Int. Ed. 2012, 51, 8529-8533. Indeed, Jayaraman et al states that LNP potency rapidly decreases if the pKa is outside of the range of 6.2 to 6.5. Further, with respect to intramuscular administration of LNP Hassett et al., Molecular Therapy: Nucleic Acids Vol. 15 Apr. 2019 states that a lipid pKa range of 6.6-6.9 is optimal.

In some embodiments, a cationic ionizable lipid with a high pKA (e.g., >7) is disclosed or described in WO2020219876; US20210162053; US20160317676; and WO2021113365 each of which is incorporated herein in their entirety. In some embodiments, a cationic ionizable lipid with a high pKA (e.g. >7 is one selected from those listed in Table 4.

TABLE 4
Exemplary cationic ionizable lipid with a high pKA

| | |
|---|---|
| 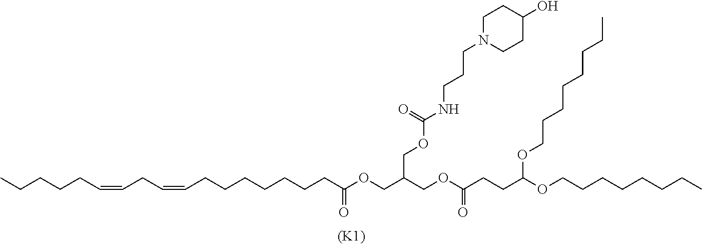 | 3-((4,4-bis(octyloxy) butanoyl)oxy)-2- ((((3-(4- hydroxypiperidin-1- yl)propyl)carbamoyl) oxy)methyl)propyl (9Z, 12Z)-octadeca- 9, 12-dienoate |
| 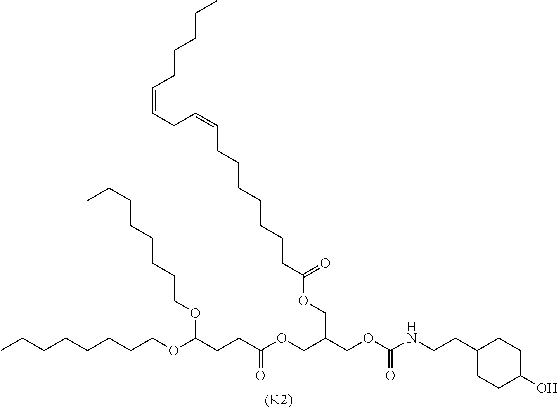 | 3-((4,4-bis(octyloxy) butanoyl)oxy)-2- ((((2-(4- hydroxypiperidin-1- yl)ethyl)carbamoyl) oxy)methyl)propyl (9Z, 12Z)-octadeca- 9, 12-dienoate |
| 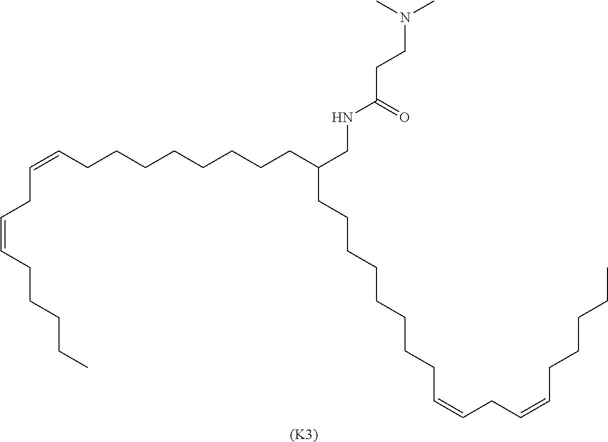 [SMILES: CCCCC/C=C\C/C=C\CCCCCCCCC(CCCCCCCC/C=C\C/C=C\CCCCC)CNC(=O)CCN(C)C ; "(K3)"] | 3-(dimethylamino)- N-((11Z, 14Z)-2- ((9Z,12Z)-octadeca- 9,12-dienyl)icosa- 11,14-dienyl) propanamide |
| 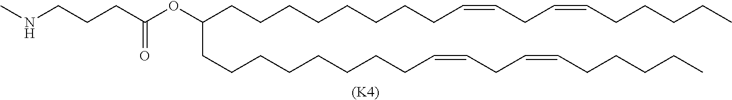 | DLin-M-C3-EA |
| 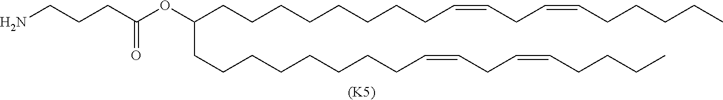 | DLin-M-C3-A |
| 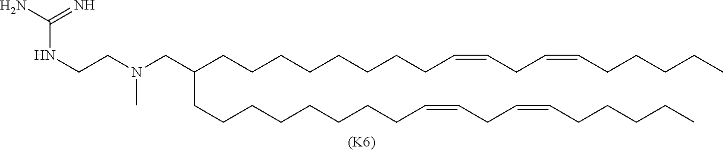 | |
| 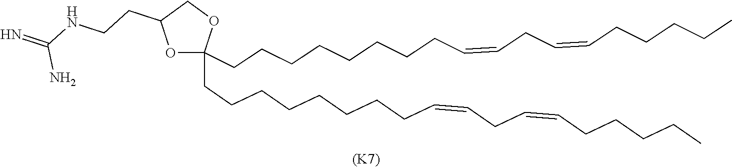 | |
| 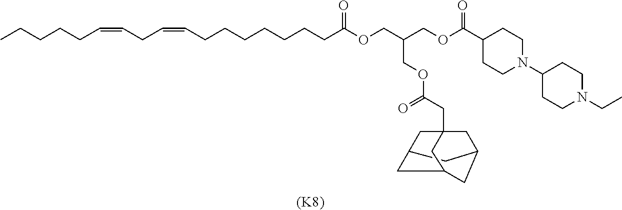 [SMILES: CCCCC/C=C\C/C=C\CCCCCCCC(=O)OCC(COC(=O)CC12CC3CC(CC(C3)C1)C2)COC(=O)C1CCN(C2CCN(CC)CC2)CC1 ; "(K8)"] | |
| 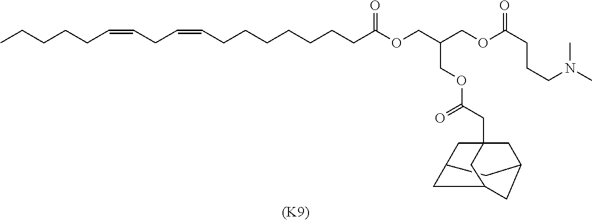 [SMILES: CCCCC/C=C\C/C=C\CCCCCCCC(=O)OCC(COC(=O)CCCN(C)C)COC(=O)CC12CC3CC(CC(C3)C1)C2 ; "(K9)"] | |
| 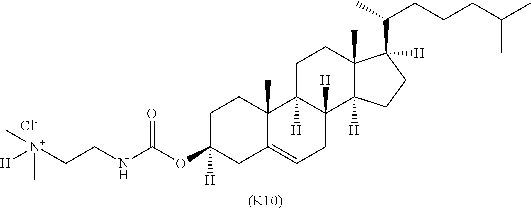 | DC-Cholesterol |

Among other things, the present disclosure demonstrates that use of a high pKa cationically ionizable lipid in LNPs as described herein may be particularly useful to achieve delivery of nucleic acids to the lung.

In some embodiments the present disclosure provides LNPs comprising a at least first cationic ionizable lipid and a second cationic ionizable lipid. In some such embodiments, more than one (e.g., each) of such at least first and second cationic ionizable lipids has a high pKa (e.g., greater than 7, e.g., greater than 7.4) as described herein. Alternatively, in some such embodiments, only one (i.e., a first) cationic ionizable lipid has such a high pKa (e.g., greater than 7, e.g., greater than 7.4) as described herein. In some embodiments, a second cationic ionizable lipid is a non-high-pKa lipid, e.g., in that it has a pKa below 7, e.g., about 6.8, 6.6, 6.4, 6.2, 6.0, 5.8, 5.6, 5.4, 5.2, 5.0 or lower. In some embodiments, a non-high-pKa lipid (e.g., a second lipid) has a pKa of about 6.4 or lower. In some embodiments, a non-high-pKa lipid (e.g., a second lipid) has a pKa of about 6.4. In some embodiments, a non-high-pKa lipid (e.g., a second lipid) has a neutral pKa.

In some embodiments, a cationic ionizable lipid with a non-high pKa (e.g., <7) is disclosed or described in Finn et al., 2018 Cell Reports 22, 2227-2235; Jayaraman et al., Angew. Chem. Int. Ed. 2012, 51, 8529-8533; Hassett et al., Molecular Therapy. Nucleic Acids Vol. 15 Apr. 2019; WO2015074085; WO2020118041; WO2020072605; WO2020252589; WO2021055849; WO2018232120; WO2021030701; WO2020146805; WO2019036000; WO2018200943; and WO2018191657 each of which is incorporated herein in their entirety. In some embodiments, a cationic ionizable lipid with a non-high pKA (e.g., <7) is one selected from those listed in Table 5.

TABLE 5
Exemplary cationic ionizable lipid with a non-high pKA

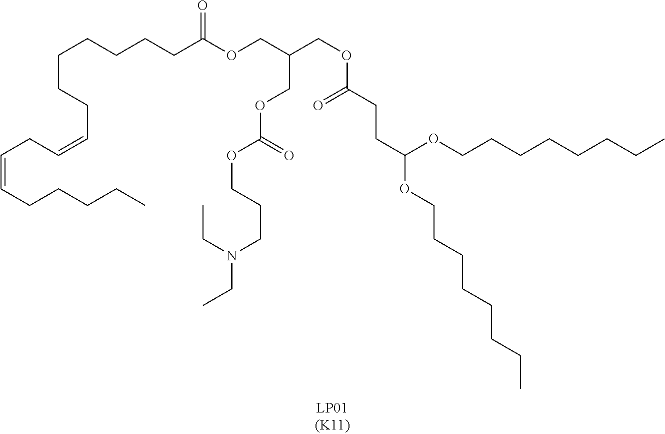 [SMILES: CCCCC/C=C\C/C=C\CCCCCCCC(=O)OCC(COC(=O)CCC(OCCCCCCCC)OCCCCCCCC)COC(=O)OCCCN(CC)CC]

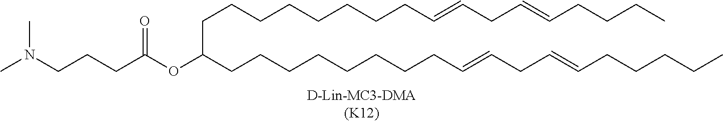 [SMILES: CCCC/C=C/C/C=C/CCCCCCCCC(CCCCCCCC/C=C/C/C=C/CCCCC)OC(=O)CCCN(C)C]

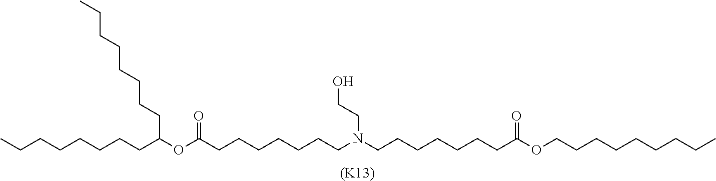

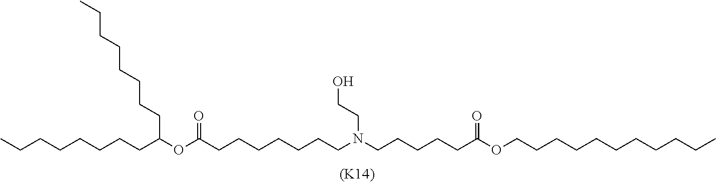

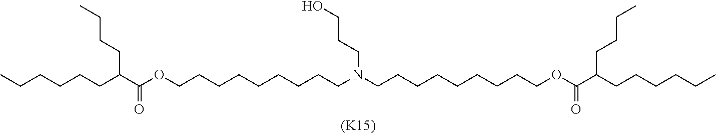

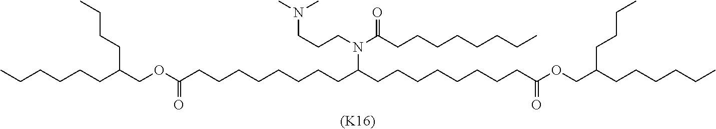 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC ; "(K16)"]

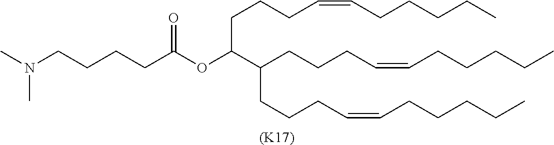

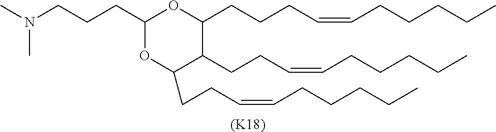

In some embodiments, an LNP comprises about 0-80 mol % of high pKa cationic ionizable lipid as described herein; in some embodiments, an LNP comprises two or more high pKa cationic ionizable lipids that, together make up such 0-80 mol % of the LNP. In some embodiments, an LNP comprises about 20-80 mol % of high pKa cationic ionizable lipid. In some embodiments, an LNP comprises about 0, 5, 10, 15, 20, 25, 30, 35, 40, 45, 50, 55, 60, 65, 7-, 75, 80 mol % of high pKa cationic ionizable lipid.

In some embodiments, an LNP comprises about 0-60 mol % of cationic ionizable lipid that is not high pKa (e.g., that is characterized by a pKa below about 7, such as a pKa of about 6.4 or a neutral pKa); in some embodiments, an LNP comprises two or more non-high pKa cationic ionizable lipids that, together, make up such 0-60 mol % of the LNP. In some embodiments, an LNP comprises about 0, 5, 10, 15, 20, 25, 30, 35, 40, 45, 50, 55, 60 mol % of a non-high-pKa cationic ionizable lipid.

In some embodiments, the present disclosure provides LNPs comprising at least one sterol. In some embodiments, an LNP comprises about 0-40 mol % of a sterol. In some embodiments, an LNP comprises about 0, 5, 10, 15, 20, 25, 30, 35, 40 mol % of a sterol.

In some embodiments, a sterol is cholesterol, or a variant or derivative thereof. In some embodiments, a cholesterol is modified, for example oxidized. Unmodified cholesterol can be acted upon by enzymes to form variants that are side-chain or ring oxidized. In some embodiments, a cholesterol can be oxidized on the beta-ring structure or on the hydrocarbon tail structure. Exemplary cholesterols that are considered for use in the disclosed LNPs include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol,beta-sitosterol, stigmasterol, brassicasterol, campesterol, or combinations thereof. In some embodiments, side-chain oxidized cholesterol can enhance cargo delivery relative to other cholesterol variants. In some embodiments, a cholesterol is an unmodified cholesterol.

In some embodiments, the present disclosure provides LNPs comprising at least one helper lipid. In some embodiments a helper lipid is a phospholipid. In some embodiments, an LNP comprises about 0-20 mol % of a helper lipid. In some embodiments, an LNP comprises about 0, 5, 10, 15, 20 mol % of a helper lipid.

Exemplary phospholipids include but are not limited to 1,2-distearoyl-snglycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl snglycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2 oleoylphosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or combinations thereof. In some embodiments, a phospholipid is DSPC. In some embodiments, a phospholipid is DMPC.

In some embodiments, a phospholipid comprises 1,2-dioleoyl-sn-glycero-3-phosphoethanolamine-N-(succinyl)(succinyl PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), 1,2-dioleoyl-sn-glycero-3-phosphoethanolamine (DOPE), 1,2-dimyristoyl-sn-glycero-3-phosphocholine (DMPC), 1,2-dipalmitoyl-sn-glycero-3-phosphocholine (DPPC), or a combination thereof.

In some embodiments, the present disclosure provides LNPs comprising at least one PEGylated lipid. In some embodiments, an LNP comprises about 0-25 mol % of a PEGylated lipid. In some embodiments, an LNP comprises about 0, 5, 10, 15, 20, 25 mol % of a PEGylated lipid.

In some embodiments, inclusion of a PEGylated lipid can be used to enhance lipid nanoparticle colloidal stability in vitro and circulation time in vivo. In some embodiments, PEGylation is reversible in that the PEG moiety is gradually released in blood circulation. Exemplary PEGylated-lipids include but are not limited to PEG conjugated to saturated or unsaturated alkyl chains having a length of C6-C20, PEG-modified-28-phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, a PEGylated-lipid may be PEG-c-DOMG, PEG-DMG, PEG-DLPE, PEG-DMPE, PEG-DPPE, PEG-DSG or a PEGDSPE lipid.

In some embodiments, the present disclosure provides LNPs comprising at least one PEG-ligand. In some embodiments, an LNP comprises about 0-2 mol % of a PEG-ligand. In some embodiments, an LNP comprises about 0, 0.01, 0.05, 0.1, 0.5, 0.75, 1, 1.5, 1.75, 2 mol % of a PEG-ligand. In some embodiments, a ligand (e.g., as included in a PEG-ligand) is or comprises: antibodies targeting cell surface proteins, hyaluronic acid, small molecules, peptides, and/or peptides that target integrins.

### Vector Delivery

In some embodiments, a translatable nucleic acid as described herein may be delivered to a subject by administration of a nucleic acid vector that encodes and/or templates the translatable nucleic acids. In some embodiments, a useful vector may be or comprise a viral vector.

In some embodiments, a vector system (e.g., a viral vector system) may be or comprise components and/or sequences found in nature (i.e., wild type components and/or sequences); in some embodiments, a vector system may be or comprise engineered components and/or sequences (i.e., components whose sequence has been modified relative to an appropriate wild type reference and/or components that are not found together in a wild type reference but may, for example, represent an assemblage of components from a plurality of different sources).

Those skilled in the art are familiar with a variety of viral vector systems that could be useful in accordance with the present disclosure.

In some embodiments, a viral vector system may be or comprise components of a virus that preferentially infects cancer cells (e.g., an oncolytic virus). Those skilled in the art are aware of a variety of oncolytic viruses including, for example, vaccinia virus, a vesicular stomatitis virus, a poliovirus, a reovirus, a senecavirus, and adenovirus.

The present disclosure provides an insight that use of an oncolytic viral vector system may have certain advantages, for example in potentially providing a complementary mechanism of killing for tumor cells.

However, as noted herein, the degree of oncoselectivity achieved in accordance with the present disclosure renders oncoselectivity of a nucleic acid delivery vector not critical to many embodiments of the disclosure.

### Subjects

As described herein, the present disclosure provides technologies that are particularly useful in the treatment of cancer.

In some embodiments, provided technologies are applied to subjects suffering from cancer. That is, in some embodiments, a translatable nucleic acid as described herein (e.g., comprising at least one oncoselective translation sequence elements and a payload-encoding sequence) is delivered to (e.g., by administration of a composition comprising the translatable nucleic acid, or of a composition that causes the translatable nucleic acid to be generated in or by the subject).

In some embodiments, a subject has received, is receiving and/or will receive other therapy (e.g., other therapy to treat the cancer and/or one or more side effects of the cancer or its treatment). In some such embodiments, a payload is or comprises a protein that increases susceptibility of cells to the other therapy.

In some embodiments, a subject is not receiving a pharmaceutical agent that is known to cause stop codon readthrough in healthy cells. In some embodiments, a subject is not receiving aminoglycosides and/or macrolides.

In some embodiments, a subject is not receiving cystic fibrosis and/or Duchenne muscular dystrophy therapy (e.g. Ataluren or PTC124).

In some embodiments, a subject is not receiving pyronaridine tetraphosphate (anti-malarial), and potassium para-aminobenzoate (PABA, used of Peyronie's disease), experimental compounds RTC13, RTC14, and NB54, and/or herbal supplement escin.

In some embodiments, a subject is not affected by ribosomopathies such as Diamond-Blackfan anemia, Dyskeratosis congenita, Shwachman-Diamond syndrome, 5q-myelodysplastic syndrome, Treacher Collins syndrome, Cartilage-hair hypoplasia, Isolated congenital asplenia, Bowen-Conradi syndrome, North American Indian childhood cirrhosis.

### Resistant or Refractory to Cancer Therapy

In some embodiments, provided technologies are applied to subjects suffering from cancer that have received or are receiving treatment. In some embodiments, a subject has received or is receiving immune checkpoint inhibitor therapy. In some embodiments, a subject are resistant or refractory to immune checkpoint therapy they have received. In some embodiments, provided technologies are applied to subjects based on diagnosis of cancer refractory or to immune checkpoint therapy. In some embodiments, a subject has demonstrated and/or been diagnosed with immune checkpoint therapy relapse (e.g. a non-beneficial response to immune checkpoint therapy; or progressive disease). In some embodiments, biomarkers or indicators of a cancer resistant or refractory to immune checkpoint therapy are described in Ren et al., Mol Cancer 19, 19 (2020) and Barrueto et al., Transl Oncol. 2020 March; 13(3): 100738.

### Method

### Delivery

The engineered nucleic acid can be readily introduced into a cell, e.g., mammalian, bacterial, yeast, or insect cell by any method in the art. For example, the engineered nucleic acid can be transferred into a host cell by physical, chemical, or biological means. In some embodiments, the engineered nucleic acid can be delivered into the cell via physical methods such as calcium phosphate precipitation, lipofection, particle bombardment, microinjection, gene gun, electroporation, and the like.

Physical methods for introducing the engineered nucleic acid encoding into the cell can include calcium phosphate precipitation, lipofection, particle bombardment, microinjection, gene gun, electroporation, and the like. One method for the introduction of the engineered nucleic acid a host cell is calcium phosphate transfection.

Chemical means for introducing the engineered nucleic acid encoding the non-naturally into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of engineered nucleic acid or vector encoding the engineered nucleic acid with targeted nanoparticles.

In the case where a non-viral delivery system is utilized, an example delivery vehicle is a liposome. The use of lipid formulations is contemplated for the introduction of the engineered nucleic acid or vector encoding the engineered nucleic acid into a cell (in vitro, ex vivo, or in vivo). In another aspect, the vector can be associated with a lipid. The vector associated with a lipid can be encapsulated in the aqueous interior of a liposome, interspersed within the lipid bilayer of a liposome, attached to a liposome via a linking molecule that is associated with both the liposome and the engineered nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the engineered nucleic acids described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e. g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the engineered nucleic acid described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some embodiments, the engineered nucleic acid can be delivered into the cell via biological methods such as the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAV vectors), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome.

In some instances, the viral vector is a chimeric viral vector, comprising viral portions from two or more viruses. In additional instances, the viral vector is a recombinant viral vector. In some cases, the vector comprises additional features. Additional features can comprise sequences such as tags, signaling peptides, intronic sequences, promoters, stuffer sequences, and the like. In some cases, the vector comprises a signaling peptide. A signaling peptide is sometimes referred to as signaling sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signaling peptides. A signaling peptide can be of any length but typically from 15-30 amino acids long. A signaling peptide can be from about: 10-15, 10-20, 10-30, 15-20, 15-25, 15-30, 20-30, or 25-30 amino acids long. Various signaling peptides can be utilized and include but are not limited to: human antibody heavy chain (Vh), human antibody light chain (VI), and aflibercept.

In an embodiment, an additional feature of the vector includes promoter. Promoter is sequences of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. In some cases, the promoters can be inducible promoters. Various promoters are contemplated and can be employed in the vectors of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF promoter, a glyceraldehyde 3-phosphage dehydrogenase (GDS) promoter, an ADH1 promoter, a CaMV35S promoter, a Ubi promoter, a human polymerase III RNA (HI) promoter, a U6 promoter, a polyadenylated construct thereof, and any combination thereof. In some cases, the promoter is the CMV promoter.

In some embodiments, the vector comprising the at least two expression cassettes under expression control of two different promoters. Such arrangement allows the two signaling transduction regulators to be expressed simultaneously or in a desired sequential order in a cell.

### Treatment

Provided herein are methods of treating a disease or condition described here. A method of treatment can comprise introducing to a subject in need a engineered nucleic acid. Also provided is a method of treating disease or condition that comprises administering a pharmaceutical composition to a subject in need thereof. A pharmaceutical composition can comprise a sequence that encodes a biologic that comprises the engineered nucleic acid. In some embodiments, administration is by any suitable mode of administration, including systemic administration (e.g., intravenous, inhalation, vitreous, or etc.). In some embodiments, the subject is human.

In some embodiments, the engineered nucleic acid is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time.

In some embodiments, the method comprises administering the engineered nucleic acid in a therapeutically-effective amount by various forms and routes including, for example, intratumoral, oral, or topical administration. In some embodiments, a composition may be administered by intratumoral, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

Actual dosage levels of an agent of the disclosure (e.g., the engineered nucleic acid or a pharmaceutical composition) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The engineered nucleic acid, the vector comprising the engineered nucleic acid, or the pharmaceutical composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of“increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20/6, at least 30%, at least 40% or more, and is preferably down to a level accepted as within the range of normal for an individual without a given disease.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method of treating a subject suffering from cancer comprising administering to the subject one or more engineered nucleic acid(s) and an immune checkpoint inhibitor wherein: the one or more engineered nucleic acid(s) comprises a nucleotide sequence that includes a sequence element that is or is a complement of an oncoselective translation sequence element.

Embodiment 2. A method of treating a subject suffering from cancer with immune checkpoint inhibitor therapy the improvement comprising administering one or more engineered nucleic acid(s) and an immune checkpoint inhibitor wherein: the one or more engineered nucleic acid(s) comprises a nucleotide sequence that includes a sequence element that is or is a complement of an oncoselective translation sequence element.

Embodiment 3. The method of Embodiment 1 or Embodiment 2, wherein the engineered nucleic acid(s) and the immune checkpoint inhibitor are administered subsequently, concomitantly, or adjunctively.

Embodiment 4. The method of any one of Embodiments 1-3, wherein the immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, or CD137.

Embodiment 5. The method of any one of Embodiments 1-4, wherein the immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

Embodiment 6. A method of treating a subject suffering from cancer wherein the subject has received or is receiving a first immune checkpoint inhibitor, the method comprising administering to the subject one or more engineered nucleic acid(s) and a second immune checkpoint inhibitor, wherein the engineered nucleic acid(s) comprises a nucleotide sequence that includes a sequence element that is or is a complement of an oncoselective translation sequence element.

Embodiment 7. The method of Embodiment 6, wherein the first immune checkpoint inhibitor and the second immune checkpoint inhibitor are the same.

Embodiment 8. The method of Embodiment 6, wherein the first immune checkpoint inhibitor and the second immune checkpoint inhibitor are different.

Embodiment 9. The method of any one of Embodiments 6-8, wherein the subject is not responding to the first immune checkpoint inhibitor.

Embodiment 10. The method of any one of Embodiments 6-8, wherein the cancer shows no response or progressive disease.

Embodiment 11. The method of any one of Embodiments 6-8, wherein the subject shows signs of relapse or resistance.

Embodiment 12. The method of any one of Embodiments 6-8, wherein the first or second immune checkpoint inhibitor comprises one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, or CD137.

Embodiment 13. The method of anyone of Embodiments 6-8, wherein the first or second immune checkpoint inhibitor comprises one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

Embodiment 14. The method of any one of the preceding Embodiments, wherein the engineered nucleic acid(s) is delivered to the subject by a lipid nano particle (LNP).

Embodiment 15. The method of any one of the preceding Embodiments, wherein the oncoselective translation sequence element is or comprises an oncoselective read-through motif within or upstream of the open reading frame.

Embodiment 16. The method of any one of the preceding Embodiments, wherein the oncoselective readthrough motif comprises an upstream flanking sequence, a stop codon, and a downstream flanking sequence.

Embodiment 17. The method of any one of the preceding Embodiments, wherein an oncoselective readthrough motif comprises a sequence selected from the group comprising: VNNNNNNMNNMWK, NNNVWNNKGHHNH, DVHVNNNCWNNNB, MWBNNNNNNNNNN, WGNNSNHNHDNNN, VNNNNNNMNNMWK or VMNNWNKNNNNNN, wherein V stands for A, C or G, M stands for A or C, W stands for A or T/U, K stands for G or T/U, H stands for A, C or T/J, D stands for A,G or T/U, B stands for C, G or T/U, S stands for G or C, N stands for any nucleotide, within the region that spans the readthrough stop codon and the first 14 nucleotides of the downstream flanking sequence.

Embodiment 18. The method of any one of the preceding Embodiments, wherein the oncoselective read through motif comprises a stem loop; a bulge loop, a pseudoknot, or a combination thereof within the first 50 nucleotides of the downstream flanking sequence and part of this stem loop located preferably within stop codon and the first 16 nucleotides of the downstream flanking sequence, or a combination thereof.

Embodiment 19. The method of any one of the preceding Embodiments, wherein a stem loop comprises more than 20 base paired nucleotides within first 50 nucleotides of the downstream flanking sequence.

Embodiment 20. The method of any one of the preceding Embodiments, wherein an oncoselective read through motif comprises a downstream flanking sequence with a GC content of more than 42%, more than 48%, preferably more than 54%.

Embodiment 21. The method of any one of the preceding Embodiments, wherein an oncoselective read through motif comprises a codon that encodes proline residue.

Embodiment 22. The method of any one of the preceding Embodiments, wherein the open reading frame encodes a suicide protein, cell surface antigen, an antibody agent, a toxin, a cytokine, a genetic modification protein, or a viral replication protein.

Embodiment 23. The method of any one of the preceding Embodiments, wherein the open reading frame encodes a suicide protein.

Embodiment 24. The method of any one of the preceding Embodiments, wherein the suicide protein induces necroptosis.

Embodiment 25. The method of any one of the preceding Embodiments, wherein the suicide protein is constitutively active MLKL.

Embodiment 26. The method of any one of the preceding Embodiments, wherein the suicide protein induces pyroptosis.

Embodiment 27. The method of any one of the preceding Embodiments, wherein the suicide protein is constitutively active gasdermin D.

Embodiment 28. The method of any one of the preceding Embodiments, wherein the engineered nucleic acid has reduced immunogenicity.

Embodiment 29. The method of any one of the preceding Embodiments, wherein the subject is not receiving aminoglycoside antibiotics, macrolide antibiotics, ataluren, or ivacaftor, ivacaftor/lumacaftor.

Embodiment 30. The method of any one of the preceding Embodiments, wherein the subject is not suffering from Diamond-Blackfan anemia, Dyskeratosis congenita, Shwachman-Diamond syndrome, 5q-myelodysplastic syndrome, Treacher Collins syndrome, Cartilage-hair hypoplasia, Isolated congenital asplenia, Bowen-Conradi syndrome, or North American Indian childhood cirrhosis.

Embodiment 31. The method of any one of the preceding Embodiments, wherein the step of administering comprises administering a plurality of doses.

Embodiment 32. A pharmaceutical composition comprising an engineered nucleic acid of any one of the preceding Embodiments and an immune checkpoint inhibitor.

Embodiment 33. A method of selecting an individual for treatment with the pharmaceutical composition of Embodiment 32, the method comprising identifying an individual that is refractory or resistant to treatment with an immune checkpoint inhibitor.

### Definitions

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumoral, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: As used herein, the term “amino acid” refers to any entity that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsT™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva. In some embodiments, a cancer may be or comprise one or more solid tumors. In some embodiments, a cancer may be or comprise one or more haematologic tumors.

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, etc. that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein in the context of polypeptides, nucleic acids, and chemical compounds, the term “corresponding to”, designates the position/identity of a structural element, e.g., of an amino acid residue, a nucleotide residue, or a chemical moiety, in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at position 190 in the reference polypeptide; those of ordinary skill in the art readily appreciate how to identify “corresponding” amino acids (see. e.g., Benson et al. Nucl. Acids Res. (1 Jan. 2013) 41 (D1): D36-D42; Pearson et al. PNAS Vol. 85, pp. 2444-2448, April 1988). Those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Expression: As used herein, the term “expression” of a nucleic acid sequence refers to the generation of a gene product from the nucleic acid sequence. In some embodiments, a gene product can be a transcript (e.g., a primary transcript or a processed transcript such as an mRNA). In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation): (3) translation of an RNA into a polypeptide or protein; and/or (4) post-translational modification of a poly peptide or protein.

Flanking Sequence: As used herein the term “flanking sequence” refers to any sequence that precedes or succeeds a sequence or domain of interest. For example, a region upstream of a stop codon can be referred to as “upstream flanking region”.

Gene: As used herein, the term “gene” refers to a DNA or RNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

Mutant: As used herein, the term “mutant” refers to an organism, a cell, or a biomolecule (e.g., a nucleic acid or a polypeptide) that has a genetic variation as compared to a reference organism, cell, or biomolecule. For example, a mutant nucleic acid or polypeptide may, in some embodiments, have, for example, a substitution of one or more residues (e.g., of one or more nucleobases or amino acids), a deletion of one or more residues (e.g., an internal deletion or a truncation), an insertion of one or more residues, an inversion of two or more residues, etc, as compared to a reference nucleic acid molecule. Those skilled in the art will be familiar with various particular types of such nucleic acid or polypeptide mutants—e.g., fusions, indels, etc. An organism or cell comprising or expressing a mutant nucleic acid or polypeptide is also sometimes referred to herein as a “mutant.” In some embodiments, a mutant comprises a genetic variant that is associated with a loss of function of a gene product. A loss of function may be a complete abolishment of function, e.g., an abolishment of activity (e.g., of binding activity, enzymatic activity, etc), or a partial loss of function, e.g., a diminished activity (e.g., binding activity, enzymatic activity, etc). In some embodiments, a mutant comprises a genetic variant that is associated with a gain of function, e.g., with enhancement of an existing activity, or gain of a new activity relative to an appropriate reference (e.g., the same entity absent the genetic variation). In some embodiments, a gain of function mutant may have gained an alteration in a characteristic or activity. In some embodiments, a gain of function mutant may have constitutive activity. In some embodiments, a loss of function mutant may have lost (or reduced relative to a reference) a desirable activity. In some embodiments, the reference organism, cell, or biomolecule relative to which a mutant's structure, level, and/or activity is compared, is a wild-type organism, cell, or biomolecule.

Nucleic acid: As used herein, the term “nucleic acid” refers to a polymer of at least three nucleotides. In some embodiments, a nucleic acid is or comprises DNA. In some embodiments, a nucleic acid is or comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Peptide: As used herein, the term “peptide” refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and/or substantially pyrogen-free.

Polypeptide: As used herein, the term “polypeptide”, refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the poly peptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 9%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein, the term “reference” refers to a standard or control relative to which a comparison is performed. For example, in some embodiments, an agent, animal, individual, population, sample, sequence, or value of interest is compared to a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” refers to a biological sample obtained or derived from a source of interest, as described herein. In some embodiments, a source of interest is or comprises an organism, such as a microbe, a plant, an animal or a human. In some embodiments, a biological sample is or comprises biological tissue or fluid, or one or more components thereof. In some embodiments, a biological sample may be or comprise bone marrow; blood; blood cells; ascites; tissue or biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a biological sample comprises cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Therapeutic agent: As used herein, the term “therapeutic agent” generally refers to an agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, therapeutic agents may be CREBBP antagonists as described herein.

Therapeutically effective amount: As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Upstream and downstream: As used herein when describing RNA the term “upstream” refers to toward or close to the 5′ end of the RNA molecule and the term “downstream” refers to toward or close to the 3′ end” of the RNA molecule. As used herein when describing DNA, “upstream” is toward the 5′ end of the coding strand and “downstream” is toward the 3′ end of the coding strand. Because of the anti-parallel orientation of DNA, this means the 3′ end of the template strand is upstream and the 5′ end is downstream.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70/6, or at least about 80%, or at least about 90% or up to and including a 100% increase or any increase between 10-100% as compared to a reference level, standard, or control. Other examples of“increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Examples

The following illustrative examples are representative of embodiments of the stimulation, systems, and methods described herein and are not meant to be limiting in any way.

### Example 1. Oncoselective mRNA Shrinks Immune Checkpoint Inhibitor Resistant Tumor

The present example demonstrates that oncoselective nucleic acids of the present disclosure produce selective killing of tumor cells. Moreover treatment with one or more oncoselective nucleic acids of the present disclosure can re-sensitize immune checkpoint inhibitor resistant tumors to immune checkpoint inhibitor therapy.

This example demonstrates the efficacy of an exemplary oncoselective mRNA. KR-333 is an mRNA cocktail encoding immunogenic cell death (ICD) protein, constitutively active Gasdermin, (ca-Gasdermin) and cytokines (interferon alpha (IFN-α) that allows simultaneous induction of multiple immune pathways. By triggering immunogenic cell death of tumor cells, KR-333 causes release of immunostimulatory signals that inflame the tumor microenvironment and activate antigen presenting cells. In the tumor microenvironment, the presence of cytokines encoded by KR-333 mRNAs allows for maximal immune cells infiltration and activation, resulting in robust anti-tumor response.

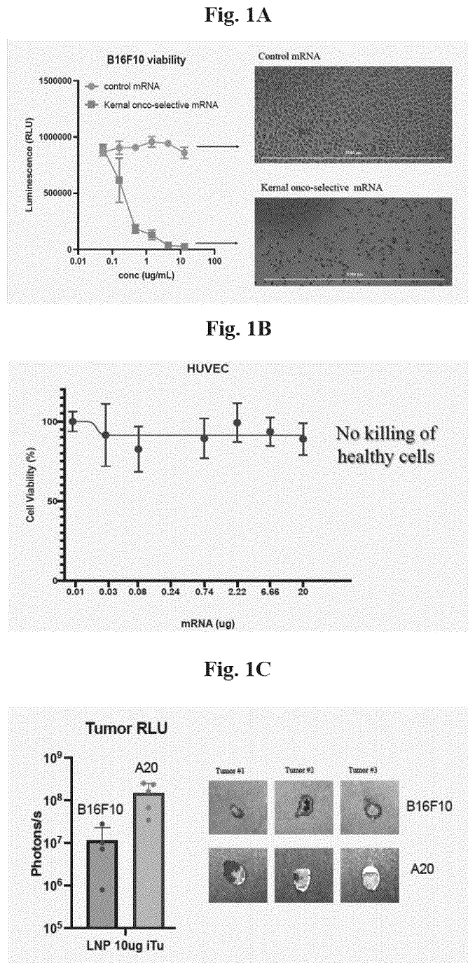

B16F10 tumor cells were incubated for 48 hours with serially diluted control mRNA or onco-selective mRNA of the present disclosure formulated with LNP Cell viability was assessed by quantitation of ATP present as indicator of metabolically active cells (viable cells) using the Cell-Titer glow luminescent cell viability assay. Results are representative of at least three independent experiments. As seen in FIG. 1A, oncoselective nucleic acids of the present disclosure selective killed B16F10 cells in culture. The oncoselective nucleic acids, however; did not kill healthy cells (FIG. 1B). FIG. 1C further demonstrates the oncoselective expression of engineered nucleic acids of the present disclosure. LNP formulated Luc mRNA was administered intratumorally in B16F10 and A20 subcutaneous tumors. FIG. 1C shows total flux (photons/s) in tumors imaged 18 hours after local administration.

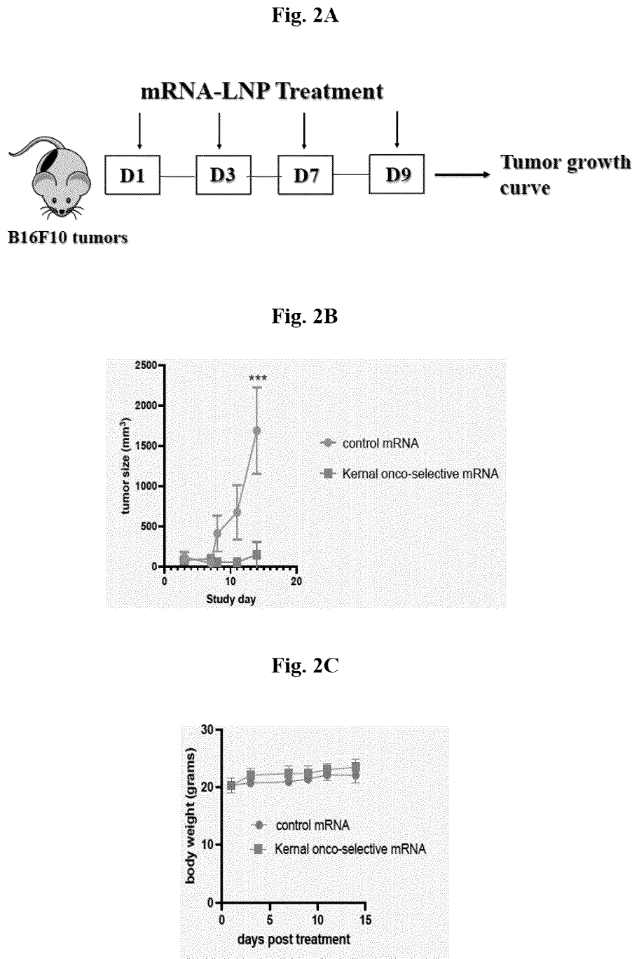

Oncoselective mRNAs of the present disclosure selectively kill tumor cells in animal models. Female B16 albino mice were inoculated subcutaneously with B16F10 tumor cells. When tumors reached approximately 50-60 mm3, mice received intra-tumoral injections of either control mRNA or KR-333 onco-selective mRNA formulated with LNP on days 1, 3, 7, 9, and intraperitoneal injections of anti-PD1 (200 μg/mouse) on days 1, 3, 9, and then bi-weekly (FIG. 2A). Tumor volumes and body weights were measured three times a week (FIG. 2B and FIG. 2C). Data illustrated in FIG. 2B shows tumor growth over the course of 14 days from treatment start. Data illustrated in FIG. 2C shows mouse body weight over the course of the treatment. ***P=0.0001, two-tailed unpaired Student's test comparing mRNA control vs onco-selective mRNA on day 14 of the study.

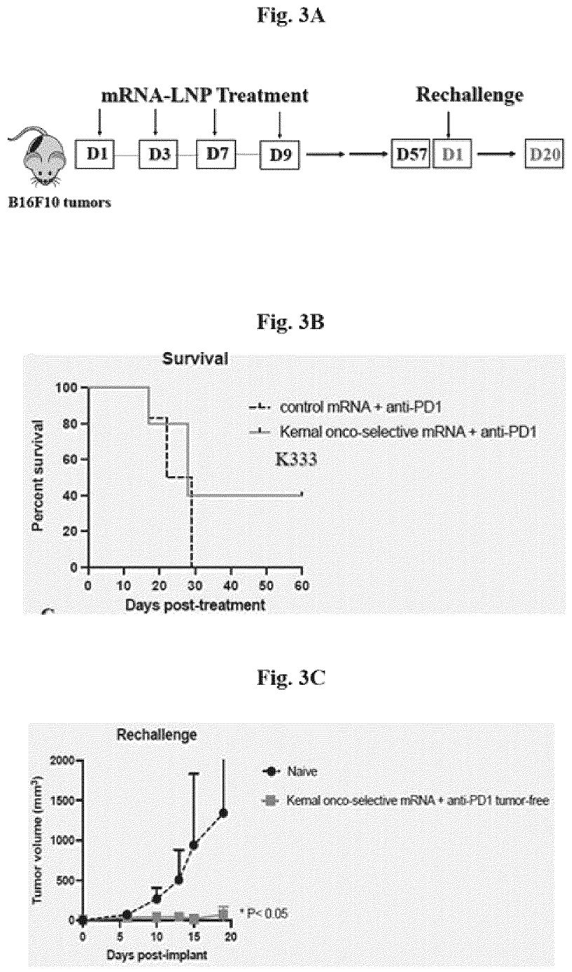

To further demonstrate the power of the engineered nucleic acids of the present disclosure, oncoselective mRNAs were administered in combination with immune checkpoint inhibitors. Female B16 albino mice were treated as described in FIG. 3A and survival curve is shown in FIG. 3B. Mice that remained tumor free by study day 57 were rechallenged by subcutaneous injection of B16F10 cells on the contralateral flank alongside naïve age-matched controls (FIG. 3C). Data represents means+/−SEM. In combination with anti-PD1 immunotherapy, KR-333 results in tumor regression, improved overall survival and complete responses (CRs) in tumor models refractory to anti-PD1/PD-L1 blockade. The presently disclosed onco-selective mRNA platform allows for activation of immune pathways that complements the activity of immune checkpoint blockers providing the opportunity to treat a variety of cancers with poor T cells infiltration and immunosuppressive tumor microenvironment, major obstacles in cancer immunotherapy.

### Example 2. Oncoselective mRNA in Human Cells

To further demonstrate the efficacy of oncoselective mRNAs of the present disclosure, particularly in combination with immune checkpoint inhibitors, experiments are performed on human tumors. Leukemia cell lines, solid tumor cells or cell lines, or PBMCs are incubated with LNPs comprising control mRNAs or one or more oncoselective mRNAs (e.g., an oncoselective mRNA encoding a constitutively active Gasdermin and/or an oncoselective mRNA encoding IFNα.)

### Example 3. Exemplary Oncoselective mRNA Constructs and Payloads

This example provides sequences of oncoselective nucleic acids used in the present examples.

Example 4. Oncoselective composition for treating tumor

| | |
|---|---|
| ca- | AGGUCUUCUGGUCCCCACAGACUCAGAGAGAACCCGCCACCaugGGCAGCGCCUUCGAGCG |
| human- | CGUCGUCCGCCGCGUCGUCCAGGAGCUCGACCACGGCGGCGAGUUCAUCCCCGUCACCAG |
| Gasdermin | CCUCCAGAGCAGCACCGGCUUCCAGCCCUACUGCCUCGUCGUCCGCAAGCCCAGCAGCAGC |
| (SEQ | UGGUUCUGGAAGCCCCGCUACAAGUGCGUGAACCUGAGCAUCAAGGACAUCCUCGAGCCG |
| ID NO. | GACGCCGCCGAGCCCGACGUCCAGCGCGGCCGCAGCUUCCACUUCUACGACGCCAUGGACG |
| 24) | GCCAGAUCCAGGGCAGCGUCGAGCUGGCCGCCCCCGGCCAGGCCAAGAUCGCCGGCGGCG |
| | CCGCCGUCAGCGACAGCAGCAGCACCAGCAUGAACGUCUACAGCCUCAGCGUCGACCCCAA |
| | CACCUGGCAGACCCUCCUCCACGAGCGCCACCUCCGCCAGCCCGAGCACAAGGUCCUCCAG |
| | CAGCUCCGCAGCCGGGGCGACAACGUCUACGUCGUCACCGAGGUCCUCCAGACCCAGAAG |
| | GAGGUCGAGGUCACCCGGACCCACAAGCGCGAGGGCAGCGGCCGCUUCAGCCUCCCCGGC |
| | GCCACCUGCCUCCAGGGCGAGGGCCAGGGCCACCUCAGCCAGAAGAAGACCGUCACCAUCC |
| | CCAGCGGCAGCACCCUCGCCUUCCGCGUCGCCCAGCUCGUCAUCGACAGCGACCUCGACGU |
| | CCUCCUCUUCCCCGACAAGAAGCAGCGCACCUUCCAGCCCCCCGCCACCGGCCACAAGCGCA |
| | GCACCAGCGAGGGCGCCUGGCCCCAGCUCCCCAGCGGCCUCAGCAUGAUGCGCUGCCUCCA |
| | CAACUUCCUCACCGACUGAUUAAACCGGUCUGCAGCUUUUGCCUUGGUUCUUCCUAGUG |
| | CCUACAAUGGGAAAACUUCACUCCAAAGAGAAACCUAUUAAGUCAUCAUCUCCAAACUAA |
| | ACCCUCACAAGUAACAGUUGAAGAAAAAAUGGCAAGAGAUCAUAUCCUCAGACCAGGUGG |
| | AAUUACUUAAAUUUUAAAGCCUGAAAAUUCCAAUUUGGGGGUGGGAGGUGGAAAAGGA |
| | ACCCAAUUUUCUUAUGAACAGAUAUUUUUAACUUAAUGGCACAAAGUCUUAGAAUAUUA |
| | UUAUGUGCCCCGUGUUCCCUGUUCUUCGUUGCUGAAUUCGAUAUCCAGCGGCCGCGCUA |
| | GCGCUG |
| human- | AGGUCUUCUGGUCCCCACAGACUCAGAGAGAACCCGCCACCaugGCCCUGACCUUCGCCCU |
| IFN-A | GUUAGUGGCGCUGCUGGUGCUGAGCUGCAAGAGCAGCUGCAGCGUGGGCUGCGACCUG |
| (SEQ | CCUCAGACCCACAGCCUGGGCAGCCGGCGGACCCUGAUGCUGCUGGCCCAGAUGCGGAAG |
| ID NO. | AUCAGCCUGUUCAGCUGUCUGAAGGACCGGCACGACUUCGGCUUCCCCCAGGAGGAAUU |
| 25) | CGGCAACCAGUUCCAGAAGGCCGAGACCAUCCCCGUGCUGCACGAGAUGAUCCAGCAGAU |
| | CUUCAACCUGUUCAGCACCAAGGAUAGCAGCGCCGCUUGGGACGAGACCCUUCUGGACAA |
| | GUUCUACACCGAGCUGUACCAGCAGCUGAACGACCUGGAGGCCUGCGUGAUCCAGGGCG |
| | UGGGCGUGACCGAGACCCCUCUGAUGAAGGAGGACAGCAUCCUGGCCGUGCGGAAGUAC |
| | UUUCAGCGGAUCACCCUGUACCUGAAGGAGAAGAAGUACAGCCCAUGCGCCUGGGAGGU |
| | GGUGCGGGCCGAGAUCAUGCGGAGCUUCAGCCUGAGCACCAACCUGCAGGAGAGCCUGC |
| | GGAGCAAGGAGUGAUUAAACCGGUCUGCAGCUUUUGCCUUGGUUCUUCCUAGUGCCUAC |
| | AAUGGGAAAACUUCACUCCAAAGAGAAACCUAUUAAGUCAUCAUCUCCAAACUAAACCCU |
| | CACAAGUAACAGUUGAAGAAAAAAUGGCAAGAGAUCAUAUCCUCAGACCAGGUGGAAUU |
| | ACUUAAAUUUUAAAGCCUGAAAAUUCCAAUUUGGGGGUGGGAGGUGGAAAAGGAACCC |
| | AAUUUUCUUAUGAACAGAUAUUUUUAACUUAAUGGCACAAAGUCUUAGAAUAUUAUUA |
| | UGUGCCCCGUGUUCCCUGUUCUUCGUUGCUGAAUUCGAUAUCCAGCGGCCGCGCUAGCG |
| | CUG |
| mouse- | AGGUCUUCUGGUCCCCACAGACUCAGAGAGAACCCGCCACCaugCCAAGCGCCUUCGAGAA |
| Gasdermin | GGUGGUGAAGAACGUGAUCAAGGAGGUCUCCGGCAGCCGGGGCGACCUGAUCCCCGUGG |
| (SEQ | ACAGCCUGCGGAACAGCACCAGCUUCCGGCCCUACUGCCUGCUGAACCGGAAGUUCAGCA |
| ID NO. | GCAGCCGGUUCUGGAAGCCCCGGUACAGCUGCGUGAACCUGAGCAUCAAGGACAUCCUG |
| 26) | GAGCCCAGCGCCCCCGAGCCCGAGCCUGAGUGUUUCGGCAGCUUCAAGGUGAGCGACGU |
| | GGUGGACGGCAACAUCCAGGGCCGGGUGAUGCUGAGCGGCAUGGGCGAGGGAAAGAUCA |
| | GCGGGGGCGCCGCCGUGAGCGACAGCAGCAGCGCCAGCAUGAACGUGUGCAUCCUGCGG |
| | GUGACCCAGAAGACCUGGGAGACCAUGCAGCACGAGCGGCACCUGCAGCAGCCUGAGAAU |
| | AAGAUCCUGCAGCAGCUGCGGAGCCGGGGCGAUGACCUGUUCGUGGUGACCGAAGUGCU |
| | GCAGACCAAGGAGGAGGUGCAGAUCACCGAGGUGCACAGCCAGGAGGGCAGCGGCCAGU |
| | UCACCCUGCCCGGCGCCCUGUGCCUGAAGGGCGAGGGCAAGGGACACCAGAGCCGGAAGA |
| | AGAUGGUGACCAUCCCCGCCGGCAGCAUCCUGGCCUUCCGGGUGGCCCAGCUGCUGAUCG |
| | GCAGCAAGUGGGACAUUCUGCUGGUGAGCGACGAGAAGCAGCGGACCUUCGAGCCCAGC |
| | AGCGGCGACCGGAAGGCCGUGGGCCAGCGGCACCACGGCCUGAACGUGCUGGCCGCCCUG |
| | UGUAGCAUCGGCAAGCAGCUAAGCCUGCUGAGCGACUGAUUAAACCGGUCUGCAGCUUU |
| | UGCCUUGGUUCUUCCUAGUGCCUACAAUGGGAAAACUUCACUCCAAAGAGAAACCUAUU |
| | AAGUCAUCAUCUCCAAACUAAACCCUCACAAGUAACAGUUGAAGAAAAAAUGGCAAGAGA |
| | UCAUAUCCUCAGACCAGGUGGAAUUACUUAAAUUUUAAAGCCUGAAAAUUCCAAUUUGG |
| | GGGUGGGAGGUGGAAAAGGAACCCAAUUUUCUUAUGAACAGAUAUUUUUAACUUAAUG |
| | GCACAAAGUCUUAGAAUAUUAUUAUGUGCCCCGUGUUCCCUGUUCUUCGUUGCUGAAU |
| | UCGAUAUCCAGCGGCCGCGCUAGCGCUG |
| mouse- | AGGUCUUCUGGUCCCCACAGACUCAGAGAGAACCCGCCACCaugGCUCGGCUGUGCGCCU |
| IFN-A | UCCUGAUGAUCCUGGUGAUGAUGAGCUACUACUGGAGCGCAUGCAGCCUGGGCUGCGAC |
| (SEQ | CUGCCACACACCUACAACCUGGGCAAUAAGCGGGCUCUGACCGUGCUGGAGGAGAUGCGG |
| ID NO. | CGGCUGCCCCCCCUGAGCUGCCUGAAGGACCGGAAGGACUUCGGCUUCCCCCUGGAGAAG |
| 27) | GUGGACAACCAGCAGAUCCAAAAGGCACAGGCCAUCCUGGUGCUGCGGGACCUGACCCAG |
| | CAGAUCCUGAACCUGUUCACCAGCAAGGACCUGAGCGCCACCUGGAACGCCACCCUGCUG |
| | GACAGCUUCUGCAACGACCUGCACCAGCAGCUGAACGACCUGAAGGCCUGCGUCAUGCAG |
| | GAGCCACCACUGACCCAGGAGGACAGCCUGUUAGCUGUGCGGACUUACUUCCACCGGAUC |
| | ACCGUGUACCUGCGGAAGAAGAAGCACAGCCUGUGCGCUUGGGAGGUGAUCCGGGCCGA |
| | GGUGUGGCGGGCCCUGAGCAGCAGCACCAACCUGCUGGCCCGGCUGAGCGAGGAGAAGG |
| | AGUGAUUAAACCGGUCUGCAGCUUUUGCCUUGGUUCUUCCUAGUGCCUACAAUGGGAAA |
| | ACUUCACUCCAAAGAGAAACCUAUUAAGUCAUCAUCUCCAAACUAAACCCUCACAAGUAAC |
| | AGUUGAAGAAAAAAUGGCAAGAGAUCAUAUCCUCAGACCAGGUGGAAUUACUUAAAUUU |
| | UAAAGCCUGAAAAUUCCAAUUUGGGGGUGGGAGGUGGAAAAGGAACCCAAUUUUCUUA |
| | UGAACAGAUAUUUUUAACUUAAUGGCACAAAGUCUUAGAAUAUUAUUAUGUGCCCCGUG |
| | UUCCCUGUUCUUCGUUGCUGAAUUCGAUAUCCAGCGGCCGCGCUAGCGCUG |
| U-27- | AGGUCUUCUGGUCCCCACAGACUCAGAGAGAACCCGCCACCaugAAGUGAGGCUCUCCUCC |
| ca- | CGCCCCGCCCCUCCCACGCCUCACCAGCCCCCCGCGCGCCCACCCUCCGGCGGGUGACAGCU |
| human- | CCGGGAUCAGCAACCCUUCCUGCUGCUGCUACUGCUGCUGCUGCUGCCGCCGCCGCCGCC |
| Gasdermin | GCCGCUGCCCUUGGGUCCCCCCGAGUCUCCGGGACUGCCCUCUCGACUGUCAGUGGGGCA |
| (SEQ | GCCUCUCCGACUCUGCACCCGCCUCGACCUCCCCACCCGCUCCCACACCCCUGUGCCCUCAU |
| ID NO. | GUGGAGCCUAAGAGAACAGAACAGGCCGUGAAGCCAGCAGAGAAAggaagcggagcuacuaacuu |
| 28) | cagccugcugaagcaggcuggagacguggaggagaacccuggaccuuacucgagcagACGCGUag |
| | aucugGGCUCCGGCGAGGGCAGGGGAAGUCUUCUAACAUGCGGGGACGUGGAGGAAAAUCCCGGC |
| | CCAGGCAGCGCCUUCGAGCGCGUCGUCCGCCGCGUCGUCCAGGAGCUCGACCACGGCGGC |
| | GAGUUCAUCCCCGUCACCAGCCUCCAGAGCAGCACCGGCUUCCAGCCCUACUGCCUCGUCG |
| | UCCGCAAGCCCAGCAGCAGCUGGUUCUGGAAGCCCCGCUACAAGUGCGUGAACCUGAGCA |
| | UCAAGGACAUCCUCGAGCCGGACGCCGCCGAGCCCGACGUCCAGCGCGGCCGCAGCUUCCA |
| | CUUCUACGACGCCAUGGACGGCCAGAUCCAGGGCAGCGUCGAGCUGGCCGCCCCCGGCCA |
| | GGCCAAGAUCGCCGGCGGCGCCGCCGUCAGCGACAGCAGCAGCACCAGCAUGAACGUCUA |
| | CAGCCUCAGCGUCGACCCCAACACCUGGCAGACCCUCCUCCACGAGCGCCACCUCCGCCAGC |
| | CCGAGCACAAGGUCCUCCAGCAGCUCCGCAGCCGGGGCGACAACGUCUACGUCGUCACCG |
| | AGGUCCUCCAGACCCAGAAGGAGGUCGAGGUCACCCGGACCCACAAGCGCGAGGGCAGCG |
| | GCCGCUUCAGCCUCCCCGGCGCCACCUGCCUCCAGGGCGAGGGCCAGGGCCACCUCAGCCA |
| | GAAGAAGACCGUCACCAUCCCCAGCGGCAGCACCCUCGCCUUCCGCGUCGCCCAGCUCGUC |
| | AUCGACAGCGACCUCGACGUCCUCCUCUUCCCCGACAAGAAGCAGCGCACCUUCCAGCCCC |
| | CCGCCACCGGCCACAAGCGCAGCACCAGCGAGGGCGCCUGGCCCCAGCUCCCCAGCGGCCU |
| | CAGCAUGAUGCGCUGCCUCCACAACUUCCUCACCGACUGAUUAAACCGGUCUGCAGCUUU |
| | UGCCUUGGUUCUUCCUAGUGCCUACAAUGGGAAAACUUCACUCCAAAGAGAAACCUAUU |
| | AAGUCAUCAUCUCCAAACUAAACCCUCACAAGUAACAGUUGAAGAAAAAAUGGCAAGAGA |
| | UCAUAUCCUCAGACCAGGUGGAAUUACUUAAAUUUUAAAGCCUGAAAAUUCCAAUUUGG |
| | GGGUGGGAGGUGGAAAAGGAACCCAAUUUUCUUAUGAACAGAUAUUUUUAACUUAAUG |
| | GCACAAAGUCUUAGAAUAUUAUUAUGUGCCCCGUGUUCCCUGUUCUUCGUUGCUGAAU |
| | UCGAUAUCCAGCGGCCGCGCUAGCGCUG |
| U-27- | AGGUCUUCUGGUCCCCACAGACUCAGAGAGAACCCGCCACCaugAAGUGAGGCUCUCCUCC |
| hlFN-A | CGCCCCGCCCCUCCCACGCCUCACCAGCCCCCCGCGCGCCCACCCUCCGGCGGGUGACAGCU |
| (SEQ | CCGGGAUCAGCAACCCUUCCUGCUGCUGCUACUGCUGCUGCUGCUGCCGCCGCCGCCGCC |
| ID NO. | GCCGCUGCCCUUGGGUCCCCCCGAGUCUCCGGGACUGCCCUCUCGACUGUCAGUGGGGCA |
| 29) | GCCUCUCCGACUCUGCACCCGCCUCGACCUCCCCACCCGCUCCCACACCCCUGUGCCCUCAU |
| | GUGGAGCCUAAGAGAACAGAACAGGCCGUGAAGCCAGCAGAGAAAggaagcggagcuacuaacuu |
| | cagccugcugaagcaggcuggagacguggaggagaacccuggaccuuacucgagcagACGCGUa |
| | gaucugGGCUCCGGCGAGGGCAGGGGAAGUCUUCUAACAUGCGGGGACGUGGAGGAAAAUCCCG |
| | GCCCAGCCCUGACCUUCGCCCUGUUAGUGGCGCUGCUGGUGCUGAGCUGCAAGAGCAGCUG |
| | CAGCGUGGGCUGCGACCUGCCUCAGACCCACAGCCUGGGCAGCCGGCGGACCCUGAUGCU |
| | GCUGGCCCAGAUGCGGAAGAUCAGCCUGUUCAGCUGUCUGAAGGACCGGCACGACUUCG |
| | GCUUCCCCCAGGAGGAAUUCGGCAACCAGUUCCAGAAGGCCGAGACCAUCCCCGUGCUGC |
| | ACGAGAUGAUCCAGCAGAUCUUCAACCUGUUCAGCACCAAGGAUAGCAGCGCCGCUUGGG |
| | ACGAGACCCUUCUGGACAAGUUCUACACCGAGCUGUACCAGCAGCUGAACGACCUGGAGG |
| | CCUGCGUGAUCCAGGGCGUGGGCGUGACCGAGACCCCUCUGAUGAAGGAGGACAGCAUC |
| | CUGGCCGUGCGGAAGUACUUUCAGCGGAUCACCCUGUACCUGAAGGAGAAGAAGUACAG |
| | CCCAUGCGCCUGGGAGGUGGUGCGGGCCGAGAUCAUGCGGAGCUUCAGCCUGAGCACCA |
| | ACCUGCAGGAGAGCCUGCGGAGCAAGGAGUGAUUAAACCGGUCUGCAGCUUUUGCCUUG |
| | GUUCUUCCUAGUGCCUACAAUGGGAAAACUUCACUCCAAAGAGAAACCUAUUAAGUCAUC |
| | AUCUCCAAACUAAACCCUCACAAGUAACAGUUGAAGAAAAAAUGGCAAGAGAUCAUAUCC |
| | UCAGACCAGGUGGAAUUACUUAAAUUUUAAAGCCUGAAAAUUCCAAUUUGGGGGUGGG |
| | AGGUGGAAAAGGAACCCAAUUUUCUUAUGAACAGAUAUUUUUAACUUAAUGGCACAAAG |
| | UCUUAGAAUAUUAUUAUGUGCCCCGUGUUCCCUGUUCUUCGUUGCUGAAUUCGAUAUC |
| | CAGCGGCCGCGCUAGCGCUG |
| U-27- | AGGUCUUCUGGUCCCCACAGACUCAGAGAGAACCCGCCACCaugAAGUGAGGCUCUCCUCC |
| Mouse- | CGCCCCGCCCCUCCCACGCCUCACCAGCCCCCCGCGCGCCCACCCUCCGGCGGGUGACAGCU |
| Gasdermin | CCGGGAUCAGCAACCCUUCCUGCUGCUGCUACUGCUGCUGCUGCUGCCGCCGCCGCCGCC |
| (SEQ | GCCGCUGCCCUUGGGUCCCCCCGAGUCUCCGGGACUGCCCUCUCGACUGUCAGUGGGGCA |
| ID NO. | GCCUCUCCGACUCUGCACCCGCCUCGACCUCCCCACCCGCUCCCACACCCCUGUGCCCUCAU |
| 30) | GUGGAGCCUAAGAGAACAGAACAGGCCGUGAAGCCAGCAGAGAAÅggaagcggagcuacuaacuu |
| | cagccugcugaagcaggcuggagacguggaggagaacccuggaccuuacucgagcagACGCGUa |
| | gaucugGGCUCCGGCGAGGGCAGGGGAAGUCUUCUAACAUGCGGGGACGUGGAGGAAAAUCCCG |
| | CCACCAAGCGCCUUCGAGAAGGUGGUGAAGAACGUGAUCAAGGAGGUCUCCGGCAGCCGGC |
| | GGGCGACCUGAUCCCCGUGGACAGCCUGCGGAACAGCACCAGCUUCCGGCCCUACUGCCU |
| | GCUGAACCGGAAGUUCAGCAGCAGCCGGUUCUGGAAGCCCCGGUACAGCUGCGUGAACC |
| | UGAGCAUCAAGGACAUCCUGGAGCCCAGCGCCCCCGAGCCCGAGCCUGAGUGUUUCGGCA |
| | GCUUCAAGGUGAGCGACGUGGUGGACGGCAACAUCCAGGGCCGGGUGAUGCUGAGCGGC |
| | AUGGGCGAGGGAAAGAUCAGCGGGGGCGCCGCCGUGAGCGACAGCAGCAGCGCCAGCAU |
| | GAACGUGUGCAUCCUGCGGGUGACCCAGAAGACCUGGGAGACCAUGCAGCACGAGCGGC |
| | ACCUGCAGCAGCCUGAGAAUAAGAUCCUGCAGCAGCUGCGGAGCCGGGGCGAUGACCUG |
| | UUCGUGGUGACCGAAGUGCUGCAGACCAAGGAGGAGGUGCAGAUCACCGAGGUGCACAG |
| | CCAGGAGGGCAGCGGCCAGUUCACCCUGCCCGGCGCCCUGUGCCUGAAGGGCGAGGGCAA |
| | GGGACACCAGAGCCGGAAGAAGAUGGUGACCAUCCCCGCCGGCAGCAUCCUGGCCUUCCG |
| | GGUGGCCCAGCUGCUGAUCGGCAGCAAGUGGGACAUUCUGCUGGUGAGCGACGAGAAGC |
| | AGCGGACCUUCGAGCCCAGCAGCGGCGACCGGAAGGCCGUGGGCCAGCGGCACCACGGCC |
| | UGAACGUGCUGGCCGCCCUGUGUAGCAUCGGCAAGCAGCUAAGCCUGCUGAGCGACUGA |
| | UUAAACCGGUCUGCAGCUUUUGCCUUGGUUCUUCCUAGUGCCUACAAUGGGAAAACUUC |
| | ACUCCAAAGAGAAACCUAUUAAGUCAUCAUCUCCAAACUAAACCCUCACAAGUAACAGUU |
| | GAAGAAAAAAUGGCAAGAGAUCAUAUCCUCAGACCAGGUGGAAUUACUUAAAUUUUAAA |
| | GCCUGAAAAUUCCAAUUUGGGGGUGGGAGGUGGAAAAGGAACCCAAUUUUCUUAUGAA |
| | CAGAUAUUUUUAACUUAAUGGCACAAAGUCUUAGAAUAUUAUUAUGUGCCCCGUGUUCC |
| | CUGUUCUUCGUUGCUGAAUUCGAUAUCCAGCGGCCGCGCUAGCGCUG |
| U27- | AGGUCUUCUGGUCCCCACAGACUCAGAGAGAACCCGCCACCaugAAGUGAGGCUCUCCUCC |
| mouse- | CGCCCCGCCCCUCCCACGCCUCACCAGCCCCCCGCGCGCCCACCCUCCGGCGGGUGACAGCU |
| IFN-A | CCGGGAUCAGCAACCCUUCCUGCUGCUGCUACUGCUGCUGCUGCUGCCGCCGCCGCCGCC |
| (SEQ | GCCGCUGCCCUUGGGUCCCCCCGAGUCUCCGGGACUGCCCUCUCGACUGUCAGUGGGGCA |
| ID NO. | GCCUCUCCGACUCUGCACCCGCCUCGACCUCCCCACCCGCUCCCACACCCCUGUGCCCUCAU |
| 31) | GUGGAGCCUAAGAGAACAGAACAGGCCGUGAAGCCAGCAGAGAAAggaagcggagcuacuaac |
| | uucagccugcugaagcaggcuggagacguggaggagaacccuggaccuuacucgagcagACGCG |
| | UagaucugGGCUCCGGCGAGGGCAGGGGAAGUCUUCUAACAUGCGGGGACGUGGAGGAAAAUCC |
| | CCAGCUCGGCUGUGCGCCUUCCUGAUGAUCCUGGUGAUGAUGAGCUACUACUGGAGCGCCGGC |
| | AUGCAGCCUGGGCUGCGACCUGCCACACACCUACAACCUGGGCAAUAAGCGGGCUCUGAC |
| | CGUGCUGGAGGAGAUGCGGCGGCUGCCCCCCCUGAGCUGCCUGAAGGACCGGAAGGACU |
| | UCGGCUUCCCCCUGGAGAAGGUGGACAACCAGCAGAUCCAAAAGGCACAGGCCAUCCUGG |
| | UGCUGCGGGACCUGACCCAGCAGAUCCUGAACCUGUUCACCAGCAAGGACCUGAGCGCCA |
| | CCUGGAACGCCACCCUGCUGGACAGCUUCUGCAACGACCUGCACCAGCAGCUGAACGACC |
| | UGAAGGCCUGCGUCAUGCAGGAGCCACCACUGACCCAGGAGGACAGCCUGUUAGCUGUG |
| | CGGACUUACUUCCACCGGAUCACCGUGUACCUGCGGAAGAAGAAGCACAGCCUGUGCGCU |
| | UGGGAGGUGAUCCGGGCCGAGGUGUGGCGGGCCCUGAGCAGCAGCACCAACCUGCUGGC |
| | CCGGCUGAGCGAGGAGAAGGAGUGAUUAAACCGGUCUGCAGCUUUUGCCUUGGUUCUU |
| | CCUAGUGCCUACAAUGGGAAAACUUCACUCCAAAGAGAAACCUAUUAAGUCAUCAUCUCC |
| | AAACUAAACCCUCACAAGUAACAGUUGAAGAAAAAAUGGCAAGAGAUCAUAUCCUCAGAC |
| | CAGGUGGAAUUACUUAAAUUUUAAAGCCUGAAAAUUCCAAUUUGGGGGUGGGAGGUGG |
| | AAAAGGAACCCAAUUUUCUUAUGAACAGAUAUUUUUAACUUAAUGGCACAAAGUCUUAG |
| | AAUAUUAUUAUGUGCCCCGUGUUCCCUGUUCUUCGUUGCUGAAUUCGAUAUCCAGCGGC |
| | CGCGCUAGCGCUG |

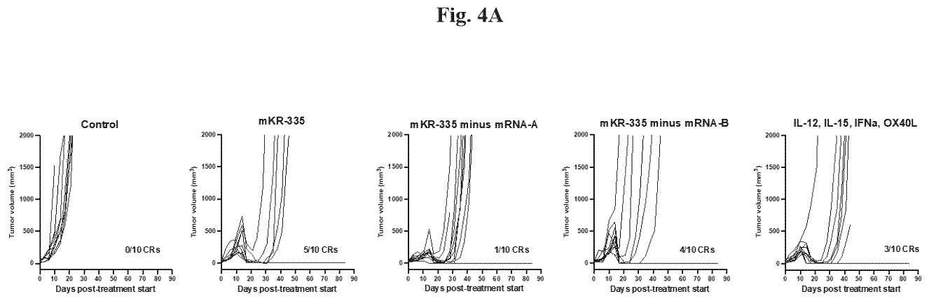

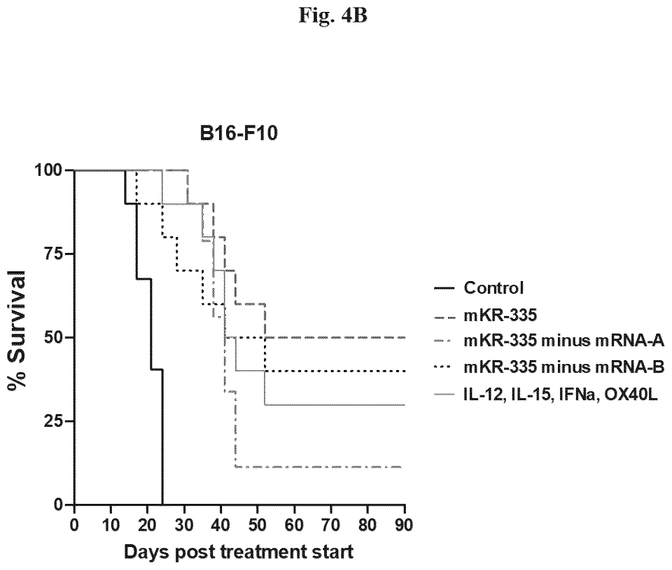

Example 4 illustrates treatment of melanoma tumor with a composition comprising oncoselective mRNA encoding IL-2, IL-12, IL-15, and IFNα compared to other mRNA treatment combinations. Female C57BL/6NCrl mice (8-10 weeks of age) were implanted subcutaneously with 2.5×10⁵ B16-F10 cells into the right flank and randomized into treatment groups (n=10) on day 8 post implant based on tumor size. Mice received six intratumoral injections of either control mRNA or oncoselective combination mRNA treatment formulated in LNPs (once every 3 days for 6 cycles, Q3Dx6). The proportion of complete responders (CRs) is indicated. FIG. 4A illustrates tumor volume increase measured across days post-treatment. The group receiving oncoselective mRNA encoding IL-2, IL-12, IL-15, and IFNα (mKR-335) showed that the increase of tumor volume was delayed and included the most number of complete responders (five out of ten) compared to other groups: control group receiving mRNA encoding non-translated mCherry; mKR-335 minus mRNA-A group receiving mRNA encoding IL-12, IL-15, and IFNα; mKR-335 minus mRNA-B group receiving mRNA encoding IL-2, IL-12, and IFNα; and group receiving mRNA encoding IL-2, IL-12, IL-15, and IFNα. FIG. 4B illustrates survival after treatment with mKR-335, a control mRNA (p<0.0001; Cox regression) and other mRNA combination treatments lacking an individual mRNA from mKR-335, cut-off 2000 mm³.

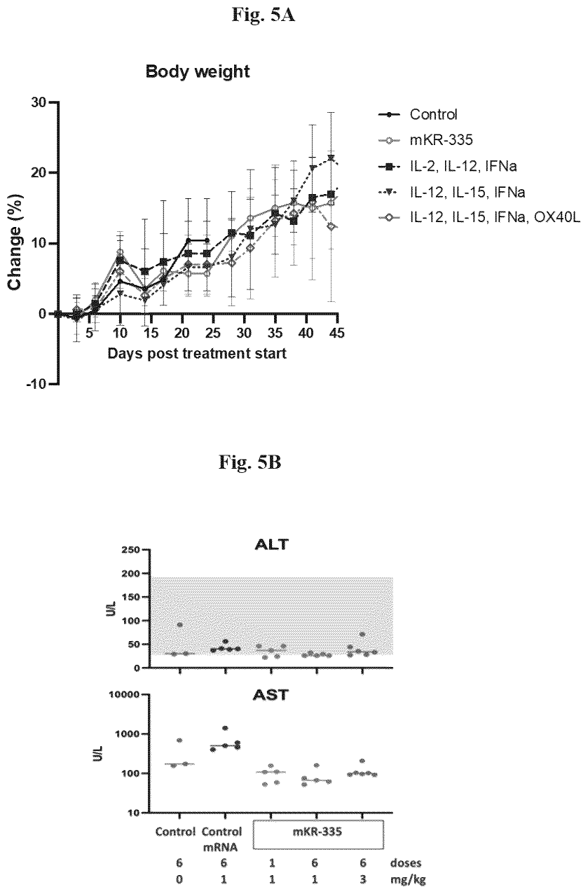

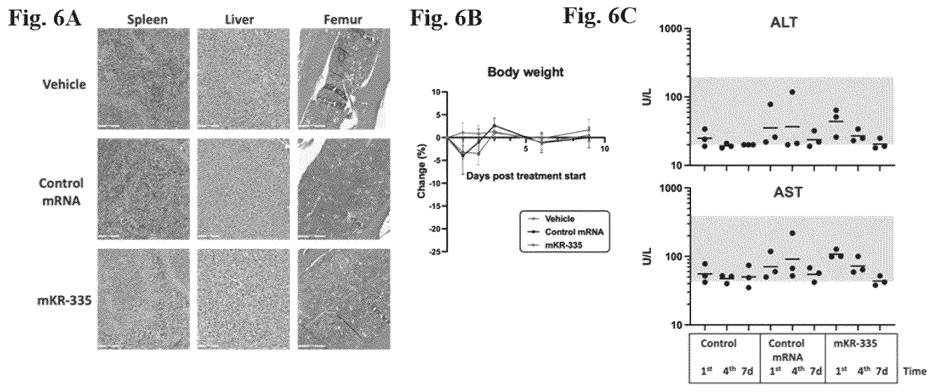

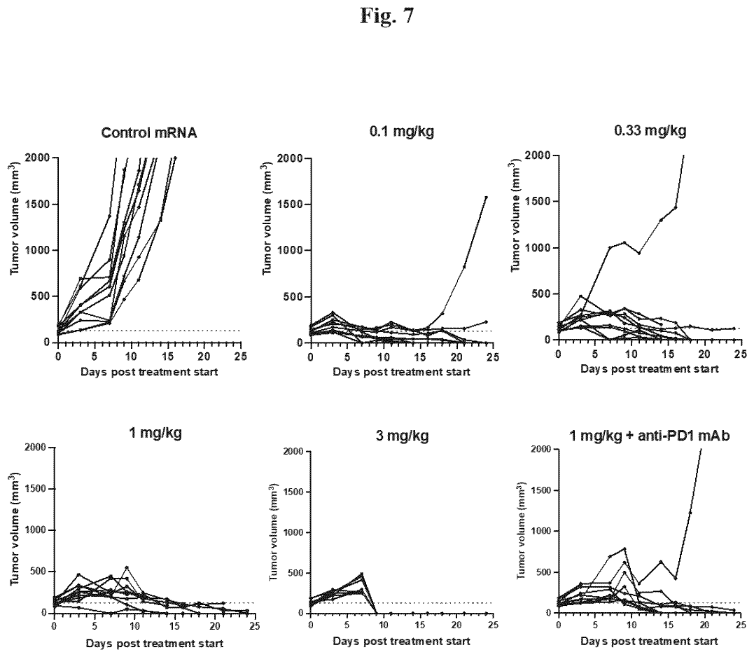

FIG. 5A-5B illustrate tolerability of intratumoral mKR-335 mRNA therapy. FIG. 5A illustrates percentage body weight changes after once every 3 days for 6 cycles (Q3Dx6) intratumoral B16-F10 dosing with a total of 20 mg (˜1 mg/kg) control mRNA or mKR-335 LNPs (mean+/−SD). FIG. 5B illustrates liver function test: normal aspartate transaminase (AST) and alanine transaminase (ALT) serum levels 24 hours after a single or six doses with mKR-335. There was no statistic significant difference across the groups in both the body weights and the liver function tests. FIG. 6 illustrates tolerability of systemic LNP administration. Female C57BL/6NCrl mice were treated subcutaneously with 1 mg/kg control mRNA LNPs or mKR-335. FIG. 6A illustrates hematoxylin and eosin (H&E) sections of 24 hours post single dose. FIG. 6B illustrates percentage body weight changes during an once every 3 days for 4 cycles (Q3Dx4) dosing regimen (mean+/−SD). FIG. 6C illustrates liver function test: normal AST and ALT serum levels 24 hours after a single or four doses and 7 days after last dose with mKR-335. There was no statistic significant difference across the groups in H&E staining, body weight, and liver function test. FIG. 7 illustrates efficacy and dose relationship of mKR-335. Dose titration treatment of subcutaneous B16-F10 melanoma tumors with mKR-335 LNPs, control mRNA LNP or mKR-335 in combination with anti-PD1 mAb treatment: 200 mg administered iv on day 0 (d0), day 3 (d3), day 10 (d10). Intratumoral injections started on day 0 in established tumors at an average size of 130 mm³. mKR-335 treatments were administered on days 0, 2, 4, 7, 9 and 11 (n=10/group).

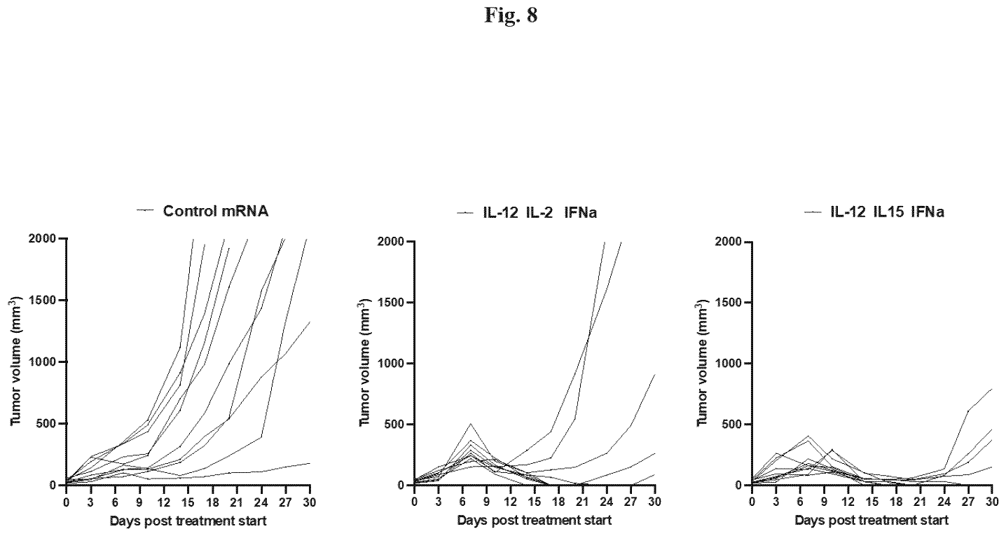

Female C57BL/6NCrl mice (8-10 weeks of age) were implanted subcutaneously with 2.5×10⁶ MC38-GFP cells into the right flank and randomized into treatment groups (n=10) on day 8 post implant based on tumor size. Mice received four intratumoral injections of either control mRNA (non-translated mCherry) or combination mRNA treatment formulated in LNPs with a regiment of once every 3 days for 4 cycles (Q3Dx4) at 1 mg/kg. Tumor growth in treatment groups is significantly inhibited in both treatment groups. (P<0.001 2-way ANOVA). The proportion of complete responders (CRs) is indicated. FIG. 8 illustrates efficacious treatment of subcutaneous syngeneic murine MC38-GFP colon adenocarcinoma tumors with cytokine mRNA LNP combinations (IL-2, IL-12, IL-15, and IFNα).

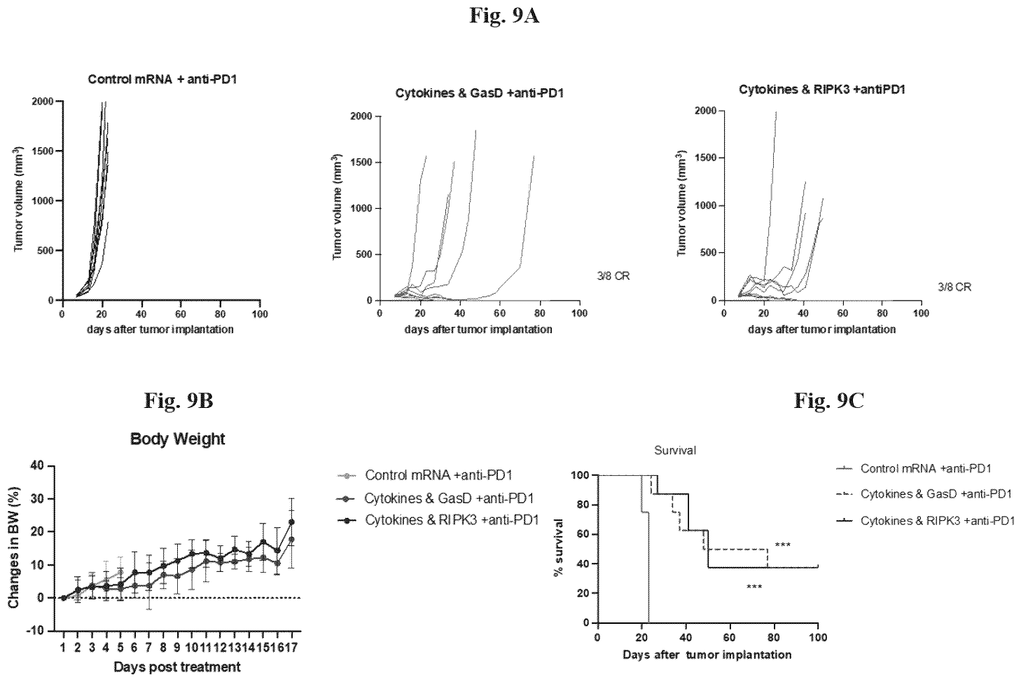

To examine the efficacy of subcutaneous treatment, female C57BL/6NCrl mice (8-10 weeks of age) were implanted subcutaneously with 2.5×10⁶ B16-F10 cells into the right flank and randomized into treatment groups (n=10) on day 8 post implant based on tumor size. Mice received intertumoral injections of either control mRNA LNP (non-translated mCherry) at 1 mg/kg dose or cytokine mRNA LNP (on d0, d6, or d10) at 1 mg/kg dose and immunogenic cell death (either GasD or RIPK3) mRNA (on d3, d9, or d12) at 0.75 mg/kg dose. All groups also received intraperitoneal injections of anti-PD1 mAb (200 mg administered on d0, d3, or d10; Invivogen #mpd1-mab15). The proportions of complete responders (CRs) are indicated (FIG. 9) FIG. 9 illustrates efficacious treatment of subcutaneous syngeneic murine B16-F10 melanoma tumors with cytokine (IL-12, IL-15, IFN-α) and immunogenic cell death (Gasdermin D or RIPK3) mRNA LNPs in combination with anti-PD1. FIG. 9A. Tumor volumes of individual animals after tumor implantation. FIG. 9B. Body weight changes of animals following initiation of treatment. Values depict mean+/−S.D. FIG. 9C. Kaplan Meier survival curves of animals after tumor implantation. Statistical significance values (***) between GasD/RIPK3 mRNA treated groups vs. the control group were obtained log-rank (Mantel-Cox) tests. n=8/group.

To examine pharmacodynamic analysis of mRNAs encoding cytokines (IL-12, IL-15, IFN-alpha), female C57BL/6NCrl mice (8-10 weeks of age) were implanted subcutaneously with 2.5×10⁵ B16-F10 cells into the right flank and randomized into treatment groups (n=10) when tumors reached ˜50-70 mm³ volume. Mice received a single intratumoral injection of either control mRNA (non-translated mCherry) or cytokine (IL-12, IL-15, IFN-alpha cocktail) mRNA treatment formulated in LNPs or in saline. Sera were collected 6 hours later and cytokine measurements were carried out via ELISA assays. IFN-gamma and IP-10 were measured as surrogate PD markers for IL-12 mRNA.

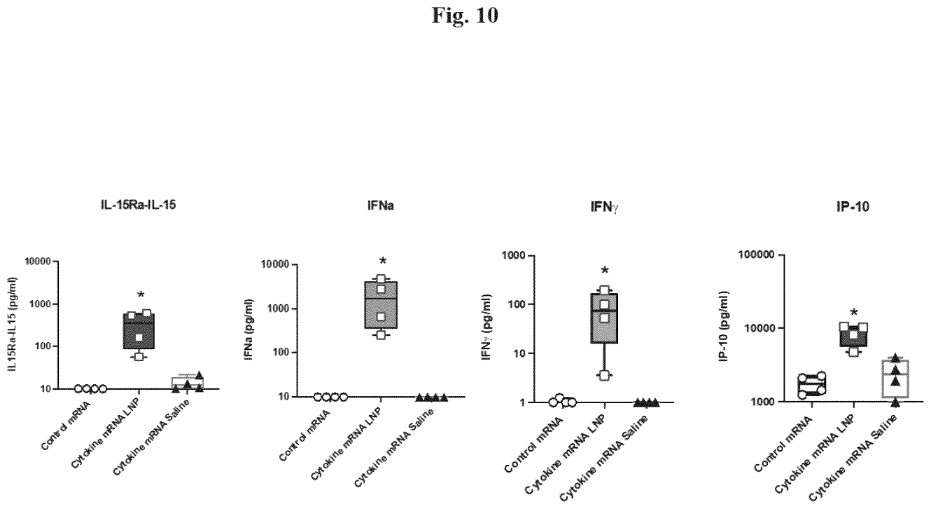

FIG. 10 illustrates pharmacodynamic analysis of mRNAs encoding cytokines (IL-12, IL-15, IFN-alpha) formulated in saline vs. LNPs in subcutaneous B16-F10 melanoma tumors. Control mRNALNP: K143-001. Cytokine mRNALNPs: K155-001, K156-001, or K157-001. Cytokine mRNA in Saline. Same mRNAs as Cytokine mRNA LNPs, without the LNP formulation.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A composition comprising: at least one engineered nucleic acid encoding at least one therapeutic, wherein the at least one engineered nucleic acid comprises at least one oncoselective modification comprising a nucleic acid sequence of any one of or a combination of any one of SEQ ID NO: 41-110.

2. The composition of claim 1, wherein the at least one therapeutic comprises at least one cytokine.

3. The composition of claim 2, wherein the at least one cytokine comprises at least one interleukin or at least one interferon.

4. (canceled)

5. (canceled)

6. (canceled)

7. (canceled)

8. The composition of claim 2, wherein the at least one cytokine comprises IL-2, IL-12, IL-15, or IFNα.

9. (canceled)

10. The composition of claim 2, wherein the at least one cytokine comprises a modified cytokine.

11. The composition of claim 10, wherein the modified cytokine comprises secreted cytokine, membrane tethered cytokine, masked cytokine, cytokine fusion, or a combination thereof.

12. The composition of claim 11, wherein the cytokine fusion comprises a cytokine coupled to an antibody or fragment thereof or to an Fc region of the antibody or fragment thereof.

13. (canceled)

14. The composition of claim 1 further comprising at least one additional active ingredient.

15. The composition of claim 14, wherein the at least one additional active ingredient comprises an immune checkpoint inhibitor comprising: one or more agents targeting CTLA-4, PD-1, PD-L1, GITR, OX40, LAG-3, KIR, TIM-3, TIGIT, BTLA, CBLB, CISH, VISTA, B7-H3, CSF-1R, GITR CD28, CD40, CD137, or a combination thereof; or one or more of pembrolizumab, nivolumab, atezolizumab, avelumab, durvalumab, ipilimumab, cemiplimab, spartalizumab, camrelizumab, cabiralizumab, dostarlimab, sintilmab, tisleliumab, or toripalimab.

16. (canceled)

17. (canceled)

18. The composition of claim 14, wherein the at least one additional active ingredient comprises an oncolytic mRNA.

19. (canceled)

20. (canceled)

21. The composition of claim 1, wherein the composition comprises the at least on engineered nucleic acid and a lipid, wherein the at least one engineered nucleic acid is in contact with the lipid.

22. The composition of claim 21, wherein the lipid comprises a lipid nanoparticle (LNP).

23. The composition of claim 1, wherein the at least one oncoselective modification comprises an oncoselective sequence motif.

24. (canceled)

25. (canceled)

26. The composition of claim 1, wherein the engineered nucleic acid comprises an open reading frame that encodes a suicide protein, cell surface antigen, an antibody agent, a toxin, a cytokine, a genetic modification protein, or a viral replication protein.

27. (canceled)

28. (canceled)

29. (canceled)

30. (canceled)

31. (canceled)

32. The composition of claim 1, wherein the least one oncoselective modification increases expression of the at least one therapeutic in a cancer cell compared to expression of the at least one therapeutic in a normal cell.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A method for treating a disease or condition in a subject, comprising administering to the subject at least one engineered nucleic acid, said at least one engineered nucleic acid: comprises at least one oncoselective modification; and encodes at least one therapeutic, wherein the at least one oncoselective modification comprises a nucleic acid sequence of any one of or a combination of any one of SEO ID NO: 41-110.

46. The method of claim 45, wherein the at least one therapeutic comprises least one cytokine comprising least one interleukin or at least one interferon.

47. (canceled)

48. (canceled)

49. (canceled)

50. The method of claim 45, wherein the at least one cytokine comprises IL-2, IL-12, IL-15, or IFNα.

51. (canceled)

52. (canceled)

53. The method of claim 45, wherein the disease or condition comprises cancer.

54. (canceled)

55. (canceled)

56. (canceled)

57. The method of claim 45, wherein the at least one oncoselective modification increases expression of the at least one therapeutic in a cancer cell compared to expression of the at least one therapeutic in a cell.

58. (canceled)


SEQUENCE LISTING (110 sequences)

SEQ ID NO: 1 - RNA, 72 nt, synthetic construct
  source 1..72: mol_type = other RNA
aaacgcaagcaagaggaacaaatggaaactgagcattttgctctgtaaagcatttcagatgcagcttgtgtg

SEQ ID NO: 2 - RNA, 306 nt, synthetic construct
  source 1..306: mol_type = other RNA
gcgctgacccagctgatccagctctatcatcgcttccaccgggtgctgtcccagccgcagctccgagccctccctgcccgggctgagctcatcaacattcaccaccttatggtggagctcaagaagcataagcccaacttctgatgtgccagaaaccgccctgagatctgccggtcatctccatggacttctgcaccccattccatacccttcttcacctggggtaccccttccagttttccccttgcttcccaggcccttgacatggcttacctgccttcactcccagcaccttgcccaacagga

SEQ ID NO: 3 - RNA, 126 nt, synthetic construct
  source 1..126: mol_type = other RNA
cttccagcagaggagaacatgtctaacacgtgcctcaaaagcactggggagttagtagtgcagtggcatctgaaacctgtggagcagaaagcacatgagtcctaatgccccagcagcttccgattg

SEQ ID NO: 4 - RNA, 222 nt, synthetic construct
  source 1..222: mol_type = other RNA
atagtataccctggaattgctgtatttttccagaatgccttcatcttttttggagggctggtttttaagtttggccgcactgaagacttatggcagtgaacacatctgatttcccacagcacaacagccctgcatgggtttgtttgtttttttactgctcactcccaaccttttgtaatgccattttctaaacttatttctgagtgtagtctcagcttaaag

SEQ ID NO: 5 - RNA, 201 nt, synthetic construct
  source 1..201: mol_type = other RNA
ggtgacaaatccattcgtttccgtcccacgctggtcttcagggatcaccacgctcacctgttcctcaatattttcagtgacatcttagcagacttcaagtaaagaagccatttccactacagtgagaaagcccggatcccaacagttgtcaaattgattagtttgcctaattcatgttttcacttaaaagtatcagaggtg

SEQ ID NO: 6 - RNA, 264 nt, synthetic construct
  source 1..264: mol_type = other RNA
tgtcctttgggctctgtggagagctttagccttgcacggcggcgctgggaggcattgcctgccatgcccactgcccgctgctcctgctctagtctgcaggctgggccccggctgtttgttattgggggtgtggcccagggccccagtcaagccgtggaggcactgtgtctgcgtgatggggtctgaaggcttggtgggagctgtccactggagcagctcattgccagaggcagctatttctatggctccttttgctgctgagga

SEQ ID NO: 7 - RNA, 201 nt, synthetic construct
  source 1..201: mol_type = other RNA
atatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacct

SEQ ID NO: 8 - RNA, 201 nt, synthetic construct
  source 1..201: mol_type = other RNA
gaggttctccaggaccttaggtttgatgcggaatctgccgagtgatggcggctccccagggatgcgccgagggagatgggaaacggggcggatggcgcccagcccagccctaactgccagctggctggggttgcgccccactgcgctgctgaccttcctgcagttccagacacctcccacaataaagagctcctcctctgt

SEQ ID NO: 9 - RNA, 198 nt, synthetic construct
  source 1..198: mol_type = other RNA
ggtgaggtcaaggcaggagagaagagcctgagccagcacgtggaggccgtggacaagcggctggaacagagccagcccgagtacaaggcgctcttcaaagagatcttctccaggatccagaagaccaaggctgacatcaacgccaccaaagtcaagacgcacagcagcaagtgacccttctccggcctgcagcctccc

SEQ ID NO: 10 - RNA, 213 nt, synthetic construct
  source 1..213: mol_type = other RNA
gaggcgatcagcgaggttctccaggaccttaggtttgatgcggaatctgccgagtgatggcggctccccagggatgcgccgagggagatgggaaacggggcggatggcgcccagcccagccctaactgccagctggctggggttgcgccccactgcgctgctgaccttcctgcagttccagacacctcccacaataaagagctcctcctctgt

SEQ ID NO: 11 - RNA, 201 nt, synthetic construct; identical to SEQ ID NO: 8
  source 1..201: mol_type = other RNA

SEQ ID NO: 12 - RNA, 201 nt, synthetic construct
  source 1..201: mol_type = other RNA
atcctagcttcggtgctggcagtgtcccaacaggaatacctagacagtatgaagaaaaacaaagtgcacagagacccgcccccagacaagagttgatggagacccagggattggacaccatctcccaaccccagtactcctgctctccggtgccacctcaccttctttggcttcttccctcttgcctccttctgttctttc

SEQ ID NO: 13 - RNA, 201 nt, synthetic construct
  source 1..201: mol_type = other RNA
actgtttcctatgatcctaggaaacccactgtgaagaaaattgccccaatgatggccaagacaattaaagctttcaagaacagattctcccgacgataaactgaggacttgccttggaaatggaatctggggaggcaggaatacaaggacagtgggggttggggaatggaattctacaggagactggagtcttgctttgtg

SEQ ID NO: 14 - RNA, 159 nt, synthetic construct
  source 1..159: mol_type = other RNA
ttgtcatgtgtacaggaaatcagtgatgtggtgcagaggtagccactgttagcctggtgggaaaatgcacacatttctgaggggagagggaaaaggacttgttttcctgtgttcttgttttcagaaaatgaaagactcatacttgagtgtgtttatgtg

SEQ ID NO: 15 - skipped

SEQ ID NO: 16 - RNA, 228 nt, synthetic construct
  source 1..228: mol_type = other RNA
gtgcagcacaaaaagcccgccgacatccctcagggctccttggcctacctggagcaggcatctgccaacatccctgcacctctgaagccaacgtgagcaaagggcagaggcagttggcctatgagtgggctgatgcgtgaggttggccacacattccttcctgtggacttgacattttggaagaactctttgccagataatgagttcattttagttttatgctcccat

SEQ ID NO: 17 - RNA, 264 nt, synthetic construct
  source 1..264: mol_type = other RNA
tggggtccccagtggggaatgaacgggtacttcctcatcgagcgcggaaagaacatgtgtggcctggctgcctgcgcctcctaccccatccctctggtgtgagccgtggcagccgcagcgcagactggcggagaaggagaggaacgggcagcctgggcctgggtggaaatcctgccctggaggaagttgtggggagatccactgggacccccaacattctgccctcacctctgtgcccagcctggaaacctacagacaaggagg

SEQ ID NO: 18 - RNA, 249 nt, synthetic construct
  source 1..249: mol_type = other RNA
gaagaaacaaagaataaactcaaagccggcaatgccctggcatttttgggtcttgagagaaaacaatttgaatgactgaatttactacaaaggcaaactttcaaaaggatatctcttttttgtttccaaatatgtatcaacaggtatcaacaaaatcctattttgaactattttactcagaaaagaatatcccaaatatcccaaattattcataataaaaatgatttgaaagtgttttcattcttaaaa

SEQ ID NO: 19 - RNA, 195 nt, synthetic construct
  source 1..195: mol_type = other RNA
ttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaagatgtgcaaggagatttaaggatttcaaagaaaataaaggttctttgtttt

SEQ ID NO: 20 - RNA, 306 nt, synthetic construct
  source 1..306: mol_type = other RNA
aagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaa

SEQ ID NO: 21 - RNA, 230 nt, synthetic construct
  source 1..230: mol_type = other RNA
ccccgccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgaga

SEQ ID NO: 22 - RNA, 1908 nt, synthetic construct
  source 1..1908: mol_type = other RNA
gaaataagagagaaaagaagagtaagaagaaatataagagccaccatggaagacgccaaaaacataaagaaaggcccggcgccattctatccgctggaagatggaaccgctggatagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagctgccttctgcggggcttgccttctggccatgcccttcttctctcccttgcacctgtacctcttggtctttgaataaagcctgagtaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - RNA, 55 nt, synthetic construct
  source 1..55: mol_type = other RNA
ctatccgctggaagatggaaccgctggatagcaactgcataaggctatgaagaga

SEQ ID NO: 24 - RNA, 1211 nt, synthetic construct
  source 1..1211: mol_type = other RNA
aggtcttctggtccccacagactcagagagaacccgccaccatgggcagcgccttcgagcgcgtcgtccgccgcgtcgtccaggagctcgaccacggcggcgagttcatccccgtcaccagcctccagagcagcaccggcttccagccctactgcctcgtcgtccgcaagcccagcagcagctggttctggaagccccgctacaagtgcgtgaacctgagcatcaaggacatcctcgagccggacgccgccgagcccgacgtccagcgcggccgcagcttccacttctacgacgccatggacggccagatccagggcagcgtcgagctggccgcccccggccaggccaagatcgccggcggcgccgccgtcagcgacagcagcagcaccagcatgaacgtctacagcctcagcgtcgaccccaacacctggcagaccctcctccacgagcgccacctccgccagcccgagcacaaggtcctccagcagctccgcagccggggcgacaacgtctacgtcgtcaccgaggtcctccagacccagaaggaggtcgaggtcacccggacccacaagcgcgagggcagcggccgcttcagcctccccggcgccacctgcctccagggcgagggccagggccacctcagccagaagaagaccgtcaccatccccagcggcagcaccctcgccttccgcgtcgcccagctcgtcatcgacagcgacctcgacgtcctcctcttccccgacaagaagcagcgcaccttccagccccccgccaccggccacaagcgcagcaccagcgagggcgcctggccccagctccccagcggcctcagcatgatgcgctgcctccacaacttcctcaccgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 25 - RNA, 950 nt, synthetic construct
  source 1..950: mol_type = other RNA
aggtcttctggtccccacagactcagagagaacccgccaccatggccctgaccttcgccctgttagtggcgctgctggtgctgagctgcaagagcagctgcagcgtgggctgcgacctgcctcagacccacagcctgggcagccggcggaccctgatgctgctggcccagatgcggaagatcagcctgttcagctgtctgaaggaccggcacgacttcggcttcccccaggaggaattcggcaaccagttccagaaggccgagaccatccccgtgctgcacgagatgatccagcagatcttcaacctgttcagcaccaaggatagcagcgccgcttgggacgagacccttctggacaagttctacaccgagctgtaccagcagctgaacgacctggaggcctgcgtgatccagggcgtgggcgtgaccgagacccctctgatgaaggaggacagcatcctggccgtgcggaagtactttcagcggatcaccctgtacctgaaggagaagaagtacagcccatgcgcctgggaggtggtgcgggccgagatcatgcggagcttcagcctgagcaccaacctgcaggagagcctgcggagcaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 26 - RNA, 1214 nt, synthetic construct
  source 1..1214: mol_type = other RNA
aggtcttctggtccccacagactcagagagaacccgccaccatgccaagcgccttcgagaaggtggtgaagaacgtgatcaaggaggtctccggcagccggggcgacctgatccccgtggacagcctgcggaacagcaccagcttccggccctactgcctgctgaaccggaagttcagcagcagccggttctggaagccccggtacagctgcgtgaacctgagcatcaaggacatcctggagcccagcgcccccgagcccgagcctgagtgtttcggcagcttcaaggtgagcgacgtggtggacggcaacatccagggccgggtgatgctgagcggcatgggcgagggaaagatcagcgggggcgccgccgtgagcgacagcagcagcgccagcatgaacgtgtgcatcctgcgggtgacccagaagacctgggagaccatgcagcacgagcggcacctgcagcagcctgagaataagatcctgcagcagctgcggagccggggcgatgacctgttcgtggtgaccgaagtgctgcagaccaaggaggaggtgcagatcaccgaggtgcacagccaggagggcagcggccagttcaccctgcccggcgccctgtgcctgaagggcgagggcaagggacaccagagccggaagaagatggtgaccatccccgccggcagcatcctggccttccgggtggcccagctgctgatcggcagcaagtgggacattctgctggtgagcgacgagaagcagcggaccttcgagcccagcagcggcgaccggaaggccgtgggccagcggcaccacggcctgaacgtgctggccgccctgtgtagcatcggcaagcagctaagcctgctgagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 27 - RNA, 944 nt, synthetic construct
  source 1..944: mol_type = other RNA
aggtcttctggtccccacagactcagagagaacccgccaccatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 28 - RNA, 1670 nt, synthetic construct
  source 1..1670: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccaggcagcgccttcgagcgcgtcgtccgccgcgtcgtccaggagctcgaccacggcggcgagttcatccccgtcaccagcctccagagcagcaccggcttccagccctactgcctcgtcgtccgcaagcccagcagcagctggttctggaagccccgctacaagtgcgtgaacctgagcatcaaggacatcctcgagccggacgccgccgagcccgacgtccagcgcggccgcagcttccacttctacgacgccatggacggccagatccagggcagcgtcgagctggccgcccccggccaggccaagatcgccggcggcgccgccgtcagcgacagcagcagcaccagcatgaacgtctacagcctcagcgtcgaccccaacacctggcagaccctcctccacgagcgccacctccgccagcccgagcacaaggtcctccagcagctccgcagccggggcgacaacgtctacgtcgtcaccgaggtcctccagacccagaaggaggtcgaggtcacccggacccacaagcgcgagggcagcggccgcttcagcctccccggcgccacctgcctccagggcgagggccagggccacctcagccagaagaagaccgtcaccatccccagcggcagcaccctcgccttccgcgtcgcccagctcgtcatcgacagcgacctcgacgtcctcctcttccccgacaagaagcagcgcaccttccagccccccgccaccggccacaagcgcagcaccagcgagggcgcctggccccagctccccagcggcctcagcatgatgcgctgcctccacaacttcctcaccgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 29 - RNA, 1409 nt, synthetic construct
  source 1..1409: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccagccctgaccttcgccctgttagtggcgctgctggtgctgagctgcaagagcagctgcagcgtgggctgcgacctgcctcagacccacagcctgggcagccggcggaccctgatgctgctggcccagatgcggaagatcagcctgttcagctgtctgaaggaccggcacgacttcggcttcccccaggaggaattcggcaaccagttccagaaggccgagaccatccccgtgctgcacgagatgatccagcagatcttcaacctgttcagcaccaaggatagcagcgccgcttgggacgagacccttctggacaagttctacaccgagctgtaccagcagctgaacgacctggaggcctgcgtgatccagggcgtgggcgtgaccgagacccctctgatgaaggaggacagcatcctggccgtgcggaagtactttcagcggatcaccctgtacctgaaggagaagaagtacagcccatgcgcctgggaggtggtgcgggccgagatcatgcggagcttcagcctgagcaccaacctgcaggagagcctgcggagcaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 30 - RNA, 1673 nt, synthetic construct
  source 1..1673: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccaccaagcgccttcgagaaggtggtgaagaacgtgatcaaggaggtctccggcagccggggcgacctgatccccgtggacagcctgcggaacagcaccagcttccggccctactgcctgctgaaccggaagttcagcagcagccggttctggaagccccggtacagctgcgtgaacctgagcatcaaggacatcctggagcccagcgcccccgagcccgagcctgagtgtttcggcagcttcaaggtgagcgacgtggtggacggcaacatccagggccgggtgatgctgagcggcatgggcgagggaaagatcagcgggggcgccgccgtgagcgacagcagcagcgccagcatgaacgtgtgcatcctgcgggtgacccagaagacctgggagaccatgcagcacgagcggcacctgcagcagcctgagaataagatcctgcagcagctgcggagccggggcgatgacctgttcgtggtgaccgaagtgctgcagaccaaggaggaggtgcagatcaccgaggtgcacagccaggagggcagcggccagttcaccctgcccggcgccctgtgcctgaagggcgagggcaagggacaccagagccggaagaagatggtgaccatccccgccggcagcatcctggccttccgggtggcccagctgctgatcggcagcaagtgggacattctgctggtgagcgacgagaagcagcggaccttcgagcccagcagcggcgaccggaaggccgtgggccagcggcaccacggcctgaacgtgctggccgccctgtgtagcatcggcaagcagctaagcctgctgagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 31 - RNA, 1403 nt, synthetic construct
  source 1..1403: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccagctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 32-40 - skipped (9 entries)

SEQ ID NO: 41 - RNA, 2189 nt, synthetic construct
  source 1..2189: mol_type = other RNA
aggtcttctggtccccacagactcagagagaacccgccaccatgagcagcgtgaagctgtggcctaccggcgccagcgccgtgcccctggtgagccgggaggagttaaagaagctggagttcgtgggcaagggcggcttcggcgtggtgttccgggcccaccaccggacttggaaccacgatgtggccgtgaagatcgtgaatagcaagaagatcagctgggaggtgaaggccatggtgaacctgcggaacgagaacgtgctgctgctgctgggcgtgaccgaggacctgcagtgggacttcgtgagcggccaggccctggtgacccggttcatggagaatggcagcctggccggcctgctgcagcccgagtgccctcggccctggcccctcctgtgccggctgctgcaggaggtggtgctgggcatgtgctacctgcacagcctgaacccacccctgctgcacagggacctgaagcccagcaacatcctgctggaccctgagctgcacgccaagctggccgatttcggcctgagcaccttccagggcggcagccagagcggcagcggcagcggaagcggctcacgggacagcggcggcaccctggcctacctggaccccgagctgctgttcgacgtgaacctgaaggccagcaaagccagcgacgtgtacagcttcggcatcctggtgtgggccgtgctggcaggccgggaggctgagctggtggacaagaccagcctgatccgggagaccgtgtgcgaccggcagagccggccccccctgaccgagctgccccccggcagccccgagacccccggcctggagaagctcaaggagctgatgatccactgctggggctcacagtccgagaatcgtccaagcttccaggactgcgagcccaagaccaacgaggtgtacaacctggtgaaggacaaggtggacgccgccgtgagcgaggtgaagcactacctgagccagcaccggagcagcggccggaacctgagcgcccgggagcccagccagcggggcaccgagatggactgcccccgggagaccatggtgagcaagatgctggaccggctgcacctggaggagcccagcggacctgtgcccggcaagtgccccgagcggcaggcccaggacaccagcgtgggccccgccacccccgcccggaccagcagcgaccccgtggccggcacccctcagatcccccacaccctgcccttccggggcaccacccccggccccgtgttcaccgagaccccaggcccccacccccagaggaatcagggcgacggccggcacggcaccccatggtacccttggaccccccccaaccccatgaccggcccccccgcactggtgtttaataactgtagcgaggtgcagatcggcaactacaacagcctggtcgcccccccccggaccaccgccagcagcagcgccaagtacgaccaggcccagttcggccggggccggggctggcagcccttccacaagggcaccaagtacgagatcgaacggatgctgaaggagctgaaggacctgctggaagagctgaagcggatgctggaggagcttaaacggagcctgaaggaactgaagaagaaccccagcgaggacgccctggtggagaataaccggctgatcgtgaaggtgctggaggtgatcgtggaggtgctgcgggccatcatcgagctggccgagctgatcatccggagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - RNA, 2285 nt, synthetic construct
  source 1..2285: mol_type = other RNA
aggtcttctggtccccacagactcagagagaacccgccaccatgagctgcgtgaagctgtggcccagcggcgcccccgccccactggtgagcatcgaggagctggagaaccaggagctggtgggcaagggcggcttcggcaccgtgttccgggcccagcaccggaagtggggctacgacgtggccgtgaagatcgtgaacagcaaggcaatcagccgggaggtgaaggccatggccagcctggacaacgagttcgtgctgcggctggagggcgtgatcgagaaggtgaactgggaccaggacccaaagcctgccctggtgaccaagttcatggagaacggcagcctgagcggactgctgcagagccagtgcccccggccctggccactgctgtgccggctgctgaaggaggtggtgctgggcatgttctacctgcacgatcagaaccccgtgctgctgcaccgtgacctgaagcccagcaacgtgctgctggaccccgagctgcacgtgaagctggctgacttcggcctgagcaccttccagggcggcagccagagcggcaccggcagcggcgagcccggcggcaccctgggctacctggcccccgagctgttcgtgaatgtgaaccggaaggccagcaccgccagcgacgtgtacagcttcggcatcctgatgtgggccgtgctggccggccgggaggtcgagctgcccaccgagcccagcctggtgtacgaggctgtgtgcaaccggcagaaccggcccagcctggcagagctgccccaggccggccccgagacccccggcctggagggccttaaggagctgatgcagctgtgctggagcagcgagcctaaggaccggcccagcttccaggagtgcctgcccaagaccgacgaggtgttccagatggtcgagaataatatgaacgcagctgtgagcaccgtgaaggacttcctgagccagctgcggagcagcaaccggcggttcagcatccccgagagcggccagggaggcaccgagatggatggcttccggcggaccatcgagaaccagcacagcaggaatgacgtgatggtgagcgagtggctgaacaagctgaacctggaggagccccccagcagcgtgccaaagaagtgccccagcctgaccaagcggagccgggcccaggaggagcaggtgccccaggcctggaccgccggcaccagcagcgacagcatggcccagcccccccagaccccagagacctcaaccttccggaaccagatgcccagccccaccagcaccggcaccccaagccccggcccccggggcaaccagggcgccgagaggcagggcatgaactggagctgccggacccccgagcccaaccccgtgaccggccggcccctggtgaacatctacaactgcagcggcgtgcaggtgggagacaacaactacctgaccatgcagcagaccaccgctctgcccacctggggcctggccccaagcggcaagggccggggcctgcagcaccccccccccgtgggcagccaggagggccccaaggacccagaggcctggagccggccccagggctggtacaaccacagcggcaaggggaccaagtacgagatcgagcggatgctgaaggaactgaaggacctgctggaggagctgaaacggatgctcgaggagctgaagcggagcctgaaggagctgaagaagaacccaagcgaggacgccctggtggagaacaaccggctgatcgtgaaggtgctggaggtgatcgtggaggtgctgcgggccatcatcgagctggccgagctgataatccggagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - RNA, 965 nt, synthetic construct
  source 1..965: mol_type = other RNA
aggtcttctggtccccacagactcagagagaacccgccaccatgtaccggatgcagctgctgagctgcattgccctgagcctggccctggtgaccaacagcgcccccaccagcagcagcaccaagaagacccagctgcagctagagcacctgctgctggacctgcagatgatcctgaatggcatcaacaactacaagaaccctaagctgacccggatgctgaccgcaaagttcgccatgcccaagaaggccactgagctgaagcacctgcagtgcctggaggaggagctgaagcccctggaggaggtgctgaacggcgcccagagcaagaacttccacctgcggccccgggacctgatcagcaacattaacgtgatcgtgctggagctgaagggcagcgagaccaccttcatgtgcgaatacgccgacgaaaccgctacaatcgtggaattcctgaatcggtggatcaccttctgccagagcatcatcagcaccctgacctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - RNA, 2648 nt, synthetic construct
  source 1..2648: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccaagcagcgtgaagctgtggcctaccggcgccagcgccgtgcccctggtgagccgggaggagttaaagaagctggagttcgtgggcaagggcggcttcggcgtggtgttccgggcccaccaccggacttggaaccacgatgtggccgtgaagatcgtgaatagcaagaagatcagctgggaggtgaaggccatggtgaacctgcggaacgagaacgtgctgctgctgctgggcgtgaccgaggacctgcagtgggacttcgtgagcggccaggccctggtgacccggttcatggagaatggcagcctggccggcctgctgcagcccgagtgccctcggccctggcccctcctgtgccggctgctgcaggaggtggtgctgggcatgtgctacctgcacagcctgaacccacccctgctgcacagggacctgaagcccagcaacatcctgctggaccctgagctgcacgccaagctggccgatttcggcctgagcaccttccagggcggcagccagagcggcagcggcagcggaagcggctcacgggacagcggcggcaccctggcctacctggaccccgagctgctgttcgacgtgaacctgaaggccagcaaagccagcgacgtgtacagcttcggcatcctggtgtgggccgtgctggcaggccgggaggctgagctggtggacaagaccagcctgatccgggagaccgtgtgcgaccggcagagccggccccccctgaccgagctgccccccggcagccccgagacccccggcctggagaagctcaaggagctgatgatccactgctggggctcacagtccgagaatcgtccaagcttccaggactgcgagcccaagaccaacgaggtgtacaacctggtgaaggacaaggtggacgccgccgtgagcgaggtgaagcactacctgagccagcaccggagcagcggccggaacctgagcgcccgggagcccagccagcggggcaccgagatggactgcccccgggagaccatggtgagcaagatgctggaccggctgcacctggaggagcccagcggacctgtgcccggcaagtgccccgagcggcaggcccaggacaccagcgtgggccccgccacccccgcccggaccagcagcgaccccgtggccggcacccctcagatcccccacaccctgcccttccggggcaccacccccggccccgtgttcaccgagaccccaggcccccacccccagaggaatcagggcgacggccggcacggcaccccatggtacccttggaccccccccaaccccatgaccggcccccccgcactggtgtttaataactgtagcgaggtgcagatcggcaactacaacagcctggtcgcccccccccggaccaccgccagcagcagcgccaagtacgaccaggcccagttcggccggggccggggctggcagcccttccacaagggcaccaagtacgagatcgaacggatgctgaaggagctgaaggacctgctggaagagctgaagcggatgctggaggagcttaaacggagcctgaaggaactgaagaagaaccccagcgaggacgccctggtggagaataaccggctgatcgtgaaggtgctggaggtgatcgtggaggtgctgcgggccatcatcgagctggccgagctgatcatccggagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - RNA, 2744 nt, synthetic construct
  source 1..2744: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccaagctgcgtgaagctgtggcccagcggcgcccccgccccactggtgagcatcgaggagctggagaaccaggagctggtgggcaagggcggcttcggcaccgtgttccgggcccagcaccggaagtggggctacgacgtggccgtgaagatcgtgaacagcaaggcaatcagccgggaggtgaaggccatggccagcctggacaacgagttcgtgctgcggctggagggcgtgatcgagaaggtgaactgggaccaggacccaaagcctgccctggtgaccaagttcatggagaacggcagcctgagcggactgctgcagagccagtgcccccggccctggccactgctgtgccggctgctgaaggaggtggtgctgggcatgttctacctgcacgatcagaaccccgtgctgctgcaccgtgacctgaagcccagcaacgtgctgctggaccccgagctgcacgtgaagctggctgacttcggcctgagcaccttccagggcggcagccagagcggcaccggcagcggcgagcccggcggcaccctgggctacctggcccccgagctgttcgtgaatgtgaaccggaaggccagcaccgccagcgacgtgtacagcttcggcatcctgatgtgggccgtgctggccggccgggaggtcgagctgcccaccgagcccagcctggtgtacgaggctgtgtgcaaccggcagaaccggcccagcctggcagagctgccccaggccggccccgagacccccggcctggagggccttaaggagctgatgcagctgtgctggagcagcgagcctaaggaccggcccagcttccaggagtgcctgcccaagaccgacgaggtgttccagatggtcgagaataatatgaacgcagctgtgagcaccgtgaaggacttcctgagccagctgcggagcagcaaccggcggttcagcatccccgagagcggccagggaggcaccgagatggatggcttccggcggaccatcgagaaccagcacagcaggaatgacgtgatggtgagcgagtggctgaacaagctgaacctggaggagccccccagcagcgtgccaaagaagtgccccagcctgaccaagcggagccgggcccaggaggagcaggtgccccaggcctggaccgccggcaccagcagcgacagcatggcccagcccccccagaccccagagacctcaaccttccggaaccagatgcccagccccaccagcaccggcaccccaagccccggcccccggggcaaccagggcgccgagaggcagggcatgaactggagctgccggacccccgagcccaaccccgtgaccggccggcccctggtgaacatctacaactgcagcggcgtgcaggtgggagacaacaactacctgaccatgcagcagaccaccgctctgcccacctggggcctggccccaagcggcaagggccggggcctgcagcaccccccccccgtgggcagccaggagggccccaaggacccagaggcctggagccggccccagggctggtacaaccacagcggcaaggggaccaagtacgagatcgagcggatgctgaaggaactgaaggacctgctggaggagctgaaacggatgctcgaggagctgaagcggagcctgaaggagctgaagaagaacccaagcgaggacgccctggtggagaacaaccggctgatcgtgaaggtgctggaggtgatcgtggaggtgctgcgggccatcatcgagctggccgagctgataatccggagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - RNA, 1424 nt, synthetic construct
  source 1..1424: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccataccggatgcagctgctgagctgcattgccctgagcctggccctggtgaccaacagcgcccccaccagcagcagcaccaagaagacccagctgcagctagagcacctgctgctggacctgcagatgatcctgaatggcatcaacaactacaagaaccctaagctgacccggatgctgaccgcaaagttcgccatgcccaagaaggccactgagctgaagcacctgcagtgcctggaggaggagctgaagcccctggaggaggtgctgaacggcgcccagagcaagaacttccacctgcggccccgggacctgatcagcaacattaacgtgatcgtgctggagctgaagggcagcgagaccaccttcatgtgcgaatacgccgacgaaaccgctacaatcgtggaattcctgaatcggtggatcaccttctgccagagcatcatcagcaccctgacctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - RNA, 1307 nt, synthetic construct
  source 1..1307: mol_type = other RNA
aggtcttctggtccccacagactcagagagaacccgccaccatggccccccggcgggcccggggctgccggaccctgggcctgcctgccctgctgctgctgcttctgctgcggccccccgccacccggggcatcacctgtcccccacctatgagcgtggagcacgccgacatctgggtgaagagctacagcctgtacagccgggagcggtacatctgcaacagcggcttcaagcggaaggcaggcaccagcagcctgaccgagtgcgtgctgaacaaggccaccaacgtggcccactggaccacccccagccttaagtgcatccgggaccctgcgctggtccaccagcggcctgccccccccagcaccgtgaccaccgcaggagtgaccccccagcccgagagcctgagccccagcggcaaggagcccgccgccagctcacccagcagcaacaacaccgccgccaccaccgccgccatcgtgcccggcagccagctgatgcctagcaagagccccagcaccggcaccaccgagatcagcagccacgagagcagccacggcacccccagccagaccaccgccaagaattgggagctgaccgccagcgccagccaccagcccccaggcgtgtacccccaggggcacagcgacaccaccgtggccatcagcaccagcaccgtgctgctgtgcggcctgagcgcagtgagcctgctggcctgctacctgaagagccggcagaccccacccctggctagcgtggagatggaggccatggaggccctgcccgtgacctggggaaccagcagccgggacgaggacctggagaactgcagccaccacctgtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - RNA, 1175 nt, synthetic construct
  source 1..1175: mol_type = other RNA
aggtcttctggtccccacagactcagagagaacccgccaccatggagttcggcctgagctgggtgttcctggtggccctgttccggggagtgcagtgtatcacctgccccccccccatgagcgtggagcacgctgacatctgggtgaagagctacagcctgtacagccgggagcggtacatctgcaacagcggcttcaagcggaaggccggcaccagctccctgaccgagtgcgtgctgaacaaggccaccaacgtggcccactggaccacccccagcctgaagtgcatccgggaccccgccctggtgggcggctcaggcggcagcggcggcagcgggggcagcggaggcagcggcggaagcggcggcaactgggtgaacgtgatcagcgacctgaagaagatcgaggacctgatccaaagcatgcacattgacgccaccctgtacaccgagagcgacgtgcaccccagctgcaaggtgaccgcaatgaagtgcttcctgctggagctgcaggtgatcagcctggagagcggcgacgccagcatccacgacaccgtggagaacctgatcatcctggcgaacaacagcctgagcagcaacggcaacgtgaccgagagcggctgtaaggagtgcgaggagctggaggagaagaatatcaaggagttcctgcagagcttcgtgcacatcgtgcagatgttcatcaacaccagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - RNA, 1175 nt, synthetic construct
  source 1..1175: mol_type = other RNA
aggtcttctggtccccacagactcagagagaacccgccaccatggagttcggcctgagctgggtgtttctggtggccctgttccggggcgtgcagtgcaactgggtgaacgtgatcagcgacctgaagaagatcgaggacctgatccagtcaatgcacatcgatgcaaccctgtacaccgagagcgacgtgcaccctagctgtaaggtgaccgcaatgaagtgcttcctgctggagctgcaggtgataagcctggagagcggcgacgccagcatccacgacacagtcgagaatctgatcatcctggctaacaacagcctgagcagcaatggtaatgtgaccgagagcggctgcaaggagtgcgaggagctggaggagaagaacatcaaggagttcctgcagagcttcgtgcacatcgtgcagatgttcatcaacaccagcggcggcagcgggggcagcggcggctcaggtggcagcggaggcagcggcggaagcggcggcatcacctgccctccccccatgagcgtggagcacgcagacatctgggtgaagagctacagcctgtacagccgggagcggtacatctgcaacagcggcttcaagcggaaggccggcaccagcagcctgaccgagtgtgtgctgaacaaggccaccaacgtggcccactggacaacccccagcctgaagtgcatcagggaccccgccctggtgtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - RNA, 1841 nt, synthetic construct
  source 1..1841: mol_type = other RNA
aggtcttctggtccccacagactcagagagaacccgccaccatggctagcccccagctgcggggctacggcgtgcaggccatccctgtgctgctgttactgctgctgctcctgctgctgcccctgcgggtgacccccggcaccacctgccccccccccgtgagcatcgagcacgccgacatccgggtgaagaactacagcgtgaacagccgggagaggtacgtgtgcaatagcggcttcaagcggaaggccggcaccagcaccctgatcgagtgcgtgatcaacaagaacaccaacgtggcccactggaccacccccagcctgaagtgcatccgggaccccagcttagcccactacagccccgtgcccaccgtggtgaccccaaaggtgaccagccagcccgagagccccagccccagcgccaaggagcctgaggccttctccccaaagagcgacaccgccatgaccaccgagaccgccatcatgcccggcagccggctgacccccagccagaccaccagcgccggcaccacaggcaccggcagccacaagagcagccgggcacccagcctggccgccaccatgaccctggagcctaccgctagcaccagcctgcggatcaccgagatcagcccccacagcagcaagatgaccaaggtggccatcagcaccagcgtgctgctggtgggcgccggcgtggtgatggctttcctggcctggtacatcaagagccggcagcccagccagccttgccgggtggaggtggagaccatggagactgtgcccatgaccgtgcgggccagcagcaaggaggacgaggacaccggcgccgggggcagcggcggtagcggaggcagcggcggcagcggcggaagcggcggctccggcggcatgaagatcctgaagccctacatgcggaacaccagcatcagctgctacctgtgcttcctgctgaacagccacttcctgaccgaggccggcatccacgtgttcatcctgggctgcgtgagcgtgggcctgcctaagaccgaggccaactggatcgacgtgcggtacgacctggagaagatcgagagcctgatccagagcatccacatcgacaccaccctgtacaccgacagcgacttccaccccagctgcaaggtgaccgccatgaactgctttctgctggagctgcaggtgatcctgcacgagtacagcaacatgaccctgaacgagaccgtgcggaacgtgctgtacctggccaacagcaccctgagcagcaacaagaacgtggccgagagcggctgcaaggagtgcgaggagctggaggagaagaccttcaccgagttcctgcagagcttcatccggatcgtgcagatgttcatcaacaccagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - RNA, 1178 nt, synthetic construct
  source 1..1178: mol_type = other RNA
aggtcttctggtccccacagactcagagagaacccgccaccatgagcgtgctgacccaggtgctggccctgctgctgctgtggctgaccggcgcccgttgcaccacctgcccccccccagtgagcatcgagcacgccgatatccgggtgaagaactacagcgtgaacagccgggagcggtacgtgtgcaacagcggcttcaagcggaaggccggcacaagcaccctgatcgagtgcgtgatcaacaagaacaccaatgtggcccactggaccacccccagcctgaagtgtatccgggaccctagcctggccggcggcagcggcgggagcggtggcagcggcggaagcggcggtagcggaggcagcggcggcaactggatcgacgtgcggtacgacctagagaagatcgagagcctgatccagagcatccacatcgacaccaccctgtacaccgacagcgacttccaccccagctgcaaggtgaccgccatgaactgcttcctgctggagctgcaggtgatcctgcacgagtacagcaacatgacactgaacgagaccgtgcggaacgtgctgtacctggcaaacagcaccctgagcagcaacaagaatgtggccgagagcggctgcaaggaatgcgaggagctggaggagaagaccttcaccgagttcctccagagcttcatccggatcgtgcagatgttcatcaacaccagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - RNA, 1178 nt, synthetic construct
  source 1..1178: mol_type = other RNA
aggtcttctggtccccacagactcagagagaacccgccaccatgagcgtgctgacccaggtgctggctctactgctgctgtggttaacaggcgcccggtgcaactggatcgacgtgcggtacgacctggagaagatcgagagcctgatccagagcatccacatcgacaccaccctgtacaccgacagcgacttccaccctagctgcaaggtgaccgccatgaactgcttcctgctggagctgcaggtgatcctgcacgagtacagcaatatgaccctgaacgagaccgtgcggaacgtgctgtacctggccaacagcaccctgagcagcaataagaatgtggccgagagcggctgcaaggagtgtgaggagctggaggagaagaccttcactgagttcctgcagagctttatccggatcgtgcagatgttcatcaacactagcggcggaagcggcggctcaggcggcagcggaggcagcggcggcagcgggggcagcggcggaaccacctgtcccccccccgtgagcatcgagcacgccgacatccgggtgaagaactacagcgtgaactcacgggagcggtacgtgtgcaactcaggcttcaagcggaaggccggcaccagcaccctgatcgagtgcgtgatcaacaagaacacaaacgtggcccactggaccacccccagcctgaagtgcatccgggaccccagcctggcctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - RNA, 1748 nt, synthetic construct
  source 1..1748: mol_type = other RNA
aggtcttctggtccccacagactcagagagaacccgccaccatgagcgtgctgacccaggtgctggccctgctgctgctgtggctgaccggcgcccggtgcaactggatcgacgtgcggtacgatctggagaagatcgagagcctgatccagagcatccacatcgacaccaccctgtacaccgatagcgacttccaccccagctgcaaggtgaccgccatgaactgcttcctgctggaactgcaggtgatcctgcacgagtacagcaacatgaccctgaacgagaccgtgcggaacgtcctgtacctggccaacagcacactgagcagcaacaagaacgtggccgagagcggctgcaaggagtgcgaggagctggaggagaagaccttcaccgagttcctgcagagcttcatccgtatcgtgcagatgttcatcaacaccagcgggggcagcggcggaagcgggggcagcggcggaagcggcggcagcggaggcagcggaggcaccacatgccccccacccgtgagcatcgagcacgccgacatacgggtgaagaattacagcgtgaacagccgggagcggtacgtgtgcaacagcggcttcaagcggaaggccggcaccagcaccctgatcgagtgcgtgatcaacaagaacactaacgtggcacactggaccacccccagcctcaagtgcatccgggacccaagcctggccgggggcggcggctccggcggaggcggcagcggcggtggaggcagcggccctacaatcaagccctgccccccctgcaagtgccccgcccccaatgccgccggcggccccagcgtgttcatcttcccccccaagatcaaggacgtgctgatgatctcactaagccccatagtgacctgcgtggtggtggacgtgagcgaggacgaccctgacgtgcagatcagctggttcgtgaacaacgtggaggtgcacaccgcccagacccagacccaccgggaggactacaacagcaccctgcgggtggtgagcgccctgcccatccagcaccaggactggatgagcggcaaggagttcaagtgtaaggtgaataataaggacctgggcgccccaatagagcggaccattagcaagcccaaggggagcgtgcgggccccccaggtgtacgtgctgccaccccctgaggaggagatgaccaaaaagcaggtgaccctgacctgcatggtgaccgatttcatgccagaggacatctacgtggagtggaccaacaacggcaagaccgagctgaactacaagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - RNA, 2282 nt, synthetic construct
  source 1..2282: mol_type = other RNA
aggtcttctggtccccacagactcagagagaacccgccaccatgtgccaccagcagctggtgatcagctggttcagcctggtgttcctcgccagccccctggtggctatctgggagctgaagaaggacgtgtacgtggtggagctggactggtaccccgacgcccccggcgagatggtggtgctcacctgcgatacccccgaggaggacggaatcacctggaccctggaccagagcagcgaggtgttaggcagcggcaagaccctgaccatccaggtgaaggagttcggcgacgccggccagtacacctgccacaagggcggggaggtgctgagccacagcctgctgctgctgcacaagaaggaggacggcatctggagcaccgacatcctgaaggaccagaaggagcccaagaacaagaccttcctgcggtgcgaggccaagaattacagcggccggttcacctgctggtggctgaccaccatcagcaccgacctgaccttcagcgtgaagagcagccggggcagcagcgacccccagggcgtgacctgcggcgccgccaccctgagcgccgagcgggtgaggggcgacaataaggagtacgagtacagcgtggagtgtcaggaggatagcgcatgccccgccgccgaggagagcctgcccatcgaggtgatggtggacgccgtgcacaagctgaagtacgagaactacaccagcagcttcttcatccgggacatcatcaagcccgacccccccaagaacctgcagctgaagcccctgaagaacagccggcaggtggaggtgtcatgggagtaccccgacacctggagcaccccccacagctacttcagcctgacattctgtgtgcaggtgcagggcaagagcaagcgggagaagaaggaccgggtgttcaccgacaagaccagcgccaccgtgatctgccggaagaatgccagcatcagcgtgcgggcccaggaccggtactacagcagcagctggagcgagtgggccagcgtgccttgctccggcggcggaggaagcggaggcgggggcagcgggggcggcggaagccggaacctgcccgtggccaccccagaccccggcatgttcccctgcctgcaccacagccagaacctgctgcgggccgtgagcaacatgctgcagaaggctcggcagaccctggagttctacccctgcaccagcgaggagatcgaccacgaggacatcaccaaggacaagactagcaccgtggaggcctgcctgcccctggagctgaccaagaatgagagctgcctgaacagccgggagaccagctttatcaccaacggcagctgcctggccagccggaagacctcattcatgatggccctgtgcctgagcagcatctacgaggacctgaagatgtaccaggtggagttcaagaccatgaacgccaagctgctgatggaccctaagcggcaaatcttcctggaccagaatatgctggccgtgatcgacgagctcatgcaggccctgaactttaacagcgagaccgtgccccagaagagcagcctggaggagccagacttctacaagaccaagatcaagctgtgcatcctgctgcacgctttccggatccgggccgtgaccatcgaccgggtgatgagctacctgaacgcgagcagcggaggcggcagcggtggcggcggctcaggcggcggaggcagcggcggaggcggaagcggcggcggcagcctgcaggtggtgatcagcgccatcctggctctggtggtgctgaccatcatctccctgatcatcctgatcggcggaggtggcagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 55 - RNA, 2333 nt, synthetic construct
  source 1..2333: mol_type = other RNA
aggtcttctggtccccacagactcagagagaacccgccaccatgtgcccccagaagctgaccatcagctggttcgccatcgtgctgctggtgagccctctgatggccatgtgggagctggagaaggacgtgtacgtggtggaggtggattggacccccgacgcacccggcgagaccgtgaacctgacctgcgatacccctgaggaggacgacatcacctggactagcgatcagcggcacggcgtgatcggcagcggcaagaccctgaccatcaccgtgaaggagttcctggatgccggccagtacacctgccacaagggcggggagaccctgagccacagccacctgctgctgcacaaaaaggagaacggcatctggagcaccgagatcctgaagaacttcaagaacaagacctttctgaagtgcgaggctcccaactacagcggtcggttcacctgcagctggctggtgcagcggaatatggacctgaagtttaacatcaagtcaagcagcagcagccccgacagccgggccgtgacctgtggcatggccagcctgagcgccgagaaggtgaccctggaccagagggattacgagaagtacagcgtgagctgccaggaggacgtgacctgcccaaccgccgaggagaccctgcccatcgagctggccctggaggcccggcagcagaacaagtacgagaactacagcaccagcttcttcatccgggacatcatcaagcctgacccccccaagaacctgcagatgaagcccctgaagaacagccaggtggaggtgagctgggagtaccccgacagctggagcaccccccacagctacttcagcctgaagttcttcgtgcggatccagcggaagaaggagaagatgaaggagaccgaggagggctgcaaccagaagggcgccttcctggtggagaagaccagcaccgaggtgcagtgtaagggcggcaacgtgtgcgtgcaggcccaggaccggtactacaacagctcatgcagcaagtgggcctgcgtgccctgccgggtgaggagcggcggcggcggctccggcggaggaggcagcggaggaggcggcagccgggtgatccccgtgagcggccctgcccggtgcctgagccagagccggaacctgctgaagaccaccgacgacatggtgaagaccgctcgggagaagctgaagcactacagctgcaccgccgaggacatcgaccacgaggacatcacccgggaccagaccagcaccttaaagacctgcctgcccctggagctgcacaagaatgagagctgcctggccacccgggagaccagcagcaccacccggggcagctgcctgcccccccagaagaccagcctgatgatgaccctgtgcctgggcagcatctacgaggacctgaagatgtaccagaccgagttccaggcaatcaacgctgctctgcagaatcacaaccaccagcagatcatcctggacaagggaatgctggtggccatcgacgagctgatgcagagcctgaaccacaacggcgagaccctgcggcagaagccccccgtgggcgaggccgacccctaccgtgtgaagatgaagctgtgcatcctgctgcacgctttcagcacccgggtggtgaccatcaaccgggtgatgggctacctgagcagcgcaagcggtggcggcagcggcggcgggggctccggcggcggtgggagcggcggaggcggttcaggcggcggcagcctgcaggtggtgatcagcgccatcctggccctggtggtgctcaccgtgatcagcctgatcatcctgatcgggggcggcggctcaggcaagcccatcccaaatccactgctgggcctggacagcacctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 56 - RNA, 2333 nt, synthetic construct
  source 1..2333: mol_type = other RNA
aggtcttctggtccccacagactcagagagaacccgccaccatgtgcccccagaagctgaccatcagctggttcgccatcgtgctgctggtgagccctctgatggccatgtgggagctcgagaaggacgtgtacgtggtggaggtggactggacccccgacgcccccggcgagaccgtgaacctgacctgtgacacccccgaggaggacgacatcacctggaccagcgaccagcggcacggcgtgatcggaagcggcaagaccctgaccatcaccgtcaaggagttcctggacgccggccagtacacctgccacaagggcggcgagacactgagccacagccacctgctgttacacaagaaggagaacggcatctggagcaccgagattctgaagaacttcaagaacaagaccttcctgaagtgcgaagcccccaattacagcggccggttcacctgcagctggctggtgcagcggaacatggacctgaagttcaatatcaagtcaagcagcagcagccccgatagccgggccgtgacctgcggcatggccagcctgagcgccgagaaggtgaccctggaccagagggactacgagaagtacagcgtgagctgtcaggaggacgtgacatgcccaaccgccgaggagaccctgcccatcgagctggccctggaggcccggcagcagaataagtacgagaattacagcaccagcttcttcatccgggacatcatcaagcccgacccccctaagaacctgcagatgaagcccctgaagaatagccaggtcgaggtgagctgggagtaccccgatagctggagcaccccccacagctacttcagcctgaagttcttcgtgcggatccagcggaagaaggagaagatgaaggagaccgaggagggctgcaatcagaagggcgcattcctggtggagaagaccagcaccgaggtgcagtgcaagggcggcaacgtgtgcgtgcaggcccaggatcggtactacaacagcagctgcagcaagtgggcctgcgtgccatgccgggtgcggagcggcggcggaggctcaggtggcggcggctcaggcgggggcggcagccgggtgatccccgtgagcggccccgcccggtgccttagccagagccggaatctgctgaagaccaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggcaacccgggagaccagcagcaccacccggggcagctgcctccccccccagaagaccagcctgatgatgaccctgtgcctggggagcatctacgaggacctgaagatgtaccagaccgagttccaggctataaacgccgccctgcagaaccacaaccaccagcagatcatcctggacaagggcatgctggtggccatcgacgagctgatgcagagcctgaaccacaacggcgagactctgcggcagaagccccctgtgggcgaggccgatccctaccgggtgaagatgaagctgtgcatcctgctgcacgccttcagcacccgggtggtgaccatcaacagggtgatgggctacctgagcagcgctagcggcggaggtagcggaggcggcggctccggcggcggcggcagcggtggaggcggcagcggcggcggtagcctgcaagtggtgatcagcgccatcctggccctggtggtgctgaccgtgatcagcctgatcatcctgatcggcggtggcgggagcggcaagcccatccccaacccactgctgggcctggacagcacctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - RNA, 1054 nt, synthetic construct
  source 1..1054: mol_type = other RNA
aggtcttctggtccccacagactcagagagaacccgccacctggagcgggtgcagcccctggaggagaacgtgggcaacgccgcccggccccggttcgagcggaacaagctgctgctggtggccagcgtgatccagggcctgggcctgctgctgtgcttcacctacatctgcctgcacttcagcgccctgcaggtgagccacaggtacccccggatccagagcatcaaggtgcagttcaccgagtacaagaaggagaagggcttcatcctgaccagccagaaggaggacgagatcatgaaggtgcagaacaacagcgtgatcatcaactgcgacggcttctacctgatctcactgaagggctacttcagccaggaggtgaacatcagcctgcactaccaaaaggacgaggagcccctgttccagctgaagaaggtgcggagcgtgaacagcctgatggtggcctcactgacctacaaggacaaggtgtaccttaacgtgaccaccgacaacaccagcctggacgacttccacgtgaacggcggcgagctgatcctgattcaccagaaccctggcgagttctgcgtgctgtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - RNA, 1100 nt, synthetic construct
  source 1..1100: mol_type = other RNA
aggtcttctggtccccacagactcagagagaacccgccaccatggagggcgagggcgtgcagccactggatgagaacctggagaacggcagccggccccggttcaagtggaagaagaccttacgtctggtggtgagcggcatcaagggcgccggcatgctgctgtgcttcatctacgtgtgtctgcagctgagcagcagccccgccaaggacccccctatccagcggctgcggggcgccgtgacccggtgcgaggacggccagctgttcatcagcagctacaagaacgagtaccagaccatggaggtgcagaacaacagcgtggtgatcaagtgcgacggcctgtacatcatctacctgaagggcagcttcttccaggaagtgaagatcgacctacacttcagggaggatcacaaccccatcagcatccccatgctgaacgacggccggcggatcgtgttcaccgtggtggccagcctggccttcaaggacaaggtgtacctgaccgtgaacgcccccgacaccctgtgtgagcacctgcagatcaacgacggcgagctgatcgtggtgcagctgactcccggctactgcgcccccgagggcagctaccacagcaccgtgaaccaggtgcccctttgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - RNA, 1070 nt, synthetic construct
  source 1..1070: mol_type = other RNA
aggtcttctggtccccacagactcagagagaacccgccaccatggccctgaccttcgccctgttagtggcgctgctggtgctgagctgcaagagcagctgcagcgtgggctgcgacctgcctcagacccacagcctgggcagccggcggaccctgatgctgctggcccagatgcggaagatcagcctgttcagctgtctgaaggaccggcacgacttcggcttcccccaggaggaattcggcaaccagttccagaaggccgagaccatccccgtgctgcacgagatgatccagcagatcttcaacctgttcagcaccaaggatagcagcgccgcttgggacgagacccttctggacaagttctacaccgagctgtaccagcagctgaacgacctggaggcctgcgtgatccagggcgtgggcgtgaccgagacccctctgatgaaggaggacagcatcctggccgtgcggaagtactttcagcggatcaccctgtacctgaaggagaagaagtacagcccatgcgcctgggaggtggtgcgggccgagatcatgcggagcttcagcctgagcaccaacctgcaggagagcctgcggagcaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - RNA, 1064 nt, synthetic construct
  source 1..1064: mol_type = other RNA
aggtcttctggtccccacagactcagagagaacccgccaccatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 61 - RNA, 956 nt, synthetic construct
  source 1..956: mol_type = other RNA
aggtcttctggtccccacagactcagagagaacccgccaccatgtaccggatgcagctgctgagctgcatcgccctgagcctggccctggtgaccaacagcgcccccaccagcagcagcaccaagaagacccagctgcaactggagcacctgctgctggatctgcagatgatcctgaacggcatcaacaattacaagaatcccaagctgacccggatgctgaccttcaagttctacatgcccaagaaggctaccgagctgaagcacctgcagtgcctggaggaggagctgaagcccctggaggaggtgctgaacctggcccagagcaagaacttccacttcgacccccgggacgtggtgagcaacatcaacgtgttcgtgctggagctgaagggcagcgagaccaccttcatgtgcgagtacgccgacgagaccgccaccatcgtggagttcctgaaccggtggatcaccttctgccagagcatcatcagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - RNA, 1013 nt, synthetic construct
  source 1..1013: mol_type = other RNA
aggtcttctggtccccacagactcagagagaacccgccaccatgtacagcatgcagctggcaagctgcgtgaccctgaccctggtgctgctagtgaacagcgcccccaccagcagcagcaccagcagctccaccgccgaggcccagcagcaacaacagcaacagcagcagcagcaacagcacctggagcagctcctgatggacctgcaggagctgctgagccggatggagaactaccggaacctgaagctgccccggatgctgaccttcaagttctacctgcccaagcaggccacagagctgaaggacctgcagtgcctggaggacgagctgggccccctgcggcacgtgctggatctgacccagagcaagagcttccagctggaggacgccgagaacttcatcagcaacatccgggtgaccgtggtgaagctgaagggcagcgacaacaccttcgagtgccagttcgacgacgagagcgcaaccgtggtggacttcctgcggcggtggatcgccttctgccagagcatcatcagcaccagcccccagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - RNA, 1226 nt, synthetic construct
  source 1..1226: mol_type = other RNA
aggtcttctggtccccacagactcagagagaacccgccaccatggccccccggcgggcccggggctgccggaccctgggcctgcctgccctgctgctgctgcttctgctgcggccccccgccacccggggcatcacctgtcccccacctatgagcgtggagcacgccgacatctgggtgaagagctacagcctgtacagccgggagcggtacatctgcaacagcggcttcaagcggaaggcaggcaccagcagcctgaccgagtgcgtgctgaacaaggccaccaacgtggcccactggaccacccccagccttaagtgcatccgggaccctgcgctggtccaccagcggcctgccccccccagcaccgtgaccaccgcaggagtgaccccccagcccgagagcctgagccccagcggcaaggagcccgccgccagctcacccagcagcaacaacaccgccgccaccaccgccgccatcgtgcccggcagccagctgatgcctagcaagagccccagcaccggcaccaccgagatcagcagccacgagagcagccacggcacccccagccagaccaccgccaagaattgggagctgaccgccagcgccagccaccagcccccaggcgtgtacccccaggggcacagcgacaccaccgtggccatcagcaccagcaccgtgctgctgtgcggcctgagcgcagtgagcctgctggcctgctacctgaagagccggcagaccccacccctggctagcgtggagatggaggccatggaggccctgcccgtgacctggggaaccagcagccgggacgaggacctggagaactgcagccaccacctgtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 64 - RNA, 1094 nt, synthetic construct
  source 1..1094: mol_type = other RNA
aggtcttctggtccccacagactcagagagaacccgccaccatggagttcggcctgagctgggtgttcctggtggccctgttccggggagtgcagtgtatcacctgccccccccccatgagcgtggagcacgctgacatctgggtgaagagctacagcctgtacagccgggagcggtacatctgcaacagcggcttcaagcggaaggccggcaccagctccctgaccgagtgcgtgctgaacaaggccaccaacgtggcccactggaccacccccagcctgaagtgcatccgggaccccgccctggtgggcggctcaggcggcagcggcggcagcgggggcagcggaggcagcggcggaagcggcggcaactgggtgaacgtgatcagcgacctgaagaagatcgaggacctgatccaaagcatgcacattgacgccaccctgtacaccgagagcgacgtgcaccccagctgcaaggtgaccgcaatgaagtgcttcctgctggagctgcaggtgatcagcctggagagcggcgacgccagcatccacgacaccgtggagaacctgatcatcctggcgaacaacagcctgagcagcaacggcaacgtgaccgagagcggctgtaaggagtgcgaggagctggaggagaagaatatcaaggagttcctgcagagcttcgtgcacatcgtgcagatgttcatcaacaccagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - RNA, 1094 nt, synthetic construct
  source 1..1094: mol_type = other RNA
aggtcttctggtccccacagactcagagagaacccgccaccatggagttcggcctgagctgggtgtttctggtggccctgttccggggcgtgcagtgcaactgggtgaacgtgatcagcgacctgaagaagatcgaggacctgatccagtcaatgcacatcgatgcaaccctgtacaccgagagcgacgtgcaccctagctgtaaggtgaccgcaatgaagtgcttcctgctggagctgcaggtgataagcctggagagcggcgacgccagcatccacgacacagtcgagaatctgatcatcctggctaacaacagcctgagcagcaatggtaatgtgaccgagagcggctgcaaggagtgcgaggagctggaggagaagaacatcaaggagttcctgcagagcttcgtgcacatcgtgcagatgttcatcaacaccagcggcggcagcgggggcagcggcggctcaggtggcagcggaggcagcggcggaagcggcggcatcacctgccctccccccatgagcgtggagcacgcagacatctgggtgaagagctacagcctgtacagccgggagcggtacatctgcaacagcggcttcaagcggaaggccggcaccagcagcctgaccgagtgtgtgctgaacaaggccaccaacgtggcccactggacaacccccagcctgaagtgcatcagggaccccgccctggtgtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - RNA, 1760 nt, synthetic construct
  source 1..1760: mol_type = other RNA
aggtcttctggtccccacagactcagagagaacccgccaccatggctagcccccagctgcggggctacggcgtgcaggccatccctgtgctgctgttactgctgctgctcctgctgctgcccctgcgggtgacccccggcaccacctgccccccccccgtgagcatcgagcacgccgacatccgggtgaagaactacagcgtgaacagccgggagaggtacgtgtgcaatagcggcttcaagcggaaggccggcaccagcaccctgatcgagtgcgtgatcaacaagaacaccaacgtggcccactggaccacccccagcctgaagtgcatccgggaccccagcttagcccactacagccccgtgcccaccgtggtgaccccaaaggtgaccagccagcccgagagccccagccccagcgccaaggagcctgaggccttctccccaaagagcgacaccgccatgaccaccgagaccgccatcatgcccggcagccggctgacccccagccagaccaccagcgccggcaccacaggcaccggcagccacaagagcagccgggcacccagcctggccgccaccatgaccctggagcctaccgctagcaccagcctgcggatcaccgagatcagcccccacagcagcaagatgaccaaggtggccatcagcaccagcgtgctgctggtgggcgccggcgtggtgatggctttcctggcctggtacatcaagagccggcagcccagccagccttgccgggtggaggtggagaccatggagactgtgcccatgaccgtgcgggccagcagcaaggaggacgaggacaccggcgccgggggcagcggcggtagcggaggcagcggcggcagcggcggaagcggcggctccggcggcatgaagatcctgaagccctacatgcggaacaccagcatcagctgctacctgtgcttcctgctgaacagccacttcctgaccgaggccggcatccacgtgttcatcctgggctgcgtgagcgtgggcctgcctaagaccgaggccaactggatcgacgtgcggtacgacctggagaagatcgagagcctgatccagagcatccacatcgacaccaccctgtacaccgacagcgacttccaccccagctgcaaggtgaccgccatgaactgctttctgctggagctgcaggtgatcctgcacgagtacagcaacatgaccctgaacgagaccgtgcggaacgtgctgtacctggccaacagcaccctgagcagcaacaagaacgtggccgagagcggctgcaaggagtgcgaggagctggaggagaagaccttcaccgagttcctgcagagcttcatccggatcgtgcagatgttcatcaacaccagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - RNA, 1097 nt, synthetic construct
  source 1..1097: mol_type = other RNA
aggtcttctggtccccacagactcagagagaacccgccaccatgagcgtgctgacccaggtgctggccctgctgctgctgtggctgaccggcgcccgttgcaccacctgcccccccccagtgagcatcgagcacgccgatatccgggtgaagaactacagcgtgaacagccgggagcggtacgtgtgcaacagcggcttcaagcggaaggccggcacaagcaccctgatcgagtgcgtgatcaacaagaacaccaatgtggcccactggaccacccccagcctgaagtgtatccgggaccctagcctggccggcggcagcggcgggagcggtggcagcggcggaagcggcggtagcggaggcagcggcggcaactggatcgacgtgcggtacgacctagagaagatcgagagcctgatccagagcatccacatcgacaccaccctgtacaccgacagcgacttccaccccagctgcaaggtgaccgccatgaactgcttcctgctggagctgcaggtgatcctgcacgagtacagcaacatgacactgaacgagaccgtgcggaacgtgctgtacctggcaaacagcaccctgagcagcaacaagaatgtggccgagagcggctgcaaggaatgcgaggagctggaggagaagaccttcaccgagttcctccagagcttcatccggatcgtgcagatgttcatcaacaccagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - RNA, 1097 nt, synthetic construct
  source 1..1097: mol_type = other RNA
aggtcttctggtccccacagactcagagagaacccgccaccatgagcgtgctgacccaggtgctggctctactgctgctgtggttaacaggcgcccggtgcaactggatcgacgtgcggtacgacctggagaagatcgagagcctgatccagagcatccacatcgacaccaccctgtacaccgacagcgacttccaccctagctgcaaggtgaccgccatgaactgcttcctgctggagctgcaggtgatcctgcacgagtacagcaatatgaccctgaacgagaccgtgcggaacgtgctgtacctggccaacagcaccctgagcagcaataagaatgtggccgagagcggctgcaaggagtgtgaggagctggaggagaagaccttcactgagttcctgcagagctttatccggatcgtgcagatgttcatcaacactagcggcggaagcggcggctcaggcggcagcggaggcagcggcggcagcgggggcagcggcggaaccacctgtcccccccccgtgagcatcgagcacgccgacatccgggtgaagaactacagcgtgaactcacgggagcggtacgtgtgcaactcaggcttcaagcggaaggccggcaccagcaccctgatcgagtgcgtgatcaacaagaacacaaacgtggcccactggaccacccccagcctgaagtgcatccgggaccccagcctggcctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - RNA, 1667 nt, synthetic construct
  source 1..1667: mol_type = other RNA
aggtcttctggtccccacagactcagagagaacccgccaccatgagcgtgctgacccaggtgctggccctgctgctgctgtggctgaccggcgcccggtgcaactggatcgacgtgcggtacgatctggagaagatcgagagcctgatccagagcatccacatcgacaccaccctgtacaccgatagcgacttccaccccagctgcaaggtgaccgccatgaactgcttcctgctggaactgcaggtgatcctgcacgagtacagcaacatgaccctgaacgagaccgtgcggaacgtcctgtacctggccaacagcacactgagcagcaacaagaacgtggccgagagcggctgcaaggagtgcgaggagctggaggagaagaccttcaccgagttcctgcagagcttcatccgtatcgtgcagatgttcatcaacaccagcgggggcagcggcggaagcgggggcagcggcggaagcggcggcagcggaggcagcggaggcaccacatgccccccacccgtgagcatcgagcacgccgacatacgggtgaagaattacagcgtgaacagccgggagcggtacgtgtgcaacagcggcttcaagcggaaggccggcaccagcaccctgatcgagtgcgtgatcaacaagaacactaacgtggcacactggaccacccccagcctcaagtgcatccgggacccaagcctggccgggggcggcggctccggcggaggcggcagcggcggtggaggcagcggccctacaatcaagccctgccccccctgcaagtgccccgcccccaatgccgccggcggccccagcgtgttcatcttcccccccaagatcaaggacgtgctgatgatctcactaagccccatagtgacctgcgtggtggtggacgtgagcgaggacgaccctgacgtgcagatcagctggttcgtgaacaacgtggaggtgcacaccgcccagacccagacccaccgggaggactacaacagcaccctgcgggtggtgagcgccctgcccatccagcaccaggactggatgagcggcaaggagttcaagtgtaaggtgaataataaggacctgggcgccccaatagagcggaccattagcaagcccaaggggagcgtgcgggccccccaggtgtacgtgctgccaccccctgaggaggagatgaccaaaaagcaggtgaccctgacctgcatggtgaccgatttcatgccagaggacatctacgtggagtggaccaacaacggcaagaccgagctgaactacaagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - RNA, 2201 nt, synthetic construct
  source 1..2201: mol_type = other RNA
aggtcttctggtccccacagactcagagagaacccgccaccatgtgccaccagcagctggtgatcagctggttcagcctggtgttcctcgccagccccctggtggctatctgggagctgaagaaggacgtgtacgtggtggagctggactggtaccccgacgcccccggcgagatggtggtgctcacctgcgatacccccgaggaggacggaatcacctggaccctggaccagagcagcgaggtgttaggcagcggcaagaccctgaccatccaggtgaaggagttcggcgacgccggccagtacacctgccacaagggcggggaggtgctgagccacagcctgctgctgctgcacaagaaggaggacggcatctggagcaccgacatcctgaaggaccagaaggagcccaagaacaagaccttcctgcggtgcgaggccaagaattacagcggccggttcacctgctggtggctgaccaccatcagcaccgacctgaccttcagcgtgaagagcagccggggcagcagcgacccccagggcgtgacctgcggcgccgccaccctgagcgccgagcgggtgaggggcgacaataaggagtacgagtacagcgtggagtgtcaggaggatagcgcatgccccgccgccgaggagagcctgcccatcgaggtgatggtggacgccgtgcacaagctgaagtacgagaactacaccagcagcttcttcatccgggacatcatcaagcccgacccccccaagaacctgcagctgaagcccctgaagaacagccggcaggtggaggtgtcatgggagtaccccgacacctggagcaccccccacagctacttcagcctgacattctgtgtgcaggtgcagggcaagagcaagcgggagaagaaggaccgggtgttcaccgacaagaccagcgccaccgtgatctgccggaagaatgccagcatcagcgtgcgggcccaggaccggtactacagcagcagctggagcgagtgggccagcgtgccttgctccggcggcggaggaagcggaggcgggggcagcgggggcggcggaagccggaacctgcccgtggccaccccagaccccggcatgttcccctgcctgcaccacagccagaacctgctgcgggccgtgagcaacatgctgcagaaggctcggcagaccctggagttctacccctgcaccagcgaggagatcgaccacgaggacatcaccaaggacaagactagcaccgtggaggcctgcctgcccctggagctgaccaagaatgagagctgcctgaacagccgggagaccagctttatcaccaacggcagctgcctggccagccggaagacctcattcatgatggccctgtgcctgagcagcatctacgaggacctgaagatgtaccaggtggagttcaagaccatgaacgccaagctgctgatggaccctaagcggcaaatcttcctggaccagaatatgctggccgtgatcgacgagctcatgcaggccctgaactttaacagcgagaccgtgccccagaagagcagcctggaggagccagacttctacaagaccaagatcaagctgtgcatcctgctgcacgctttccggatccgggccgtgaccatcgaccgggtgatgagctacctgaacgcgagcagcggaggcggcagcggtggcggcggctcaggcggcggaggcagcggcggaggcggaagcggcggcggcagcctgcaggtggtgatcagcgccatcctggctctggtggtgctgaccatcatctccctgatcatcctgatcggcggaggtggcagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - RNA, 2252 nt, synthetic construct
  source 1..2252: mol_type = other RNA
aggtcttctggtccccacagactcagagagaacccgccaccatgtgcccccagaagctgaccatcagctggttcgccatcgtgctgctggtgagccctctgatggccatgtgggagctggagaaggacgtgtacgtggtggaggtggattggacccccgacgcacccggcgagaccgtgaacctgacctgcgatacccctgaggaggacgacatcacctggactagcgatcagcggcacggcgtgatcggcagcggcaagaccctgaccatcaccgtgaaggagttcctggatgccggccagtacacctgccacaagggcggggagaccctgagccacagccacctgctgctgcacaaaaaggagaacggcatctggagcaccgagatcctgaagaacttcaagaacaagacctttctgaagtgcgaggctcccaactacagcggtcggttcacctgcagctggctggtgcagcggaatatggacctgaagtttaacatcaagtcaagcagcagcagccccgacagccgggccgtgacctgtggcatggccagcctgagcgccgagaaggtgaccctggaccagagggattacgagaagtacagcgtgagctgccaggaggacgtgacctgcccaaccgccgaggagaccctgcccatcgagctggccctggaggcccggcagcagaacaagtacgagaactacagcaccagcttcttcatccgggacatcatcaagcctgacccccccaagaacctgcagatgaagcccctgaagaacagccaggtggaggtgagctgggagtaccccgacagctggagcaccccccacagctacttcagcctgaagttcttcgtgcggatccagcggaagaaggagaagatgaaggagaccgaggagggctgcaaccagaagggcgccttcctggtggagaagaccagcaccgaggtgcagtgtaagggcggcaacgtgtgcgtgcaggcccaggaccggtactacaacagctcatgcagcaagtgggcctgcgtgccctgccgggtgaggagcggcggcggcggctccggcggaggaggcagcggaggaggcggcagccgggtgatccccgtgagcggccctgcccggtgcctgagccagagccggaacctgctgaagaccaccgacgacatggtgaagaccgctcgggagaagctgaagcactacagctgcaccgccgaggacatcgaccacgaggacatcacccgggaccagaccagcaccttaaagacctgcctgcccctggagctgcacaagaatgagagctgcctggccacccgggagaccagcagcaccacccggggcagctgcctgcccccccagaagaccagcctgatgatgaccctgtgcctgggcagcatctacgaggacctgaagatgtaccagaccgagttccaggcaatcaacgctgctctgcagaatcacaaccaccagcagatcatcctggacaagggaatgctggtggccatcgacgagctgatgcagagcctgaaccacaacggcgagaccctgcggcagaagccccccgtgggcgaggccgacccctaccgtgtgaagatgaagctgtgcatcctgctgcacgctttcagcacccgggtggtgaccatcaaccgggtgatgggctacctgagcagcgcaagcggtggcggcagcggcggcgggggctccggcggcggtgggagcggcggaggcggttcaggcggcggcagcctgcaggtggtgatcagcgccatcctggccctggtggtgctcaccgtgatcagcctgatcatcctgatcgggggcggcggctcaggcaagcccatcccaaatccactgctgggcctggacagcacctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 72 - RNA, 2252 nt, synthetic construct
  source 1..2252: mol_type = other RNA
aggtcttctggtccccacagactcagagagaacccgccaccatgtgcccccagaagctgaccatcagctggttcgccatcgtgctgctggtgagccctctgatggccatgtgggagctcgagaaggacgtgtacgtggtggaggtggactggacccccgacgcccccggcgagaccgtgaacctgacctgtgacacccccgaggaggacgacatcacctggaccagcgaccagcggcacggcgtgatcggaagcggcaagaccctgaccatcaccgtcaaggagttcctggacgccggccagtacacctgccacaagggcggcgagacactgagccacagccacctgctgttacacaagaaggagaacggcatctggagcaccgagattctgaagaacttcaagaacaagaccttcctgaagtgcgaagcccccaattacagcggccggttcacctgcagctggctggtgcagcggaacatggacctgaagttcaatatcaagtcaagcagcagcagccccgatagccgggccgtgacctgcggcatggccagcctgagcgccgagaaggtgaccctggaccagagggactacgagaagtacagcgtgagctgtcaggaggacgtgacatgcccaaccgccgaggagaccctgcccatcgagctggccctggaggcccggcagcagaataagtacgagaattacagcaccagcttcttcatccgggacatcatcaagcccgacccccctaagaacctgcagatgaagcccctgaagaatagccaggtcgaggtgagctgggagtaccccgatagctggagcaccccccacagctacttcagcctgaagttcttcgtgcggatccagcggaagaaggagaagatgaaggagaccgaggagggctgcaatcagaagggcgcattcctggtggagaagaccagcaccgaggtgcagtgcaagggcggcaacgtgtgcgtgcaggcccaggatcggtactacaacagcagctgcagcaagtgggcctgcgtgccatgccgggtgcggagcggcggcggaggctcaggtggcggcggctcaggcgggggcggcagccgggtgatccccgtgagcggccccgcccggtgccttagccagagccggaatctgctgaagaccaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggcaacccgggagaccagcagcaccacccggggcagctgcctccccccccagaagaccagcctgatgatgaccctgtgcctggggagcatctacgaggacctgaagatgtaccagaccgagttccaggctataaacgccgccctgcagaaccacaaccaccagcagatcatcctggacaagggcatgctggtggccatcgacgagctgatgcagagcctgaaccacaacggcgagactctgcggcagaagccccctgtgggcgaggccgatccctaccgggtgaagatgaagctgtgcatcctgctgcacgccttcagcacccgggtggtgaccatcaacagggtgatgggctacctgagcagcgctagcggcggaggtagcggaggcggcggctccggcggcggcggcagcggtggaggcggcagcggcggcggtagcctgcaagtggtgatcagcgccatcctggccctggtggtgctgaccgtgatcagcctgatcatcctgatcggcggtggcgggagcggcaagcccatccccaacccactgctgggcctggacagcacctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 73 - RNA, 973 nt, synthetic construct
  source 1..973: mol_type = other RNA
aggtcttctggtccccacagactcagagagaacccgccacctggagcgggtgcagcccctggaggagaacgtgggcaacgccgcccggccccggttcgagcggaacaagctgctgctggtggccagcgtgatccagggcctgggcctgctgctgtgcttcacctacatctgcctgcacttcagcgccctgcaggtgagccacaggtacccccggatccagagcatcaaggtgcagttcaccgagtacaagaaggagaagggcttcatcctgaccagccagaaggaggacgagatcatgaaggtgcagaacaacagcgtgatcatcaactgcgacggcttctacctgatctcactgaagggctacttcagccaggaggtgaacatcagcctgcactaccaaaaggacgaggagcccctgttccagctgaagaaggtgcggagcgtgaacagcctgatggtggcctcactgacctacaaggacaaggtgtaccttaacgtgaccaccgacaacaccagcctggacgacttccacgtgaacggcggcgagctgatcctgattcaccagaaccctggcgagttctgcgtgctgtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 74 - RNA, 1019 nt, synthetic construct
  source 1..1019: mol_type = other RNA
aggtcttctggtccccacagactcagagagaacccgccaccatggagggcgagggcgtgcagccactggatgagaacctggagaacggcagccggccccggttcaagtggaagaagaccttacgtctggtggtgagcggcatcaagggcgccggcatgctgctgtgcttcatctacgtgtgtctgcagctgagcagcagccccgccaaggacccccctatccagcggctgcggggcgccgtgacccggtgcgaggacggccagctgttcatcagcagctacaagaacgagtaccagaccatggaggtgcagaacaacagcgtggtgatcaagtgcgacggcctgtacatcatctacctgaagggcagcttcttccaggaagtgaagatcgacctacacttcagggaggatcacaaccccatcagcatccccatgctgaacgacggccggcggatcgtgttcaccgtggtggccagcctggccttcaaggacaaggtgtacctgaccgtgaacgcccccgacaccctgtgtgagcacctgcagatcaacgacggcgagctgatcgtggtgcagctgactcccggctactgcgcccccgagggcagctaccacagcaccgtgaaccaggtgcccctttgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 75 - RNA, 989 nt, synthetic construct
  source 1..989: mol_type = other RNA
aggtcttctggtccccacagactcagagagaacccgccaccatggccctgaccttcgccctgttagtggcgctgctggtgctgagctgcaagagcagctgcagcgtgggctgcgacctgcctcagacccacagcctgggcagccggcggaccctgatgctgctggcccagatgcggaagatcagcctgttcagctgtctgaaggaccggcacgacttcggcttcccccaggaggaattcggcaaccagttccagaaggccgagaccatccccgtgctgcacgagatgatccagcagatcttcaacctgttcagcaccaaggatagcagcgccgcttgggacgagacccttctggacaagttctacaccgagctgtaccagcagctgaacgacctggaggcctgcgtgatccagggcgtgggcgtgaccgagacccctctgatgaaggaggacagcatcctggccgtgcggaagtactttcagcggatcaccctgtacctgaaggagaagaagtacagcccatgcgcctgggaggtggtgcgggccgagatcatgcggagcttcagcctgagcaccaacctgcaggagagcctgcggagcaaggagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 76 - RNA, 983 nt, synthetic construct
  source 1..983: mol_type = other RNA
aggtcttctggtccccacagactcagagagaacccgccaccatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 77 - RNA, 875 nt, synthetic construct
  source 1..875: mol_type = other RNA
aggtcttctggtccccacagactcagagagaacccgccaccatgtaccggatgcagctgctgagctgcatcgccctgagcctggccctggtgaccaacagcgcccccaccagcagcagcaccaagaagacccagctgcaactggagcacctgctgctggatctgcagatgatcctgaacggcatcaacaattacaagaatcccaagctgacccggatgctgaccttcaagttctacatgcccaagaaggctaccgagctgaagcacctgcagtgcctggaggaggagctgaagcccctggaggaggtgctgaacctggcccagagcaagaacttccacttcgacccccgggacgtggtgagcaacatcaacgtgttcgtgctggagctgaagggcagcgagaccaccttcatgtgcgagtacgccgacgagaccgccaccatcgtggagttcctgaaccggtggatcaccttctgccagagcatcatcagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 78 - RNA, 932 nt, synthetic construct
  source 1..932: mol_type = other RNA
aggtcttctggtccccacagactcagagagaacccgccaccatgtacagcatgcagctggcaagctgcgtgaccctgaccctggtgctgctagtgaacagcgcccccaccagcagcagcaccagcagctccaccgccgaggcccagcagcaacaacagcaacagcagcagcagcaacagcacctggagcagctcctgatggacctgcaggagctgctgagccggatggagaactaccggaacctgaagctgccccggatgctgaccttcaagttctacctgcccaagcaggccacagagctgaaggacctgcagtgcctggaggacgagctgggccccctgcggcacgtgctggatctgacccagagcaagagcttccagctggaggacgccgagaacttcatcagcaacatccgggtgaccgtggtgaagctgaagggcagcgacaacaccttcgagtgccagttcgacgacgagagcgcaaccgtggtggacttcctgcggcggtggatcgccttctgccagagcatcatcagcaccagcccccagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 79 - RNA, 1616 nt, synthetic construct
  source 1..1616: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggccccccggcgggcccggggctgccggaccctgggcctgcctgccctgctgctgctgcttctgctgcggccccccgccacccggggcatcacctgtcccccacctatgagcgtggagcacgccgacatctgggtgaagagctacagcctgtacagccgggagcggtacatctgcaacagcggcttcaagcggaaggcaggcaccagcagcctgaccgagtgcgtgctgaacaaggccaccaacgtggcccactggaccacccccagccttaagtgcatccgggaccctgcgctggtccaccagcggcctgccccccccagcaccgtgaccaccgcaggagtgaccccccagcccgagagcctgagccccagcggcaaggagcccgccgccagctcacccagcagcaacaacaccgccgccaccaccgccgccatcgtgcccggcagccagctgatgcctagcaagagccccagcaccggcaccaccgagatcagcagccacgagagcagccacggcacccccagccagaccaccgccaagaattgggagctgaccgccagcgccagccaccagcccccaggcgtgtacccccaggggcacagcgacaccaccgtggccatcagcaccagcaccgtgctgctgtgcggcctgagcgcagtgagcctgctggcctgctacctgaagagccggcagaccccacccctggctagcgtggagatggaggccatggaggccctgcccgtgacctggggaaccagcagccgggacgaggacctggagaactgcagccaccacctgtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 80 - RNA, 1484 nt, synthetic construct
  source 1..1484: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggagttcggcctgagctgggtgttcctggtggccctgttccggggagtgcagtgtatcacctgccccccccccatgagcgtggagcacgctgacatctgggtgaagagctacagcctgtacagccgggagcggtacatctgcaacagcggcttcaagcggaaggccggcaccagctccctgaccgagtgcgtgctgaacaaggccaccaacgtggcccactggaccacccccagcctgaagtgcatccgggaccccgccctggtgggcggctcaggcggcagcggcggcagcgggggcagcggaggcagcggcggaagcggcggcaactgggtgaacgtgatcagcgacctgaagaagatcgaggacctgatccaaagcatgcacattgacgccaccctgtacaccgagagcgacgtgcaccccagctgcaaggtgaccgcaatgaagtgcttcctgctggagctgcaggtgatcagcctggagagcggcgacgccagcatccacgacaccgtggagaacctgatcatcctggcgaacaacagcctgagcagcaacggcaacgtgaccgagagcggctgtaaggagtgcgaggagctggaggagaagaatatcaaggagttcctgcagagcttcgtgcacatcgtgcagatgttcatcaacaccagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 81 - RNA, 1484 nt, synthetic construct
  source 1..1484: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggagttcggcctgagctgggtgtttctggtggccctgttccggggcgtgcagtgcaactgggtgaacgtgatcagcgacctgaagaagatcgaggacctgatccagtcaatgcacatcgatgcaaccctgtacaccgagagcgacgtgcaccctagctgtaaggtgaccgcaatgaagtgcttcctgctggagctgcaggtgataagcctggagagcggcgacgccagcatccacgacacagtcgagaatctgatcatcctggctaacaacagcctgagcagcaatggtaatgtgaccgagagcggctgcaaggagtgcgaggagctggaggagaagaacatcaaggagttcctgcagagcttcgtgcacatcgtgcagatgttcatcaacaccagcggcggcagcgggggcagcggcggctcaggtggcagcggaggcagcggcggaagcggcggcatcacctgccctccccccatgagcgtggagcacgcagacatctgggtgaagagctacagcctgtacagccgggagcggtacatctgcaacagcggcttcaagcggaaggccggcaccagcagcctgaccgagtgtgtgctgaacaaggccaccaacgtggcccactggacaacccccagcctgaagtgcatcagggaccccgccctggtgtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 82 - RNA, 2150 nt, synthetic construct
  source 1..2150: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggctagcccccagctgcggggctacggcgtgcaggccatccctgtgctgctgttactgctgctgctcctgctgctgcccctgcgggtgacccccggcaccacctgccccccccccgtgagcatcgagcacgccgacatccgggtgaagaactacagcgtgaacagccgggagaggtacgtgtgcaatagcggcttcaagcggaaggccggcaccagcaccctgatcgagtgcgtgatcaacaagaacaccaacgtggcccactggaccacccccagcctgaagtgcatccgggaccccagcttagcccactacagccccgtgcccaccgtggtgaccccaaaggtgaccagccagcccgagagccccagccccagcgccaaggagcctgaggccttctccccaaagagcgacaccgccatgaccaccgagaccgccatcatgcccggcagccggctgacccccagccagaccaccagcgccggcaccacaggcaccggcagccacaagagcagccgggcacccagcctggccgccaccatgaccctggagcctaccgctagcaccagcctgcggatcaccgagatcagcccccacagcagcaagatgaccaaggtggccatcagcaccagcgtgctgctggtgggcgccggcgtggtgatggctttcctggcctggtacatcaagagccggcagcccagccagccttgccgggtggaggtggagaccatggagactgtgcccatgaccgtgcgggccagcagcaaggaggacgaggacaccggcgccgggggcagcggcggtagcggaggcagcggcggcagcggcggaagcggcggctccggcggcatgaagatcctgaagccctacatgcggaacaccagcatcagctgctacctgtgcttcctgctgaacagccacttcctgaccgaggccggcatccacgtgttcatcctgggctgcgtgagcgtgggcctgcctaagaccgaggccaactggatcgacgtgcggtacgacctggagaagatcgagagcctgatccagagcatccacatcgacaccaccctgtacaccgacagcgacttccaccccagctgcaaggtgaccgccatgaactgctttctgctggagctgcaggtgatcctgcacgagtacagcaacatgaccctgaacgagaccgtgcggaacgtgctgtacctggccaacagcaccctgagcagcaacaagaacgtggccgagagcggctgcaaggagtgcgaggagctggaggagaagaccttcaccgagttcctgcagagcttcatccggatcgtgcagatgttcatcaacaccagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 83 - RNA, 1487 nt, synthetic construct
  source 1..1487: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgagcgtgctgacccaggtgctggccctgctgctgctgtggctgaccggcgcccgttgcaccacctgcccccccccagtgagcatcgagcacgccgatatccgggtgaagaactacagcgtgaacagccgggagcggtacgtgtgcaacagcggcttcaagcggaaggccggcacaagcaccctgatcgagtgcgtgatcaacaagaacaccaatgtggcccactggaccacccccagcctgaagtgtatccgggaccctagcctggccggcggcagcggcgggagcggtggcagcggcggaagcggcggtagcggaggcagcggcggcaactggatcgacgtgcggtacgacctagagaagatcgagagcctgatccagagcatccacatcgacaccaccctgtacaccgacagcgacttccaccccagctgcaaggtgaccgccatgaactgcttcctgctggagctgcaggtgatcctgcacgagtacagcaacatgacactgaacgagaccgtgcggaacgtgctgtacctggcaaacagcaccctgagcagcaacaagaatgtggccgagagcggctgcaaggaatgcgaggagctggaggagaagaccttcaccgagttcctccagagcttcatccggatcgtgcagatgttcatcaacaccagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - RNA, 1487 nt, synthetic construct
  source 1..1487: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgagcgtgctgacccaggtgctggctctactgctgctgtggttaacaggcgcccggtgcaactggatcgacgtgcggtacgacctggagaagatcgagagcctgatccagagcatccacatcgacaccaccctgtacaccgacagcgacttccaccctagctgcaaggtgaccgccatgaactgcttcctgctggagctgcaggtgatcctgcacgagtacagcaatatgaccctgaacgagaccgtgcggaacgtgctgtacctggccaacagcaccctgagcagcaataagaatgtggccgagagcggctgcaaggagtgtgaggagctggaggagaagaccttcactgagttcctgcagagctttatccggatcgtgcagatgttcatcaacactagcggcggaagcggcggctcaggcggcagcggaggcagcggcggcagcgggggcagcggcggaaccacctgtcccccccccgtgagcatcgagcacgccgacatccgggtgaagaactacagcgtgaactcacgggagcggtacgtgtgcaactcaggcttcaagcggaaggccggcaccagcaccctgatcgagtgcgtgatcaacaagaacacaaacgtggcccactggaccacccccagcctgaagtgcatccgggaccccagcctggcctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 85 - RNA, 2057 nt, synthetic construct
  source 1..2057: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgagcgtgctgacccaggtgctggccctgctgctgctgtggctgaccggcgcccggtgcaactggatcgacgtgcggtacgatctggagaagatcgagagcctgatccagagcatccacatcgacaccaccctgtacaccgatagcgacttccaccccagctgcaaggtgaccgccatgaactgcttcctgctggaactgcaggtgatcctgcacgagtacagcaacatgaccctgaacgagaccgtgcggaacgtcctgtacctggccaacagcacactgagcagcaacaagaacgtggccgagagcggctgcaaggagtgcgaggagctggaggagaagaccttcaccgagttcctgcagagcttcatccgtatcgtgcagatgttcatcaacaccagcgggggcagcggcggaagcgggggcagcggcggaagcggcggcagcggaggcagcggaggcaccacatgccccccacccgtgagcatcgagcacgccgacatacgggtgaagaattacagcgtgaacagccgggagcggtacgtgtgcaacagcggcttcaagcggaaggccggcaccagcaccctgatcgagtgcgtgatcaacaagaacactaacgtggcacactggaccacccccagcctcaagtgcatccgggacccaagcctggccgggggcggcggctccggcggaggcggcagcggcggtggaggcagcggccctacaatcaagccctgccccccctgcaagtgccccgcccccaatgccgccggcggccccagcgtgttcatcttcccccccaagatcaaggacgtgctgatgatctcactaagccccatagtgacctgcgtggtggtggacgtgagcgaggacgaccctgacgtgcagatcagctggttcgtgaacaacgtggaggtgcacaccgcccagacccagacccaccgggaggactacaacagcaccctgcgggtggtgagcgccctgcccatccagcaccaggactggatgagcggcaaggagttcaagtgtaaggtgaataataaggacctgggcgccccaatagagcggaccattagcaagcccaaggggagcgtgcgggccccccaggtgtacgtgctgccaccccctgaggaggagatgaccaaaaagcaggtgaccctgacctgcatggtgaccgatttcatgccagaggacatctacgtggagtggaccaacaacggcaagaccgagctgaactacaagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 86 - RNA, 2591 nt, synthetic construct
  source 1..2591: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtgccaccagcagctggtgatcagctggttcagcctggtgttcctcgccagccccctggtggctatctgggagctgaagaaggacgtgtacgtggtggagctggactggtaccccgacgcccccggcgagatggtggtgctcacctgcgatacccccgaggaggacggaatcacctggaccctggaccagagcagcgaggtgttaggcagcggcaagaccctgaccatccaggtgaaggagttcggcgacgccggccagtacacctgccacaagggcggggaggtgctgagccacagcctgctgctgctgcacaagaaggaggacggcatctggagcaccgacatcctgaaggaccagaaggagcccaagaacaagaccttcctgcggtgcgaggccaagaattacagcggccggttcacctgctggtggctgaccaccatcagcaccgacctgaccttcagcgtgaagagcagccggggcagcagcgacccccagggcgtgacctgcggcgccgccaccctgagcgccgagcgggtgaggggcgacaataaggagtacgagtacagcgtggagtgtcaggaggatagcgcatgccccgccgccgaggagagcctgcccatcgaggtgatggtggacgccgtgcacaagctgaagtacgagaactacaccagcagcttcttcatccgggacatcatcaagcccgacccccccaagaacctgcagctgaagcccctgaagaacagccggcaggtggaggtgtcatgggagtaccccgacacctggagcaccccccacagctacttcagcctgacattctgtgtgcaggtgcagggcaagagcaagcgggagaagaaggaccgggtgttcaccgacaagaccagcgccaccgtgatctgccggaagaatgccagcatcagcgtgcgggcccaggaccggtactacagcagcagctggagcgagtgggccagcgtgccttgctccggcggcggaggaagcggaggcgggggcagcgggggcggcggaagccggaacctgcccgtggccaccccagaccccggcatgttcccctgcctgcaccacagccagaacctgctgcgggccgtgagcaacatgctgcagaaggctcggcagaccctggagttctacccctgcaccagcgaggagatcgaccacgaggacatcaccaaggacaagactagcaccgtggaggcctgcctgcccctggagctgaccaagaatgagagctgcctgaacagccgggagaccagctttatcaccaacggcagctgcctggccagccggaagacctcattcatgatggccctgtgcctgagcagcatctacgaggacctgaagatgtaccaggtggagttcaagaccatgaacgccaagctgctgatggaccctaagcggcaaatcttcctggaccagaatatgctggccgtgatcgacgagctcatgcaggccctgaactttaacagcgagaccgtgccccagaagagcagcctggaggagccagacttctacaagaccaagatcaagctgtgcatcctgctgcacgctttccggatccgggccgtgaccatcgaccgggtgatgagctacctgaacgcgagcagcggaggcggcagcggtggcggcggctcaggcggcggaggcagcggcggaggcggaagcggcggcggcagcctgcaggtggtgatcagcgccatcctggctctggtggtgctgaccatcatctccctgatcatcctgatcggcggaggtggcagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 87 - RNA, 2642 nt, synthetic construct
  source 1..2642: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtgcccccagaagctgaccatcagctggttcgccatcgtgctgctggtgagccctctgatggccatgtgggagctggagaaggacgtgtacgtggtggaggtggattggacccccgacgcacccggcgagaccgtgaacctgacctgcgatacccctgaggaggacgacatcacctggactagcgatcagcggcacggcgtgatcggcagcggcaagaccctgaccatcaccgtgaaggagttcctggatgccggccagtacacctgccacaagggcggggagaccctgagccacagccacctgctgctgcacaaaaaggagaacggcatctggagcaccgagatcctgaagaacttcaagaacaagacctttctgaagtgcgaggctcccaactacagcggtcggttcacctgcagctggctggtgcagcggaatatggacctgaagtttaacatcaagtcaagcagcagcagccccgacagccgggccgtgacctgtggcatggccagcctgagcgccgagaaggtgaccctggaccagagggattacgagaagtacagcgtgagctgccaggaggacgtgacctgcccaaccgccgaggagaccctgcccatcgagctggccctggaggcccggcagcagaacaagtacgagaactacagcaccagcttcttcatccgggacatcatcaagcctgacccccccaagaacctgcagatgaagcccctgaagaacagccaggtggaggtgagctgggagtaccccgacagctggagcaccccccacagctacttcagcctgaagttcttcgtgcggatccagcggaagaaggagaagatgaaggagaccgaggagggctgcaaccagaagggcgccttcctggtggagaagaccagcaccgaggtgcagtgtaagggcggcaacgtgtgcgtgcaggcccaggaccggtactacaacagctcatgcagcaagtgggcctgcgtgccctgccgggtgaggagcggcggcggcggctccggcggaggaggcagcggaggaggcggcagccgggtgatccccgtgagcggccctgcccggtgcctgagccagagccggaacctgctgaagaccaccgacgacatggtgaagaccgctcgggagaagctgaagcactacagctgcaccgccgaggacatcgaccacgaggacatcacccgggaccagaccagcaccttaaagacctgcctgcccctggagctgcacaagaatgagagctgcctggccacccgggagaccagcagcaccacccggggcagctgcctgcccccccagaagaccagcctgatgatgaccctgtgcctgggcagcatctacgaggacctgaagatgtaccagaccgagttccaggcaatcaacgctgctctgcagaatcacaaccaccagcagatcatcctggacaagggaatgctggtggccatcgacgagctgatgcagagcctgaaccacaacggcgagaccctgcggcagaagccccccgtgggcgaggccgacccctaccgtgtgaagatgaagctgtgcatcctgctgcacgctttcagcacccgggtggtgaccatcaaccgggtgatgggctacctgagcagcgcaagcggtggcggcagcggcggcgggggctccggcggcggtgggagcggcggaggcggttcaggcggcggcagcctgcaggtggtgatcagcgccatcctggccctggtggtgctcaccgtgatcagcctgatcatcctgatcgggggcggcggctcaggcaagcccatcccaaatccactgctgggcctggacagcacctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 88 - RNA, 2642 nt, synthetic construct
  source 1..2642: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtgcccccagaagctgaccatcagctggttcgccatcgtgctgctggtgagccctctgatggccatgtgggagctcgagaaggacgtgtacgtggtggaggtggactggacccccgacgcccccggcgagaccgtgaacctgacctgtgacacccccgaggaggacgacatcacctggaccagcgaccagcggcacggcgtgatcggaagcggcaagaccctgaccatcaccgtcaaggagttcctggacgccggccagtacacctgccacaagggcggcgagacactgagccacagccacctgctgttacacaagaaggagaacggcatctggagcaccgagattctgaagaacttcaagaacaagaccttcctgaagtgcgaagcccccaattacagcggccggttcacctgcagctggctggtgcagcggaacatggacctgaagttcaatatcaagtcaagcagcagcagccccgatagccgggccgtgacctgcggcatggccagcctgagcgccgagaaggtgaccctggaccagagggactacgagaagtacagcgtgagctgtcaggaggacgtgacatgcccaaccgccgaggagaccctgcccatcgagctggccctggaggcccggcagcagaataagtacgagaattacagcaccagcttcttcatccgggacatcatcaagcccgacccccctaagaacctgcagatgaagcccctgaagaatagccaggtcgaggtgagctgggagtaccccgatagctggagcaccccccacagctacttcagcctgaagttcttcgtgcggatccagcggaagaaggagaagatgaaggagaccgaggagggctgcaatcagaagggcgcattcctggtggagaagaccagcaccgaggtgcagtgcaagggcggcaacgtgtgcgtgcaggcccaggatcggtactacaacagcagctgcagcaagtgggcctgcgtgccatgccgggtgcggagcggcggcggaggctcaggtggcggcggctcaggcgggggcggcagccgggtgatccccgtgagcggccccgcccggtgccttagccagagccggaatctgctgaagaccaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggcaacccgggagaccagcagcaccacccggggcagctgcctccccccccagaagaccagcctgatgatgaccctgtgcctggggagcatctacgaggacctgaagatgtaccagaccgagttccaggctataaacgccgccctgcagaaccacaaccaccagcagatcatcctggacaagggcatgctggtggccatcgacgagctgatgcagagcctgaaccacaacggcgagactctgcggcagaagccccctgtgggcgaggccgatccctaccgggtgaagatgaagctgtgcatcctgctgcacgccttcagcacccgggtggtgaccatcaacagggtgatgggctacctgagcagcgctagcggcggaggtagcggaggcggcggctccggcggcggcggcagcggtggaggcggcagcggcggcggtagcctgcaagtggtgatcagcgccatcctggccctggtggtgctgaccgtgatcagcctgatcatcctgatcggcggtggcgggagcggcaagcccatccccaacccactgctgggcctggacagcacctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 89 - RNA, 1363 nt, synthetic construct
  source 1..1363: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaatggagcgggtgcagcccctggaggagaacgtgggcaacgccgcccggccccggttcgagcggaacaagctgctgctggtggccagcgtgatccagggcctgggcctgctgctgtgcttcacctacatctgcctgcacttcagcgccctgcaggtgagccacaggtacccccggatccagagcatcaaggtgcagttcaccgagtacaagaaggagaagggcttcatcctgaccagccagaaggaggacgagatcatgaaggtgcagaacaacagcgtgatcatcaactgcgacggcttctacctgatctcactgaagggctacttcagccaggaggtgaacatcagcctgcactaccaaaaggacgaggagcccctgttccagctgaagaaggtgcggagcgtgaacagcctgatggtggcctcactgacctacaaggacaaggtgtaccttaacgtgaccaccgacaacaccagcctggacgacttccacgtgaacggcggcgagctgatcctgattcaccagaaccctggcgagttctgcgtgctgtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 90 - RNA, 1409 nt, synthetic construct
  source 1..1409: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggagggcgagggcgtgcagccactggatgagaacctggagaacggcagccggccccggttcaagtggaagaagaccttacgtctggtggtgagcggcatcaagggcgccggcatgctgctgtgcttcatctacgtgtgtctgcagctgagcagcagccccgccaaggacccccctatccagcggctgcggggcgccgtgacccggtgcgaggacggccagctgttcatcagcagctacaagaacgagtaccagaccatggaggtgcagaacaacagcgtggtgatcaagtgcgacggcctgtacatcatctacctgaagggcagcttcttccaggaagtgaagatcgacctacacttcagggaggatcacaaccccatcagcatccccatgctgaacgacggccggcggatcgtgttcaccgtggtggccagcctggccttcaaggacaaggtgtacctgaccgtgaacgcccccgacaccctgtgtgagcacctgcagatcaacgacggcgagctgatcgtggtgcagctgactcccggctactgcgcccccgagggcagctaccacagcaccgtgaaccaggtgcccctttgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 91 - RNA, 1379 nt, synthetic construct
  source 1..1379: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggccctgaccttcgccctgttagtggcgctgctggtgctgagctgcaagagcagctgcagcgtgggctgcgacctgcctcagacccacagcctgggcagccggcggaccctgatgctgctggcccagatgcggaagatcagcctgttcagctgtctgaaggaccggcacgacttcggcttcccccaggaggaattcggcaaccagttccagaaggccgagaccatccccgtgctgcacgagatgatccagcagatcttcaacctgttcagcaccaaggatagcagcgccgcttgggacgagacccttctggacaagttctacaccgagctgtaccagcagctgaacgacctggaggcctgcgtgatccagggcgtgggcgtgaccgagacccctctgatgaaggaggacagcatcctggccgtgcggaagtactttcagcggatcaccctgtacctgaaggagaagaagtacagcccatgcgcctgggaggtggtgcgggccgagatcatgcggagcttcagcctgagcaccaacctgcaggagagcctgcggagcaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 92 - RNA, 1373 nt, synthetic construct
  source 1..1373: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 93 - RNA, 1265 nt, synthetic construct
  source 1..1265: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtaccggatgcagctgctgagctgcatcgccctgagcctggccctggtgaccaacagcgcccccaccagcagcagcaccaagaagacccagctgcaactggagcacctgctgctggatctgcagatgatcctgaacggcatcaacaattacaagaatcccaagctgacccggatgctgaccttcaagttctacatgcccaagaaggctaccgagctgaagcacctgcagtgcctggaggaggagctgaagcccctggaggaggtgctgaacctggcccagagcaagaacttccacttcgacccccgggacgtggtgagcaacatcaacgtgttcgtgctggagctgaagggcagcgagaccaccttcatgtgcgagtacgccgacgagaccgccaccatcgtggagttcctgaaccggtggatcaccttctgccagagcatcatcagctgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - RNA, 1322 nt, synthetic construct
  source 1..1322: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtacagcatgcagctggcaagctgcgtgaccctgaccctggtgctgctagtgaacagcgcccccaccagcagcagcaccagcagctccaccgccgaggcccagcagcaacaacagcaacagcagcagcagcaacagcacctggagcagctcctgatggacctgcaggagctgctgagccggatggagaactaccggaacctgaagctgccccggatgctgaccttcaagttctacctgcccaagcaggccacagagctgaaggacctgcagtgcctggaggacgagctgggccccctgcggcacgtgctggatctgacccagagcaagagcttccagctggaggacgccgagaacttcatcagcaacatccgggtgaccgtggtgaagctgaagggcagcgacaacaccttcgagtgccagttcgacgacgagagcgcaaccgtggtggacttcctgcggcggtggatcgccttctgccagagcatcatcagcaccagcccccagtgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 95 - RNA, 1535 nt, synthetic construct
  source 1..1535: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggccccccggcgggcccggggctgccggaccctgggcctgcctgccctgctgctgctgcttctgctgcggccccccgccacccggggcatcacctgtcccccacctatgagcgtggagcacgccgacatctgggtgaagagctacagcctgtacagccgggagcggtacatctgcaacagcggcttcaagcggaaggcaggcaccagcagcctgaccgagtgcgtgctgaacaaggccaccaacgtggcccactggaccacccccagccttaagtgcatccgggaccctgcgctggtccaccagcggcctgccccccccagcaccgtgaccaccgcaggagtgaccccccagcccgagagcctgagccccagcggcaaggagcccgccgccagctcacccagcagcaacaacaccgccgccaccaccgccgccatcgtgcccggcagccagctgatgcctagcaagagccccagcaccggcaccaccgagatcagcagccacgagagcagccacggcacccccagccagaccaccgccaagaattgggagctgaccgccagcgccagccaccagcccccaggcgtgtacccccaggggcacagcgacaccaccgtggccatcagcaccagcaccgtgctgctgtgcggcctgagcgcagtgagcctgctggcctgctacctgaagagccggcagaccccacccctggctagcgtggagatggaggccatggaggccctgcccgtgacctggggaaccagcagccgggacgaggacctggagaactgcagccaccacctgtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 96 - RNA, 1403 nt, synthetic construct
  source 1..1403: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggagttcggcctgagctgggtgttcctggtggccctgttccggggagtgcagtgtatcacctgccccccccccatgagcgtggagcacgctgacatctgggtgaagagctacagcctgtacagccgggagcggtacatctgcaacagcggcttcaagcggaaggccggcaccagctccctgaccgagtgcgtgctgaacaaggccaccaacgtggcccactggaccacccccagcctgaagtgcatccgggaccccgccctggtgggcggctcaggcggcagcggcggcagcgggggcagcggaggcagcggcggaagcggcggcaactgggtgaacgtgatcagcgacctgaagaagatcgaggacctgatccaaagcatgcacattgacgccaccctgtacaccgagagcgacgtgcaccccagctgcaaggtgaccgcaatgaagtgcttcctgctggagctgcaggtgatcagcctggagagcggcgacgccagcatccacgacaccgtggagaacctgatcatcctggcgaacaacagcctgagcagcaacggcaacgtgaccgagagcggctgtaaggagtgcgaggagctggaggagaagaatatcaaggagttcctgcagagcttcgtgcacatcgtgcagatgttcatcaacaccagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 97 - RNA, 1403 nt, synthetic construct
  source 1..1403: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggagttcggcctgagctgggtgtttctggtggccctgttccggggcgtgcagtgcaactgggtgaacgtgatcagcgacctgaagaagatcgaggacctgatccagtcaatgcacatcgatgcaaccctgtacaccgagagcgacgtgcaccctagctgtaaggtgaccgcaatgaagtgcttcctgctggagctgcaggtgataagcctggagagcggcgacgccagcatccacgacacagtcgagaatctgatcatcctggctaacaacagcctgagcagcaatggtaatgtgaccgagagcggctgcaaggagtgcgaggagctggaggagaagaacatcaaggagttcctgcagagcttcgtgcacatcgtgcagatgttcatcaacaccagcggcggcagcgggggcagcggcggctcaggtggcagcggaggcagcggcggaagcggcggcatcacctgccctccccccatgagcgtggagcacgcagacatctgggtgaagagctacagcctgtacagccgggagcggtacatctgcaacagcggcttcaagcggaaggccggcaccagcagcctgaccgagtgtgtgctgaacaaggccaccaacgtggcccactggacaacccccagcctgaagtgcatcagggaccccgccctggtgtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 98 - RNA, 2069 nt, synthetic construct
  source 1..2069: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggctagcccccagctgcggggctacggcgtgcaggccatccctgtgctgctgttactgctgctgctcctgctgctgcccctgcgggtgacccccggcaccacctgccccccccccgtgagcatcgagcacgccgacatccgggtgaagaactacagcgtgaacagccgggagaggtacgtgtgcaatagcggcttcaagcggaaggccggcaccagcaccctgatcgagtgcgtgatcaacaagaacaccaacgtggcccactggaccacccccagcctgaagtgcatccgggaccccagcttagcccactacagccccgtgcccaccgtggtgaccccaaaggtgaccagccagcccgagagccccagccccagcgccaaggagcctgaggccttctccccaaagagcgacaccgccatgaccaccgagaccgccatcatgcccggcagccggctgacccccagccagaccaccagcgccggcaccacaggcaccggcagccacaagagcagccgggcacccagcctggccgccaccatgaccctggagcctaccgctagcaccagcctgcggatcaccgagatcagcccccacagcagcaagatgaccaaggtggccatcagcaccagcgtgctgctggtgggcgccggcgtggtgatggctttcctggcctggtacatcaagagccggcagcccagccagccttgccgggtggaggtggagaccatggagactgtgcccatgaccgtgcgggccagcagcaaggaggacgaggacaccggcgccgggggcagcggcggtagcggaggcagcggcggcagcggcggaagcggcggctccggcggcatgaagatcctgaagccctacatgcggaacaccagcatcagctgctacctgtgcttcctgctgaacagccacttcctgaccgaggccggcatccacgtgttcatcctgggctgcgtgagcgtgggcctgcctaagaccgaggccaactggatcgacgtgcggtacgacctggagaagatcgagagcctgatccagagcatccacatcgacaccaccctgtacaccgacagcgacttccaccccagctgcaaggtgaccgccatgaactgctttctgctggagctgcaggtgatcctgcacgagtacagcaacatgaccctgaacgagaccgtgcggaacgtgctgtacctggccaacagcaccctgagcagcaacaagaacgtggccgagagcggctgcaaggagtgcgaggagctggaggagaagaccttcaccgagttcctgcagagcttcatccggatcgtgcagatgttcatcaacaccagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 99 - RNA, 1406 nt, synthetic construct
  source 1..1406: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgagcgtgctgacccaggtgctggccctgctgctgctgtggctgaccggcgcccgttgcaccacctgcccccccccagtgagcatcgagcacgccgatatccgggtgaagaactacagcgtgaacagccgggagcggtacgtgtgcaacagcggcttcaagcggaaggccggcacaagcaccctgatcgagtgcgtgatcaacaagaacaccaatgtggcccactggaccacccccagcctgaagtgtatccgggaccctagcctggccggcggcagcggcgggagcggtggcagcggcggaagcggcggtagcggaggcagcggcggcaactggatcgacgtgcggtacgacctagagaagatcgagagcctgatccagagcatccacatcgacaccaccctgtacaccgacagcgacttccaccccagctgcaaggtgaccgccatgaactgcttcctgctggagctgcaggtgatcctgcacgagtacagcaacatgacactgaacgagaccgtgcggaacgtgctgtacctggcaaacagcaccctgagcagcaacaagaatgtggccgagagcggctgcaaggaatgcgaggagctggaggagaagaccttcaccgagttcctccagagcttcatccggatcgtgcagatgttcatcaacaccagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 100 - RNA, 1406 nt, synthetic construct
  source 1..1406: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgagcgtgctgacccaggtgctggctctactgctgctgtggttaacaggcgcccggtgcaactggatcgacgtgcggtacgacctggagaagatcgagagcctgatccagagcatccacatcgacaccaccctgtacaccgacagcgacttccaccctagctgcaaggtgaccgccatgaactgcttcctgctggagctgcaggtgatcctgcacgagtacagcaatatgaccctgaacgagaccgtgcggaacgtgctgtacctggccaacagcaccctgagcagcaataagaatgtggccgagagcggctgcaaggagtgtgaggagctggaggagaagaccttcactgagttcctgcagagctttatccggatcgtgcagatgttcatcaacactagcggcggaagcggcggctcaggcggcagcggaggcagcggcggcagcgggggcagcggcggaaccacctgtcccccccccgtgagcatcgagcacgccgacatccgggtgaagaactacagcgtgaactcacgggagcggtacgtgtgcaactcaggcttcaagcggaaggccggcaccagcaccctgatcgagtgcgtgatcaacaagaacacaaacgtggcccactggaccacccccagcctgaagtgcatccgggaccccagcctggcctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - RNA, 1976 nt, synthetic construct
  source 1..1976: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgagcgtgctgacccaggtgctggccctgctgctgctgtggctgaccggcgcccggtgcaactggatcgacgtgcggtacgatctggagaagatcgagagcctgatccagagcatccacatcgacaccaccctgtacaccgatagcgacttccaccccagctgcaaggtgaccgccatgaactgcttcctgctggaactgcaggtgatcctgcacgagtacagcaacatgaccctgaacgagaccgtgcggaacgtcctgtacctggccaacagcacactgagcagcaacaagaacgtggccgagagcggctgcaaggagtgcgaggagctggaggagaagaccttcaccgagttcctgcagagcttcatccgtatcgtgcagatgttcatcaacaccagcgggggcagcggcggaagcgggggcagcggcggaagcggcggcagcggaggcagcggaggcaccacatgccccccacccgtgagcatcgagcacgccgacatacgggtgaagaattacagcgtgaacagccgggagcggtacgtgtgcaacagcggcttcaagcggaaggccggcaccagcaccctgatcgagtgcgtgatcaacaagaacactaacgtggcacactggaccacccccagcctcaagtgcatccgggacccaagcctggccgggggcggcggctccggcggaggcggcagcggcggtggaggcagcggccctacaatcaagccctgccccccctgcaagtgccccgcccccaatgccgccggcggccccagcgtgttcatcttcccccccaagatcaaggacgtgctgatgatctcactaagccccatagtgacctgcgtggtggtggacgtgagcgaggacgaccctgacgtgcagatcagctggttcgtgaacaacgtggaggtgcacaccgcccagacccagacccaccgggaggactacaacagcaccctgcgggtggtgagcgccctgcccatccagcaccaggactggatgagcggcaaggagttcaagtgtaaggtgaataataaggacctgggcgccccaatagagcggaccattagcaagcccaaggggagcgtgcgggccccccaggtgtacgtgctgccaccccctgaggaggagatgaccaaaaagcaggtgaccctgacctgcatggtgaccgatttcatgccagaggacatctacgtggagtggaccaacaacggcaagaccgagctgaactacaagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 102 - RNA, 2510 nt, synthetic construct
  source 1..2510: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtgccaccagcagctggtgatcagctggttcagcctggtgttcctcgccagccccctggtggctatctgggagctgaagaaggacgtgtacgtggtggagctggactggtaccccgacgcccccggcgagatggtggtgctcacctgcgatacccccgaggaggacggaatcacctggaccctggaccagagcagcgaggtgttaggcagcggcaagaccctgaccatccaggtgaaggagttcggcgacgccggccagtacacctgccacaagggcggggaggtgctgagccacagcctgctgctgctgcacaagaaggaggacggcatctggagcaccgacatcctgaaggaccagaaggagcccaagaacaagaccttcctgcggtgcgaggccaagaattacagcggccggttcacctgctggtggctgaccaccatcagcaccgacctgaccttcagcgtgaagagcagccggggcagcagcgacccccagggcgtgacctgcggcgccgccaccctgagcgccgagcgggtgaggggcgacaataaggagtacgagtacagcgtggagtgtcaggaggatagcgcatgccccgccgccgaggagagcctgcccatcgaggtgatggtggacgccgtgcacaagctgaagtacgagaactacaccagcagcttcttcatccgggacatcatcaagcccgacccccccaagaacctgcagctgaagcccctgaagaacagccggcaggtggaggtgtcatgggagtaccccgacacctggagcaccccccacagctacttcagcctgacattctgtgtgcaggtgcagggcaagagcaagcgggagaagaaggaccgggtgttcaccgacaagaccagcgccaccgtgatctgccggaagaatgccagcatcagcgtgcgggcccaggaccggtactacagcagcagctggagcgagtgggccagcgtgccttgctccggcggcggaggaagcggaggcgggggcagcgggggcggcggaagccggaacctgcccgtggccaccccagaccccggcatgttcccctgcctgcaccacagccagaacctgctgcgggccgtgagcaacatgctgcagaaggctcggcagaccctggagttctacccctgcaccagcgaggagatcgaccacgaggacatcaccaaggacaagactagcaccgtggaggcctgcctgcccctggagctgaccaagaatgagagctgcctgaacagccgggagaccagctttatcaccaacggcagctgcctggccagccggaagacctcattcatgatggccctgtgcctgagcagcatctacgaggacctgaagatgtaccaggtggagttcaagaccatgaacgccaagctgctgatggaccctaagcggcaaatcttcctggaccagaatatgctggccgtgatcgacgagctcatgcaggccctgaactttaacagcgagaccgtgccccagaagagcagcctggaggagccagacttctacaagaccaagatcaagctgtgcatcctgctgcacgctttccggatccgggccgtgaccatcgaccgggtgatgagctacctgaacgcgagcagcggaggcggcagcggtggcggcggctcaggcggcggaggcagcggcggaggcggaagcggcggcggcagcctgcaggtggtgatcagcgccatcctggctctggtggtgctgaccatcatctccctgatcatcctgatcggcggaggtggcagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 103 - RNA, 2561 nt, synthetic construct
  source 1..2561: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtgcccccagaagctgaccatcagctggttcgccatcgtgctgctggtgagccctctgatggccatgtgggagctggagaaggacgtgtacgtggtggaggtggattggacccccgacgcacccggcgagaccgtgaacctgacctgcgatacccctgaggaggacgacatcacctggactagcgatcagcggcacggcgtgatcggcagcggcaagaccctgaccatcaccgtgaaggagttcctggatgccggccagtacacctgccacaagggcggggagaccctgagccacagccacctgctgctgcacaaaaaggagaacggcatctggagcaccgagatcctgaagaacttcaagaacaagacctttctgaagtgcgaggctcccaactacagcggtcggttcacctgcagctggctggtgcagcggaatatggacctgaagtttaacatcaagtcaagcagcagcagccccgacagccgggccgtgacctgtggcatggccagcctgagcgccgagaaggtgaccctggaccagagggattacgagaagtacagcgtgagctgccaggaggacgtgacctgcccaaccgccgaggagaccctgcccatcgagctggccctggaggcccggcagcagaacaagtacgagaactacagcaccagcttcttcatccgggacatcatcaagcctgacccccccaagaacctgcagatgaagcccctgaagaacagccaggtggaggtgagctgggagtaccccgacagctggagcaccccccacagctacttcagcctgaagttcttcgtgcggatccagcggaagaaggagaagatgaaggagaccgaggagggctgcaaccagaagggcgccttcctggtggagaagaccagcaccgaggtgcagtgtaagggcggcaacgtgtgcgtgcaggcccaggaccggtactacaacagctcatgcagcaagtgggcctgcgtgccctgccgggtgaggagcggcggcggcggctccggcggaggaggcagcggaggaggcggcagccgggtgatccccgtgagcggccctgcccggtgcctgagccagagccggaacctgctgaagaccaccgacgacatggtgaagaccgctcgggagaagctgaagcactacagctgcaccgccgaggacatcgaccacgaggacatcacccgggaccagaccagcaccttaaagacctgcctgcccctggagctgcacaagaatgagagctgcctggccacccgggagaccagcagcaccacccggggcagctgcctgcccccccagaagaccagcctgatgatgaccctgtgcctgggcagcatctacgaggacctgaagatgtaccagaccgagttccaggcaatcaacgctgctctgcagaatcacaaccaccagcagatcatcctggacaagggaatgctggtggccatcgacgagctgatgcagagcctgaaccacaacggcgagaccctgcggcagaagccccccgtgggcgaggccgacccctaccgtgtgaagatgaagctgtgcatcctgctgcacgctttcagcacccgggtggtgaccatcaaccgggtgatgggctacctgagcagcgcaagcggtggcggcagcggcggcgggggctccggcggcggtgggagcggcggaggcggttcaggcggcggcagcctgcaggtggtgatcagcgccatcctggccctggtggtgctcaccgtgatcagcctgatcatcctgatcgggggcggcggctcaggcaagcccatcccaaatccactgctgggcctggacagcacctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 104 - RNA, 2561 nt, synthetic construct
  source 1..2561: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtgcccccagaagctgaccatcagctggttcgccatcgtgctgctggtgagccctctgatggccatgtgggagctcgagaaggacgtgtacgtggtggaggtggactggacccccgacgcccccggcgagaccgtgaacctgacctgtgacacccccgaggaggacgacatcacctggaccagcgaccagcggcacggcgtgatcggaagcggcaagaccctgaccatcaccgtcaaggagttcctggacgccggccagtacacctgccacaagggcggcgagacactgagccacagccacctgctgttacacaagaaggagaacggcatctggagcaccgagattctgaagaacttcaagaacaagaccttcctgaagtgcgaagcccccaattacagcggccggttcacctgcagctggctggtgcagcggaacatggacctgaagttcaatatcaagtcaagcagcagcagccccgatagccgggccgtgacctgcggcatggccagcctgagcgccgagaaggtgaccctggaccagagggactacgagaagtacagcgtgagctgtcaggaggacgtgacatgcccaaccgccgaggagaccctgcccatcgagctggccctggaggcccggcagcagaataagtacgagaattacagcaccagcttcttcatccgggacatcatcaagcccgacccccctaagaacctgcagatgaagcccctgaagaatagccaggtcgaggtgagctgggagtaccccgatagctggagcaccccccacagctacttcagcctgaagttcttcgtgcggatccagcggaagaaggagaagatgaaggagaccgaggagggctgcaatcagaagggcgcattcctggtggagaagaccagcaccgaggtgcagtgcaagggcggcaacgtgtgcgtgcaggcccaggatcggtactacaacagcagctgcagcaagtgggcctgcgtgccatgccgggtgcggagcggcggcggaggctcaggtggcggcggctcaggcgggggcggcagccgggtgatccccgtgagcggccccgcccggtgccttagccagagccggaatctgctgaagaccaccgacgacatggtgaagaccgcccgggagaagctgaagcactacagctgcaccgccgaggacatcgaccacgaggacatcacccgggaccagaccagcaccctgaagacctgcctgcccctggagctgcacaagaacgagagctgcctggcaacccgggagaccagcagcaccacccggggcagctgcctccccccccagaagaccagcctgatgatgaccctgtgcctggggagcatctacgaggacctgaagatgtaccagaccgagttccaggctataaacgccgccctgcagaaccacaaccaccagcagatcatcctggacaagggcatgctggtggccatcgacgagctgatgcagagcctgaaccacaacggcgagactctgcggcagaagccccctgtgggcgaggccgatccctaccgggtgaagatgaagctgtgcatcctgctgcacgccttcagcacccgggtggtgaccatcaacagggtgatgggctacctgagcagcgctagcggcggaggtagcggaggcggcggctccggcggcggcggcagcggtggaggcggcagcggcggcggtagcctgcaagtggtgatcagcgccatcctggccctggtggtgctgaccgtgatcagcctgatcatcctgatcggcggtggcgggagcggcaagcccatccccaacccactgctgggcctggacagcacctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 105 - RNA, 1282 nt, synthetic construct
  source 1..1282: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaatggagcgggtgcagcccctggaggagaacgtgggcaacgccgcccggccccggttcgagcggaacaagctgctgctggtggccagcgtgatccagggcctgggcctgctgctgtgcttcacctacatctgcctgcacttcagcgccctgcaggtgagccacaggtacccccggatccagagcatcaaggtgcagttcaccgagtacaagaaggagaagggcttcatcctgaccagccagaaggaggacgagatcatgaaggtgcagaacaacagcgtgatcatcaactgcgacggcttctacctgatctcactgaagggctacttcagccaggaggtgaacatcagcctgcactaccaaaaggacgaggagcccctgttccagctgaagaaggtgcggagcgtgaacagcctgatggtggcctcactgacctacaaggacaaggtgtaccttaacgtgaccaccgacaacaccagcctggacgacttccacgtgaacggcggcgagctgatcctgattcaccagaaccctggcgagttctgcgtgctgtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 106 - RNA, 1328 nt, synthetic construct
  source 1..1328: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggagggcgagggcgtgcagccactggatgagaacctggagaacggcagccggccccggttcaagtggaagaagaccttacgtctggtggtgagcggcatcaagggcgccggcatgctgctgtgcttcatctacgtgtgtctgcagctgagcagcagccccgccaaggacccccctatccagcggctgcggggcgccgtgacccggtgcgaggacggccagctgttcatcagcagctacaagaacgagtaccagaccatggaggtgcagaacaacagcgtggtgatcaagtgcgacggcctgtacatcatctacctgaagggcagcttcttccaggaagtgaagatcgacctacacttcagggaggatcacaaccccatcagcatccccatgctgaacgacggccggcggatcgtgttcaccgtggtggccagcctggccttcaaggacaaggtgtacctgaccgtgaacgcccccgacaccctgtgtgagcacctgcagatcaacgacggcgagctgatcgtggtgcagctgactcccggctactgcgcccccgagggcagctaccacagcaccgtgaaccaggtgcccctttgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 107 - RNA, 1298 nt, synthetic construct
  source 1..1298: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggccctgaccttcgccctgttagtggcgctgctggtgctgagctgcaagagcagctgcagcgtgggctgcgacctgcctcagacccacagcctgggcagccggcggaccctgatgctgctggcccagatgcggaagatcagcctgttcagctgtctgaaggaccggcacgacttcggcttcccccaggaggaattcggcaaccagttccagaaggccgagaccatccccgtgctgcacgagatgatccagcagatcttcaacctgttcagcaccaaggatagcagcgccgcttgggacgagacccttctggacaagttctacaccgagctgtaccagcagctgaacgacctggaggcctgcgtgatccagggcgtgggcgtgaccgagacccctctgatgaaggaggacagcatcctggccgtgcggaagtactttcagcggatcaccctgtacctgaaggagaagaagtacagcccatgcgcctgggaggtggtgcgggccgagatcatgcggagcttcagcctgagcaccaacctgcaggagagcctgcggagcaaggagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 108 - RNA, 1292 nt, synthetic construct
  source 1..1292: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatggctcggctgtgcgccttcctgatgatcctggtgatgatgagctactactggagcgcatgcagcctgggctgcgacctgccacacacctacaacctgggcaataagcgggctctgaccgtgctggaggagatgcggcggctgccccccctgagctgcctgaaggaccggaaggacttcggcttccccctggagaaggtggacaaccagcagatccaaaaggcacaggccatcctggtgctgcgggacctgacccagcagatcctgaacctgttcaccagcaaggacctgagcgccacctggaacgccaccctgctggacagcttctgcaacgacctgcaccagcagctgaacgacctgaaggcctgcgtcatgcaggagccaccactgacccaggaggacagcctgttagctgtgcggacttacttccaccggatcaccgtgtacctgcggaagaagaagcacagcctgtgcgcttgggaggtgatccgggccgaggtgtggcgggccctgagcagcagcaccaacctgctggcccggctgagcgaggagaaggagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 109 - RNA, 1184 nt, synthetic construct
  source 1..1184: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtaccggatgcagctgctgagctgcatcgccctgagcctggccctggtgaccaacagcgcccccaccagcagcagcaccaagaagacccagctgcaactggagcacctgctgctggatctgcagatgatcctgaacggcatcaacaattacaagaatcccaagctgacccggatgctgaccttcaagttctacatgcccaagaaggctaccgagctgaagcacctgcagtgcctggaggaggagctgaagcccctggaggaggtgctgaacctggcccagagcaagaacttccacttcgacccccgggacgtggtgagcaacatcaacgtgttcgtgctggagctgaagggcagcgagaccaccttcatgtgcgagtacgccgacgagaccgccaccatcgtggagttcctgaaccggtggatcaccttctgccagagcatcatcagctgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 110 - RNA, 1241 nt, synthetic construct
  source 1..1241: mol_type = other RNA
aggtcttctggtccccacagactcagagagaacccgccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaatgtacagcatgcagctggcaagctgcgtgaccctgaccctggtgctgctagtgaacagcgcccccaccagcagcagcaccagcagctccaccgccgaggcccagcagcaacaacagcaacagcagcagcagcaacagcacctggagcagctcctgatggacctgcaggagctgctgagccggatggagaactaccggaacctgaagctgccccggatgctgaccttcaagttctacctgcccaagcaggccacagagctgaaggacctgcagtgcctggaggacgagctgggccccctgcggcacgtgctggatctgacccagagcaagagcttccagctggaggacgccgagaacttcatcagcaacatccgggtgaccgtggtgaagctgaagggcagcgacaacaccttcgagtgccagttcgacgacgagagcgcaaccgtggtggacttcctgcggcggtggatcgccttctgccagagcatcatcagcaccagcccccagtgattaaaccggtaccggttgataatagcagacaccattgtcacactccagctggagcctcggtggccatgcttcttgccccttgggcccagacaccattgtcacactccatccccccagcccctcctccccttcctgcacccgtaccccccagacaccattgtcacactccagtggtctttgaataaagtctgagtgggcggcgaattcgatatccagcggccgcgctagcgctggctagcgaattcgatatccagcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa